SEQUENCE LISTING (81 sequences)

SEQ ID NO: 1 - protein, 8 aa, synthetic construct
  REGION 1..8: note = Consensus CDR -H1
  VARIANT 3: note = Thr may be replaced by Ser
  VARIANT 8: note = Tyr may be replaced by Phe
GYTFTSYY

SEQ ID NO: 2 - protein, 8 aa, synthetic construct
  REGION 1..8: note = Consensus CDR-H2
IWPGDGST

SEQ ID NO: 3 - protein, 13 aa, synthetic construct
  REGION 1..13: note = Consensus CDR-H3
ASPMITPNYAMDY

SEQ ID NO: 4 - protein, 6 aa, synthetic construct
  REGION 1..6: note = Consensus CDR-L1
  VARIANT 4: note = Ser may be replaced by Asn
QDISKY

SEQ ID NO: 5 - skipped

SEQ ID NO: 6 - protein, 9 aa, synthetic construct
  REGION 1..9: note = Consensus CDR-L3
  VARIANT 5: note = Thr may be replaced by Ala
QQGSTLPYT

SEQ ID NO: 7 - protein, 8 aa, synthetic construct
  REGION 1..8: note = CDR-H1
GYTFTSYY

SEQ ID NO: 8 - protein, 8 aa, synthetic construct
  REGION 1..8: note = CDR-H1
GYSFTSYF

SEQ ID NO: 9 - protein, 6 aa, synthetic construct
  REGION 1..6: note = CDR-L1
QDISKY

SEQ ID NO: 10 - protein, 6 aa, synthetic construct
  REGION 1..6: note = CDR-L1
QDINKY

SEQ ID NO: 11 - protein, 9 aa, synthetic construct
  REGION 1..9: note = CDR-L3
QQGSTLPYT

SEQ ID NO: 12 - protein, 9 aa, synthetic construct
  REGION 1..9: note = CDR-L3
QQGSALPYT

SEQ ID NO: 13 - protein, 120 aa, synthetic construct
  REGION 1..120: note = c208F2, heavy chain, VH
QVQLQQSGPELVKPGASVKMSCKASGYTFTSYYIYWVKQRPGQGLEWLGWIWPGDGSTKYNEKFKDKTTLTADKSSNTAYMFLSSLTSEDSAVYFCASPMITPNYAMDYWGQGASVTVSS

SEQ ID NO: 14 - protein, 120 aa, synthetic construct
  REGION 1..120: note = c212A11, heavy chain, VH
QVQLQQSGPELVKPGASVKMSCKASGYTFTSYYIHWVKQRPGQGLEWIGWIWPGDGSTKYNEKFKGKTTLTADKSSSTAYMFLSSLTSEDSAVYFCASPMITPNYAMDYWGQGASVTVSS

SEQ ID NO: 15 - protein, 120 aa, synthetic construct
  REGION 1..120: note = c214F8, heavy chain, VH
QVQLQQSGSELVKPGASVKLSCKASGYTFTSYYIHWVKQRPGQGLEWIGWIWPGDGSTKYNERFKGKTTLTADKSSSTAYMFLSSLTSEDSAVYFCASPMITPNYAMDYWGQGASVTVSS

SEQ ID NO: 16 - protein, 120 aa, synthetic construct
  REGION 1..120: note = c219D6, heavy chain, VH
QVQLQQSGPELVKPGDSVKMSCKASGYSFTSYFIHWVKQRPGQGLEWIGWIWPGDGSTKYNEKFKGKTTLTTDKSSSTAYMFLNSLTSEDSAVYFCASPMITPNYAMDYWGQGASVTVSS

SEQ ID NO: 17 - protein, 120 aa, synthetic construct
  REGION 1..120: note = c213B10, heavy chain, VH
QVQLQQSGSELVKPGASVKMSCKASGYTFTSYYIHWVKQRPGQGLEWIGWIWPGDGSTKYNERFKGKTTLTADKSSSTAYMFLSSLTSEDSAVYFCASPMITPNYAMDYWGQGASVTVSS

SEQ ID NO: 18 - protein, 107 aa, synthetic construct
  REGION 1..107: note = c208F2, light chain, VL
DIQMTQTTSSLSASLGDRVTISCRASQDISKYLNWYQQQPDGTIKLLIYYTSRLHSGVPSRFSGRGSGTDYSLTISNVEQEDIATYFCQQGSTLPYTFGGGTKLEIK

SEQ ID NO: 19 - protein, 107 aa, synthetic construct
  REGION 1..107: note = c212A11, light chain, VL
DIQMTQTTSSLSASLGDRVTISCRASQDINKYLNWYQQQPDGTVKLLIYYTSRLHSGVPSRFSGRGSGTDYSLTISNLEQEDIATYFCQQGSTLPYTFGGGTKLEIK

SEQ ID NO: 20 - protein, 107 aa, synthetic construct
  REGION 1..107: note = c214F8, light chain, VL
DIQMTQTTSSLSASLGDRVTFSCRASQDISKYLNWYQQQPDGTIKLLIYYTSRLHSGVPSRFSGRGSGTDYSLTITNLEQEDIATYFCQQGSALPYTFGGGTKLEIK

SEQ ID NO: 21 - protein, 107 aa, synthetic construct
  REGION 1..107: note = c219D6, light chain, VL
DIQMTQTTSSLSASLGDRVTISCRASQDISKYLNWYQQQPDGTVKLLIYYTSRLHSGVPSRFSGRGSGTDYSLTISNLEQEDIATYFCQQGSTLPYTFGGGTKLEIK

SEQ ID NO: 22 - protein, 107 aa, synthetic construct
  REGION 1..107: note = c213B10, light chain, VL
DIQMTQTTSSLSASLGDRVTISCRASQDISKYLNWYQQQPDGTIKLLIYYTSRLHSGVPSRFSGRGSGTDYSLTITNLEQEDIATYFCQQGSALPYTFGGGTKLEIK

SEQ ID NO: 23 - protein, 449 aa, synthetic construct
  REGION 1..449: note = c208F2, heavy chain, full length
QVQLQQSGPELVKPGASVKMSCKASGYTFTSYYIYWVKQRPGQGLEWLGWIWPGDGSTKYNEKFKDKTTLTADKSSNTAYMFLSSLTSEDSAVYFCASPMITPNYAMDYWGQGASVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 24 - protein, 449 aa, synthetic construct
  REGION 1..449: note = c212A11, heavy chain, full length
QVQLQQSGPELVKPGASVKMSCKASGYTFTSYYIHWVKQRPGQGLEWIGWIWPGDGSTKYNEKFKGKTTLTADKSSSTAYMFLSSLTSEDSAVYFCASPMITPNYAMDYWGQGASVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 25 - protein, 449 aa, synthetic construct
  REGION 1..449: note = c214F8, heavy chain, full length
QVQLQQSGSELVKPGASVKLSCKASGYTFTSYYIHWVKQRPGQGLEWIGWIWPGDGSTKYNERFKGKTTLTADKSSSTAYMFLSSLTSEDSAVYFCASPMITPNYAMDYWGQGASVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 26 - protein, 449 aa, synthetic construct
  REGION 1..449: note = c219D6, heavy chain, full length
QVQLQQSGPELVKPGDSVKMSCKASGYSFTSYFIHWVKQRPGQGLEWIGWIWPGDGSTKYNEKFKGKTTLTTDKSSSTAYMFLNSLTSEDSAVYFCASPMITPNYAMDYWGQGASVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 27 - protein, 449 aa, synthetic construct
  REGION 1..449: note = c213B10, heavy chain, full length
QVQLQQSGSELVKPGASVKMSCKASGYTFTSYYIHWVKQRPGQGLEWIGWIWPGDGSTKYNERFKGKTTLTADKSSSTAYMFLSSLTSEDSAVYFCASPMITPNYAMDYWGQGASVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 28 - protein, 214 aa, synthetic construct
  REGION 1..214: note = c208F2, light chain, full length
DIQMTQTTSSLSASLGDRVTISCRASQDISKYLNWYQQQPDGTIKLLIYYTSRLHSGVPSRFSGRGSGTDYSLTISNVEQEDIATYFCQQGSTLPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 214 aa, synthetic construct
  REGION 1..214: note = c212A11, light chain, full length
DIQMTQTTSSLSASLGDRVTISCRASQDINKYLNWYQQQPDGTVKLLIYYTSRLHSGVPSRFSGRGSGTDYSLTISNLEQEDIATYFCQQGSTLPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 214 aa, synthetic construct
  REGION 1..214: note = c214F8, light chain, full length
DIQMTQTTSSLSASLGDRVTFSCRASQDISKYLNWYQQQPDGTIKLLIYYTSRLHSGVPSRFSGRGSGTDYSLTITNLEQEDIATYFCQQGSALPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 214 aa, synthetic construct
  REGION 1..214: note = c219D6, light chain, full length
DIQMTQTTSSLSASLGDRVTISCRASQDISKYLNWYQQQPDGTVKLLIYYTSRLHSGVPSRFSGRGSGTDYSLTISNLEQEDIATYFCQQGSTLPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 214 aa, synthetic construct
  REGION 1..214: note = c213B10, light chain, full length
DIQMTQTTSSLSASLGDRVTISCRASQDISKYLNWYQQQPDGTIKLLIYYTSRLHSGVPSRFSGRGSGTDYSLTITNLEQEDIATYFCQQGSALPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 120 aa, synthetic construct
  REGION 1..120: note = hz208F2 (var.1) heavy chain, VH
QVQLVQSGAEVKKPGASVKVSCKASGYTFTSYYMHWVRQAPGQGLEWMGIIWPGDGSTSYAQKFQGRVTMTRDTSTSTVYMELSSLRSEDTAVYYCASPMITPNYAMDYWGQGTLVTVSS

SEQ ID NO: 34 - protein, 120 aa, synthetic construct
  REGION 1..120: note = hz208F2 (var. 3), VH
QVQLVQSGAEVKKPGASVKMSCKASGYTFTSYYIYWVKQAPGQGLEWLGWIWPGDGSTKYNEKFQGRVTLTADKSSNTAYMFLSSLRSEDTAVYFCASPMITPNYAMDYWGQGTLVTVSS

SEQ ID NO: 35 - protein, 107 aa, synthetic construct
  REGION 1..107: note = hz208F2 (var. 1), VL
DIQMTQSPSSLSASVGDRVTITCRASQDISKYLNWYQQKPGKAPKLLIYYTSSLQSGVPSRFSGSGSGTDFTLTISSLQPEDFATYYCQQGSTLPYTFGGGTKVEIK

SEQ ID NO: 36 - protein, 107 aa, synthetic construct
  REGION 1..107: note = hz208F2 (var.3), VL
DIQMTQSPSSLSASVGDRVTISCRASQDISKYLNWYQQKPGKAPKLLIYYTSRLHSGVPSRFSGRGSGTDYSLTISNLQPEDFATYFCQQGSTLPYTFGGGTKVEIK

SEQ ID NO: 37 - protein, 449 aa, synthetic construct
  REGION 1..449: note = hz208F2 (var. 1), heavy chain, full length
QVQLVQSGAEVKKPGASVKVSCKASGYTFTSYYMHWVRQAPGQGLEWMGIIWPGDGSTSYAQKFQGRVTMTRDTSTSTVYMELSSLRSEDTAVYYCASPMITPNYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 38 - protein, 449 aa, synthetic construct
  REGION 1..449: note = hz208F2 (var.3), heavy chain full length
QVQLVQSGAEVKKPGASVKMSCKASGYTFTSYYIYWVKQAPGQGLEWLGWIWPGDGSTKYNEKFQGRVTLTADKSSNTAYMFLSSLRSEDTAVYFCASPMITPNYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 39 - protein, 214 aa, synthetic construct
  REGION 1..214: note = hz208F2 (var. 1), light chain, full length
DIQMTQSPSSLSASVGDRVTITCRASQDISKYLNWYQQKPGKAPKLLIYYTSSLQSGVPSRFSGSGSGTDFTLTISSLQPEDFATYYCQQGSTLPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 214 aa, synthetic construct
  REGION 1..214: note = hz208F2 (var.3), light chain, full length
DIQMTQSPSSLSASVGDRVTISCRASQDISKYLNWYQQKPGKAPKLLIYYTSRLHSGVPSRFSGRGSGTDYSLTISNLQPEDFATYFCQQGSTLPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 120 aa, synthetic construct; identical to SEQ ID NO: 34
  REGION 1..120: note = hz208F2 (var.2) heavy chain, VH
  VARIANT 20: note = Met may be replaced by Val
  VARIANT 34: note = Ile may be replaced by Met
  VARIANT 35: note = Tyr may be replaced by His
  VARIANT 38: note = Lys may be replaced by Arg
  VARIANT 48: note = Leu may be replaced by Met
  VARIANT 50: note = Trp may be replaced by Ile
  VARIANT 59: note = Lys may be replaced by Ser
  VARIANT 61: note = Asn may be replaced by Ala
  VARIANT 62: note = Glu may be replaced by Gln
  VARIANT 70: note = Leu may be replaced by Met
  VARIANT 72: note = Ala may be replaced by Arg
  VARIANT 74: note = Lys may be replaced by Thr
  VARIANT 76: note = Ser may be replaced by Thr
  VARIANT 77: note = Asn may be replaced by Ser
  VARIANT 79: note = Ala may be replaced by Val
  VARIANT 82: note = Phe may be replaced by Glu
  VARIANT 95: note = Phe may be replaced by Tyr

SEQ ID NO: 42 - protein, 107 aa, synthetic construct; identical to SEQ ID NO: 36
  REGION 1..107: note = hz208F2 (var. 2), light chain, VL
  VARIANT 22: note = Ser may be replaced by Thr
  VARIANT 53: note = Arg may be replaced by Ser
  VARIANT 55: note = His may be replaced by Gln
  VARIANT 65: note = Arg may be replaced by Ser
  VARIANT 71: note = Tyr may be replaced by Phe
  VARIANT 72: note = Ser may be replaced by Thr
  VARIANT 77: note = Asn may be replaced by Ser
  VARIANT 87: note = Phe may be replaced by Tyr

SEQ ID NO: 43 - protein, 329 aa, synthetic construct
  REGION 1..329: note = Constant domain (VH) IgG1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 44 - protein, 326 aa, synthetic construct
  REGION 1..326: note = Constant domain (VH) IgG4 (S228P)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 45 - protein, 107 aa, synthetic construct [sequence omitted]

SEQ ID NO: 46 - protein, 98 aa, synthetic construct
  REGION 1..98: note = Human germline IGHV1-46*01
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAR

SEQ ID NO: 47 - protein, 95 aa, synthetic construct
  REGION 1..95: note = Human germline IGKV1-39*01
DIQMTQSPSSLSASVGDRVTITCRASQSISSYLNWYQQKPGKAPKLLIYAASSLQSGVPSRFSGSGSGTDFTLTISSLQPEDFATYYCQQSYSTP

SEQ ID NO: 48 - protein, 15 aa, synthetic construct
  REGION 1..15: note = Human germline IGHJ4*01
YFDYWGQGTLVTVSS

SEQ ID NO: 49 - protein, 12 aa, synthetic construct
  REGION 1..12: note = Human germline IGKJ4*01
LTFGGGTKVEIK

SEQ ID NO: 50 - protein, 1367 aa, synthetic construct
  REGION 1..1367: note = IGF-1R (human)
MKSGSGGGSPTSLWGLLFLSAALSLWPTSGEICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSTCGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTSKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRTNASVPSIPLDVLSASNSSSQLIVKWNPPSLPNGNLSYYIVRWQRQPQDGYLYRHNYCSKDKIPIRKYADGTIDIEEVTENPKTEVCGGEKGPCCACPKTEAEKQAEKEEAEYRKVFENFLHNSIFVPRPERKRRDVMQVANTTMSSRSRNTTAADTYNITDPEELETEYPFFESRVDNKERTVISNLRPFTLYRIDIHSCNHEAEKLGCSASNFVFARTMPAEGADDIPGPVTWEPRPENSIFLKWPEPENPNGLILMYEIKYGSQVEDQRECVSRQEYRKYGGAKLNRLNPGNYTARIQATSLSGNGSWTDPVFFYVQAKTGYENFIHLIIALPVAVLLIVGGLVIMLYVFHRKRNNSRLGNGVLYASVNPEYFSAADVYVPDEWEVAREKITMSRELGQGSFGMVYEGVAKGVVKDEPETRVAIKTVNEAASMRERIEFLNEASVMKEFNCHHVVRLLGVVSQGQPTLVIMELMTRGDLKSYLRSLRPEMENNPVLAPPSLSKMIQMAGEIADGMAYLNANKFVHRDLAARNCMVAEDFTVKIGDFGMTRDIYETDYYRKGGKGLLPVRWMSPESLKDGVFTTYSDVWSFGVVLWEIATLAEQPYQGLSNEQVLRFVMEGGLLDKPDNCPDMLFELMRMCWQYNPKMRPSFLEIISSIKEEMEPGFREVSFYYSEENKLPEPEELDLEPENMESVPLDPSASSSSLPLPDRHSGHKAENGPGPGVLVLRASFDERQPYAHMNGGRKNERALPLPQSSTC

SEQ ID NO: 51 - protein, 932 aa, synthetic construct
  REGION 1..932: note = IGF-1R ECD (human)
MKSGSGGGSPTSLWGLLFLSAALSLWPTSGEICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSTCGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTSKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRTNASVPSIPLDVLSASNSSSQLIVKWNPPSLPNGNLSYYIVRWQRQPQDGYLYRHNYCSKDKIPIRKYADGTIDIEEVTENPKTEVCGGEKGPCCACPKTEAEKQAEKEEAEYRKVFENFLHNSIFVPRPERKRRDVMQVANTTMSSRSRNTTAADTYNITDPEELETEYPFFESRVDNKERTVISNLRPFTLYRIDIHSCNHEAEKLGCSASNFVFARTMPAEGADDIPGPVTWEPRPENSIFLKWPEPENPNGLILMYEIKYGSQVEDQRECVSRQEYRKYGGAKLNRLNPGNYTARIQATSLSGNGSWTDPVFFYVQAKTGYEN

SEQ ID NO: 52 - protein, 512 aa, synthetic construct
  REGION 1..512: note = IGF-1R ECD Nterminal (human)
MKSGSGGGSPTSLWGLLFLSAALSLWPTSGEICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSTCGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTSKNRIIITWHRY

SEQ ID NO: 53 - protein, 4 aa, synthetic construct
  REGION 1..4: note = tetrapeptide (linker)
GFLG

SEQ ID NO: 54 - protein, 4 aa, synthetic construct
  REGION 1..4: note = tetrapeptide (linker)
ALAL

SEQ ID NO: 55 - protein, 5 aa, synthetic construct
  REGION 1..5: note = tetrapeptide (linker)
PVGVV

SEQ ID NO: 56 - protein, 120 aa, synthetic construct
  REGION 1..120: note = hz208F2 heavy chain H037, VH
QVQLVQSGAEVKKPGASVKVSCKASGYTFTSYYMHWVRQAPGQGLEWMGWIWPGDGSTKYAQKFQGRVTMTRDKSSSTVYMELSSLRSEDTAVYFCASPMITPNYAMDYWGQGTLVTVSS

SEQ ID NO: 57 - protein, 107 aa, synthetic construct
  REGION 1..107: note = hz208F2 light chain L018, VL
DIQMTQSPSSLSASVGDRVTITCRASQDISKYLNWYQQKPGKAPKLLIYYTSRLQSGVPSRFSGRGSGTDYSLTISSLQPEDFATYFCQQGSTLPYTFGGGTKVEIK

SEQ ID NO: 58 - protein, 449 aa, synthetic construct
  REGION 1..449: note = hz208F2 heavy chain H037 full length
QVQLVQSGAEVKKPGASVKVSCKASGYTFTSYYMHWVRQAPGQGLEWMGWIWPGDGSTKYAQKFQGRVTMTRDKSSSTVYMELSSLRSEDTAVYFCASPMITPNYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 59 - protein, 214 aa, synthetic construct
  REGION 1..214: note = hz208F2 light chain L018 full length
DIQMTQSPSSLSASVGDRVTITCRASQDISKYLNWYQQKPGKAPKLLIYYTSRLQSGVPSRFSGRGSGTDYSLTISSLQPEDFATYFCQQGSTLPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 107 aa, synthetic construct
  REGION 1..107: note = hz208F2 light chain L021, VL
DIQMTQSPSSLSASVGDRVTITCRASQDISKYLNWYQQKPGKAPKLLIYYTSRLHSGVPSRFSGRGSGTDFSLTISSLQPEDFATYFCQQGSTLPYTFGGGTKVEIK

SEQ ID NO: 61 - protein, 214 aa, synthetic construct
  REGION 1..214: note = hz208F2 light chain L021 full length
DIQMTQSPSSLSASVGDRVTITCRASQDISKYLNWYQQKPGKAPKLLIYYTSRLHSGVPSRFSGRGSGTDFSLTISSLQPEDFATYFCQQGSTLPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 120 aa, synthetic construct
  REGION 1..120: note = hz208F2 heavy chain H047, VH
QVQLVQSGAEVKKPGASVKVSCKASGYTFTSYYIHWVRQAPGQGLEWMGWIWPGDGSTKYAQKFQGRVTMTRDTSSNTVYMELSSLRSEDTAVYYCASPMITPNYAMDYWGQGTLVTVSS

SEQ ID NO: 63 - protein, 449 aa, synthetic construct
  REGION 1..449: note = hz208F2 heavy chain H047 full length
QVQLVQSGAEVKKPGASVKVSCKASGYTFTSYYIHWVRQAPGQGLEWMGWIWPGDGSTKYAQKFQGRVTMTRDTSSNTVYMELSSLRSEDTAVYYCASPMITPNYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 64 - protein, 120 aa, synthetic construct
  REGION 1..120: note = hz208F2 heavy chain H049, VH
QVQLVQSGAEVKKPGASVKVSCKASGYTFTSYYMHWVRQAPGQGLEWMGWIWPGDGSTKYAQKFQGRVTMTRDKSSSTVYMELSSLRSEDTAVYYCASPMITPNYAMDYWGQGTLVTVSS

SEQ ID NO: 65 - protein, 449 aa, synthetic construct
  REGION 1..449: note = hz208F2 heavy chain H049 full length
QVQLVQSGAEVKKPGASVKVSCKASGYTFTSYYMHWVRQAPGQGLEWMGWIWPGDGSTKYAQKFQGRVTMTRDKSSSTVYMELSSLRSEDTAVYYCASPMITPNYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 66 - protein, 120 aa, synthetic construct
  REGION 1..120: note = hz208F2 heavy chain H051, VH
QVQLVQSGAEVKKPGASVKVSCKASGYTFTSYYMHWVRQAPGQGLEWMGWIWPGDGSTKYAQKFQGRVTMTRDTSSNTVYMELSSLRSEDTAVYFCASPMITPNYAMDYWGQGTLVTVSS

SEQ ID NO: 67 - protein, 449 aa, synthetic construct
  REGION 1..449: note = hz208F2 heavy chain H051 full length
QVQLVQSGAEVKKPGASVKVSCKASGYTFTSYYMHWVRQAPGQGLEWMGWIWPGDGSTKYAQKFQGRVTMTRDTSSNTVYMELSSLRSEDTAVYFCASPMITPNYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 68 - protein, 120 aa, synthetic construct
  REGION 1..120: note = hz208F2 heavy chain H052, VH
QVQLVQSGAEVKKPGASVKVSCKASGYTFTSYYMHWVRQAPGQGLEWMGWIWPGDGSTKYAQKFQGRVTMTRDTSTSTVYMELSSLRSEDTAVYFCASPMITPNYAMDYWGQGTLVTVSS

SEQ ID NO: 69 - protein, 449 aa, synthetic construct
  REGION 1..449: note = hz208F2 heavy chain H052 full length
QVQLVQSGAEVKKPGASVKVSCKASGYTFTSYYMHWVRQAPGQGLEWMGWIWPGDGSTKYAQKFQGRVTMTRDTSTSTVYMELSSLRSEDTAVYFCASPMITPNYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 70 - protein, 120 aa, synthetic construct
  REGION 1..120: note = hz208F2 heavy chain H057, VH
QVQLVQSGAEVKKPGASVKVSCKASGYTFTSYYIHWVRQAPGQGLEWMGWIWPGDGSTKYAQKFQGRVTMTRDKSTNTVYMELSSLRSEDTAVYYCASPMITPNYAMDYWGQGTLVTVSS

SEQ ID NO: 71 - protein, 449 aa, synthetic construct
  REGION 1..449: note = hz208F2 heavy chain H057 full length
QVQLVQSGAEVKKPGASVKVSCKASGYTFTSYYIHWVRQAPGQGLEWMGWIWPGDGSTKYAQKFQGRVTMTRDKSTNTVYMELSSLRSEDTAVYYCASPMITPNYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 72 - protein, 120 aa, synthetic construct
  REGION 1..120: note = hz208F2 heavy chain H068, VH
QVQLVQSGAEVKKPGASVKVSCKASGYTFTSYYMHWVRQAPGQGLEWMGWIWPGDGSTKYAQKFQGRVTMTRDTSTNTVYMELSSLRSEDTAVYFCASPMITPNYAMDYWGQGTLVTVSS

SEQ ID NO: 73 - protein, 449 aa, synthetic construct
  REGION 1..449: note = hz208F2 heavy chain H068 full length
QVQLVQSGAEVKKPGASVKVSCKASGYTFTSYYMHWVRQAPGQGLEWMGWIWPGDGSTKYAQKFQGRVTMTRDTSTNTVYMELSSLRSEDTAVYFCASPMITPNYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 74 - protein, 120 aa, synthetic construct
  REGION 1..120: note = hz208F2 heavy chain H070, VH
QVQLVQSGAEVKKPGASVKVSCKASGYTFTSYYMHWVRQAPGQGLEWMGWIWPGDGSTKYAQKFQGRVTMTRDTSTSTVYMELSSLRSEDTAVYYCASPMITPNYAMDYWGQGTLVTVSS

SEQ ID NO: 75 - protein, 449 aa, synthetic construct
  REGION 1..449: note = hz208F2 heavy chain H070 full length
QVQLVQSGAEVKKPGASVKVSCKASGYTFTSYYMHWVRQAPGQGLEWMGWIWPGDGSTKYAQKFQGRVTMTRDTSTSTVYMELSSLRSEDTAVYYCASPMITPNYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 76 - protein, 120 aa, synthetic construct
  REGION 1..120: note = hz208F2 heavy chain H071, VH
QVQLVQSGAEVKKPGASVKVSCKASGYTFTSYYMHWVRQAPGQGLEWMGWIWPGDGSTKYAQKFQGRVTMTRDTSTNTVYMELSSLRSEDTAVYYCASPMITPNYAMDYWGQGTLVTVSS

SEQ ID NO: 77 - protein, 449 aa, synthetic construct
  REGION 1..449: note = hz208F2 heavy chain H071 full length
QVQLVQSGAEVKKPGASVKVSCKASGYTFTSYYMHWVRQAPGQGLEWMGWIWPGDGSTKYAQKFQGRVTMTRDTSTNTVYMELSSLRSEDTAVYYCASPMITPNYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 78 - protein, 120 aa, synthetic construct
  REGION 1..120: note = hz208F2 heavy chain H076, VH
QVQLVQSGAEVKKPGASVKVSCKASGYTFTSYYIHWVRQAPGQGLEWMGWIWPGDGSTKYAQKFQGRVTMTRDTSTSTVYMELSSLRSEDTAVYYCASPMITPNYAMDYWGQGTLVTVSS

SEQ ID NO: 79 - protein, 449 aa, synthetic construct
  REGION 1..449: note = hz208F2 heavy chain H076 full length
QVQLVQSGAEVKKPGASVKVSCKASGYTFTSYYIHWVRQAPGQGLEWMGWIWPGDGSTKYAQKFQGRVTMTRDTSTSTVYMELSSLRSEDTAVYYCASPMITPNYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 80 - protein, 120 aa, synthetic construct
  REGION 1..120: note = hz208F2 heavy chain H077, VH
QVQLVQSGAEVKKPGASVKVSCKASGYTFTSYYIHWVRQAPGQGLEWMGWIWPGDGSTKYAQKFQGRVTMTRDTSTSTVYMELSSLRSEDTAVYFCASPMITPNYAMDYWGQGTLVTVSS

SEQ ID NO: 81 - protein, 449 aa, synthetic construct
  REGION 1..449: note = hz208F2 heavy chain H077 full length
QVQLVQSGAEVKKPGASVKVSCKASGYTFTSYYIHWVRQAPGQGLEWMGWIWPGDGSTKYAQKFQGRVTMTRDTSTSTVYMELSSLRSEDTAVYFCASPMITPNYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2024/0350657 A1
(43) Pub. Date: Oct. 24, 2024

(54) SULFOMALEIMIDE-BASED LINKERS AND CORRESPONDING CONJUGATES

(71) Applicant: PIERRE FABRE MEDICAMENT, BOULOGNE-BILLANCOURT (FR)

(72) Inventors: Michel PEREZ, CASTRES (FR); Frédéric MARION, TOULOUSE (FR); Jean-François HAEUW, BEAUMONT (FR); Cyrille DREYFUS, AMANCY (FR)

(73) Assignee: PIERRE FABRE MEDICAMENT, BOULOGNE-BILLANCOURT (FR)

(21) Appl. No.: 18/622,613

(22) Filed: Mar. 29, 2024

Related U.S. Application Data
(62) Division of application No. 17/280,800, filed on Mar. 26, 2021, now Pat. No. 12,011,485.

(30) Foreign Application Priority Data
Sep. 27, 2018 (EP) 18306270.2

(51) Int. Cl. A61K 47/68; A61K 38/07; A61K 38/12; A61K 47/54; A61P 35/00

(52) U.S. Cl. CPC A61K 47/6889; A61K 38/07; A61K 38/12; A61K 47/545; A61K 47/68031; A61K 47/6809; A61K 47/6811; A61K 47/6849; A61P 35/00

(57) ABSTRACT

The present invention relates to a linker of the following formula (I) or a salt thereof:

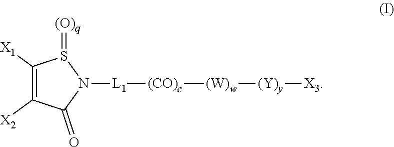

The present invention relates to a linker-drug conjugate of the following formula (II) or a salt thereof:

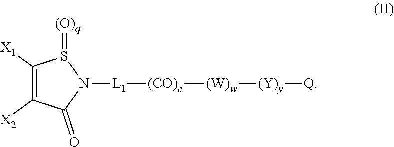

The present invention relates also to a binding unit-drug conjugate, such as an antibody-drug conjugate, of the following formula (III) or (IV) or a salt thereof:

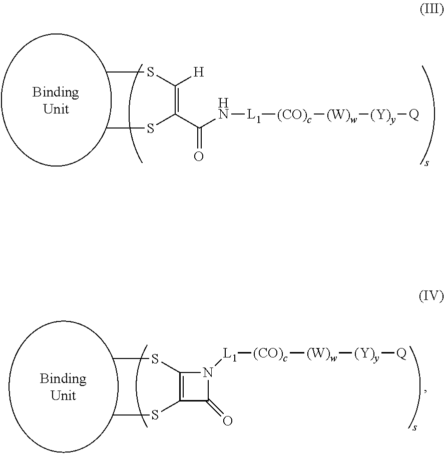

as well as a pharmaceutical composition comprising such a binding unit-drug conjugate and its use in the treatment of cancer.

### CROSS-REFERENCE TO RELATED APPLICATIONS

This application is a Divisional of copending application Ser. No. 17/280,800, filed on Mar. 26, 2021, which is the National Phase under 35 U.S.C. § 371 of International Application No. PCT/IB2019/001114, filed on Sep. 27, 2019, which claims the benefit under 35 U.S.C. § 119(a) to patent application Ser. No. 18/306,270.2, filed in Europe on Sep. 27, 2018, all of which are hereby expressly incorporated by reference into the present application.

### REFERENCE TO ELECTRONIC SEQUENCE LISTING

The application contains a Sequence Listing which has been submitted electronically in .XML format and is hereby incorporated by reference in its entirety. Said .XML copy, created on Jul. 1, 2024, is named “2024-07-01_SequenceListing_3493-0763PUS2” and is 129,355 bytes in size. The sequence listing contained in this .XML file is part of the specification and is hereby incorporated by reference herein in its entirety.

### TECHNICAL FIELD

The present invention relates to a sulfomaleimide-based linker useful in the preparation of conjugates such as antibody-drug conjugates (ADCs) by covalently linking drug molecule(s) to a binding unit, which is advantageously an antibody.

### BACKGROUND

ADCs are all controlled mixtures of different drug-loaded species (from 0 to 8 drug molecules per antibody=DAR) and have a typical average DAR of 3.5 or 4. Unconjugated species are generally not active and are in competition with the drug-loaded species for binding to the antigen. In addition, species that have a DAR of more than 4 have been shown to lead to lower tolerability, higher plasma clearance rates and decreased efficacy. Most of the ADCs that are currently on the market and in clinical trials share common structural features, such as a thiosuccinimide linkage, which is formed through the reaction of thiols and alkyl maleimides. This type of chemistry is widely used because the reaction of maleimides and thiols is very rapid under physiological conditions and is quantitative (without a large excess of both original species). However, thiosuccinimide formation is slowly reversible under physiological conditions. ADCs that contain alkyl maleimides can result in measurable drug loss during prolonged circulation. The pharmacological consequences of this maleimide elimination from ADCs (via a retro-Michael reaction) include diminished antitumour activity due to reduced exposure to the antibody-conjugated form of the drug and greater toxicity, which arises from the non-targeted release of the drug and the linker. This has been described both for cysteine linked ADCs and lysine-linked ADCs via the thioether linker SMCC (succinimidyl 4-(N-maleimidomethyl)cyclohexane-1-carboxylate).

The present invention relates thus to compounds useful for the conjugation of drug to binding units, the obtained conjugates being more stable and more efficient.

### SUMMARY OF THE INVENTION

The present invention thus relates to a linker of the following formula (I):

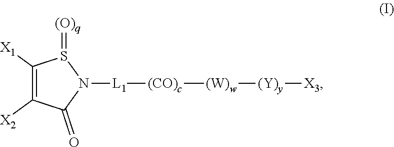

preferably of the following formula (Ia):

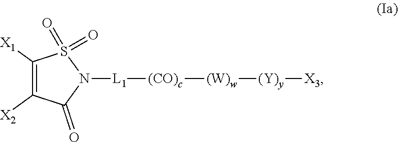

or a salt thereof,
wherein:
X₁ and X₂ represent, independently of each other, H, a halogen atom, a (C₁-C₆)alkoxy, an aryloxy optionally substituted, or —O—(CH₂CH₂O)ᵣH (—O-PEG), provided that X₁ and X₂ do not represent H at the same time;
L₁ represents a group of formula L₁′-(CO—Z′)_{z}′ with L₁′ being —(CH₂)ₙ—, —(CH₂CH₂O)ₘ—CH₂—CH₂—, arylene, heteroarylene, cycloalkanediyl, —(CH₂)ₙ-arylene-, —(CH₂)ₙ-heteroarylene-, —(CH₂)ₙ-cycloalkanediyl-, -arylene-(CH₂)ₚ—, -heteroarylene-(CH₂)ₚ—, -cycloalkanediyl-(CH₂)ₚ—, —(CH₂)ₙ-arylene-(CH₂)ₚ—, —(CH₂)ₙ-heteroarylene-(CH₂)ₚ—, —(CH₂)ₙ-cycloalkanediyl-(CH₂)ₚ—, —(CH₂CH₂O)ₘ—CH₂—CH₂-arylene-(CH₂)ₚ—, —(CH₂CH₂O)ₘ—CH₂—CH₂-heteroarylene-(CH₂)ₚ—, —(CH₂CH₂O)ₘ—CH₂—CH₂-cycloalkanediyl-(CH₂)ₚ—, —(CH₂)ₙ-arylene-CH₂—CH₂—(OCH₂CH₂)ₘ—, —(CH₂)ₙ-heteroarylene-CH₂—CH₂—(OCH₂CH₂)ₘ—, or —(CH₂)ₙ-cycloalkanediyl-CH₂—CH₂—(OCH₂CH₂)ₘ—;
each W independently represents an amino acid unit;
Y is —PAB—CO—(Z)_{z}—, with PAB being

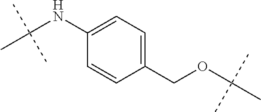

(the oxygen of the PAB unit being linked to CO—(Z)_{z});
Z is —NR₄—(CH₂)ᵤ—NR₅— or —NR₄—(CH₂)ᵤ—NR₅—CO—, or even else —NR₄—(CH₂)ᵤ—NR₅—CO—(CH₂)ᵥ— or —NR₄—(CH₂)ᵤ—NR₅—CO—(CH₂)ᵥ—CO— (the NR₄ group being linked to the CO group of PAB—CO);
Z′ is —NR₄—(CH₂)ᵤ—NR₅— or —NR₄—(CH₂)ᵤ—NR₅—CO—(CH₂)ᵥ— (the NR₄ group being linked to the CO group of CO—Z′);
R₄ and R₅ are independently H or a (C₁-C₆)alkyl group;
c is 0 or 1, preferably 1;
m is an integer from 1 to 15;
n is an integer from 1 to 6;
p is an integer from 1 to 6;
q is 0, 1 or 2, preferably 2;
r is an integer from 1 to 24, notably from 1 to 12;
u is an integer from 1 to 6;
v is an integer from 1 to 6;
w is an integer from 0 to 5, preferably 0 or 2;
y is 0 or 1 (preferably y is 0 when w is 0 and y is 0 or 1 when w is an integer from 1 to 5);
z is 0 or 1;
z′ is 0 or 1, notably 0; and
X₃ represents H when y=z=1 and Z is —NR₄—(CH₂)ᵤ—NR₅— or when c=w=y=0, z′=1 and Z′ is —NR₄—(CH₂)ᵤ—NR₅— and in the other cases, X₃ represents OH, NH₂ or a leaving group.

The leaving group is more particularly a halogen atom, a sulfonate of formula —OSO₂—R_{LG}, N-succinimidyloxy, 4-nitro-phenyloxy, pentafluorophenoxy or N-benzotriazoloxy, R_{LG} representing a (C₁-C₆)alkyl, aryl, aryl-(C₁-C₆)alkyl or (C₁-C₆)alkyl-aryl group, the said group being optionally substituted with one or several halogen atoms such as fluorine atoms.

Preferably, the compound of formula (I) is not a compound of formula (I) for which:
X₁ is Cl, X₂ is H, q is 0, L₁ is

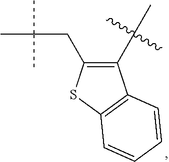

c is 0, w is 0, y is 0 and X₃ is Cl;
X₁ is Cl, X₂ is Cl, g is 0, L₁ is

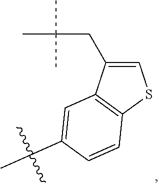

c is 0, w is 0, y is 0 and X₃ is Cl;
X₁ is Cl, X₂ is H, q is 0, L₁ is

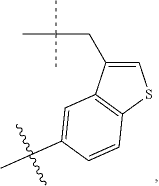

c is 0, w is 0, y is 0 and X₃ is Cl;
X₁ is Cl, X₂ is H, g is 0, L₁ is

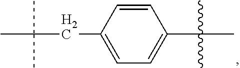

c is 0, w is 0, y is 0 and X₃ is Cl;
X₁ is Cl, X₂ is H q is 0 L₁ is

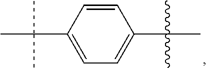

c is 0, w is 0, y is 0 and X₃ is Cl;
X₁ is Cl, X₂ is H, g is 0, L₁ is

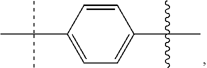

c is 0, w is 0, y is 0 and X₃ is Br;
X₁ is Cl, X₂ is H, g is 0, L₁ is

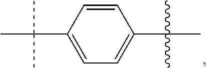

c is 0, w is 0, y is 0 and X₃ is I;
X₁ is H, X₂ is Cl, q is 0, L₁ is

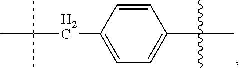

c is 0, w is 0, y is 0 and X₃ is Cl;
X₁ is H, X₂ is Br, q is 0, L₁ is

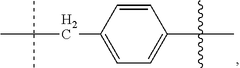

c is 0, w is 0, y is 0 and X₃ is Cl;
X₁ is H X₂ is Br, q is 0 L₁ is

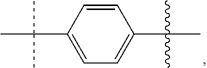

c is 0, w is 0, y is 0 and X₃ is Cl;
X₁ is Cl, X₂ is Cl, q is 0, L₁ is

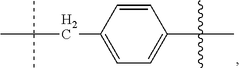

c is 0, w is 0, y is 0 and X₃ is Cl;
X₁ is Cl, X₂ is Cl, q is 0, L₁ is

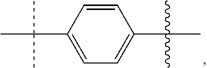

c is 0, w is 0, y is 0 and X₃ is Cl;
X₁ is Cl, X₂ is Cl, q is 0, L₁ is

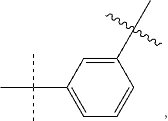

c is 0, w is 0, y is 0 and X₃ is Cl;
X₁ is Cl, X₂ is Br, q is 0 L₁ is

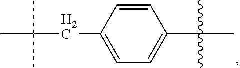

c is 0, w is 0, y is 0 and X₃ is Cl;
X₁ is Cl, X₂ is Br, q is 0 L₁ is

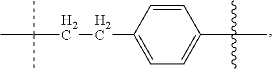

c is 0, w is 0, y is 0 and X₃ is Cl;
X₁ is Cl, X₂ is Br, q is 0, L₁ is

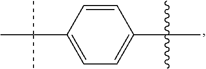

c is 0, w is 0, y is 0 and X₃ is Cl; or
X₁ is Br, X₂ is Br, q is 0, L₁ is

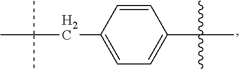

c is 0, w is 0, y is 0 and X₃ is Cl; wherein the dashed line indicates the point of attachment of L₁ to the nitrogen atom of

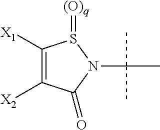

and the wavy line indicates the point of attachment of L₁ to X₃.
   These compounds are disclosed in WO 2007/001932 or in U.S. Pat. No. 4,127,687 but not as a linker which is more particularly intended to the preparation of conjugates such as ADCs.

The present invention relates also to a linker-drug conjugate of the following formula (II):

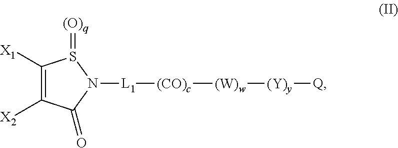

preferably of the following formula (IIa):

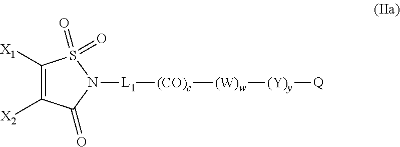

or a salt thereof,
wherein:
X₁ and X₂ represent, independently of each other, H, a halogen atom, a (C₁-C₆)alkoxy, an aryloxy optionally substituted, or —O—(CH₂CH₂O)ᵣH, provided that X₁ and X₂ do not represent H at the same time;
L₁ represents a group of formula L₁′-(CO—Z′)_{z}′ with L₁′ being —(CH₂)ₙ—, —(CH₂CH₂O)ₘ—CH₂—CH₂—, arylene, heteroarylene, cycloalkanediyl, —(CH₂)ₙ-arylene-, —(CH₂)ₙ-heteroarylene-, —(CH₂)ₙ-cycloalkanediyl-, -arylene-(CH₂)ₚ—, -heteroarylene-(CH₂)ₚ—, -cycloalkanediyl-(CH₂)ₚ—, —(CH₂)ₙ-arylene-(CH₂)ₚ—, —(CH₂)ₙ-heteroarylene-(CH₂)ₚ—, —(CH₂)ₙ-cycloalkanediyl-(CH₂)ₚ—, —(CH₂CH₂O)ₘ—CH₂—CH₂-arylene-(CH₂)ₚ—, —(CH₂CH₂O)ₘ—CH₂—CH₂-heteroarylene-(CH₂)ₚ—, —(CH₂CH₂O)ₘ—CH₂—CH₂-cycloalkanediyl-(CH₂)ₚ—, —(CH₂)ₙ-arylene-CH₂—CH₂—(OCH₂CH₂)ₘ—, —(CH₂)ₙ-heteroarylene-CH₂—CH₂—(OCH₂CH₂)ₘ—, or —(CH₂)ₙ-cycloalkanediyl-CH₂—CH₂—(OCH₂CH₂)ₘ—;
each W independently represents an amino acid unit;
Y is —PAB—CO—(Z)_{z}—, with PAB being

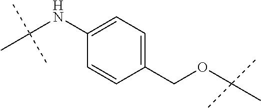

(the oxygen of the PAB unit being linked to CO—(Z)_{z});
Z is —NR₄—(CH₂)ᵤ—NR₅— or —NR₄—(CH₂)ᵤ—NR₅—CO—, or even else —NR₄—(CH₂)ᵤ—NR₅—CO—(CH₂)ᵥ— or —NR₄—(CH₂)ᵤ—NR₅—CO—(CH₂)ᵥ—CO— (the NR₄ group being linked to the CO group of PAB—CO);
Z′ is —NR₄—(CH₂)ᵤ—NR₅— or —NR₄—(CH₂)ᵤ—NR₅—CO—(CH₂)ᵥ— (the NR₄ group being linked to the CO group of CO—Z′);
R₄ and R₅ are independently H or a (C₁-C₆)alkyl group;
Q represents a drug moiety;
c is 0 or 1, preferably 1;
m is an integer from 1 to 15;
n is an integer from 1 to 6;
p is an integer from 1 to 6;
q is 0, 1 or 2, preferably 2;
r is an integer from 1 to 24, notably from 1 to 12;
u is an integer from 1 to 6;
v is an integer from 1 to 6;
w is an integer from 0 to 5, preferably 0 or 2;
y is 0 or 1 (preferably y is 0 when w is 0 and y is 0 or 1 when w is an integer from 1 to 5);
z is 0 or 1; and
z′ is 0 or 1, notably 0.

The present invention relates also to a binding unit-drug conjugate of the following formula (III) or (IV):

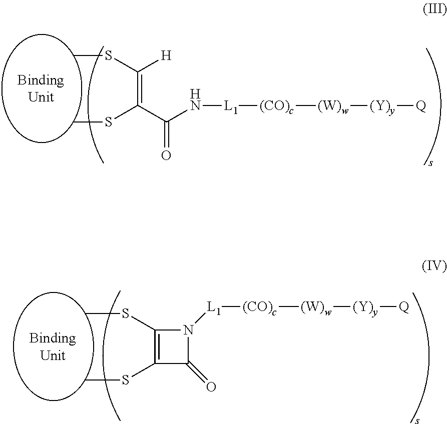

or a salt thereof, preferably a pharmaceutically acceptable salt thereof,
wherein:
the binding unit is a peptide, a protein (e.g. an engineered protein), an antibody, (e.g. a monoclonal antibody) or an antigen binding fragment thereof,
L₁ represents a group of formula L₁′-(CO—Z′)_{z}′ with L₁′ being —(CH₂)ₙ—, —(CH₂CH₂O)ₘ—CH₂—CH₂—, arylene, heteroarylene, cycloalkanediyl, —(CH₂)ₙ-arylene-, —(CH₂)ₙ-heteroarylene-, —(CH₂)ₙ-cycloalkanediyl-, -arylene-(CH₂)ₚ—, -heteroarylene-(CH₂)ₚ—, -cycloalkanediyl-(CH₂)ₚ—, —(CH₂)ₙ-arylene-(CH₂)ₚ—, —(CH₂)ₙ-heteroarylene-(CH₂)ₚ—, —(CH₂)ₙ-cycloalkanediyl-(CH₂)ₚ—, —(CH₂CH₂O)ₘ—CH₂—CH₂-arylene-(CH₂)ₚ—, —(CH₂CH₂O)ₘ—CH₂—CH₂-heteroarylene-(CH₂)ₚ—, —(CH₂CH₂O)ₘ—CH₂—CH₂-cycloalkanediyl-(CH₂)ₚ—, —(CH₂)ₙ-arylene-CH₂—CH₂—(OCH₂CH₂)ₘ—, —(CH₂)ₙ-heteroarylene-CH₂—CH₂—(OCH₂CH₂)ₘ—, or —(CH₂)ₙ-cycloalkanediyl-CH₂—CH₂—(OCH₂CH₂)ₘ—;
each W independently represents an amino acid unit;
Y is —PAB—CO—(Z)_{z}—, with PAB being

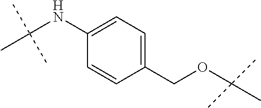

(the oxygen of the PAB unit being linked to CO—(Z)_{z};
Z is —NR₄—(CH₂)ᵤ—NR₅— or —NR₄—(CH₂)ᵤ—NR₅—CO—, or even else —NR₄—(CH₂)ᵤ—NR₅—CO—(CH₂)ᵥ— or —NR₄—(CH₂)ᵤ—NR₅—CO—(CH₂)ᵥ—CO— (the NR₄ group being linked to the CO group of PAB—CO);
Z′ is —NR₄—(CH₂)ᵤ—NR₅— or —NR₄—(CH₂)ᵤ—NR₅—CO—(CH₂)ᵥ— (the NR₄ group being linked to the CO group of CO—Z′);
R₄ and R₅ are independently H or a (C₁-C₆)alkyl group;
Q represents a drug moiety;
c is 0 or 1, preferably 1;
m is an integer from 1 to 15;
n is an integer from 1 to 6;
p is an integer from 1 to 6;
s is an integer from 1 to 8;
u is an integer from 1 to 6;
v is an integer from 1 to 6;
w is an integer from 0 to 5, preferably 0 or 2;
y is 0 or 1 (preferably y is 0 when w is 0 and y is 0 or 1 when w is an integer from 1 to 5);
z is 0 or 1; and
z′ is 0 or 1, notably 0.

According to a preferred embodiment, the binding unit is an IGF-1R antibody, a HER2 antibody (e.g. trastuzumab) or an antigen binding fragment thereof.

The present invention relates also to the use of a linker of formula (I) or a drug-linker conjugate of formula (II), preferably in which q=2, for covalently linking a drug to a binding unit, such as an antibody (e.g. a monoclonal antibody) or an antigen binding fragment thereof. Such a covalent link is thus made by means of a linker moiety.

Indeed, the compounds of formula (I) or (II), preferably for which q=2, are useful for covalently linking a drug to a binding unit, such as an antibody (e.g. a monoclonal antibody) or an antigen binding fragment thereof.

The compounds of formula (I) or (II) for which q=0 or 1 can also be used as synthesis intermediates for preparing compounds of formula (I) or (II) for which q=2. In consequence, the present invention relates also to the compounds of formula (I) or (II) as defined above, for which q=0 or 1, as synthesis intermediate.

The present invention relates also to methods for preparing the linker of formula (I) or the conjugates of formula (II), (III) or (IV).

The present invention relates also to a pharmaceutical composition comprising a binding unit-drug conjugate of formula (III) or (IV) and at least one pharmaceutically acceptable excipient.

The present invention relates also to a binding unit-drug conjugate of formula (III) or (IV) or a pharmaceutical composition comprising a binding unit-drug conjugate of formula (III) or (IV) and at least one pharmaceutically acceptable excipient for use in the treatment of cancer.

The present invention relates also to the use of a binding unit-drug conjugate of formula (III) or (IV) for the manufacture of a medicament intended to be used in the treatment of cancer.

The present invention relates also to a method for treating cancer comprising the administration to a person in need thereof of an effective amount of a binding unit-drug conjugate of formula (III) or (IV) or of a pharmaceutical composition comprising a binding unit-drug conjugate of formula (III) or (IV) and at least one pharmaceutically acceptable excipient.

### Definitions

For the purpose of the invention, the term “pharmaceutically acceptable” is intended to mean what is useful to the preparation of a pharmaceutical composition, and what is generally safe and non toxic, for a pharmaceutical use.

The term “pharmaceutically acceptable salt” is intended to mean, in the framework of the present invention, a salt of a compound which is pharmaceutically acceptable, as defined above, and which possesses the pharmacological activity of the corresponding compound.

The pharmaceutically acceptable salts comprise:

(1) acid addition salts formed with inorganic acids such as hydrochloric, hydrobromic, sulfuric, nitric and phosphoric acid and the like; or formed with organic acids such as acetic, benzenesulfonic, fumaric, glucoheptonic, gluconic, glutamic, glycolic, hydroxynaphtoic, 2-hydroxyethanesulfonic, lactic, maleic, malic, mandelic, methanesulfonic, muconic, 2-naphtalenesulfonic, propionic, succinic, dibenzoyl-L-tartaric, tartaric, p-toluenesulfonic, trimethylacetic, and trifluoroacetic acid and the like, and

(2) salts formed when an acid proton present in the compound is either replaced by a metal ion, such as an alkali metal ion, an alkaline-earth metal ion, or an aluminium ion; or coordinated with an organic or inorganic base. Acceptable organic bases comprise diethanolamine, ethanolamine, N-methylglucamine, triethanolamine, tromethamine and the like. Acceptable inorganic bases comprise aluminium hydroxide, calcium hydroxide, potassium hydroxide, sodium carbonate and sodium hydroxide.

The term “halogen”, as used in the present invention, refers to a fluorine, bromine, chlorine or iodine atom.

The term “(C₁-C₆)alkyl”, as used in the present invention, refers to a monovalent straight or branched saturated hydrocarbon chain containing from 1 to 6 carbon atoms including, but not limited to, methyl, ethyl, n-propyl, iso-propyl, n-butyl, iso-butyl, sec-butyl, t-butyl, n-pentyl, n-hexyl, and the like.

The term “(C₁-C₆)alkoxy”, as used in the present invention, refers to a (C₁-C₆)alkyl group as defined above bound to the molecule via an oxygen atom, including, but not limited to, methoxy, ethoxy, n-propoxy, iso-propoxy, n-butoxy, iso-butoxy, sec-butoxy, t-butoxy, n-pentoxy, n-hexoxy, and the like.

The term “(C₂-C₆)alkenyl”, as used in the present invention, refers to a straight or branched monovalent unsaturated hydrocarbon chain containing from 2 to 6 carbon atoms and comprising at least one double bond including, but not limited to, ethenyl, propenyl, butenyl, pentenyl, hexenyl and the like.

The term “cycloalkanediyl”, as used in the present invention, refers to a bivalent saturated hydrocarbon ring having advantageously 4 to 10 carbon atoms, notably 5 or 6 carbon atoms including, but not limited to, cyclopentanediyl, cyclohexandiyl and the like. Preferably, it is a cyclohexanediyl group.

The term “aryl”, as used in the present invention, refers to a monovalent aromatic hydrocarbon group comprising preferably 6 to 10 carbon atoms and comprising one or more fused rings, such as, for example, a phenyl or naphtyl group. Advantageously, it will be a phenyl group.

The term “aryl-(C₁-C₆)alkyl”, as used in the present invention, refers to a (C₁-C₆)alkyl group as defined above substituted with an aryl group as defined above. In particular, it can be a benzyl group.

The term “(C₁-C₆)alkyl-aryl”, as used in the present invention, refers to an aryl group as defined above substituted with a (C₁-C₆)alkyl group as defined above. In particular, it can be a tolyl group (CH₃Ph).

The term “aryloxy”, as used in the present invention, refers to an aryl group as defined above bound to the molecule via an oxygen atom, including, but not limited to phenyloxy.

The term “arylene”, as used in the present invention, refers to a bivalent aromatic hydrocarbon group comprising preferably 6 to 10 carbon atoms and comprising one or more fused rings, such as, for example, a phenylene or naphtylene group. Advantageously, it will be a phenylene group.

The term “heteroarylene”, as used in the present invention, refers to a bivalent aromatic group comprising one or several, notably one or two, fused hydrocarbon cycles in which one or several, notably one to four, advantageously one to three, carbon atoms each have been replaced with a heteroatom selected from a sulfur atom, an oxygen atom and a nitrogen atom, preferably selected from an oxygen atom and a nitrogen atom, more preferably a nitrogen atom. Advantageously, it is a bivalent 1,2,3-triazole, such as a bivalent III-1,2,3-triazole.

The term “leaving group” as used in the present invention refers to a chemical group which can be easily replaced with a nucleophile (such as an amine or an alcohol respectively bearing a functional group NH or OH) during a nucleophile substitution reaction. Such a leaving group can be in particular a halogen atom, a sulfonate, N-succinimidyloxy, 4-nitro-phenyloxy, pentafluorophenoxy or N-benzotriazoloxy. The sulfonate is in particular a group —OSO₂—R_{LG} with R_{LG} representing a (C₁-C₆)alkyl, aryl, aryl-(C₁-C₆)alkyl or (C₁-C₆)alkyl-aryl group, the said group being optionally substituted with one or several halogen atoms such as fluorine atoms. The sulfonate can be notably a mesylate (OMs, CH₃—S(O₂)O—), a triflate (OTf, CF₃—S(O)₂O—) or a tosylate (OTs, p-Me-C₆H₄—S(O)₂O—). The leaving group can be in particular Cl, Br, I, OTf, OMs, OTf, N-succinimidyloxy, 4-nitro-phenyloxy or N-benzotriazoloxy.

The term “trialkylsilyl group”, as used in the present invention, refers to a group —SiAlk₁Alk₂Alk₃ in which Alk₁, Alk₂ and Alk₃, identical or different, represent a (C₁-C₆)-alkyl group as defined above. For example, it can be a trimethylsilyl or triethylsilyl group.

The term “protected form” of a molecule means that at least an OH or NH function present on said molecule is protected with an O-protecting group or an N-protecting group respectively.

The term “protecting group”, as used in the present invention, refers to a chemical group which selectively blocks a reactive site in a multifunctional compound so as to allow selectively performing a chemical reaction on another unprotected reactive site. The term “O-protecting group” as used in the present invention refers to a substituent which protects hydroxyl groups (OH) against undesirable reactions during synthetic procedures such as those O-protecting groups disclosed in “Greene's Protective Groups In Organic Synthesis”, 4^{th} edition, 2007, John Wiley & Sons, Hoboken, New Jersey. A hydroxyl group protected by a O-protecting group can be for example an ether, an ester, a carbonate, an acetal and the like. In particular, O-protecting groups can be a (C₁-C₆)alkyl optionally substituted with one or several (notably 1 to 3) halogen atoms (such as chlorine atoms), such as methyl, ethyl, tert-butyl or 2,2,2-trichloroethyl; an aryl-(C₁-C₆)alkyl, such as a benzyl, the aryl moiety being optionally substituted with one or several methoxy groups, such as benzyl (Bn) or p-methoxybenzyl (PMB); a trityl derivative of formula —CAᵣ₁Aᵣ₂Aᵣ₃ such as triphenylmethyl (also called trityl—Tr), (4-methoxyphenyl)diphenylmethyl (also called methoxytrityl—NMT) or bis-(4-methoxyphenyl)phenylmethyl (also called dimethoxytrityl—DMT); a substituted methyl group of formula —CH₂OR_{GP2} or —CH₂SR_{GP2} (in particular —CH₂OR_{GP2}), for example, methoxymethyl (MOM), benzyloxymethyl, 2-methoxyethoxymethyl (MEM), 2-(trimethylsilyl)ethoxymethyl or methylthiomethyl; a substituted ethyl group of formula —CH₂CH₂OR_{GP2} or —CH₂CH₂SR_{GP2} (in particular —CH₂CH₂OR_{GP2}), for example, ethoxyethyl (EE); a silyl group of formula —SiR_{GP3}R_{GP4}R_{GP5}, for example, trimethylsilyl (TMS), triethylsilyl (TES), t-butyldimethylsilyl (TBS or TBDMS) and t-butyldiphenylsilyl (TBDPS); a carbonylated group of formula —CO—R_{GP6} such as acetyl (Ac), pivaloyl (Piv or Pv) or benzoyl (Bz) or of formula —CO₂—R_{GP7} such as allyloxycarbonyl (Alloc) or 9-fluorenylmethyloxycarbonyl (Fmoc); or a tetrahydropyranyl

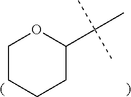

(THP) or tetrahydrofuranyl

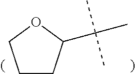

group; with Ar₁, Ar₂ and Ar₃ representing, independently from one another, an aryl, such as a phenyl, optionally substituted with one or several methoxy groups; R_{GP2} representing a (C₁-C₆)alkyl (such as methyl or ethyl) optionally substituted with an aryl (such as phenyl), a (C₁-C₆)alkoxy (such as methoxy) or a trialkylsilyl group (such as SiMe₃); R_{GP3}, R_{GP4} and R_{GP5} representing, independently from one another, a (C₁-C₆)alkyl or aryl (such as phenyl) group; and R_{GP6} and R_{GP7} representing, independently of each other, a (C₁-C₆)alkyl, a (C₂-C₆)alkenyl, an aryl, an aryl-(C₁-C₆)alkyl or a 9-fluorenylmethyl group.

The term “N-protecting group”, as used in the present invention, refers to those groups intended to protect an amine function (notably a primary amine function) against undesirable reactions during synthetic procedures. Commonly used N-protecting groups are disclosed in “Greene's Protective Groups In Organic Synthesis”, 4^{th} edition, 2007, John Wiley & Sons, Hoboken, New Jersey. An amine function protected by a N-protecting group can be a carbamate, an amide, a sulfonamide, an N-alkyl derivative, an amino acetal derivative, a N-benzyl derivative, an imine derivative, an enamine derivative or a N-heteroatom derivative. In particular, N-protecting groups can be formyl; an aryl, such as a phenyl, optionally substituted with one or several methoxy groups such as p-methoxyphenyl (PMP); an aryl-(C₁-C₆)alkyl, such as a benzyl, the aryl moiety being optionally substituted with one or several methoxy groups, such as benzyl (Bn), p-methoxybenzyl (PMB) or 3,4-dimethoxybenzyl (DMPM); —CO—R_{GP1} such as acetyl (Ac), pivaloyl (Piv or Pv), benzoyl (Bz) or p-methoxybenzylcarbonyl (Moz); —CO₂—R_{GP1} such as tbutyloxycarbonyl (Boc), trichloroethoxycarbonyl (TROC), allyloxycarbonyl (Alloc), benzyloxycarbonyl (Cbz or Z) or 9-fluorenylmethyloxycarbonyl (Fmoc); —SO₂-R_{GP1} such as phenylsulfonyl, tosyl (Ts or Tos) or 2-nitrobenzenesulfonyl (also called nosyl—Nos or Ns); and the like,

with R_{GP1} representing a (C₁-C₆)alkyl optionally substituted with one or several halogen atoms such as F or C₁; a (C₂-C₆)alkenyl such as an allyl; an aryl, such as a phenyl, optionally substituted with one or several groups chosen among OMe (methoxy) and NO₂ (nitro); an aryl-(C₁-C₆)alkyl, such as a benzyl, the aryl moiety being optionally substituted with one or several methoxy groups; or a 9-fluorenylmethyl group.

The terms “antibody”, “antibodies” “ab”, “Ab”, “MAb” or “immunoglobulin” are used interchangeably in the broadest sense and include monoclonal antibodies, isolated, engineered or recombinant antibodies (e.g. full length or intact monoclonal antibodies), polyclonal antibodies, multivalent antibodies or multispecific antibodies (e.g. bispecific antibodies) and also antibody fragment thereof, so long as they exhibit the desired biological activity.

The term “recombinant antibody” refers to an antibody that results from the expression of recombinant DNA within living cells. A recombinant antibody according to the invention is obtained by using laboratory methods of genetic recombination, well known by a person skilled in the art, creating DNA sequences that would not be found in biological organisms.

The term “antigen binding fragment” of an antibody according to the invention is intended to indicate any peptide, polypeptide, or protein retaining the ability to bind to the target (also generally referred as antigen) of the antibody.

By “binding”, “binds”, or the like, it is intended that the antibody, or any antigen binding fragment thereof, forms a complex with an antigen that is relatively stable under physiologic conditions. Specific binding can be characterized by an equilibrium dissociation constant of at least about 1×10⁻⁶ M. Methods for determining whether two molecules bind are well known in the art and include, for example, equilibrium dialysis, surface plasmon resonance, radiolabelled assays and the like. For the avoidance of doubt, it does not mean that the said antibody could not bind or interfere, at a low level, to another antigen. Nevertheless, as an embodiment, the said antibody binds only to the said antigen.

As used in the present specification, the expression “IGF-1R antibody” should be interpreted as similar to “anti-IGF-1R antibody” and means an antibody capable of binding to IGF-1R.

As used in the present specification, the expression “HER2 antibody” should be interpreted as similar to “anti-HER2 antibody” and means an antibody capable of binding to HER2.

The term half maximal effective concentration (EC₅₀) corresponds to the concentration of a drug, antibody or toxicant which induces a response halfway between the baseline and maximum after some specified exposure time. It is commonly used as a measure of drug's potency. The EC₅₀ of a graded dose response curve therefore represents the concentration of a compound where 50% of its maximal effect is observed. The EC₅₀ of a quantal dose response curve represents the concentration of a compound where 50% of the population exhibits a response, after specified exposure duration. Concentration measures typically follow a sigmoidal curve, increasing rapidly over a relatively small change in concentration. This can be determined mathematically by derivation of the best-fit line.

As a preferred embodiment, the EC₅₀, determined in the present invention, characterizes the potency of antibody to bind on the IGF-1R ECD exposed on human tumor cells. The EC₅₀ parameter is determined using FACS analysis. The EC₅₀ parameter reflects the antibody concentration for which 50% of the maximal binding on the human IGF-1R expressed on human tumor cells is obtained. Each EC₅₀ value was calculated as the midpoint of the dose response curve using a four-parameter regression curve fitting program (Prism Software), This parameter has been selected as to be representative of physiological/pathological conditions.

The term “epitope” is a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

The term “monoclonal antibody” or “Mab” as used herein refers to an antibody obtained from a population of substantially homogeneous antibodies, i.e. the individual antibodies of the population are identical except for possible naturally occurring mutations that may be present in minor amounts. Monoclonal antibodies are highly specific, being directed against a single epitope. Such monoclonal antibody may be produced by a single clone of B cells or hybridoma. Monoclonal antibodies may also be recombinant, i.e. produced by protein engineering or chemical synthesis. Monoclonal antibodies may also be isolated from phage antibody libraries. In addition, in contrast with preparations of polyclonal antibodies which typically include various antibodies directed against various determinants, or epitopes, each monoclonal antibody is directed against a single epitope of the antigen. The monoclonal antibody herein includes murine, chimeric and humanized antibody.

The term “chimeric antibody” relates to an antibody containing a natural variable region (light chain and heavy chain) derived from an antibody of a given species in combination with constant regions of the light chain and the heavy chain of an antibody of a species heterologous to said given species. The chimeric antibodies can be prepared by using the techniques of recombinant genetics. For example, the chimeric antibody could be produced by cloning recombinant DNA containing a promoter and a sequence coding for the variable region of a nonhuman monoclonal antibody, notably murine, and a sequence coding for heterologous species antibody constant region, preferably human. A chimeric antibody according to the invention coded by one such recombinant gene could be, for example, a mouse-human chimera, the specificity of this antibody being determined by the variable region derived from the murine DNA and its isotype determined by the constant region derived from human DNA.

The term “humanized antibodies” means an antibody that contains CDR regions derived from an antibody of nonhuman origin, the other parts of the antibody molecule being derived from one (or several) human antibodies. In addition, some of the skeleton segment residues (called FR) can be modified to preserve binding affinity. The humanized antibodies or fragments of same can be prepared by techniques known to a person skilled in the art. Such humanized antibodies are preferred for their use in methods involving in vitro diagnoses or preventive and/or therapeutic treatment in vivo. Other humanization techniques, also known to a person skilled in the art, such as, for example, the “CDR grafting” technique described by PDL in patents and patent applications EP 0 451 216, EP 0 682 040, EP 0 939 127, EP 0 566 647, U.S. Pat. Nos. 5,530,101, 6,180,370, 5,585,089, 5,693,761. U.S. Pat. Nos. 5,639,641, 6,054,297, 5,886,152 and 5,877,293 can also be cited.

Without contradictory specification in the present specification, complementarity-determining regions or CDRs, mean the hypervariable regions of the heavy and light chains of immunoglobulins as defined according to the IMGT numbering system.

Nevertheless, CDRs can also be defined according to the Kabat numbering system (Kabat et al., Sequences of proteins of immunological interest, 5^{th} Ed., U.S. Department of Health and Human Services, NIH, 1991, and later editions). There are three heavy chain CDRs and three light chain CDRs. Here, the terms “CDR” and “CDRs” are used to indicate, depending on the case, one or more, or even all, of the regions containing the majority of the amino acid residues responsible for the antibody's binding affinity for the antigen or epitope it recognizes. In order to simplify the reading of the present application, the CDRs according to Kabat are not defined. Nevertheless, it would be obvious for the person skilled in the art, using the definition of the CDRs according to IMGT, to define the CDRs according to Kabat.

In the sense of the present invention, the “identity” or “percentage identity” between two sequences of nucleic acids or amino acids means the percentage of identical nucleotides or amino acid residues between the two sequences to be compared, obtained after optimal alignment, this percentage being purely statistical and the differences between the two sequences being distributed randomly along their length. The comparison of two nucleic acid or amino acid sequences is traditionally carried out by comparing the sequences after having optimally aligned them, said comparison being able to be conducted by segment or by using an “alignment window”. Optimal alignment of the sequences for comparison can be carried out, in addition to comparison by hand, by means of the local homology algorithm of Smith and Waterman (1981) [Ad. App. Math. 2:482], by means of the local homology algorithm of Neddleman and Wunsch (1970) [J. Mol. Biol. 48:443], by means of the similarity search method of Pearson and Lipman (1988) [Proc. Natl. Acad. Sci. USA 85:2444] or by means of computer software using these algorithms (GAP, BESTFIT, FASTA and TFASTA in the Wisconsin Genetics Software Package, Genetics Computer Group, 575 Science Dr., Madison, WI, or by the comparison software BLAST NR or BLAST P).

Percentage identity is calculated by determining the number of positions at which the amino acid nucleotide or residue is identical between the two sequences, preferably between the two complete sequences, dividing the number of identical positions by the total number of positions in the alignment window and multiplying the result by 100 to obtain the percentage identity between the two sequences.

For example, the BLAST program, “BLAST 2 sequences” (Tatusova et al., “Blast 2 sequences—a new tool for comparing protein and nucleotide sequences”, FEMS Microbiol., 1999, Lett. 174:247-250) available on the site http://www.ncbi.nlm.nih.gov/gorf/bl2.html, can be used with the default parameters (notably for the parameters “open gap penalty”: 5, and “extension gap penalty”: 2; the selected matrix being for example the “BLOSUM 62” matrix proposed by the program); the percentage identity between the two sequences to compare is calculated directly by the program.

By the expressions “back-mutation” or “back mutation” it is meant a mutation or replacement of the human residue present in the germline by the corresponding residue initially present in the murine sequence.

The terms “nucleic acid”, “nucleic sequence”, “nucleic acid sequence”, “polynucleotide”, “oligonucleotide”, “polynucleotide sequence” and “nucleotide sequence”, used interchangeably in the present description, mean a precise sequence of nucleotides, modified or not, defining a fragment or a region of a nucleic acid, containing unnatural nucleotides or not, and being either a double-strand DNA, a single-strand DNA or transcription products of said DNAs.

The term “peptide” relates to a chain of amino acid monomers linked to each other by peptide (amide) bonds. The covalent peptide bonds (amides) are formed by reacting the carboxyl group (COOH) of one amino acid with the amino group (NH₂) of another amino acid. The term peptide includes oligopeptide and polypeptide.

The term “protein” is an assembly of one or several peptides as defined above that have undergone post-translational modifications and protein folding so that they are arranged in a biologically functional way.

The term “amino acid” as used in the present invention refers to natural α-amino acids (e.g. Alanine (Ala), Arginine (Arg), Asparagine (Asn), Aspartic acid (Asp), Cysteine (Cys), Glutamine (Gin), Glutamic acid (Glu), Glycine (Gly), Histidine (His), Isoleucine (Ile), Leucine (Leu), Lysine (Lys), Methionine (Met), Phenylalanine (Phe), Proline (Pro), Serine (Ser), Threonine (Thr), Tryptophan (Trp), Tyrosine (Tyr) and Valine (Val)) in the D or L form, as well as non-natural amino acid (e.g. β-alanine, allylglycine, tert-leucine, 3-amino-adipic acid, 2-aminobenzoic acid, 3-aminobenzoic acid, 4-aminobenzoic acid, 2-aminobutanoic acid, 4-amino-1-carboxymethyl piperidine, 1-amino-1-cyclobutanecarboxylic acid, 4-aminocyclohexaneacetic acid, 1-amino-1-cyclohexanecarboxyilic acid, (1R,2R)-2-aminocyclohexanecarboxylic acid, (1R,2S)-2-aminocyclohexanecarboxylic acid, (1S,2R)-2-aminocyclohexanecarboxylic acid, (1S,2S)-2-aminocyclohexanecarboxylic acid, 3-aminocyclohexanecarboxylic acid, 4-aminocyclohexanecarboxylic acid, (1R,2R)-2-aminocyclopentanecarboxylic acid, (1R,2S)-2-aminocyclopentanecarboxyilic acid, 1-amino-1-cyclopentanecarboxylic acid, 1-amino-1-cyclopropanecarboxylic acid, 4-(2-aminoethoxy)-benzoic acid, 3-aminomethylbenzoic acid, 4-aminomethylbenzoic acid, 2-aminobutanoic acid, 4-aminobutanoic acid, 6-aminohexanoic acid, 1-aminoindane-1-carboxylic acid, 4-aminomethyl-phenylacetic acid, 4-aminophenylacetic acid, 3-amino-2-naphtoic acid, 4-aminophenylbutanoic acid, 4-amino-5-(3-indolyl)-pentanoic acid, (4R,5S)-4-amino-5-methylheptanoic acid, (R)-4-amino-5-methylhexanoic acid, (R)-4-amino-6-methylthiohexanoic acid, (S)-4-amino-pentanoic acid, (R)-4-amino-5-phenylpentanoic acid, 4-aminophenylpropionic acid, (R)-4-aminopimeric acid, (4R,5R)-4-amino-5-hyroxyhexanoic acid, (R)-4-amino-5-hydroxypentanoic acid, (R)-4-amino-5-(p-hydroxyphenyl)-pentanoic acid, 8-aminooctanoic acid, (2S,4R)-4-amino-pyrrolidine-2-carboxylic acid, (2S,4S)-4-amino-pyrrolidine-2-carboxylic acid, azetidine-2-carboxylic acid, (2S,4R)-4-benzyl-pyrrolidine-2-carboxylic acid, (S)-4,8-diaminooctanoic acid, tert-butylglycine acid, γ-carboxyglutamate, β-cyclohexylalanine, citrulline, 2,3-diamino propionic acid, hippuric acid, homocyclohexylalanine, moleucine, homophenylalanine, 4-hydroxyproline, indoline-2-carboxylic acid, isonipecotic acid, α-methyl-alanine, nicopetic acid, norleucine, norvaline, octahydroindole-2-carboxylic acid, ornithine, penicillamine, phenylglycine, 4-phenyl-pyrrolidine-2-carboxylic acid, pipecolic acid, propargylglycine, 3-pyridinylalanine, 4-pyridinylalanine, 1-pyrrolidine-3-carboxylic acid, sarcosine, statines, tetrahydroisoquinoline-1-carboxylic acid, 1,2,3,4-tetrahydroisoquinoline-3-carboxylic acid, or tranexamic acid).

### DETAILED DESCRIPTION

### Linker Moiety

The linker moiety according to the present invention enables to covalently attach an antibody to at least one drug moiety.

The linker moiety may be “non cleavable” or “cleavable”.

In a preferred embodiment, it consists in a “cleavable” linker moiety facilitating the release of the drug in the cell.

For example, in some embodiments, the linker is cleavable by a cleaving agent that is present in the intracellular environment (e.g., within a lysosome or endosome or caveolae). The linker can be, for example, a peptidyl linker that is cleaved by an intracellular peptidase or protease enzyme, including, but not limited to, a lysosomal or endosomal protease. Typically, the peptidyl linker comprises at least two successive amino acids or at least three successive amino acids. Cleaving agents can include cathepsins B and D and plasmin, all of which are known to hydrolyse dipeptide drug derivatives resulting in the release of active drug inside target cells. For example, a peptidyl linker that is cleavable by the thiol-dependent protease cathepsin-B, which is highly expressed in cancerous tissue, can be used (e.g. a linker comprising Phe-Leu or Gly-Phe-Leu-Gly). In specific embodiments, the peptidyl linker cleavable by an intracellular protease comprises or is Val-Cit, Phe-Lys or Val-Ala. One advantage of using intracellular proteolytic release of the drug is that the drug is typically attenuated when conjugated and the serum stabilities of the conjugates are typically high.

The group -L₁-(CO)_{c}— represents the stretcher unit of the linker moiety, which is necessarily present. The group -L₁-(CO)_{c}— is a group of formula -L₁′-(CO—Z′)_{z}—(CO)_{c}— with z′ and c being 0 or 1, such as a group -L₁′-(CO)_{c}— when z′ is 0. Preferably, when at least one of w and y is not 0, then z′ is 0 and, in the other cases, z′ is 0 or 1.

L₁′ represents —(CH₂)ₙ—, —(CH₂CH₂O)ₘ—CH₂—CH₂—, arylene, heteroarylene, cycloalkanediyl, —(CH₂)ₙ-arylene-, —(CH₂)ₙ-heteroarylene-, —(CH₂)ₙ-cycloalkanediyl-, -arylene-(CH₂)ₚ—, -heteroarylene-(CH₂)ₚ—, -cycloalkanediyl-(CH₂)ₚ—, —(CH₂)ₙ-arylene-(CH₂)ₚ—, —(CH₂)ₙ-heteroarylene-(CH₂)ₚ—, —(CH₂)ₙ-cycloalkanediyl-(CH₂)ₚ—, —(CH₂CH₂O)ₘ—CH₂—CH₂-arylene-(CH₂)ₚ—, —(CH₂CH₂O)ₘ—CH₂—CH₂-heteroarylene-(CH₂)ₚ—, —(CH₂CH₂O)ₘ—CH₂—CH₂-cycloalkanediyl-(CH₂)ₚ—, —(CH₂)ₙ-arylene-CH₂—CH₂—(OCH₂CH₂)ₘ—, —(CH₂)ₙ-heteroarylene-CH₂—CH₂—(OCH₂CH₂)ₘ—, or —(CH₂)ₙ-cycloalkanediyl-CH₂—CH₂—(OCH₂CH₂)ₘ—. More particularly, the arylene is a phenylene; the cycloalkanediyl is a cyclohexanediyl, such as a para-cyclohexanediyl; and the heteroarylene is a bivalent 1,2,3-triazole, such as a bivalent 1H-1,2,3-triazole.

According to a particular embodiment, L₁′ represents —(CH₂)ₙ—, —(CH₂CH₂O)ₘ—CH₂—CH₂—, arylene, -cycloalkanediyl -, —(CH₂)ₙ-arylene-, -arylene-(CH₂)ₙ—, —(CH₂)ₙ-cycloalkanediyl-, -cycloalkanediyl-(CH₂)ₙ—,

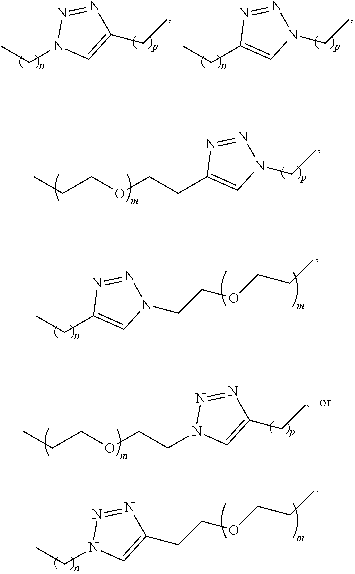

More particularly, the arylene is a phenylene; and the cycloalkanediyl is a cyclohexanediyl, such as a para-cyclohexanediyl.

According to another particular embodiment, L₁′ represents —(CH₂)ₙ— or —(CH₂CH₂O)ₘ—CH₂—CH₂—, notably —(CH₂)ₙ— such as —(CH₂)₅—.

The L₁′ stretcher unit moiety can be completed with a stretcher unit moiety CO—Z′ with Z′ being —CO—NR₄—(CH₂)ᵤ—NR₅— or —CO—NR₄—(CH₂)ᵤ—NR₅—CO—(CH₂)ᵥ—, when z′=1. R₄ and R₅ are independently H or (C₁-C₆)alkyl, such as H or Me. u and v are independently an integer from 1 to 6, such as from 1 to 4, notably 1 or 2, e.g. 2.

(W)_{w} represents the amino acid unit of the linker.

The amino acid unit of the linker can be enzymatically cleaved by an enzyme including, but not limited to, a tumor-associated protease to liberate the drug.

The amino acid unit can be designed and optimized in its selectivity for enzymatic cleavage by a particular tumor-associated protease. The suitable units are those whose cleavage is catalysed by the proteases, cathepsin B, C and D, and plasmin.

(W)_{w} may be absent (w=0) or may be a dipeptide, tripeptide, tetrapeptide or pentapeptide unit (w=1, 2, 3, 4 or 5), wherein the amino acids forming the peptide can be different from one another.

Thus (W)_{w} can be represented by the following formula: (W1)w₁(W2)w₂(W3)w₃(W4)w₄(W5)w₅, wherein each W1 to W5 represents, independently from one another, an amino acid unit and each w1 to w5 is 0 or 1.

In some embodiments, the amino acid unit (W)w may comprise amino acid residues such as those occurring naturally, as well as minor amino acids and non-naturally occurring amino acid analogs, such as citrulline.

The amino acid residues of the amino acid unit (W)w include, without limitation, alanine, valine, leucine, isoleucine, methionine, phenylalanine, tryptophan, proline, lysine protected or not with acetyl or formyl, arginine, arginine protected or not with tosyl or nitro group(s), histidine, omithine, ornithine protected with acetyl or formyl, and citrulline. Exemplary amino acid linker components include preferably a dipeptide or a tripeptide.

Exemplary dipeptides include: Val-Cit, Ala-Val, Ala-Ala, Val-Ala, Lys-Lys, Cit-Cit, Val-Lys, Ala-Phe, Phe-Lys, Ala-Lys, Phe-Cit, Leu-Cit, Ile-Cit, Trp-Cit, Phe-Ala, Phe-N⁹-tosyl-Arg, and Phe-N⁹-Nitro-Arg, preferably Val-Cit or Val-Ala.

Exemplary tripeptides include: Val-Ala-Val, Ala-Asn-Val, Val-Leu-Lys, Ala-Ala-Asn, Phe-Phe-Lys, Gly-Gly-Gly, D-Phe-Phe-Lys, Gly-Phe-Lys.

Exemplary tetrapeptide include: Gly-Phe-Leu-Gly (SEQ ID NO. 53), Ala-Leu-Ala-Leu (SEQ ID NO. 54).

Exemplary pentapeptide include: Pro-Val-Gly-Val-Val (SEQ ID NO. 55).

According to a particular embodiment, (W)w can be a dipeptide (i.e. w=2) such as Val-Cit or Val-Ala, preferably Val-Cit, or the linker lacks an amino acid unit (w=0). When the linker lacks an amino acid unit, preferably it lacks also a spacer unit Y (y=0).

According to a preferred embodiment, w=0 (i.e. (W)_{w} is a single bond) or w=2 (i.e. (W)_{w} is a dipeptide) and (W)_{w} can thus be selected from:

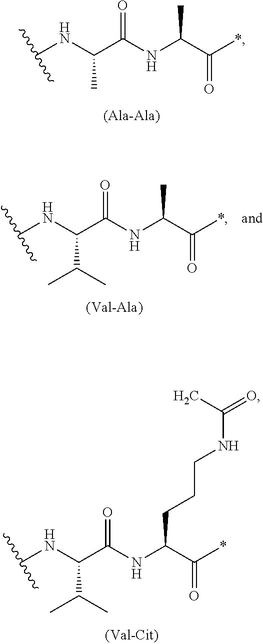

and in particular is Val-Cit,
wherein
the asterisk indicates the point of attachment to the spacer unit (Y)_{y}; and
the wavy line indicates the point of attachment to -L₁-(CO)_{c}— (CO if c=1 or L₁ if c=0).

Y represents the spacer unit of the linker.

Spacer units are of two general types: self-immolative and non self-immolative. A non self-immolative spacer unit is one in which part or all of the spacer unit remains bound to the drug after enzymatic cleavage of an amino acid unit from the antibody-drug conjugate. Examples of a non self-immolative spacer unit include, but are not limited to a (glycine-glycine) spacer unit and a glycine spacer unit. To liberate the drug, an independent hydrolysis reaction should take place within the target cell to cleave the glycine-drug unit bond.

A self-immolative spacer unit can release the drug without the need for a separate hydrolysis step. In these embodiments, (Y) is a residue of p-aminobenzyl alcohol unit (PAB) that is linked to (W)_{w} via the nitrogen atom of the PAB group, and connected directly to the drug via an ester, carbonate, carbamate or ether group. Such a linker comprising a PAB moiety can also be considered as a traceless linker.

In the present invention, the spacer unit (Y) is —PAB—CO—(Z)_{z}— with PAB being

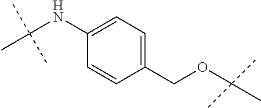

(the oxygen of the PAB unit being linked to the carbonyl), also called -para-aminobenzyl-O—CO—, and y=1 or the linker lacks a spacer unit (y=0).

The spacer Para-aminobenzyl-O—CO— can be completed with a spacer Z which is —NR₄—(CH₂)ᵤ—NR₅— or —NR₄—(CH₂)ᵤ—NR₅—CO— or even —NR₄—(CH₂)ᵤ—NR₅—CO—(CH₂)ᵥ— or —NR₄—(CH₂)ᵤ—NR₅—CO—(CH₂)ᵥ—CO—, when z=1. R₄ and R₅ are independently H or (C₁-C₆)alkyl, such as H or Me. u and v are independently an integer from 1 to 6, such as from 1 to 4, notably 1 or 2, e.g. 2.

Advantageously, y is 0 when w is 0 and y is 0 or 1 when w is an integer from 1 to 5, meaning that the spacer unit Y may be present only when an amino acid unit W is present.

Preferably, y is 0 when w is 0 and y is 1 when w is an integer from 1 to 5, meaning that the spacer unit Y is present when an amino acid unit (W)_{w} is present and is absent when the amino unit (W)_{w} is absent.

According to a particular embodiment, the group -L₁-(CO)_{c}—(W)_{w}—(Y)_{y}-represents —(CH₂)ₙ—, —(CH₂)ₙ—CO—, —(CH₂CH₂O)ₘ—CH₂—CH₂—, —(CH₂CH₂O)ₘ—CH₂—CH₂—CO—, —CH₂-para-cyclohexyl-CO—, -aryl-(CH₂)ₙ—, —(CH₂)ₙ-Val-Cit-para-aminobenzyl-O—CO—, —(CH₂)ₙ—CO-Val-Cit Para-aminobenzyl-O—CO—, —(CH₂CH₂O)ₘ—CH₂—CH₂-Val-Cit-para-aminobenzyl-O—CO—, —(CH₂CH₂O)ₘ—CH₂—CH₂—CO-Val-Cit-para-aminobenzyl-O—CO—, —CH₂-para-cyclohexyl-CO-Val-Cit-para-aminobenzyl-O—CO—, -aryl-(CH₂)ₙ-Val-Cit-Para-aminobenzyl-O—CO—, -aryl-CO-Val-Cit Para-aminobenzyl-O—CO—, —(CH₂)ₙ-Val-Ala Para-aminobenzyl-O—CO—, —(CH₂)ₙ—CO-Val-Ala Para-aminobenzyl-O—CO—, —(CH₂CH₂O)ₘ—CH₂—CH₂-Val-Ala Para-aminobenzyl-O—CO—, —(CH₂CH₂O)ₘ—CH₂—CH₂—CO-Val-Ala Para-aminobenzyl-O—CO—, —CH₂-para-cyclohexyl-CO-Val-Ala-para-aminobenzyl-O—CO—, -aryl-(CH₂)ₙ-Val-Ala-para-aminobenzyl-O—CO—, -aryl-CO-Val-Ala-para-aminobenzyl-O—CO—, —(CH₂)ₙ-Val-Cit-para-aminobenzyl-O—CO—NH—(CH₂)ᵤ—NH—, —(CH₂)ₙ—CO-Val-Cit-para-aminobenzyl-O—CO—NH—(CH₂)ᵤ—NH—, —(CH₂CH₂O)ₘ—CH₂—CH₂-Val-Cit Para-aminobenzyl-O—CO—NH—(CH₂)ᵤ—NH—, —(CH₂CH₂O)ₘ—CH₂—CH₂—CO-Val-Cit-para-aminobenzyl-O—CO—NH—(CH₂)ᵤ—NH—, —CH₂-para-cyclohexyl-CO-Val-Cit-Para-aminobenzyl-O—CO—NH—(CH₂)ᵤ—NH—, -aryl-(CH₂)ₙ-Val-Cit Para-aminobenzyl-O—CO—NH—(CH₂)ᵤ—NH—, -aryl-CO-Val-Cit Para-aminobenzyl-O—CO—NH—(CH₂)ᵤ—NH—, —(CH₂)ₙ-Val-Ala Para-aminobenzyl-O—CO—NH—(CH₂)ᵤ—NH—, —(CH₂)ₙ—CO-Val-Ala para-aminobenzyl-O—CO—NH—(CH₂)ᵤ—NH—, —(CH₂CH₂O)ₘ—CH₂—CH₂-Val-Ala para-aminobenzyl-O—CO—NH—(CH₂)ᵤ—NH—, —(CH₂CH₂O)ₘ—CH₂—CH₂—CO-Val-Ala para-aminobenzyl-O—CO—NH—(CH₂)ᵤ—NH—, —CH₂-para-cyclohexyl-CO-Val-Ala para-aminobenzyl-O—CO—NH—(CH₂)ᵤ—NH—, -aryl-(CH₂)ₙ-Val-Ala Para-aminobenzyl-O—CO—NH—(CH₂)ᵤ—NH—, -aryl-CO-Val-Ala-para-aminobenzyl-O—CO—NH—(CH₂)ᵤ—NH—, —(CH₂)ₙ-Val-Cit Para-aminobenzyl-O—CO—NCH₃—(CH₂)ᵤ—NCH₃—CO—, —(CH₂)ₙ—CO-Val-Cit para-aminobenzyl-O—CO—NCH₃—(CH₂)ᵤ—NCH₃—CO—, —(CH₂CH₂O)ₘ—CH₂—CH₂-Val-Cit para-aminobenzyl-O—CO—NCH₃—(CH₂)ᵤ—NCH₃—CO—, —(CH₂CH₂O)ₘ—CH₂—CH₂—CO-Val-Cit para-aminobenzyl-O—CO—NCH₃—(CH₂)ᵤ—NCH₃—CO—, —CH₂-para-cyclohexyl-CO-Val-Cit-para-aminobenzyl-O—CO—NCH₃—(CH₂)ᵤ—NCH₃—CO—, -aryl-(CH₂)ₙ-Val-Cit-para-aminobenzyl-O—CO—NCH₃—(CH₂)ᵤ—NCH₃—CO—, -aryl-CO-Val-Cit-para-aminobenzyl-O—CO—NCH₃—(CH₂)ᵤ—NCH₃—CO—, —(CH₂)ₙ-Val-Ala Para-aminobenzyl-O—CO—NCH₃—(CH₂)ᵤ—NCH₃—CO—, —(CH₂)ₙ—CO-Val-Ala Para-aminobenzyl-O—CO—NCH₃—(CH₂)ᵤ—NCH₃—CO—, —(CH₂CH₂O)ₘ—CH₂—CH₂-Val-Ala Para-aminobenzyl-O—CO—NCH₃—(CH₂)ᵤ—NCH₃—CO—, —(CH₂CH₂O)ₘ—CH₂—CH₂—CO-Val-Ala Para-aminobenzyl-O—CO—NCH₃—(CH₂)ᵤ—NCH₃—CO—, —CH₂-para-cyclohexyl-CO-Val-Ala Para-aminobenzyl-O—CO—NCH₃—(CH₂)ᵤ—NCH₃—CO—, -aryl-(CH₂)ₙ-Val-Ala Para-aminobenzyl-O—CO—NCH₃—(CH₂)ᵤ—NCH₃—CO—, -aryl-CO-Val-Ala para-aminobenzyl-O—CO—NCH₃—(CH₂)ᵤ—NCH₃—

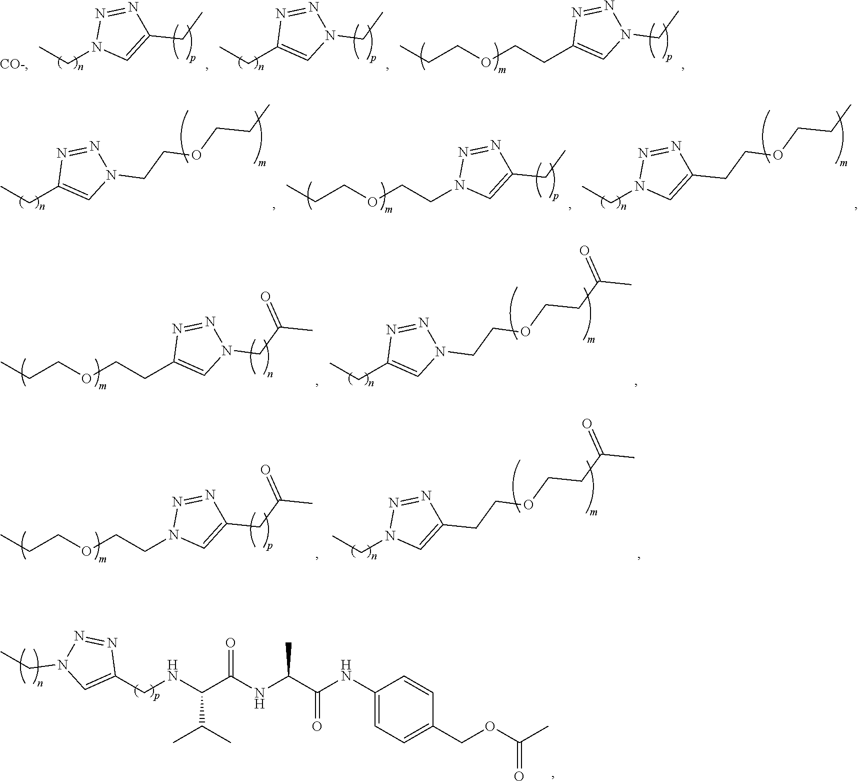

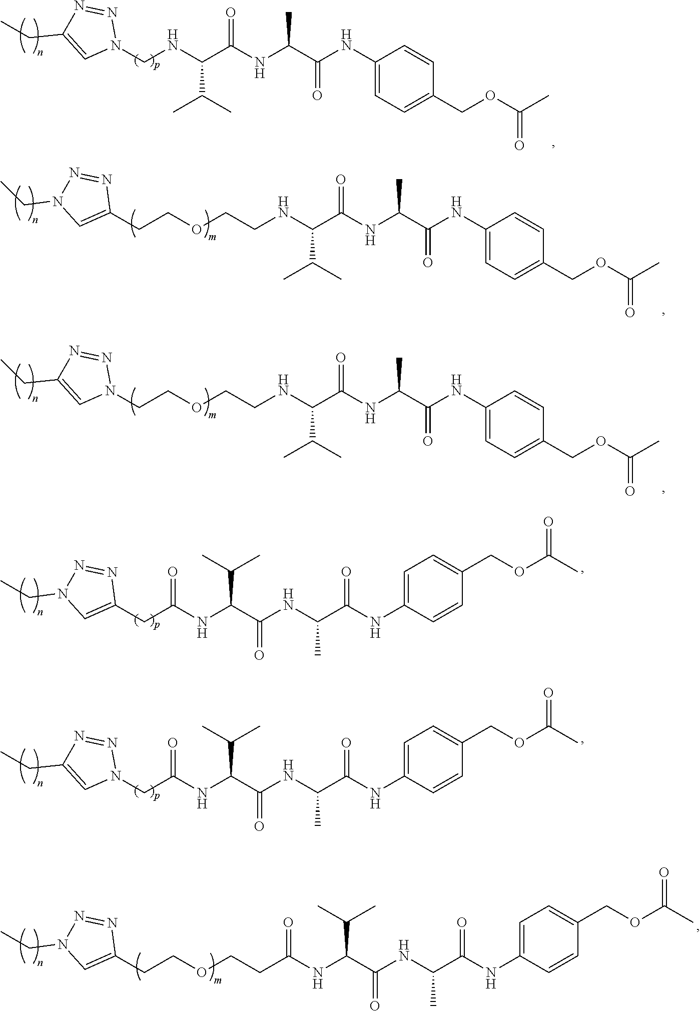

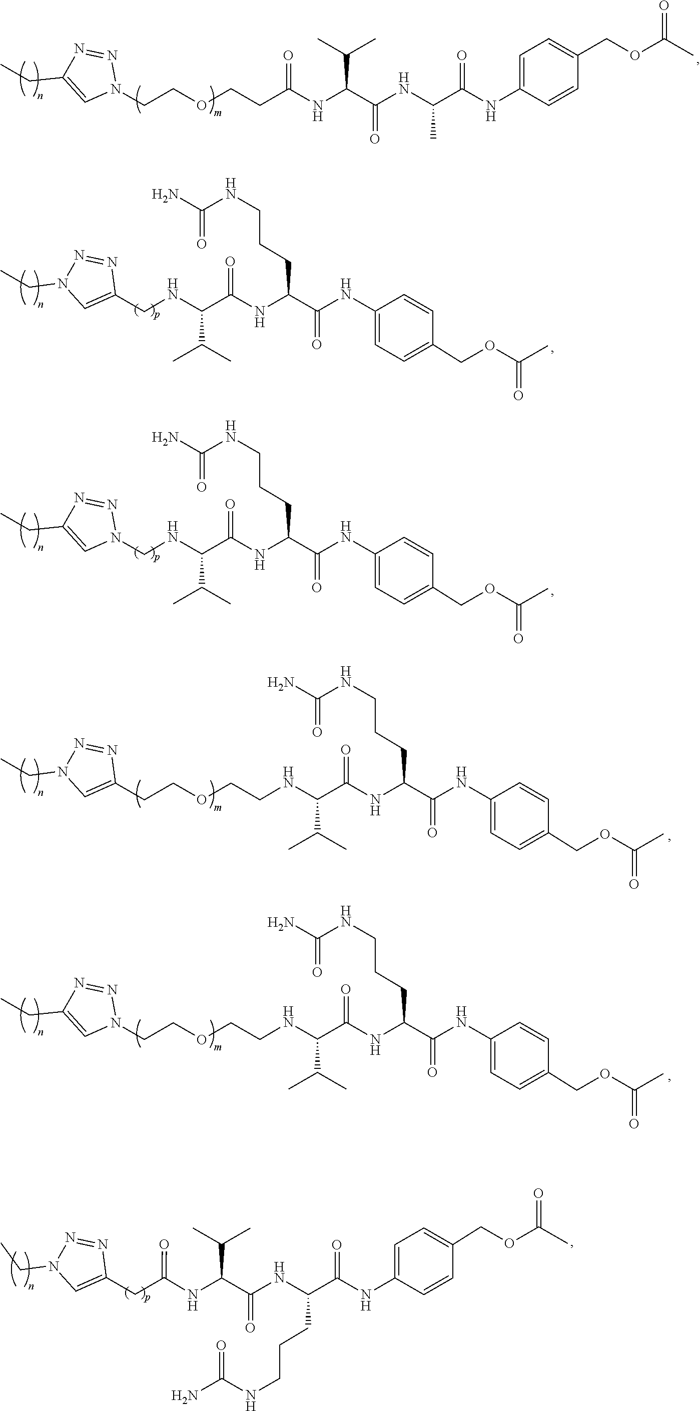

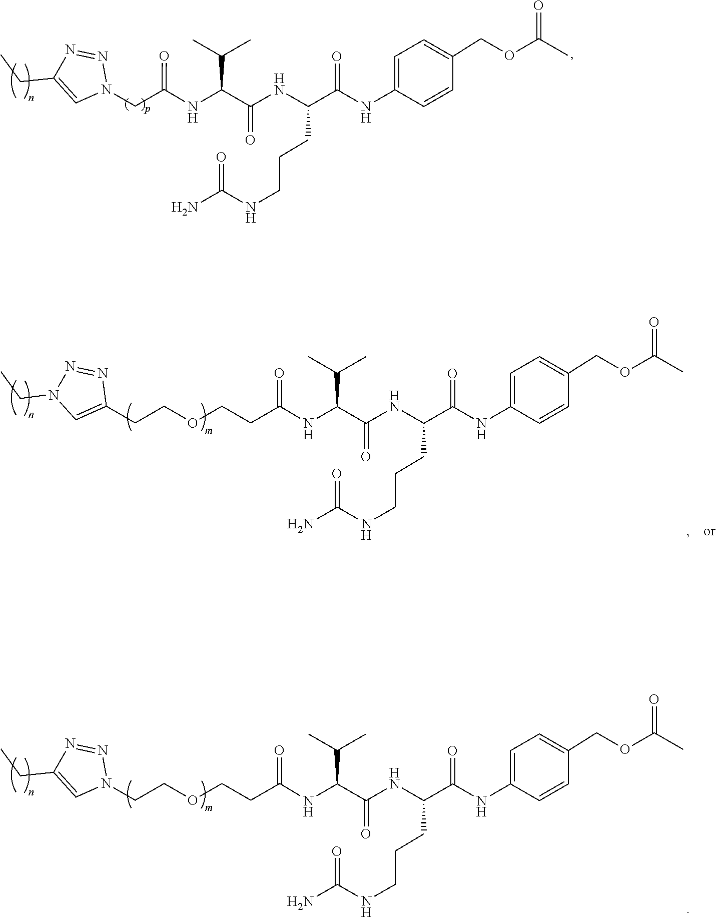

The group -L₁-(CO)_{c}—(W)_{w}—(Y)_{y}— can also represent —(CH₂)ₙ—CO—NCH₃—(CH₂)ᵤ—NCH₃—CO—(CH₂)ᵥ—, —(CH₂)ₙ—CO—NCH₃—(CH₂)ᵤ—NCH₃—CO—(CH₂)ᵥ—CO—, —(CH₂CH₂O)ₘ—CH₂—CH₂—CO—NCH₃—(CH₂)ᵤ—NCH₃—CO—(CH₂)ᵥ—, —(CH₂CH₂O)ₘ—CH₂—CH₂—CO—NCH₃—(CH₂)ᵤ—NCH₃—CO—(CH₂)ᵥ—CO—, —CH₂-para-cyclohexyl-CO—NCH₃—(CH₂)ᵤ—NCH₃—CO—(CH₂)ᵥ—, —CH₂-para-cyclohexyl-CO—NCH₃—(CH₂)ᵤ—NCH₃—CO—(CH₂)ᵥ—CO—, -aryl-(CH₂)ₙ—CO—NCH₃—(CH₂)ᵤ—NCH₃—CO—(CH₂)ᵥ—, -aryl-(CH₂)ₙ—CO—NCH₃—(CH₂)ᵤ—NCH₃—CO—(CH₂)ᵥ—CO—, —(CH₂)ₙ—CO—NCH₃—(CH₂)ᵤ—NCH₃—, —(CH₂)ₙ—CO—NCH₃—(CH₂)ᵤ—NCH₃—CO—, —(CH₂CH₂O)ₘ—CH₂—CH₂—CO—NCH₃—(CH₂)ᵤ—NCH₃—, —(CH₂CH₂O)ₘ—CH₂—CH₂—CO—NCH₃—(CH₂)ᵤ—NCH₃—, —(CH₂CH₂O)ₘ—CH₂—CH₂—CO—NCH₃—(CH₂)ᵤ—NCH₃—CO—, —CH₂-para-cyclohexyl-CO—NCH₃—(CH₂)ᵤ—NCH₃—, —CH₂-para-cyclohexyl-CO—NCH₃—(CH₂)ᵤ—NCH₃—CO—, -aryl-(CH₂)ₙ—CO—NCH₃—(CH₂)ᵤ—NCH₃—, -aryl-(CH₂)ₙ—CO—NCH₃—(CH₂)ᵤ—NCH₃—, -aryl-(CH₂)ₙ—CO—NCH₃—(CH₂)ᵤ—NCH₃—CO—, —(CH₂)ₙ—CO—NH—(CH₂)ᵤ—NH—, —(CH₂)ₙ—CO—NH—(CH₂)ᵤ—NH—CO—, —(CH₂CH₂O)ₘ—CH₂—CH₂—CO—NH—(CH₂)ᵤ—NH—, —(CH₂CH₂O)ₘ—CH₂—CH₂—CO—NH—(CH₂)ᵤ—NH—CO—, —CH₂-para-cyclohexyl-CO—NH—(CH₂)ᵤ—NH—, —CH₂-para-cyclohexyl-CO—NH—(CH₂)ᵤ—NH—CO—, -aryl-(CH₂)ₙ—CO—NH—(CH₂)ᵤ—NH—, -aryl-(CH₂)ₙ—CO—NH—(CH₂)ᵤ—NH—CO—, —(CH₂)ₙ-Val-Cit para-aminobenzyl-O—CO—NCH₃—(CH₂)ᵤ—NCH₃—CO—(CH₂)ᵥ—, —(CH₂)ₙ-Val-Cit para-aminobenzyl-O—CO—NCH₃—(CH₂)ᵤ—NCH₃—CO—(CH₂)ᵥ—CO—, —(CH₂)ₙ—CO-Val-Cit para-aminobenzyl-O—CO—NCH₃—(CH₂)ᵤ—NCH₃—CO—(CH₂)ᵥ—, —(CH₂)ₙ—CO-Val-Cit para-aminobenzyl-O—CO—NCH₃—(CH₂)ᵤ—NCH₃—CO—(CH₂)ᵥ—CO—, —(CH₂CH₂O)ₘ—CH₂—CH₂-Val-Cit-para-aminobenzyl-O—CO—NCH₃—(CH₂)ᵤ—NCH₃—CO—(CH₂)ᵥ—, —(CH₂CH₂O)ₘ—CH₂—CH₂-Val-Cit-para-aminobenzyl-O—CO—NCH₃—(CH₂)ᵤ—NCH₃—CO—(CH₂)ᵥ—CO—, —(CH₂CH₂O)ₘ—CH₂—CH₂—CO-Val-Cit-Para-aminobenzyl-O—CO—NCH₃—(CH₂)ᵤ—NCH₃—CO—(CH₂)ᵥ—, —(CH₂CH₂O)ₘ—CH₂—CH₂—CO-Val-Cit Para-aminobenzyl-O—CO—NCH₃—(CH₂)ᵤ—NCH₃—CO—(CH₂)ᵥ—CO—, —CH₂-para-cyclohexyl-CO-Val-Cit Para-aminobenzyl-O—CO—NCH₃—(CH₂)ᵤ—NCH₃—CO—(CH₂)ᵥ—, —CH₂-para-cyclohexyl-CO-Val-Cit para-aminobenzyl-O—CO—NCH₃—(CH₂)ᵤ—NCH₃—CO—(CH₂)ᵥ—CO—, -aryl-(CH₂)ₙ-Val-Cit para-aminobenzyl-O—CO—NCH₃—(CH₂)ᵤ—NCH₃—CO—(CH₂)ᵥ—, -aryl-(CH₂)ₙ-Val-Cit para-aminobenzyl-O—CO—NCH₃—(CH₂)ᵤ—NCH₃—CO—(CH₂)ᵥ—CO—, -aryl-CO-Val-Cit para-aminobenzyl-O—CO—NCH₃—(CH₂)ᵤ—NCH₃—CO—(CH₂)ᵥ—, -aryl-CO-Val-Cit para-aminobenzyl-O—CO—NCH₃—(CH₂)ᵤ—NCH₃—CO—(CH₂)ᵥ—CO—, —(CH₂)ₙ-Val-Ala para-aminobenzyl-O—CO—NCH₃—(CH₂)ᵤ—NCH₃—CO—(CH₂)ᵥ—, —(CH₂)ₙ-Val-Ala para-aminobenzyl-O—CO—NCH₃—(CH₂)ᵤ—NCH₃—CO—(CH₂)ᵥ—CO—, —(CH₂)ₙ—CO-Val-Ala para-aminobenzyl-O—CO—NCH₃—(CH₂)ᵤ—NCH₃—CO—(CH₂)ᵥ—, —(CH₂)ₙ—CO-Val-Ala para-aminobenzyl-O—CO—NCH₃—(CH₂)ᵤ—NCH₃—CO—(CH₂)ᵥ—CO—, —(CH₂CH₂O)ₘ—CH₂—CH₂-Val-Ala-para-aminobenzyl-O—CO—NCH₃—(CH₂)ᵤ—NCH₃—CO—(CH₂)ᵥ—, —(CH₂CH₂O)ₘ—CH₂—CH₂-Val-Ala-para-aminobenzyl-O—CO—NCH₃—(CH₂)ᵤ—NCH₃—CO—(CH₂)ᵥ—CO—, —(CH₂CH₂O)ₘ—CH₂—CH₂—CO-Val-Ala-Para-aminobenzyl-O—CO—NCH₃—(CH₂)ᵤ—NCH₃—CO—(CH₂)ᵥ—, —(CH₂CH₂O)ₘ—CH₂—CH₂—CO-Val-Ala Para-aminobenzyl-O—CO—NCH₃—(CH₂)ᵤ—NCH₃—CO—(CH₂)ᵥ—CO—, —CH₂-para-cyclohexyl-CO-Val-Ala Para-aminobenzyl-O—CO—NCH₃—(CH₂)ᵤ—NCH₃—CO—(CH₂)ᵥ—, —CH₂-para-cyclohexyl-CO-Val-Ala para-aminobenzyl-O—CO—NCH₃—(CH₂)ᵤ—NCH₃—CO—(CH₂)ᵥ—CO—, -aryl-(CH₂)ₙ-Val-Ala para-aminobenzyl-O—CO—NCH₃—(CH₂)ᵤ—NCH₃—CO—(CH₂)ᵥ—, -aryl-(CH₂)ₙ-Val-Ala para-aminobenzyl-O—CO—NCH₃—(CH₂)ᵤ—NCH₃—CO—(CH₂)ᵥ—CO—, -aryl-CO-Val-Ala para-aminobenzyl-O—CO—NCH₃—(CH₂)ᵤ—NCH₃—CO—(CH₂)ᵥ— or -aryl-CO-Val-Ala para-aminobenzyl-O—CO—NCH₃—(CH₂)ᵤ—NCH₃—CO—(CH₂)ᵥ—CO—.

According to another particular embodiment, the group -L₁-(CO)_{c}—(W)_{w}—(Y)_{y}-represents —(CH₂)ₙ—, —(CH₂)ₙ—CO—, —(CH₂)ₙ-Val-Cit-para-aminobenzyl-O—CO—, —(CH₂)ₙ—CO-Val-Cit-para-aminobenzyl-O—CO—, —(CH₂)ₙ-Val-Ala Para-aminobenzyl-O—CO—, —(CH₂)ₙ—CO-Val-Ala Para-aminobenzyl-O—CO—, —(CH₂)ₙ-Val-Cit Para-aminobenzyl-O—CO—NH—(CH₂)ᵤ—NH—, —(CH₂)ₙ—CO-Val-Cit-para-aminobenzyl-O—CO—NH—(CH₂)ᵤ—NH—, —(CH₂)ₙ-Val-Ala Para-aminobenzyl-O—CO—NH—(CH₂)ᵤ—NH—, —(CH₂)ₙ—CO-Val-Ala-para-aminobenzyl-O—CO—NH—(CH₂)ᵤ—NH—, —(CH₂)ₙ-Val-Cit Para-aminobenzyl-O—CO—NCH₃—(CH₂)ᵤ—NCH₃—CO—, —(CH₂)ₙ—CO-Val-Cit Para-aminobenzyl-O—CO—NCH₃—(CH₂)ᵤ—NCH₃—CO—, —(CH₂)ₙ-Val-Ala Para-aminobenzyl-O—CO—NCH₃—(CH₂)ᵤ—NCH₃—CO—, or —(CH₂)ₙ—CO-Val-Ala Para-aminobenzyl-O—CO—NCH₃—(CH₂)ᵤ—NCH₃—CO—, with n and u as defined previously and notably with n=5 and u=2.

The group -L₁-(CO)_{c}—(W)_{w}—(Y)_{y}— can also represent —(CH₂)ₙ—CO—NH—(CH₂)ᵤ—NH—, —(CH₂)ₙ—CO—NH—(CH₂)ᵤ—NH—CO—, —(CH₂)ₙ—CO—NCH₃—(CH₂)ᵤ—NCH₃—, —(CH₂)ₙ—CO—NCH₃—(CH₂)ᵤ—NCH₃—CO—, —(CH₂)ₙ—CO—NCH₃—(CH₂)ᵤ—NCH₃—CO—(CH₂)ᵥ—, —(CH₂)ₙ—CO—NCH₃—(CH₂)ᵤ—NCH₃—CO—(CH₂)ᵥ—CO—, —(CH₂)ₙ-Val-Cit Para-aminobenzyl-O—CO—NCH₃—(CH₂)ᵤ—NCH₃—CO—(CH₂)ᵥ—, —(CH₂)ₙ-Val-Cit Para-aminobenzyl-O—CO—NCH₃—(CH₂)ᵤ—NCH₃—CO—(CH₂)ᵥ—CO—, —(CH₂)ₙ—CO-Val-Cit-para-aminobenzyl-O—CO—NCH₃—(CH₂)ᵤ—NCH₃—CO—(CH₂)ᵥ—, —(CH₂)ₙ—CO-Val-Cit Para-aminobenzyl-O—CO—NCH₃—(CH₂)ᵤ—NCH₃—CO—(CH₂)ᵥ—CO—, —(CH₂)ₙ-Val-Ala Para-aminobenzyl-O—CO—NCH₃—(CH₂)ᵤ—NCH₃—CO—(CH₂)ᵥ—, —(CH₂)ₙ-Val-Ala Para-aminobenzyl-O—CO—NCH₃—(CH₂)ᵤ—NCH₃—CO—(CH₂)ᵥ—CO—, —(CH₂)ₙ—CO-Val-Ala Para-aminobenzyl-O—CO—NCH₃—(CH₂)ᵤ—NCH₃—CO—(CH₂)ᵥ—, or —(CH₂)ₙ—CO-Val-Ala Para-aminobenzyl-O—CO—NCH₃—(CH₂)ᵤ—NCH₃—CO—(CH₂)ᵥ—CO—, with n, u and v as defined previously and notably with n=5 and u=v=2.

According to a preferred embodiment, the group -L₁-(CO)_{c}—(W)_{w}—(Y)_{y}— represents —(CH₂)ₙ—CO—, —(CH₂)ₙ—CO-Val-Cit Para-aminobenzyl-O—CO—, —(CH₂)ₙ—CO-Val-Ala-para-aminobenzyl-O—CO—, —(CH₂)ₙ—CO-Val-Cit-para-aminobenzyl-O—CO—NH—(CH₂)ᵤ—NH—, —(CH₂)ₙ—CO-Val-Ala Para-aminobenzyl-O—CO—NH—(CH₂)ᵤ—NH—, —(CH₂)ₙ—CO-Val-Cit-Para-aminobenzyl-O—CO—NCH₃—(CH₂)ᵤ—NCH₃—CO—, or —(CH₂)ₙ—CO-Val-Ala-para-aminobenzyl-O—CO—NCH₃—(CH₂)ᵤ—NCH₃—CO—, with n and u as defined previously and notably with n=5 and u=2.

The group -L₁-(CO)_{c}—(W)_{w}—(Y)_{y}— can also represent —(CH₂)ₙ—CO—NH—(CH₂)ᵤ—NH—, —(CH₂)ₙ—CO—NH—(CH₂)ᵤ—NH—CO—, —(CH₂)ₙ—CO—NCH₃—(CH₂)ᵤ—NCH₃—, —(CH₂)ₙ—CO—NCH₃—(CH₂)ᵤ—NCH₃—CO—, —(CH₂)ₙ—CO—NCH₃—(CH₂)ᵤ—NCH₃—CO—(CH₂)ᵥ—, —(CH₂)ₙ—CO—NCH₃—(CH₂)ᵤ—NCH₃—CO—(CH₂)ᵥ—CO—, —(CH₂)ₙ—CO-Val-Cit Para-aminobenzyl-O—CO—NCH₃—(CH₂)ᵤ—NCH₃—CO—(CH₂)ᵥ—, —(CH₂)ₙ—CO-Val-Cit Para-aminobenzyl-O—CO—NCH₃—(CH₂)ᵤ—NCH₃—CO—(CH₂)ᵥ—CO—, —(CH₂)ₙ—CO-Val-Ala Para-aminobenzyl-O—CO—NCH₃—(CH₂)ᵤ—NCH₃—CO—(CH₂)ᵥ—, or —(CH₂)ₙ—CO-Val-Ala Para-aminobenzyl-O—CO—NCH₃—(CH₂)ᵤ—NCH₃—CO—(CH₂)ᵥ—CO—, with n, u and v as defined previously and notably with n=5 and u=v=2.

The group

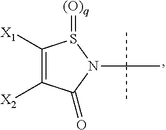

preferably

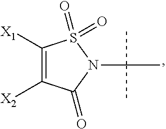

is the functional group which will react with the binding unit, such as an antibody, to attach a drug moiety on it, thanks to sulfhydryl groups present on said binding unit. Sulfhydryl groups can be generated by reduction of intramolecular disulfide bonds of the binding unit, if present, in particular in antibodies. Alternatively, sulfhydryl groups can be generated by reaction of an amino group of a lysine moiety of the binding unit with 2-iminothiolane or other sulfhydryl generating reagents. In specific embodiments, the binding unit, such as an antibody, is engineered to carry one or more lysines. More preferably, the binding unit, such as an antibody, can be engineered to carry one or more cysteines (cf. ThioMabs).

X₁ and X₂ represent, independently of each other, H, a halogen atom such as Cl or Br, a (C₁-C₆)alkoxy, an aryloxy optionally substituted, or —O—(CH₂CH₂O)ᵣH, provided that X₁ and X₂ do not represent H at the same time. The aryloxy is more particularly optionally substituted with one or several groups (e.g. one) selected from halogen, CN, NO₂ and an aryloxy (e.g. phenyloxy) optionally substituted with one or several halogen atoms such as fluorine atoms. In particular the aryloxy is optionally substituted with one or several (e.g. one) groups selected from CN, NO₂ and pentafluorophenyloxy, notably optionally substituted with CN. The aryloxy can be in particular a phenyloxy.

According to a particular embodiment, X₁ and X₂ represent, independently of each other, H, a halogen atom such as Cl or Br, a (C₁-C₆)alkoxy or an aryloxy optionally substituted, provided that X₁ and X₂ do not represent H at the same time. The aryloxy is more particularly optionally substituted with one or several groups (e.g. one) selected from halogen, CN, NO₂ and an aryloxy (e.g. phenyloxy) optionally substituted with one or several halogen atoms such as fluorine atoms. In particular the aryloxy is optionally substituted with one or several (e.g. one) groups selected from CN, NO₂ and pentafluorophenyloxy, notably optionally substituted with CN. The aryloxy can be in particular a phenyloxy.

According to another particular embodiment, X₁ and X₂ represent, independently of each other, H, Cl, Br, a methoxy or a phenyloxy substituted with CN, notably H, Cl or Br, provided that X₁ and X₂ do not represent H at the same time.

Advantageously, X₁ and X₂ are identical and not H or one of X₁ and X₂ is H and the other is not H. When X₁ and/or X₂ is not H, it is a halogen atom such as Cl or Br, a (C₁-C₆)alkoxy, an aryloxy optionally substituted, or —O—(CH₂CH₂O)ᵣH; in particular a halogen atom such as Cl or Br, a (C₁-C₆)alkoxy or an aryloxy optionally substituted; preferably Cl, Br, a methoxy or a phenyloxy substituted with CN; in particular Cl or Br.

q represents 0, 1 or 2. Preferably, q represents 2.

X₃ represents a functional group (optionally with the terminal nitrogen of Z when y=z=1 and Z is —NR₄—(CH₂)ᵤ—NR₅— or with the terminal nitrogen of Z′ when c=w=y=0 and z′=1 and Z′ is —NR₄—(CH₂)ᵤ—NR₅—) which aims to react with the drug (QH or Q-OH) in order ultimately to covalently link the drug to the binding unit, such as an antibody.

It could also be envisaged to introduce first the spacer unit Y and the amino acid unit (W)_{w}, when present, on the drug moiety, before linking the stretcher unit bearing the sulfomaleimide function. In this case, a compound of formula (I) with w=y=0 (i.e. comprising only the stretcher unit and the sulfomaleimide function) will be used and X₃ represents in this case a functional group which will react with the amino acid unit (W)_{w} or the spacer unit Y already attached on the drug unit.

X₃ represents H when y=z=1 and Z is —NR₄—(CH₂)ᵤ—NR₅— or when c=w=y=0, z′=1 and Z′ is —NR₄—(CH₂)ᵤ—NR₅— (and form a NH functional group with the terminal nitrogen of the Z or Z′ group) and in the other cases, X₃ represents OH, NH₂ or a leaving group, such as OH or a leaving group. The leaving group can be a halogen atom (e.g. C₁, Br, I), a sulfonate (e.g. OTf, OMs, OTs), N-succinimidyloxy, 4-nitro-phenyloxy, pentafluorophenyloxy or N-benzotriazoloxy. In particular, X₃ represents H when y=z=1 and Z is —NR₄—(CH₂)ᵤ—NR₅— or when c=w=y=0, z′=1 and Z′ is —NR₄—(CH₂)ᵤ—NR₅- and in the other cases, X₃ can be more particularly OH, Cl or N-succinimidyloxy.

### Drug Moiety

The drug moiety (Q) is a residue of a drug QH or of a drug Q-OH.

The drug according to the present invention can be any drug useful in human or veterinary therapy, notably for the treatment of cancer. It can be notably a cytotoxic agent. Advantageously, such a drug comprises a functional group to be able to link this drug to the linker moiety. It can also be envisaged to add such a functional group onto the drug to perform the linking. This functional group can be for example OH, SH, NH or COOH and will react with the X₃ end of the linker to link the drug to the linker moiety. The coupling reaction can be for example a nucleophilic substitution (e.g. reaction of OH, SH, NH or COOH with X₃=leaving group), a peptide coupling (e.g. reaction of COOH with X₃═NH₂ or ZX₃ or Z′X₃ ending by NH), an esterification (reaction between COOH and OH), a Mitsunobu reaction, etc.

The drug moiety Q can be for example:
a residue of an auristatin derivative such as a residue of monomethyl auristatin F (MMAF) (linked by its terminal NH or COOH group), monomethyl auristatin E (MMAE) (linked by its terminal NH or OH group), monomethyl dolastatin-10 (linked by its terminal NH group) or a derivative thereof such as a drug moiety of formula (C) as defined below;

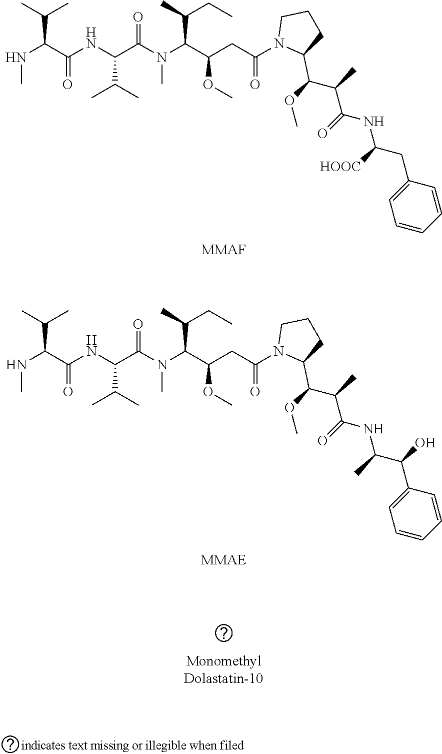

a residue of an anthracycline, such as a residue of daunorubicine, doxorubicine, epirubicine or idarubicine (linked by an NH₂ group or the OH group of —COCH₂OH), or a derivative thereof such as 2-pyrrolinodoxorubicine or pro-2-pyrrolinodoxorubicine (linked by the OH group of —COCH₂OH), or PNU-159682 (linked by the OH group of —COCH₂OH) or a derivative thereof, in particular a residue of doxorubicine (linked by an NH₂ group or the OH group of —COCH₂OH), 2-pyrrolinodoxorubicine, pro-2-pyrrolinodoxorubicine (linked by the OH group of —COCH₂OH) or PNU-159682 (linked by the OH group of —COCH₂OH) or a derivative of PNU-159682 notably as illustrated below; preferably a residue of PNU-159682 (linked by the OH group of —COCH₂OH) or a residue of a derivative of PNU-159682 as illustrated below (linked by COOH);

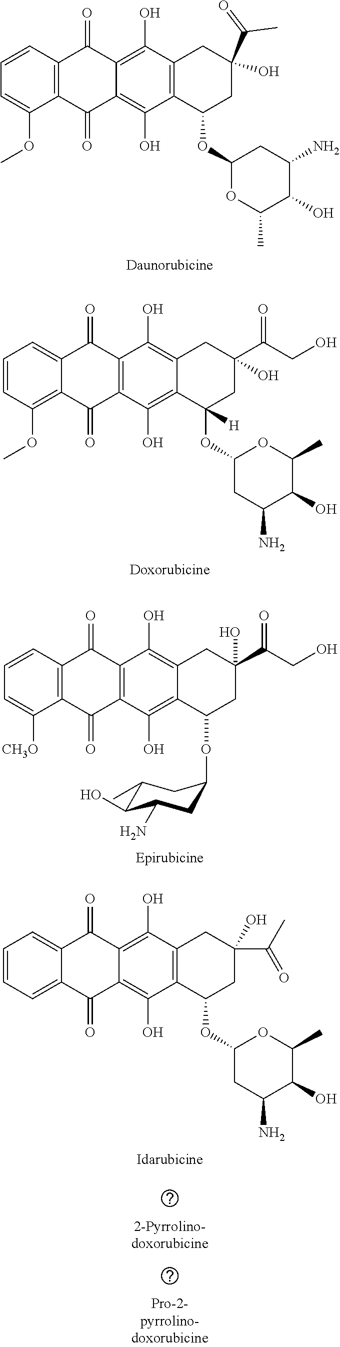

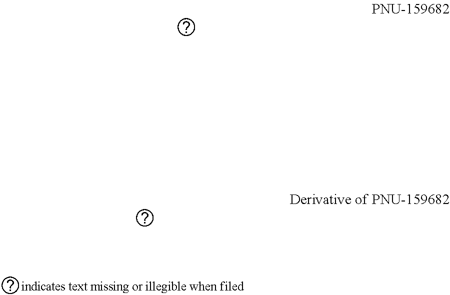

a residue of camptothecin or a derivative thereof such as SN-38 (linked by its OH group);

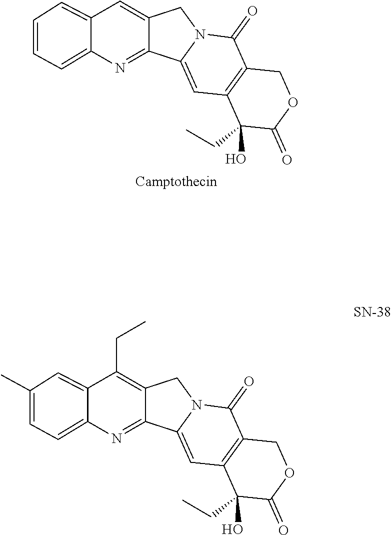 [SMILES: CC[C@@]1(O)C(=O)OCc2c1cc1n(c2=O)Cc2cc3ccccc3nc2-1 ; CCc1c2c(nc3ccc(C)cc13)-c1cc3c(c(=O)n1C2)COC(=O)[C@]3(O)CC]

a residue of a tubulysin, such as tubulysin A, tubulysin B, tubulysin C or tubulysin D (linked by a COOH group or an OH group when present);

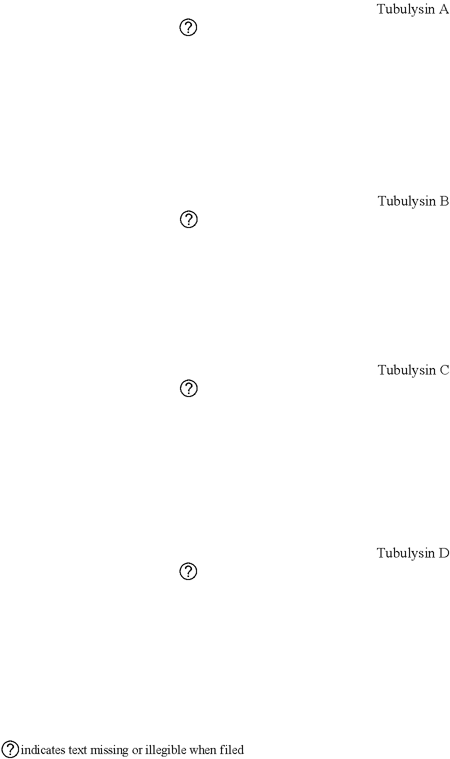

a residue of a calicheamicin, such as esperamicin or calicheamicin yl, or a derivative thereof such as N-acetyl dimethyl hydrazide calicheamicin (linked by its hydrazide moiety);

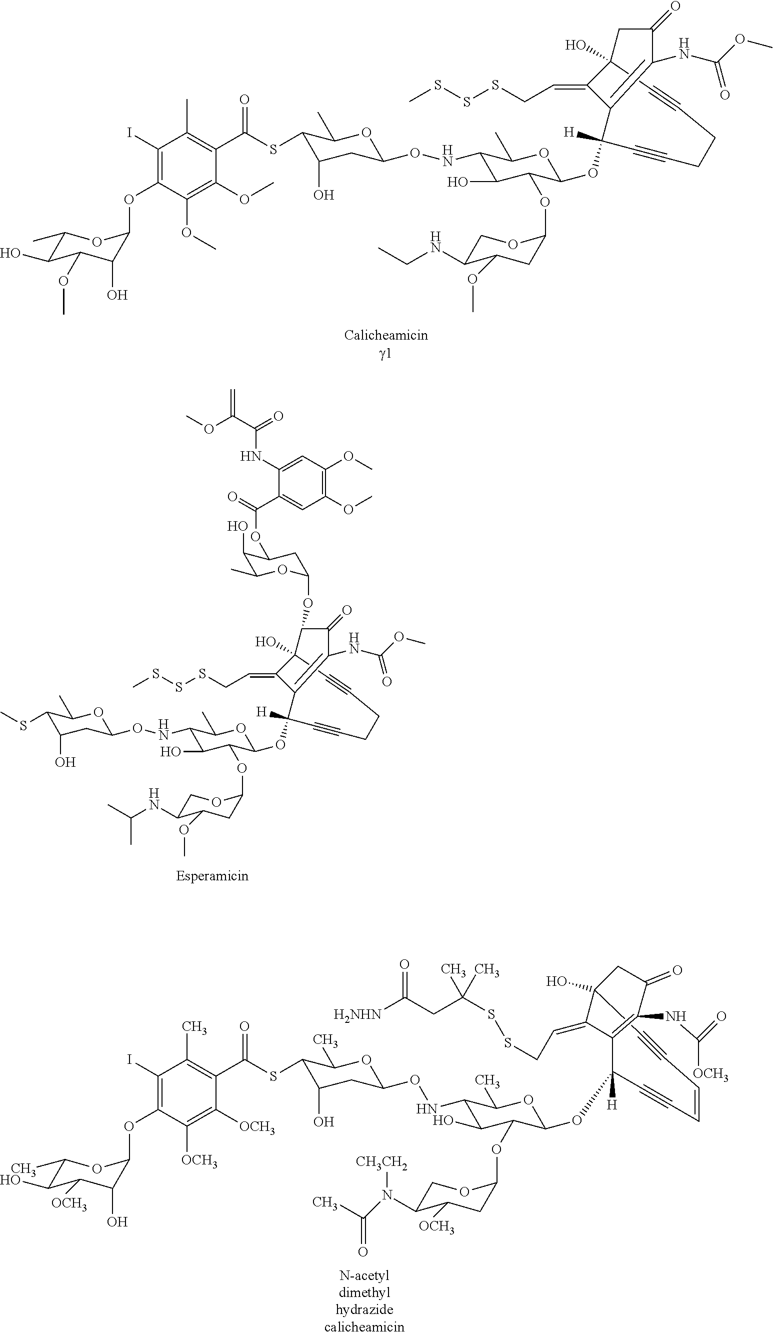

a residue of a maytansinoid, such as maytansine (also called maitansine) or a derivative thereof such as DM1 or DM4 (linked by a SH group); in particular a residue of DM1 or DM4 (linked by a SH group);

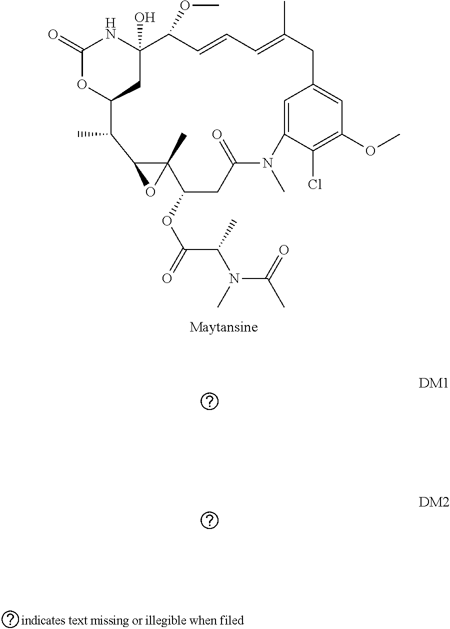 [SMILES: COc1cc2cc(c1Cl)N(C)C(=O)C[C@H](OC(=O)[C@H](C)N(C)C(C)=O)[C@]1(C)O[C@H]1[C@H](C)[C@@H]1C[C@@](O)(NC(=O)O1)[C@H](OC)/C=C/C=C(\C)C2]

a residue of a duocarmycin such as duocarmycin A, duocarmycin B1, duocarmycin B2, duocarmycin C1, duocarmycin C2, duocarmycin D duocarmycin SA, or CC-1065 (linked by a CONH₂ group); in particular a residue of CC-1065 (linked by a CONH₂ group);

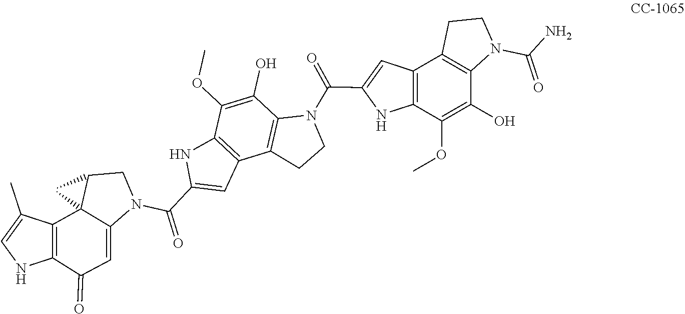

a residue of an amanitine (linked by an OH, NH, COOH or CONH₂ group, in particular an OH group) such as α-amanitine, β-amanitine, γ-amanitine or ε-amanitine; in particular a residue of α-amanitine (linked notably by a CH₂OH group);

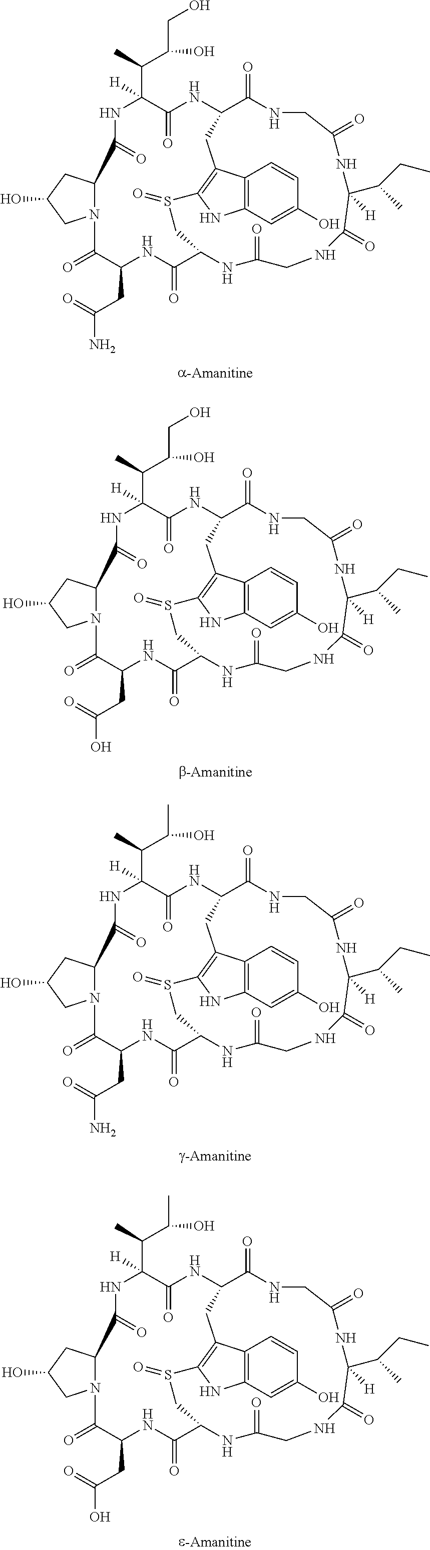

a residue of a pyrrolobenzodiazepine (PBD) such as anthramycin (linked by its OH or NH₂ group) or SGD-1882 (linked by its NH₂ group); in particular a residue of

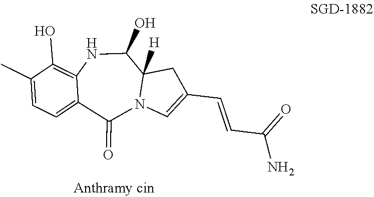 [SMILES: Cc1ccc2c(c1O)N[C@@H](O)[C@@H]1CC(/C=C/C(N)=O)=CN1C2=O]

a residue of an activator of Immune check point such as a residue of a STING (stimulator of interferon genes) agonist advantageously of formula (D) as defined below (linked by OH, SH or NH) or a residue of an IDO (indoleamine 2,3-dioxygenase) inhibitor such as epacadostat (INCB024360) or BMS-986205.

Advantageously, the drug moiety Q is:
a residue of an auristatin derivative such as a residue of MMAF (linked by its terminal NH or COOH group), MMAE (linked by its terminal NH or OH group), or monomethyl dolastatin-10 (linked by its terminal NH group) or a drug moiety of formula (C) as defined below;
a residue of a STING agonist, notably of formula (D) as defined below; or
a residue of an anthracycline, such as defined above and preferably a residue of PNU-159682 or a derivative thereof as illustrated below:

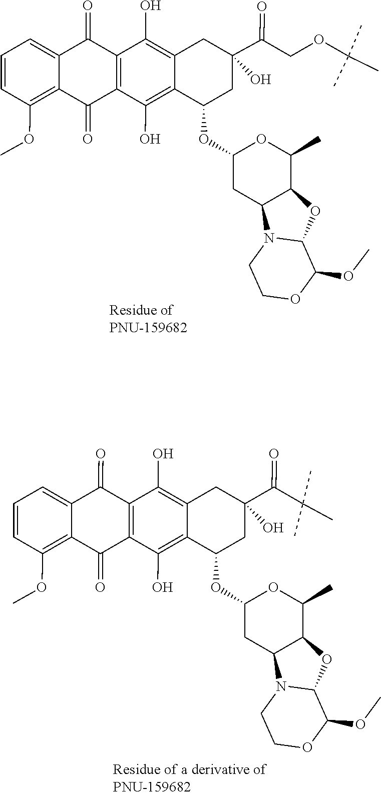

The drug moiety Q is in particular a residue of an anthracycline, such as defined above and preferably a residue of PNU-159682 or a derivative thereof as illustrated below:

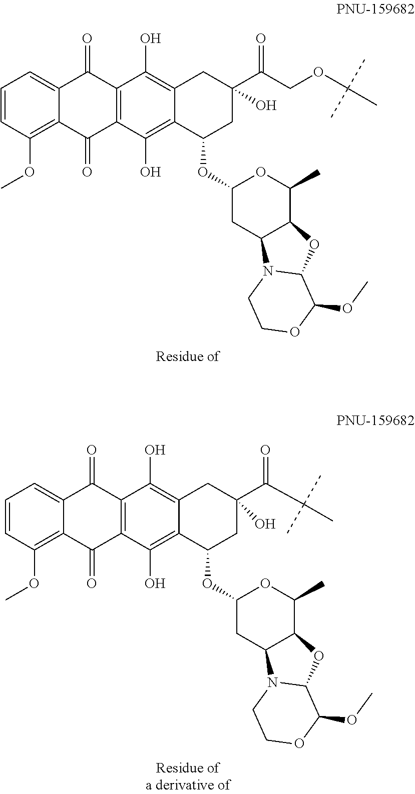

According to a first embodiment, the residue of an auristatin derivative has the following formula (C):

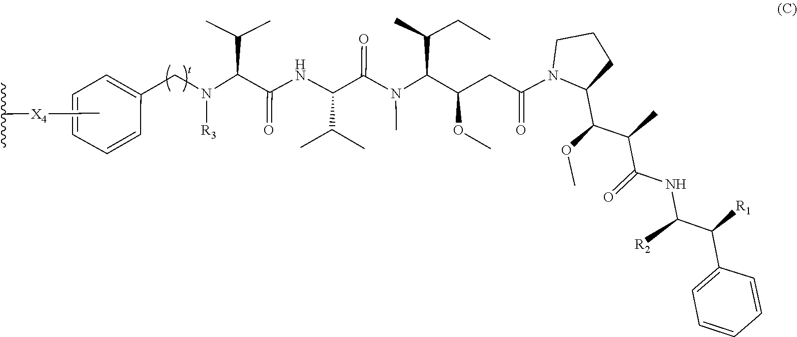

where:
R₁ is H or OH,
R₂ is a (C₁-C₆)alkyl (e.g. methyl), COOH, COO—((C₁-C₆)alkyl) (such as COOMe) or a thiazolyl (such as thiazol-2-yl),
R₃ is H or a (C₁-C₆)alkyl (such as methyl), in particular a (C₁-C₆)alkyl group,
X₄ is O or NR₉,
R₉ is H or (C₁-C₆)alkyl (such as methyl), and
t is an integer from 1 and 8, in particular from 1 to 6, advantageously from 1 to 4, preferably is 1 or 2.

According to a particular embodiment:
R₁ is OH and Reis (C₁-C₆)alkyl such as methyl; or
R₁ is H and Reis thiazolyl such as thiazol-2-yl, COO—(C₁-C₆)alkyl such as COOMe, or COOH.

Advantageously, R₁ is H and R₂ is thiazolyl such as thiazol-2-yl, COO—(C₁-C₆)alkyl such as COOMe, or COOH. Preferably R₁ is H and R₂ is COOH or COOMe, in particular COOH.

t is an integer from 1 and 8, in particular from 1 to 6, advantageously from 1 to 4, preferably is 1 or 2.

Advantageously, R₃ is a (C₁-C₆)alkyl group and preferably a methyl group.

According to a particular embodiment, R₁ is H, R₂ is COOH or COOMe (preferably COOH), R₃ is methyl and t is 1 or 2.

Advantageously, X₄ is NR₉ with R₉ being H or (C₁-C₆)alkyl, preferably being H or methyl.

In a preferred embodiment:
R₁ is H, R₂ is COOH, R₃ is methyl, X₄ is NR₉, R₉ is methyl and t is 1 or 2, or
R₁ is H, R₂ is COOH, R₃ is methyl, X₄ is NR₉, R₉ is H and t is 1 or 2.

According to a preferred embodiment, the X₄ group is located on the phenyl ring in a para position in relation to the (CH₂)ₜ group.

Advantageously, the residue of an auristatin of formula (C) is chosen from among the following moieties

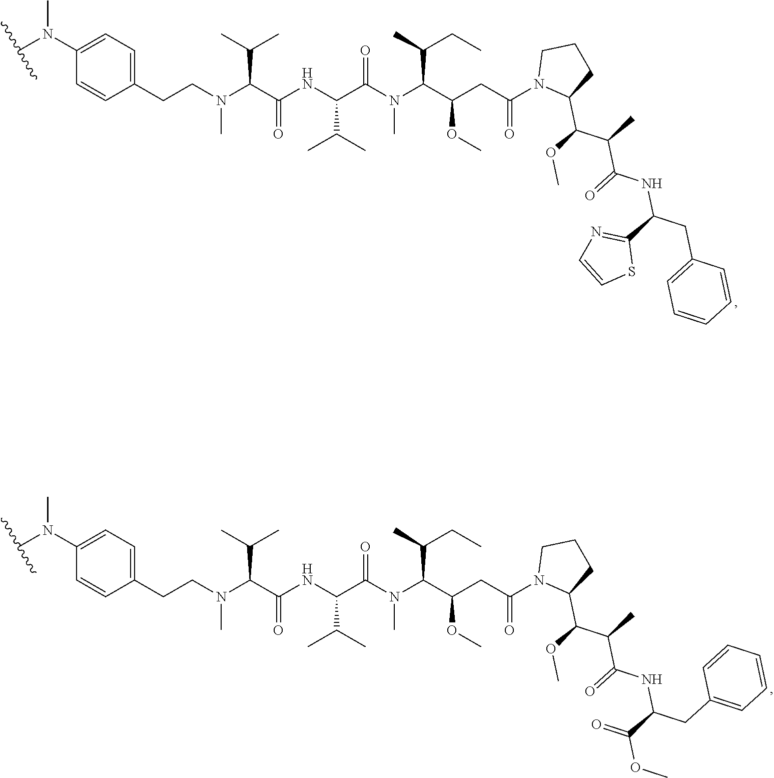 [SMILES: *N(C)c1ccc(CCN(C)[C@H](C(=O)N[C@H](C(=O)N(C)[C@@H]([C@@H](C)CC)[C@@H](CC(=O)N2CCC[C@H]2[C@H](OC)[C@@H](C)C(=O)N[C@@H](Cc2ccccc2)c2nccs2)OC)C(C)C)C(C)C)cc1 ; *N(C)c1ccc(CCN(C)[C@H](C(=O)N[C@H](C(=O)N(C)[C@@H]([C@@H](C)CC)[C@@H](CC(=O)N2CCC[C@H]2[C@H](OC)[C@@H](C)C(=O)N[C@@H](Cc2ccccc2)C(=O)OC)OC)C(C)C)C(C)C)cc1]

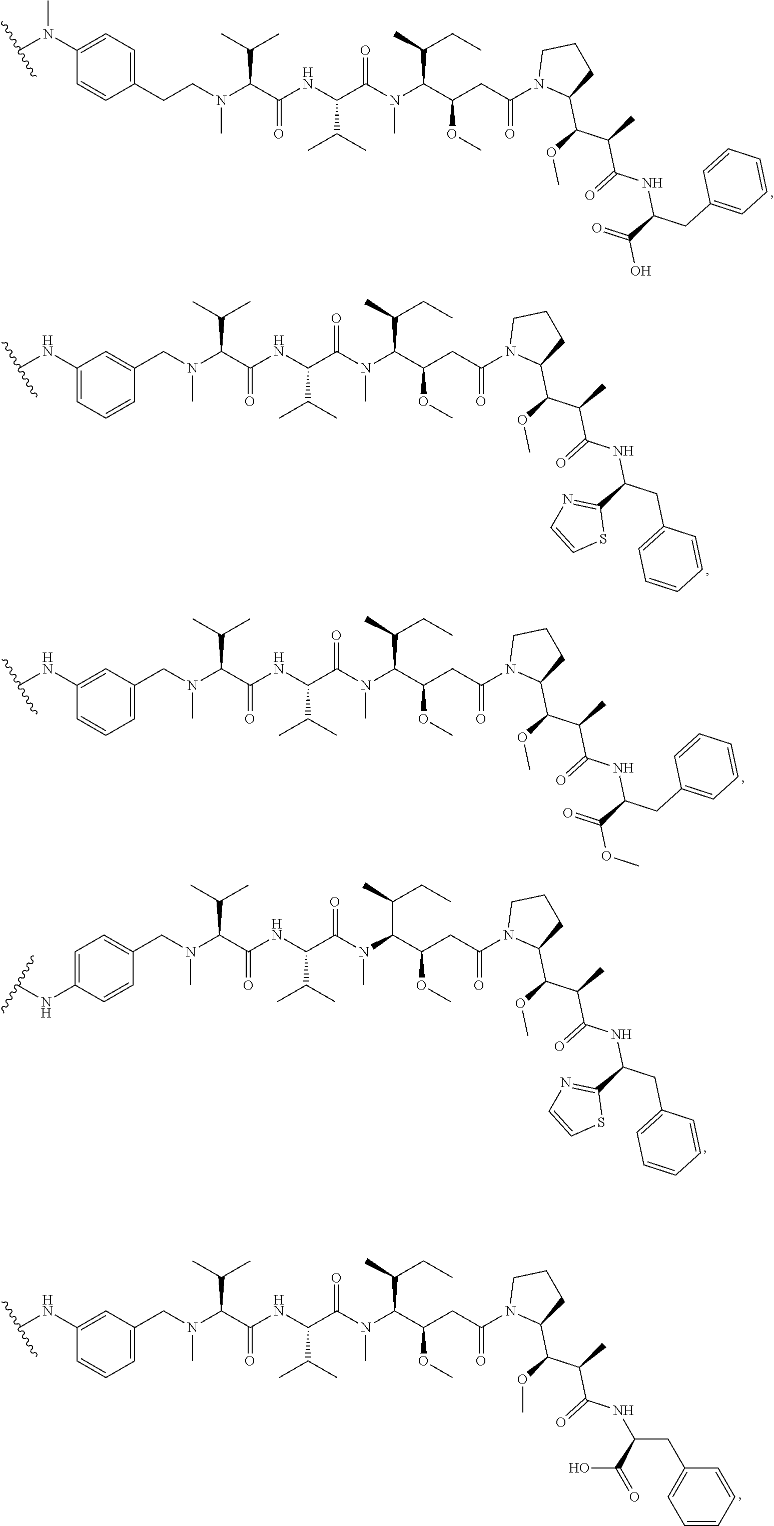

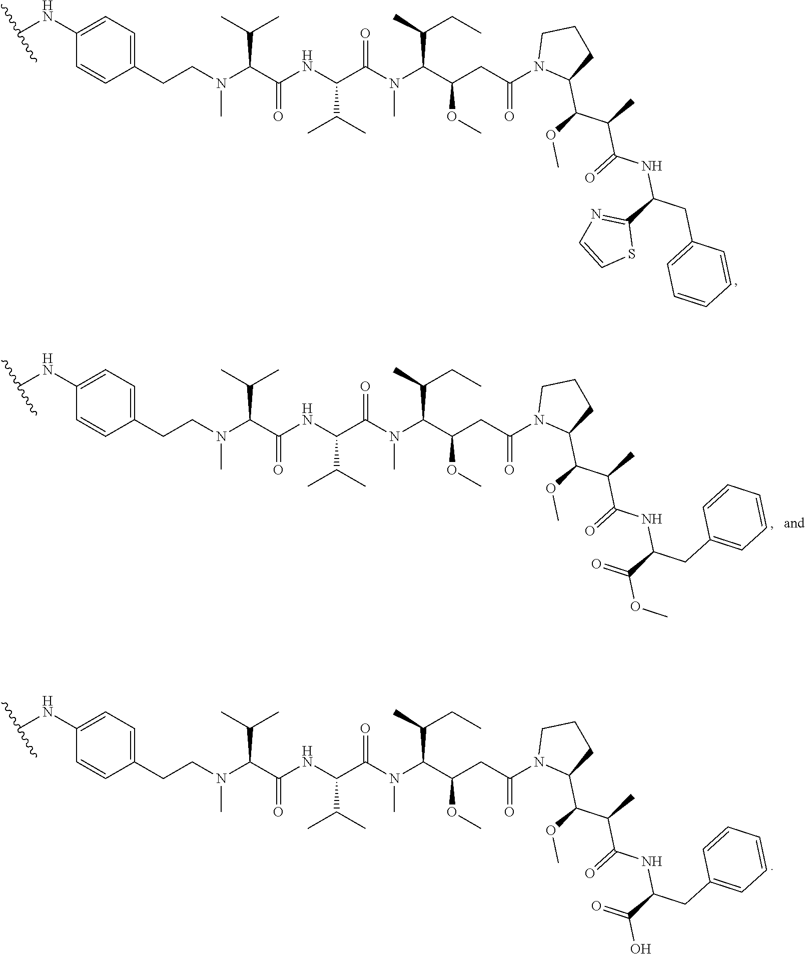 [SMILES: *Nc1ccc(CCN(C)[C@H](C(=O)N[C@H](C(=O)N(C)[C@@H]([C@@H](C)CC)[C@@H](CC(=O)N2CCC[C@H]2[C@H](OC)[C@@H](C)C(=O)N[C@@H](Cc2ccccc2)c2nccs2)OC)C(C)C)C(C)C)cc1 ; *Nc1ccc(CCN(C)[C@H](C(=O)N[C@H](C(=O)N(C)[C@@H]([C@@H](C)CC)[C@@H](CC(=O)N2CCC[C@H]2[C@H](OC)[C@@H](C)C(=O)N[C@@H](Cc2ccccc2)C(=O)OC)OC)C(C)C)C(C)C)cc1 ; *Nc1ccc(CCN(C)[C@H](C(=O)N[C@H](C(=O)N(C)[C@@H]([C@@H](C)CC)[C@@H](CC(=O)N2CCC[C@H]2[C@H](OC)[C@@H](C)C(=O)N[C@@H](Cc2ccccc2)C(=O)O)OC)C(C)C)C(C)C)cc1]

The preparation of such an auristatin derivative is disclosed in WO2014/174064 or WO2015/162293 for example.

According to a second embodiment, the STING agonist has the following formula (X):

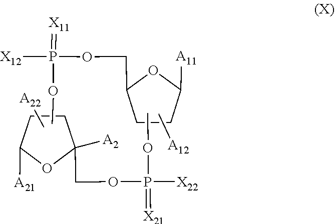

where:
X₁₁ and X₂₁ are independently O or S, preferably O,
X₁₂ and X₂₂ are independently OH or SH, preferably SH,
A₁₁ and A₂₁ are independently a group of formula:

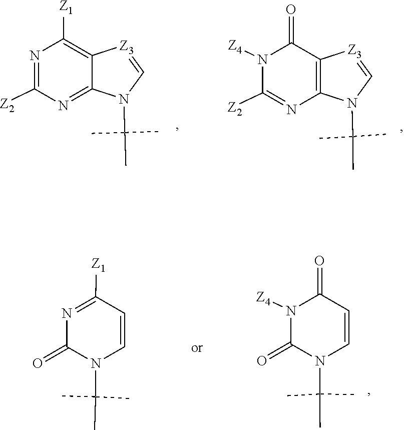

preferably

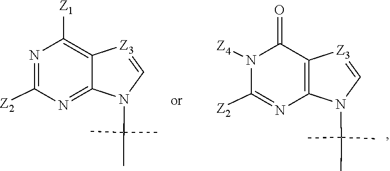

where:
   Z₁ is OR₁₁ or NR₁₁R₁₂, with R₁₁ and R₁₂ being independently H, R₁₃ or COR₁₃, with R₁₃ being (C₁-C₆)alkyl, aryl or aryl(C₁-C₆)alkyl,
Z₂ is H or NR₂₁R₂₂, with R₂₁ and R₂₂ being independently H, R₂₃ or COR₂₃, with R₂₃ being (C₁-C₆)alkyl, aryl or aryl(C₁-C₆)alkyl,
Z₃ is N or CR₃₃, preferably N, with R₃₃ being H or a halogen atom such as F or C₁, and
Z₄ is H or a (C₁-C₆)alkyl,
A₁₂ and A₂₂ are independently H, OH or F, and
A₂ is H or A₂ and A₂₂ are linked together with A₂ being CH₂ and A₂₂ being O.

When Z₁ is OH or Z₄ is H, the following tautomer forms can be obtained:

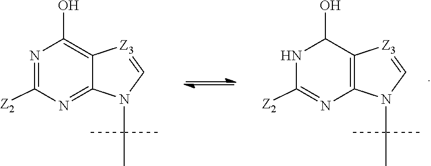

According to a particular embodiment, the STING agonist has one of the following formulas:

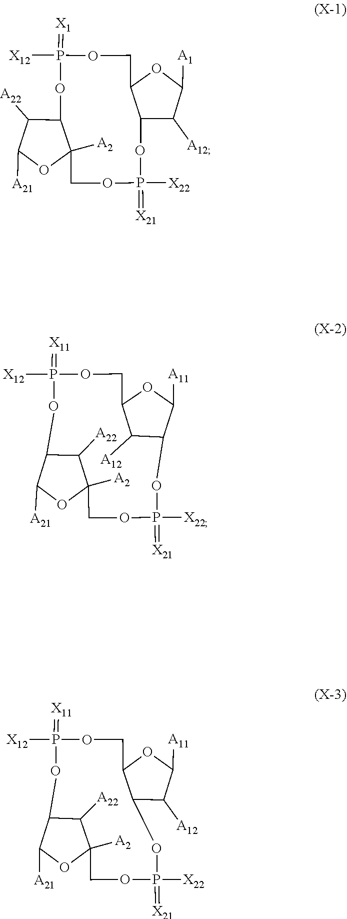

where X₁₁, X₂₁, X₁₂, X₂₂, A₁₁, A₂₁, A₁₂, A₂₂ and A₂ are as defined above or below.

According to another particular embodiment, the STING agonist has one of the following formulas:

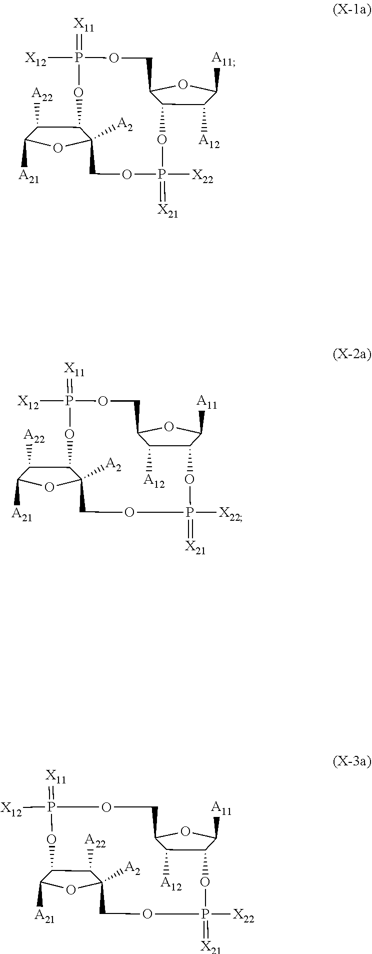

where X₁₁, X₂₁, X₁₂, X₂₂, A₁₁, A₂₁, A₁₂, A₂₂ and A₂ are as defined above or below.

Advantageously, X₁₁ and X₂₁ both are O. Advantageously, at least one of X₁₂ and X₂₂ is SH and preferably X₁₂ and X₂₂ both are SH. Preferably, X₁₁ and X₂₁ both are 0 and X₁₂ and X₂₂ both are SH.

In particular, R₁₁ and R₁₂ both are H and advantageously R₁₁, R₁₂, R₂₁ and R₂₂ each are H.

Z₃ in particular is N. Advantageously, Z₁ is OH or NH₂; Z₂ is H or NH₂; Z₃ is N; and Z₄ is H.

Preferably, An and A₂₁ are independently selected from

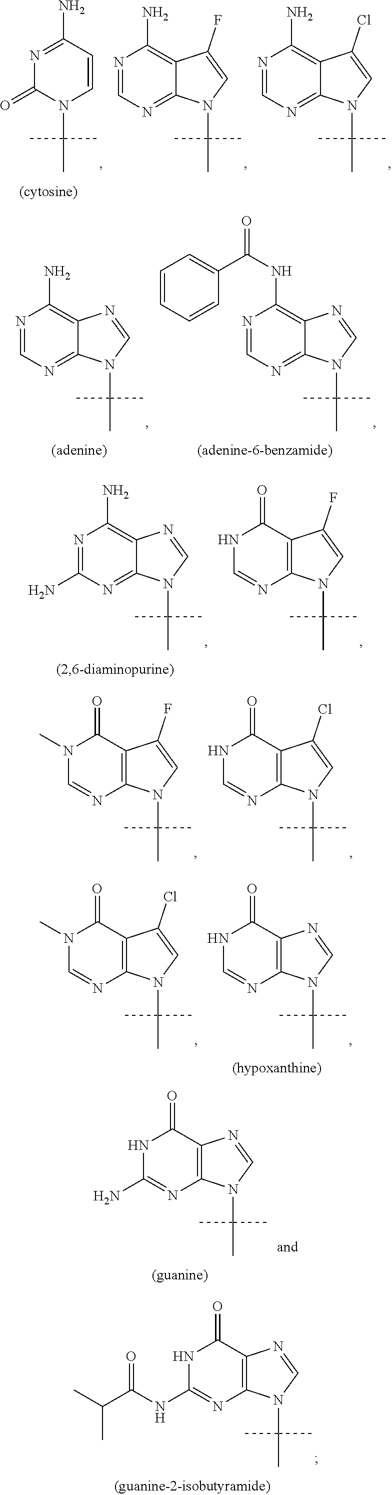

more preferably selected from cytosine, adenine, adenine-6-benzamide, 2,6-diaminopurine, hypoxanthine, guanine and guanine-2-isobutyramide; most preferably selected from adenine, hypoxanthine and guanine.

It can be in particular ADU-S 100 of following formula:

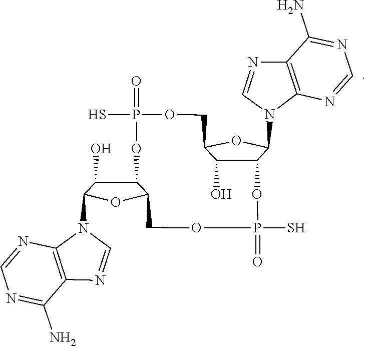 [SMILES: Nc1ncnc2c1ncn2[C@@H]1O[C@@H]2COP(=O)(S)O[C@@H]3[C@H](O)[C@@H](COP(=O)(S)O[C@H]2[C@H]1O)O[C@H]3n1cnc2c(N)ncnc21]

Alternatively it can be in particular one of the compounds specifically disclosed in Lioux et al. J. Med. Chem., 2016, 59 (22), pp 10253-10267.

The preparation of such a STING agonist is disclosed in WO2014/179335, WO2016/096174, WO2016/145102, WO2017/106740 or WO2018/100558 for example.

The said STING agonist is linked to the linker moiety by a SH, OH or NH group present on the molecule, i.e. by the group X₁₂ (OH or SH), X₂₂ (OH or SH), Z₁ when at least one of Rif and R₁₂ is H (OH, NHR₁₁ or NHR₁₂), or Z₂ when at least one of R₂₁ and R₂₂ is H (NHR₂₁ or NHR₂₂). Preferably, at least one of X₁₂ and X₂₂ is SH and the STING agonist is linked by this SH group.

In consequence, the residue of STING agonist has advantageously the following formula (D), (D-1), (D-2), (D-3), (D-1a), (D-2a) or (D-3a):

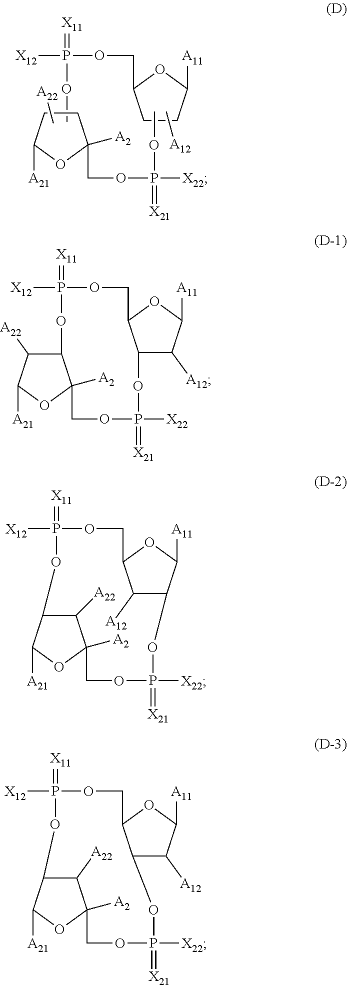

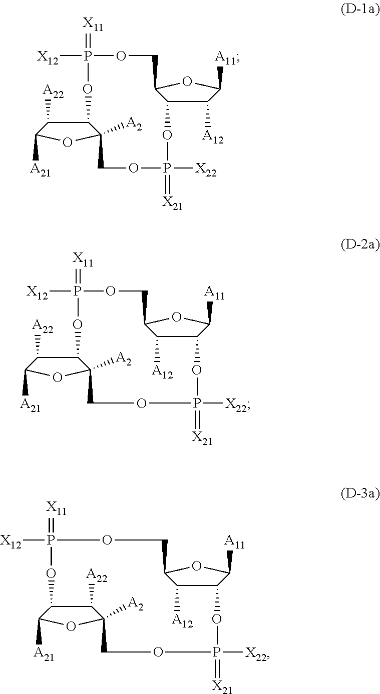

where:
X₁₁ and X₂₁ are as defined above,
X₁₂ and X₂₂ are as defined above or O or S,
A₁₁ and A₂₁ are as defined above, i.e. independently a group of formula:

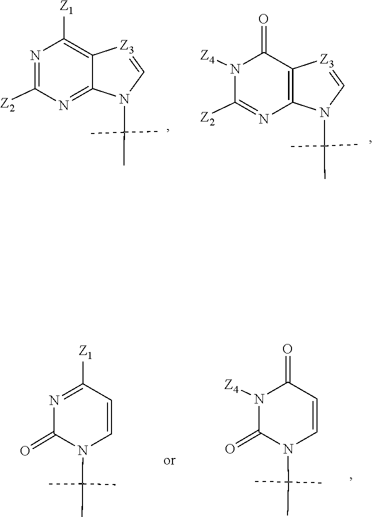

preferably

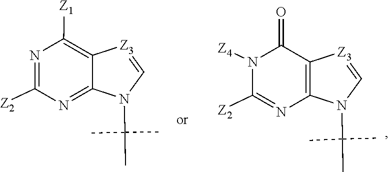

where:
   Z₁ is as defined above or O or NR₁₁,
   Z₂ is as defined above or NR₂₁,
   Z₃ is as defined above, and
   Z₄ is as defined above,
A₁₂ and A₂₂ are as defined above, and
A₂ is as defined above,
   wherein:
when X₁₂ is O or S, then X₂₂ is not O and is not S, Z₁ is not O and is not NR₁₁, Z₂ is
not NR₂₁, and the residue of the STING agonist is linked to the rest of the molecule by X₁₂;
when X₂₂ is O or S, then X₁₂ is not O and is not S, Z₁ is not O and is not NR₁₁, Z₂ is not NR₂₁, and the residue of the STING agonist is linked to the rest of the molecule by X₂₂;
when Z₁ is O or NR₁₁, then X₁₂ is not O and is not S, X₂₂ is not O and is not S, Z₂ is
not NR₂₁, and the residue of the STING agonist is linked to the rest of the molecule by Z₁;
when Z₂ is NR₂₁, then X₁₂ is not O and is not S, X₂₂ is not O and is not S, Z₁ is not O and is not NR₁₁, and the residue of the STING agonist is linked to the rest of the molecule by Z₂.

### Binding Unit Moiety

The binding unit is a peptide, a protein (e.g. an engineered protein), an antibody (e.g. a monoclonal antibody) or an antigen binding fragment thereof.

Preferably, the binding unit according to the invention is an antibody or an antigen binding fragment thereof, and thus, the binding unit-drug conjugate according to the invention is an antibody-drug conjugate (ADC). In an embodiment, the antibody of the invention consists of a recombinant antibody. In another embodiment, the antibody of the ADC of the invention consists of a chemically synthesized antibody.

More particularly, such a molecule consists of a glycoprotein comprising at least two heavy (H) chains and two light (L) chains inter-connected by disulfide bonds. Each heavy chain comprises a heavy chain variable region (or domain) (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain, CL. The VH and VL regions can be further subdivided into regions of hypervariability, termed complementarily determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g. effector cells) and the first component (C1q) of the classical complement system.

In an embodiment, the “antigen binding fragments” are selected in the group consisting of Fv, scFv (sc for single chain), Fab, F(ab′)₂, Fab′, scFv-Fc fragments or diabodies, or any fragment of which the half-life time would have been increased by chemical modification, such as the addition of poly(alkylene) glycol such as poly(ethylene) glycol (“PEGylation”) (pegylated fragments called Fv-PEG, scFv-PEG, Fab-PEG, F(ab′)₂-PEG or Fab′-PEG) (“PEG” for Poly(Ethylene) Glycol), or by incorporation in a liposome, said fragments having at least one of the characteristic CDRs of the antibody according to the invention. Preferably, said “antigen binding fragments” will be constituted or will comprise a partial sequence of the heavy or light variable chain of the antibody from which they are derived, said partial sequence being sufficient to retain the same specificity of binding as the antibody from which it is descended and a sufficient affinity, preferably at least equal to 1/100, in a more preferred manner to at least 1/10, of the affinity of the antibody from which it is descended, with respect to the target. More preferably, said “antigen binding fragments” will be constituted of or will comprise at least the three CDRs CDR-H1, CDR-H2 and CDR-H3 of the heavy variable chain and the three CDRs CDR-L1, CDR-L2 and CDR-L3 of the light variable chain of the antibody from which they are derived.

According to a preferred embodiment, the binding unit is an IGF-1R antibody, a HER2 antibody or an antigen binding fragment thereof.

The HER2 antibody is more particularly trastuzumab.

In an embodiment of the present application, the antibody is an IGF-1R antibody and the epitope of the antibody is preferentially localized into the extracellular domain of the human IGF-1R (also referred as IGF-1R ECD).

In a particular embodiment, the antibody, or any antigen binding fragment thereof, is capable of binding to IGF-1R with an EC₅₀ comprised between 10×10⁻¹⁰ to 1×10⁻¹⁰, and more preferentially between 8×10⁻¹⁰ to 2×10⁻¹⁰.

The competition for binding to IGF-1R can be determined by any methods or techniques known by the person skilled in the art such as, without limitation, radioactivity, Biacore, ELISA, Flow cytometry, etc. As “which competes for binding to IGF-1R” it is meant a competition of at least 20%, preferentially at least 50% and more preferentially at least 70%.

The determination of the binding to the same epitope can be determined by any methods or techniques known by the person skilled in the art such as, without limitation, radioactivity, Biacore, ELISA, Flow cytometry, etc. As “which bind to the same epitope of IGF-1R, it is meant a competition of at least 20%, preferentially at least 50% and more preferentially at least 70%.

As above mentioned, and contrary to the general knowledge, the present invention focuses on specific IGF-1R antibodies presenting a high ability to be internalized following IGF-1R binding. As used herein, an antibody that “is internalized” or that “internalized” (the two expressions being similar) is one that is taken up by (meaning it “enters”) the cell upon binding to IGF-1R on a mammalian cell. Such an antibody is interesting as part of the ADC, so it addresses or directs the linked cytotoxic into the targeted cancer cells. Once internalized the cytotoxic triggers cancer cell death.

Advantageously, the IGF-1R antibodies according to the invention are all presenting the same sequences for the CDR-H2, CDR-H3 and CDR-L2, the other 3 CDRs being different. This observation seems coherent as it is part of the general knowledge that, regarding the binding specificity of an antibody, the CDR-H3 is described as being the most important and the most implicated with the recognition of the epitope.

Important keys to success with ADC therapy are thought to be the target antigen specificity and the internalization of the antigen-antibody complexes into the cancer cells. Obviously non-internalizing antigens are less effective than internalizing antigens to delivers cytotoxic agents. Internalization processes are variable across antigens and depend on multiple parameters that can be influenced by antibodies.

In the ADC, the drug moiety confers the cytotoxic activity and the used antibody is responsible for the specificity against cancer cells, as well as a vector for entering within the cells to correctly address the cytotoxic. Thus, to improve the ADC, the antibody can exhibit high ability to internalize into the targeted cancer cells. The efficiency of the antibody mediated internalisation differs significantly depending on the epitope targeted. Selection of potent internalizing IGF-1R antibodies requires various experimental data studying not only IGF-1R downregulation but also following IGF-1R antibody internalization into the cells.

In an embodiment, the internalization of the antibody of the ADC according to the invention can be evaluated by immunofluorescence or FACS (Flow Cytometry) (as exemplified hereinafter in the present application) or any method or process known by the person skilled in the art specific for the internalization mechanism. In a preferred embodiment, the antibody of the ADC according to the invention can induce internalization after binding to IGF-1R of at least 30%, preferentially 50% and more preferentially 80%.

The complex IGF-1R/antibody is internalized after binding of the antibody to the ECD of said IGF-1R, and a reduction in the quantity of IGF-1R at the surface of the cells is induced. This reduction can be quantified by any method known by the person skilled in the art such as non limitative examples western-blot, FACS, and immunofluorescence.

In one embodiment, this reduction, thus reflecting the internalization, can be preferably measured by FACS and expressed as the difference or delta between the Mean Fluorescence Intensity (MFI) measured at 4° C. with the MFI measured at 37° C. after 4 hours incubation with the antibody.

As non limitative example, this delta is determined based on MFIs obtained with untreated cells and cells treated with the antibody using i) breast cancer cells MCF7 after a 4 hour incubation period with the antibody herein described and ii) a secondary antibody labelled with Alexa488. This parameter is defined as calculated with the following formula: Δ(MFI_{4° C.}-MFI_{37° C.}).

This difference between MFIs reflects the IGF-1R downregulation as MFIs are proportional to IGF-1R expressed on the cell-surface.

In an advantageous aspect, the antibodies consist of antibodies triggering a Δ(MFI_{4° C.}-MFI_{37° C.}) on MCF-7 of at least 280, preferably of at least 400.

In more details, the above mentioned delta can be measured according to the following process, which must be considered as an illustrative and non limitative example:
a) Treating and incubating tumor cells of interest with the antibody of the invention in either cold (4° C.) or warm (37° C.) complete culture medium;
b) Treating the treated cells of step a) and, in parallel, untreated cells with a secondary antibody;
c) Measuring the MFI (representative of the quantity of IGF-1R present at the surface) for the treated and the non-treated cells with a secondary labeled antibody capable of binding to the antibody of the invention; and
d) Calculating the delta as the subtraction of the MFI obtained with the treated cells from the MFI obtained with the non-treated cells.

From this delta MFI, an internalization percentage can be determined as:

100×(MFI_{4° C.}−MFI_{37° C.})/MFI_{4° C.}.

The antibodies of the ADC according to the invention, present, preferably, on MCF7 an internalization percentage comprised between 50% and 99%, 70% and 90%, preferentially between 75% and 87%.

A particular advantage of the antibodies herein described relies on their rate of internalization.

It is generally known that, for an ADC, it is desirable that the used antibodies exhibit a rapid rate of internalization, preferably within 24 hours from administration of the antibody and, more preferably within 12 hours and, even more preferably within 6 hours.

In the present invention, the internalization rate, also referred as cell surface bound antibody decrease or cell surface antibody decay, is expressed as t1/2 (half-life) and corresponds as the time necessary to obtain a decrease of 50% of the ΔMFI (this aspect will be clearly understood regarding the following examples).

A particular advantage is that the antibodies of the ADC of the invention have a t1/2 comprised between 5 and 25 minutes, and preferentially between 10 and 20 minutes.

According to a particular embodiment of the invention, the antibody comprises the three heavy chain CDRs of sequences SEQ ID Nos. 1, 2 and 3 and the three light chain CDRs of sequences SEQ ID Nos. 4, 5 and 6.

TABLE 1

| SEQ | |
|---|---|
| ID No. | Sequence |
| 1 | GYTFTSYY |
| | (position 3: T may be replaced by S; |
| | position 8: Y may be replaced by F) |
| 2 | IWPGDGST |
| 3 | ASPMITPNYAMDY |
| 4 | QDISKY |
| | (position 4: S may be replaced by N) |
| 5 | YTS |
| 6 | QQGSTLPYT |
| | (position 5: T may be replaced by A) |

According to a particular embodiment of the invention, the antibody comprises the three heavy chain CDRs comprising or consisting of the sequences SEQ ID Nos. 1, 2 and 3, or any sequence exhibiting at least 80%, preferably 85%, 90%, 95% and 98% identity with SEQ ID Nos. 1, 2 or 3; and the three light chain CDRs comprising or consisting of the sequences SEQ ID Nos. 4, 5 and 6, or any sequence exhibiting at least 80%, preferably 85%, 90%, 95% and 98% identity with SEQ ID Nos. 4, 5 or 6.

According to a particular embodiment of the invention, the binding unit is an antibody, or an antigen binding fragment thereof, capable of binding to the human IGF-1R selected from:
i) an antibody which comprises three heavy chain CDRs with CDR-H2 of sequence SEQ ID No. 2 and CDR-H3 of sequence SEQ ID No. 3, and three light chain CDRs with CDR-L2 of sequence SEQ ID No. 5;
ii) an antibody that competes for binding to IGF-1R with the antibody of i); and
iii) an antibody that binds to the same epitope of IGF-1R as the antibody of i).

According to a particular embodiment of the invention, the binding unit is an antibody, or an antigen binding fragment thereof, capable of binding to the human IGF-1R selected from:
i) an antibody which comprises the three heavy chain CDRs of sequence SEQ ID No. 1, 2 and 3 and the three light chain CDRs of sequence SEQ ID No. 4, 5 and 6;
ii) an antibody that competes for binding to IGF-1R with the antibody of i); and
iii) an antibody that binds to the same epitope of IGF-1R as the antibody of i).

In another embodiment, the antibody, or any antigen binding fragment thereof, comprises the three heavy chain CDRs comprising the sequences SEQ ID Nos. 1, 2 and 3; and the three light chain CDRs comprising the sequences SEQ ID Nos. 4, 5 and 6.

The IMGT unique numbering has been defined to compare the variable domains whatever the antigen receptor, the chain type, or the species [Lefranc M.-P., Immunology Today 18, 509 (1997)/Lefranc M.-P., The Immunologist, 7, 132-136 (1999)/Lefranc, M.-P., Pommié, C., Ruiz, M., Giudicelli, V., Foulquier, E., Truong, L., Thouvenin-Contet, V. and Lefranc, Dev. Comp. Immunol., 27, 55-77 (2003)]. In the IMGT unique numbering, the conserved amino acids always have the same position, for instance cystein 23 (1st-CYS), tryptophan 41 (CONSERVED-TRP), hydrophobic amino acid 89, cystein 104 (2nd-CYS), phenylalanine or tryptophan 118 (J-PHE or J-TRP). The IMGT unique numbering provides a standardized delimitation of the framework regions (FR1-IMGT: positions 1 to 26, FR2-IMGT: 39 to 55, FR3-IMGT: 66 to 104 and FR4-IMGT: 118 to 128) and of the complementarity determining regions: CDR1-IMGT: 27 to 38, CDR2-IMGT: 56 to 65 and CDR3-IMGT: 105 to 117. As gaps represent unoccupied positions, the CDR-IMGT lengths (shown between brackets and separated by dots, e.g. [8.8.13]) become crucial information. The IMGT unique numbering is used in 2D graphical representations, designated as IMGT Colliers de Perles [Ruiz, M. and Lefranc, M.-P., Immunogenetics, 53, 857-883 (2002)/Kaas, Q. and Lefranc, M.-P., Current Bioinformatics, 2, 21-30 (2007)], and in 3D structures in IMGT/3Dstructure-DB [Kaas, Q., Ruiz, M. and Lefranc, M.-P., T cell receptor and MHC structural data. Nucl. Acids. Res., 32, D208-D210 (2004)].

For the amino acid sequence exhibiting at least 80%, preferably 85%, 90%, 95% and 98% identity with a reference amino acid sequence, preferred examples include those containing the reference sequence, certain modifications, notably a deletion, addition or substitution of at least one amino acid, truncation or extension. In the case of substitution of one or more consecutive or non-consecutive amino acids, substitutions are preferred in which the substituted amino acids are replaced by “equivalent” amino acids. Here, the expression “equivalent amino acids” is meant to indicate any amino acids likely to be substituted for one of the structural amino acids without however modifying the biological activities of the corresponding antibodies and of those specific examples defined below.

Equivalent amino acids can be determined either on their structural homology with the amino acids for which they are substituted or on the results of comparative tests of biological activity between the various antibodies likely to be generated.

As a non-limiting example, table 1 below summarizes the possible substitutions likely to be carried out without resulting in a significant modification of the biological activity of the corresponding modified antibody; inverse substitutions are naturally possible under the same conditions.

TABLE 1

| Original residue | Substitution(s) |
|---|---|
| Ala (A) | Val, Gly, Pro |
| Arg (R) | Lys, His |
| Asn (N) | Gln |
| Asp (D) | Glu |
| Cys (C) | Ser |
| Gln (Q) | Asn |
| Glu (E) | Asp |
| Gly (G) | Ala |
| His (H) | Arg |
| Ile (I) | Leu |
| Leu (L) | Ile, Val, Met |
| Lys (K) | Arg |
| Met (M) | Leu |
| Phe (F) | Tyr |
| Pro (P) | Ala |
| Ser (S) | Thr, Cys |
| Thr (T) | Ser |
| Trp (W) | Tyr |
| Tyr (Y) | Phe, Trp |
| Val (V) | Leu, Ala |

A particular aspect of the invention is that the antibody does not bind to the Insulin receptor (IR). This aspect is of interest as the antibody herein described will not have any negative impact on the IR, meaning the Insulin metabolism.

In another embodiment, still another advantageous aspect of the antibody is that it is capable of binding not only to the human IGF-1R but also to the monkey IGF-1R, and more particularly to the cynomolgus IGF-1R. This aspect is also of interest as it will facilitate the toxicity assessment required for clinical trials.

In still another embodiment, the antibody consists of a monoclonal antibody. The monoclonal antibody herein includes murine, chimeric and humanized antibody, such as described after.

The antibody is preferably derived from an hybridoma of murine origin filed within the French collection for microorganism cultures (CNCM, Pasteur Institute, 25 rue du Docteur Roux, 75724 Paris Cedex 15, France), said hybridoma being obtained by the fusion of Balb/C immunized mice splenocytes/lymphocytes and cells of the myeloma Sp 2/0-Ag 14 cell line.

In an embodiment, the IGF-1R antibody consists of a murine antibody, then referred as m[name of the antibody].

In an embodiment, the IGF-1R antibody consists of a chimeric antibody, then referred as c[name of the antibody].

In an embodiment, the IGF-1R antibody consists of a humanized antibody, then referred as hz[name of the antibody].

For the avoidance of doubt, in the following specification, the expressions “IGF-1R antibody” and “[name of the antibody]” are similar and include (without contrary specification) the murine, the chimeric and the humanized versions of the said IGF-1R antibody or of the said “[name of the antibody]”. When necessary, the prefix m- (murine), c- (chimeric) or hz- (humanized) is used.

For more clarity, the following table 2 illustrates the CDR sequences, defined according to IMGT, for the preferred antibodies.

TABLE 2

| | | | SEQ ID |
|---|---|---|---|
| | Heavy chain | Light chain | No. |
| Consensus | CDR-H1 | | 1 |
| | CDR-H2 | | 2 |
| | CDR-H3 | | 3 |
| | | CDR-L1 | 4 |
| | | CDR-L2 | 5 |
| | | CDR-L3 | 6 |
| 208F2 | CDR-H1 | | 7 |
| | CDR-H2 | | 2 |
| | CDR-H3 | | 3 |
| | | CDR-L1 | 9 |
| | | CDR-L2 | 5 |
| | | CDR-L3 | 11 |
| 212A11 | CDR-H1 | | 7 |
| | CDR-H2 | | 2 |
| | CDR-H3 | | 3 |
| | | CDR-L1 | 10 |
| | | CDR-L2 | 5 |
| | | CDR-L3 | 11 |
| 214F8 | CDR-H1 | | 7 |
| & | CDR-H2 | | 2 |
| 213B10 | CDR-H3 | | 3 |
| | | CDR-L1 | 9 |
| | | CDR-L2 | 5 |
| | | CDR-L3 | 12 |
| 219D6 | CDR-H1 | | 8 |
| | CDR-H2 | | 2 |
| | CDR-H3 | | 3 |
| | | CDR-L1 | 9 |
| | | CDR-L2 | 5 |
| | | CDR-L3 | 11 |

It will be obvious for the man skilled in the art that any combination of 6 CDRs as above described should be considered as part of the present invention.

As can be observed from this table 2, all the antibodies herein described have the same sequences for the CDR-H2, CDR-H3 and CDR-L2, this property being of particular interest as above described.

According to a specific aspect, the antibody is a murine antibody characterized in that said antibody also comprises light chain and heavy chain constant regions derived from an antibody of a species heterologous with the mouse, notably man.

According to another specific aspect, the antibody is a chimeric (c) antibody characterized in that said antibody also comprises light chain and heavy chain constant regions derived from an antibody of a species heterologous with the mouse, notably human.

A chimeric antibody is one containing a natural variable region (light chain and heavy chain) derived from an antibody of a given species in combination with constant regions of the light chain and the heavy chain of an antibody of a species heterologous to said given species.

The chimeric antibodies can be prepared by using the techniques of recombinant genetics. For example, the chimeric antibody could be produced by cloning recombinant DNA containing a promoter and a sequence coding for the variable region of a nonhuman monoclonal antibody, notably murine, and a sequence coding for heterologous species antibody constant region, preferably human. A chimeric antibody of the ADC according to the invention coded by one such recombinant gene could be, for example, a mouse-human chimera, the specificity of this antibody being determined by the variable region derived from the murine DNA and its isotype determined by the constant region derived from human DNA.

According to an embodiment of the invention, the antibody is selected from:
a) an antibody comprising the three heavy chain CDRs of sequence SEQ ID No. 7, 2 and 3 and the three light chain CDRs of sequence SEQ ID No. 9, 5 and 11;
b) an antibody comprising the three heavy chain CDRs of sequence SEQ ID No. 7, 2 and 3 and the three light chain CDRs of sequence SEQ ID No. 10, 5 and 11;
c) an antibody comprising the three heavy chain CDRs of sequence SEQ ID No. 7, 2 and 3 and the three light chain CDRs of sequence SEQ ID No. 9, 5 and 12; and
d) an antibody comprising the three heavy chain CDRs of sequence SEQ ID No. 8, 2 and 3 and the three light chain CDRs of sequence SEQ ID No. 9, 5 and 11.

In a preferred, but not limitative, embodiment, the antibody is selected from:
a) an antibody comprising a heavy chain variable domain of sequence SEQ ID No. 13 or any sequence exhibiting at least 80% identity with SEQ ID No. 13 and the three light chain CDRs of sequences SEQ ID Nos. 9, 5 and 11;
b) an antibody comprising a heavy chain variable domain of sequence SEQ ID No. 14 or any sequence exhibiting at least 80% identity with SEQ ID No. 14 and the three light chain CDRs of sequences SEQ ID Nos. 10, 5 and 11;
c) an antibody comprising a heavy chain variable domain of sequence SEQ ID No. 15 or any sequence exhibiting at least 80% identity with SEQ ID No. 15 and the three light chain CDRs of sequences SEQ ID Nos. 9, 5 and 12;
d) an antibody comprising a heavy chain variable domain of sequence SEQ ID No. 16 or any sequence exhibiting at least 80% identity with SEQ ID No. 16 and the three light chain CDRs of sequences SEQ ID Nos. 9, 5 and 11; and
e) an antibody comprising a heavy chain variable domain of sequence SEQ ID No. 17 or any sequence exhibiting at least 80% identity with SEQ ID No. 17 and the three light chain CDRs of sequences SEQ ID Nos. 9, 5 and 12.

By “any sequence exhibiting at least 80%, preferably 85%, 90%, 95% and 98% identity with SEQ ID No. 13 to 17”, its is intended to designate the sequences exhibiting the three heavy chain CDRs SEQ ID Nos. 1, 2 and 3 and, in addition, exhibiting at least 80%, preferably 85%, 90%, 95% and 98%, identity with the full sequence SEQ ID No. 13 to 17 outside the sequences corresponding to the CDRs (i.e. SEQ ID No. 1, 2 and 3).

According to an embodiment of the invention, the antibody is selected from:
a) an antibody comprising a heavy chain variable domain of sequence SEQ ID No. 13 and the three light chain CDRs of sequence SEQ ID No. 9, 5 and 11;
b) an antibody comprising a heavy chain variable domain of sequence SEQ ID No. 14 and the three light chain CDRs of sequence SEQ ID No. 10, 5 and 11;
c) an antibody comprising a heavy chain variable domain of sequence SEQ ID No. 15 and the three light chain CDRs of sequence SEQ ID No. 9, 5 and 12;
d) an antibody comprising a heavy chain variable domain of sequence SEQ ID No. 16 and the three light chain CDRs of sequence SEQ ID No. 9, 5 and 11; and
e) an antibody comprising a heavy chain variable domain of sequence SEQ ID No. 17 and the three light chain CDRs of sequence SEQ ID No. 9, 5 and 12.

In another preferred, but not limitative, embodiment, the antibody is selected from:
a) an antibody comprising a light chain variable domain of sequence SEQ ID No. 18 or any sequence exhibiting at least 80% identity with SEQ ID No. 18 and the three heavy chain CDRs of sequences SEQ ID Nos. 7, 2 and 3;
b) an antibody comprising a light chain variable domain of sequence SEQ ID No. 19 or any sequence exhibiting at least 80% identity with SEQ ID No. 19 and the three heavy chain CDRs of sequences SEQ ID Nos. 7, 2 and 3;
c) an antibody comprising a light chain variable domain of sequence SEQ ID No. 20 or any sequence exhibiting at least 80% identity with SEQ ID No. 20 and the three heavy chain CDRs of sequences SEQ ID Nos. 7, 2 and 3;
d) an antibody comprising a light chain variable domain of sequence SEQ ID No. 21 or any sequence exhibiting at least 80% identity with SEQ ID No. 21 and the three heavy chain CDRs of sequences SEQ ID Nos. 8, 2 and 3; and
e) an antibody comprising a light chain variable domain of sequence SEQ ID No. 22 or any sequence exhibiting at least 80% identity with SEQ ID No. 22 and the three heavy chain CDRs of sequences SEQ ID Nos. 7, 2 and 3.

By “any sequence exhibiting at least 80%, preferably 85%, 90%, 95% and 98% identity with SEQ ID No. 18 to 22”, its is intended to designate respectively the sequences exhibiting the three light chain CDRs SEQ ID Nos. 4, 5 and 6 and, in addition, exhibiting at least 80%, preferably 85%, 90%, 95% and 98%, identity with the full sequence SEQ ID No. 18 to 22 outside the sequences corresponding to the CDRs (i.e. SEQ ID No. 4, 5 and 6).

According to an embodiment of the invention, the antibody is selected from:
a) an antibody comprising a light chain variable domain of sequence SEQ ID No. 18 and the three heavy chain CDRs of sequence SEQ ID No. 7, 2 and 3;
b) an antibody comprising a light chain variable domain of sequence SEQ ID No. 19 and the three heavy chain CDRs of sequence SEQ ID No. 7, 2 and 3;
c) an antibody comprising a light chain variable domain of sequence SEQ ID No. 20 and the three heavy chain CDRs of sequence SEQ ID No. 7, 2 and 3;
d) an antibody comprising a light chain variable domain of sequence SEQ ID No. 21 and the three heavy chain CDRs of sequence SEQ ID No. 8, 2 and 3; and
e) an antibody comprising a light chain variable domain of sequence SEQ ID No. 22 and the three heavy chain CDRs of sequence SEQ ID No. 7, 2 and 3.

According to an embodiment of the invention, the antibody is an antibody selected from:
a) an antibody comprising a heavy chain variable domain of sequence SEQ ID No. 13 or any sequence exhibiting at least 80% identity with SEQ ID No. 13 and a light chain variable domain of sequence SEQ ID No. 18 or any sequence exhibiting at least 80% identity with SEQ ID No. 18;
b) an antibody comprising a heavy chain variable domain of sequence SEQ ID No. 14 or any sequence exhibiting at least 80% identity with SEQ ID No. 14 and a light chain variable domain of sequence SEQ ID No. 19 or any sequence exhibiting at least 80% identity with SEQ ID NO. 19;
c) an antibody comprising a heavy chain variable domain of sequence SEQ ID No. 15 or any sequence exhibiting at least 80% identity with SEQ ID No. 15 and a light chain variable domain of sequence SEQ ID No. 20 or any sequence exhibiting at least 80% identity with SEQ ID No. 20;
d) an antibody comprising a heavy chain variable domain of sequence SEQ ID No. 16 or any sequence exhibiting at least 80% identity with SEQ ID No. 16 and a light chain variable domain of sequence SEQ ID No. 21 or any sequence exhibiting at least 80% identity with SEQ ID No. 21; and
e) an antibody comprising a heavy chain variable domain of sequence SEQ ID No. 17 or any sequence exhibiting at least 80% identity with SEQ ID No. 17 and a light chain variable domain of sequence SEQ ID No. 22 or any sequence exhibiting at least 80% identity with SEQ ID No. 22.

Chimeric antibodies herein described can be also characterized by the constant domain and, more particularly, said chimeric antibodies can be selected or designed such as, without limitation, IgG1, IgG2, IgG3, IgM, IgA, IgD or IgE. More preferably, in the context of the present invention, said chimeric antibodies are IgG1 or IgG4.

According to an embodiment of the invention, the antibody is a chimeric antibody comprising variable domains VH and VL as above described in the format IgG1. More preferably, said chimeric antibody comprises a constant domain for the VH of sequence SEQ ID No. 43 and a Kappa domain for the VL of sequence SEQ ID No. 45.

According to an embodiment of the invention, the antibody is a chimeric antibody comprising variable domains VH and VL as above described in the format IgG4. More preferably, said chimeric antibody comprises a constant domain for the VH of sequence SEQ ID No. 44 and a Kappa domain for the VL of sequence SEQ ID No. 45.

In another preferred, but not limitative, embodiment, the antibody is selected from:
a) an antibody comprising or consisting of a heavy chain of sequence SEQ ID No. 23 or any sequence exhibiting at least 80% identity with SEQ ID No. 23 and a light chain of sequence SEQ ID No. 28 or any sequence exhibiting at least 80% identity with SEQ ID No. 28;
   b) an antibody comprising or consisting of a heavy chain of sequence SEQ ID No. 24 or any sequence exhibiting at least 80% identity with SEQ ID No. 24 and a light chain of sequence SEQ ID No. 29 or any sequence exhibiting at least 80% identity with SEQ ID No. 29;
   c) an antibody comprising or consisting of a heavy chain of sequence SEQ ID No. 25 or any sequence exhibiting at least 80% identity with SEQ ID No. 25 and a light chain of sequence SEQ ID No. 30 or any sequence exhibiting at least 80% identity with SEQ ID No. 30;
   d) an antibody comprising or consisting of a heavy chain of sequence SEQ ID No. 26 or any sequence exhibiting at least 80% identity with SEQ ID No. 26 and a light chain of sequence SEQ ID No. 31 or any sequence exhibiting at least 80% identity with SEQ ID No. 31; and
   e) an antibody comprising or consisting of a heavy chain of sequence SEQ ID No. 27 or any sequence exhibiting at least 80% identity with SEQ ID No. 27 and a light chain of sequence SEQ ID No. 32 or any sequence exhibiting at least 80% identity with SEQ ID No. 32.

For more clarity, the following table 3 illustrates the sequences of the VH and VL, respectively, for the preferred chimeric antibodies.

TABLE 3

| | | | SEQ ID |
|---|---|---|---|
| | Heavy Chain | Light chain | No. |
| c208F2 | Variable domain (VH) | | 13 |
| | | Variable domain (VL) | 18 |
| | Full length | | 23 |
| | | Full length | 28 |
| c212A11 | Variable domain (VH) | | 14 |
| | | Variable domain (VL) | 19 |
| | Full length | | 24 |
| | | Full length | 29 |
| c214F8 | Variable domain (VH) | | 15 |
| | | Variable domain (VL) | 20 |
| | Full length | | 25 |
| | | Full length | 30 |
| c219D6 | Variable domain (VH) | | 16 |
| | | Variable domain (VL) | 21 |
| | Full length | | 26 |
| | | Full length | 31 |
| c213B10 | Variable domain (VH) | | 17 |
| | | Variable domain (VL) | 22 |
| | Full length | | 27 |
| | | Full length | 32 |

According to another specific aspect of the present invention, the antibody is a humanized antibody characterized in that the constant regions of the light chain and the heavy chain derived from human antibody are, respectively, the lambda or kappa region and the gamma-1, gamma-2 or gamma-4 region.

The humanized antibodies or fragments of same can be prepared by techniques known to a person skilled in the art. Such humanized antibodies are preferred for their use in methods involving in vitro diagnoses or preventive and/or therapeutic treatment in vivo. Other humanization techniques, also known to a person skilled in the art, such as, for example, the “CDR grafting” technique described by PDL in patents EP 0 451 216, EP 0 682 040, EP 0 939 127, EP 0 566 647 or U.S. Pat. Nos. 5,530,101, 6,180,370, 5,585,089 and 5,693,761. U.S. Pat. No. 5,639,641 or 6,054,297, 5,886,152 and 5,877,293 can also be cited.

In a preferred embodiment, the antibody comprises a heavy chain variable domain (VH) having:
i) the CDR-H1, CDR-H2 and CDR-H3 of sequences SEQ ID Nos. 7, 2 and 3, respectively, and
ii) the FR1. FR2 and FR3 derived from the human germline IGHV1-46*01 (SEQ ID No. 46), and
iii) the FR4 derived from the human germline IGHJ4*01 (SEQ ID No. 48).

In a preferred embodiment, the antibody comprises a light chain variable domain (VL) having:
i) the CDR-L1, CDR-L2 and CDR-L3 of sequences SEQ ID Nos. 9, 5 and 11, respectively, and
ii) the FR1. FR2 and FR3 derived from the human germline IGKV1-39*01 (SEQ ID No. 47), and
iii) the FR4 derived from the human germline IGKJ4*01 (SEQ ID No. 49).

In a preferred, but not limitative, embodiment of the invention, the antibody comprises:
a) a heavy chain having CDR-H1, CDR-H2 and CDR-H3 of sequences SEQ ID Nos. 7, 2 and 3, respectively, and FR1. FR2 and FR3 derived from the human germline IGHV1-46*01 (SEQ ID No. 46), and the FR4 derived from the human germline IGHJ4*01 (SEQ ID No. 48); and
b) a light chain having CDR-L1, CDR-L2 and CDR-L3 of sequences SEQ ID Nos. 9, 5 and 11, respectively, and FR1. FR2 and FR3 derived from the human germline IGKV1-39*01 (SEQ ID No. 47), and the FR4 derived from the human germline IGKJ4*01 (SEQ ID No. 49).

In an embodiment, the antibody comprises a heavy chain variable domain (VH) of sequence SEQ ID No. 33 and a light chain variable domain (VL) of sequence SEQ ID No. 35. Said humanized antibody will be called thereinafter hz208F2 (“Variant 1” or “Var. 1”).

In another embodiment, the antibody comprises a heavy chain variable domain (VH) of sequence SEQ ID No. 33 wherein said sequence SEQ ID No. 33 comprises at least 1 back-mutation selected from the residues 20, 34, 35, 38, 48, 50, 59, 61, 62, 70, 72, 74, 76, 77, 79, 82 and 95.

In another embodiment, the antibody comprises a heavy chain variable domain (VH) of sequence SEQ ID No. 33 wherein said sequence SEQ ID No. 33 comprises 2, 3, 4, 5, 6, 7, 8, 9, 10, 11, 12, 13, 14, 15, 16 or 17 back-mutations selected from the residues 20, 34, 35, 38, 48, 50, 59, 61, 62, 70, 72, 74, 76, 77, 79, 82 and 95.

For more clarity, the following table 4 illustrates the preferred back-mutations.

TABLE 4

| | No résidu | | | | | | | | | | | | | | | | |
|---|---|---|---|---|---|---|---|---|---|---|---|---|---|---|---|---|---|
| | 20 | 34 | 35 | 38 | 48 | 50 | 59 | 61 | 62 | 70 | 72 | 74 | 76 | 77 | 79 | 82 | 95 |
| Murin | M | I | Y | K | L | W | K | N | E | L | A | K | S | N | A | F | F |
| humain | V | M | H | R | M | I | S | A | Q | M | R | T | T | S | V | E | Y |

In an embodiment, the antibody comprises a light chain variable domain (VL) of sequence SEQ ID No. 35, wherein said sequence SEQ ID No. 35 comprises at least 1 back-mutation selected from the residues 22, 53, 55, 65, 71, 72, 77 and 87.

In an embodiment, the antibody comprises a light chain variable domain (VL) of sequence SEQ ID No. 35, wherein said sequence SEQ ID No. 35 comprises 2, 3, 4, 5, 6, 7 or 8 back-mutations selected from the residues 22, 53, 55, 65, 71, 72, 77 or 87.

In another embodiment, the antibody comprises:
a) a heavy chain variable domain (VH) of sequence SEQ ID No. 33 wherein said sequence SEQ ID No. 33 comprises at least 1 back-mutation selected from the residues 20, 34, 35, 38, 48, 50, 59, 61, 62, 70, 72, 74, 76, 77, 79, 82 and 95; and
b) a light chain variable domain (VL) of sequence SEQ ID No. 35, wherein said sequence SEQ ID No. 35 comprises at least 1 back-mutation selected from the residues 22, 53, 55, 65, 71, 72, 77 and 87.

For more clarity, the following table 5 illustrates the preferred back-mutations.

TABLE 5

| No résidu | 22 | 53 | 55 | 65 | 71 | 72 | 77 | 87 |
|---|---|---|---|---|---|---|---|---|
| Murin | S | R | H | R | Y | S | N | F |
| humain | T | S | Q | S | F | T | S | Y |

In such an embodiment, the antibody comprises all the back-mutations above mentioned and corresponds to an antibody comprising a heavy chain variable domain (VH) of sequence SEQ ID No. 34 and a light chain variable domain (VL) of sequence SEQ ID No. 36. Said humanized antibody will be called thereinafter hz208F2 (“Variant 3” or “Var. 3”).

In another embodiment, all the humanized forms comprised between the Variant 1 and the Variant 3 are also encompassed by the present invention. In other words, the antibody corresponds to an antibody comprising a heavy chain variable domain (VH) of “consensus” sequence SEQ ID No. 41 and a light chain variable domain (VL) of “consensus” sequence SEQ ID No. 42. Said humanized antibody, as a whole, will be called thereinafter hz208F2 (“Variant2” or “Var.2”).

In a preferred, but not limitative, embodiment, the antibody is selected from:
a) an antibody comprising a heavy chain variable domain of sequence SEQ ID No. 33 or any sequence exhibiting at least 80%, preferably 85%, 90%, 95% and 98% identity with SEQ ID No. 33 and the three light chain CDRs of sequences SEQ ID Nos. 9, 5 and 11; and
b) an antibody comprising a heavy chain variable domain of sequence SEQ ID No. 34 or any sequence exhibiting at least 80%, preferably 85%, 90%, 95% and 98% identity with SEQ ID No. 34 and the three light chain CDRs of sequences SEQ ID Nos. 9, 5 and 11.

By “any sequence exhibiting at least 80%, preferably 85%, 90%, 95% and 98% identity with SEQ ID No. 33 or 34”, it is intended to designate the sequences exhibiting the three heavy chain CDRs SEQ ID Nos. 1, 2 and 3 and, in addition, exhibiting at least 80%, preferably 85%, 90%, 95% and 98%, identity with the full sequence SEQ ID No. 33 or 34 outside the sequences corresponding to the CDRs (i.e. SEQ ID Nos. 1, 2 and 3).

In an embodiment of the invention, the antibody is selected from:
a) an antibody comprising a heavy chain variable domain of sequence SEQ ID No. 33 or any sequence exhibiting at least 80% identity with SEQ ID No. 33 and the three light chain CDRs of sequences SEQ ID Nos. 9, 5 and 11; and
b) an antibody comprising a heavy chain variable domain of sequence SEQ ID No. 34 or any sequence exhibiting at least 80% identity with SEQ ID No. 34 and the three light chain CDRs of sequences SEQ ID Nos. 9, 5 and 11.

If not indicated in the concerned paragraphs, in the present description, by any sequence or by a sequence exhibiting at least 80% with a particular sequence, it must be understood that said sequence exhibits at least 80% and preferably 85%, 90%, 95% and 98% identity with the referenced sequence. Whether these sequences contain CDR sequences, it is intended to designate that the sequences exhibiting at least these CDRs identically to the reference sequence CDRs, the 80%, preferably 85%, 90%, 95% and 98%, identity with the full sequence having to be calculated for the remaining sequence located outside the sequences corresponding to these CDRs.

In a preferred, but not limitative, embodiment, the antibody is selected from:
a) an antibody comprising a light chain variable domain of sequence SEQ ID No. 35 or any sequence exhibiting at least 80%, preferably 85%, 90%, 95% and 98% identity with SEQ ID No. 35 and the three heavy chain CDRs of sequences SEQ ID Nos. 7, 2 and 3; and
b) an antibody comprising a light chain variable domain of sequence SEQ ID No. 36 or any sequence exhibiting at least 80%, preferably 85%, 90%, 95% and 98% identity with SEQ ID No. 36 and the three heavy chain CDRs of sequences SEQ ID Nos. 7, 2 and 3.

By “any sequence exhibiting at least 80%, preferably 85%, 90%, 95% and 98% identity with SEQ ID No. 35 or 36”, it is intended to designate the sequences exhibiting the three light chain CDRs SEQ ID Nos. 4, 5 and 6 and, in addition, exhibiting at least 80%, preferably 85%, 90%, 95% and 98%, identity with the full sequence SEQ ID No. 35 or 36 outside the sequences corresponding to the CDRs (i.e. SEQ ID Nos. 4, 5 and 6).

In an embodiment of the invention, the antibody is selected from:
a) an antibody comprising a light chain variable domain of sequence SEQ ID No. 35 or any sequence exhibiting at least 80% identity with SEQ ID No. 35 and the three heavy chain CDRs of sequences SEQ ID Nos. 7, 2 and 3; and
b) an antibody comprising a heavy chain variable domain of sequence SEQ ID No. 36 or any sequence exhibiting at least 80% identity with SEQ ID No. 36 and the three heavy chain CDRs of sequences SEQ ID Nos. 7, 2 and 3.

Humanized antibodies herein described can be also characterized by the constant domain and, more particularly, said humanized antibodies can be selected or designed such as, without limitation, IgG1, IgG2, IgG3, IgM, IgA, IgD or IgE. More preferably, in the context of the present invention, said humanized antibodies are IgG1 or IgG4.

According to an embodiment of the invention, the antibody is a humanized antibody comprising variable domains VH and VL as above described in the format IgG1. More preferably, said humanized antibody comprises a constant domain for the VH of sequence SEQ ID No. 43 and a Kappa domain for the VL of sequence SEQ ID No. 45.

According to an embodiment of the invention, the antibody is a humanized antibody comprising variable domains VH and VL as above described in the format IgG4. More preferably, said humanized antibody comprises a constant domain for the VH of sequence SEQ ID No. 44 and a Kappa domain for the VL of sequence SEQ ID No. 45.

According to still another embodiment of the invention, the antibody is selected from:
a) an antibody comprising or consisting of a heavy chain of sequence SEQ ID No. 37 or any sequence exhibiting at least 80% identity with SEQ ID No. 37 and a light chain of sequence SEQ ID No. 39 or any sequence exhibiting at least 80% identity with SEQ ID No. 39; and
b) an antibody comprising or consisting of a heavy chain of sequence SEQ ID No. 38 or any sequence exhibiting at least 80% identity with SEQ ID No. 38 and a light chain of sequence SEQ ID No. 40 or any sequence exhibiting at least 80% identity with SEQ ID No. 40.

For more clarity, the following table 6a illustrates non limitative examples of sequences of the VH and VL for the variant 1 (Var. 1) and the variant 3 (Var. 3) of the humanized antibody hz208F2. It also comprises the consensus sequence for the variant 2 (Var. 2).

TABLE 6a

| | | | SEQ ID |
|---|---|---|---|
| | Heavy Chain | Light chain | No. |
| hz208F2 | Variable domain (VH) | | 33 |
| (var. 1) | | Variable domain (VL) | 35 |
| | Full length | | 37 |
| | | Full length | 39 |
| hz208F2 | Variable domain (VH) | | 34 |
| (Var. 3) | | Variable domain (VL) | 36 |
| | Full length | | 38 |
| | | Full length | 40 |
| hz208F2 | Variable domain (VH) | | 41 |
| (Var. 2) | | Variable domain (VL) | 42 |

In another preferred, but not limitative, embodiment, the antibody is selected from:
a) an antibody comprising a heavy chain variable domain of sequence selected from SEQ ID Nos. 56, 62, 64, 66, 68, 70, 72, 74, 76, 78 and 80 or any sequence with at least 80%, preferably 85%, 90%, 95% and 98% identity with SEQ ID No.56, 62, 64, 66, 68, 70, 72, 74, 76, 78 and 80; and the three light chain CDRs of sequences SEQ ID Nos. 9, 5 and 11;
b) an antibody comprising a light chain variable domain of sequence selected from SEQ ID Nos. 57 or 60 or any sequence with at least 80%, preferably 85%, 90%, 95% and 98% identity with SEQ ID Nos. 57 or 60; and the three heavy chain CDRs of sequences SEQ ID Nos. 7, 2 and 3; and
c) an antibody comprising a heavy chain variable domain of sequence selected from SEQ ID Nos. 56, 62, 64, 66, 68, 70, 72, 74, 76, 78 and 80 or any sequence with at least 80%, preferably 85%, 90%, 95% and 98% identity with SEQ ID Nos.56, 62, 64, 66, 68, 70, 72, 74, 76, 78 and 80; and a light chain variable domain of sequence selected from SEQ ID Nos. 57 or 60 or any sequence with at least 80%, preferably 85%, 90%, 95% and 98% identity with SEQ ID Nos. 57 or 60.

According to still another embodiment of the invention, the antibody is selected from:
a) an antibody comprising a heavy chain of sequence SEQ ID Nos. 56, 62, 64, 66, 68, 70, 72, 74, 76, 78 and 80 or any sequence exhibiting at least 80% identity with SEQ ID No. 56, 62, 64, 66, 68, 70, 72, 74, 76, 78 or 80, and a light chain of sequence SEQ ID No. 57 or any sequence exhibiting at least 80% identity with SEQ ID No. 57; and
b) an antibody comprising a heavy chain of sequence SEQ ID Nos. 56, 64, 68 and 78 or any sequence exhibiting at least 80% identity with SEQ ID No. 56, 64, 68 or 78 and a light chain of sequence SEQ ID No. 60, or any sequence exhibiting at least 80% identity with SEQ ID No. 60.

According to still another embodiment of the invention, the antibody is selected from:
a) an antibody comprising or consisting of a heavy chain of sequence SEQ ID No. 58 or any sequence exhibiting at least 80% identity with SEQ ID No. 58 and a light chain of sequence SEQ ID No. 59 or any sequence exhibiting at least 80% identity with SEQ ID No. 59;
b) an antibody comprising or consisting of a heavy chain of sequence SEQ ID No. 58 or any sequence exhibiting at least 80% identity with SEQ ID No. 58 and a light chain of sequence SEQ ID No. 61 or any sequence exhibiting at least 80% identity with SEQ ID No. 61;
c) an antibody comprising or consisting of a heavy chain of sequence SEQ ID No. 63 or any sequence exhibiting at least 80% identity with SEQ ID No. 63 and a light chain of sequence SEQ ID No. 59 or any sequence exhibiting at least 80% identity with SEQ ID No. 59;
d) an antibody comprising or consisting of a heavy chain of sequence SEQ ID No. 65 or any sequence exhibiting at least 80% identity with SEQ ID No. 65 and a light chain of sequence SEQ ID No. 59 or any sequence exhibiting at least 80% identity with SEQ ID No. 59;
e) an antibody comprising or consisting of a heavy chain of sequence SEQ ID No. 65 or any sequence exhibiting at least 80% identity with SEQ ID No. 65 and a light chain of sequence SEQ ID No. 61 or any sequence exhibiting at least 80% identity with SEQ ID No. 61;
f) an antibody comprising or consisting of a heavy chain of sequence SEQ ID No. 67 or any sequence exhibiting at least 80% identity with SEQ ID No. 67 and a light chain of sequence SEQ ID No. 59 or any sequence exhibiting at least 80% identity with SEQ ID No. 59;
g) an antibody comprising or consisting of a heavy chain of sequence SEQ ID No. 69 or any sequence exhibiting at least 80% identity with SEQ ID No. 69 and a light chain of sequence SEQ ID No. 59 or any sequence exhibiting at least 80% identity with SEQ ID No. 59;
h) an antibody comprising or consisting of a heavy chain of sequence SEQ ID No. 69 or any sequence exhibiting at least 80% identity with SEQ ID No. 69 and a light chain of sequence SEQ ID No. 61 or any sequence exhibiting at least 80% identity with SEQ ID No. 61;
i) an antibody comprising or consisting of a heavy chain of sequence SEQ ID No. 71 or any sequence exhibiting at least 80% identity with SEQ ID No. 71 and a light chain of sequence SEQ ID No. 59 or any sequence exhibiting at least 80% identity with SEQ ID No. 59;
j) an antibody comprising or consisting of a heavy chain of sequence SEQ ID No. 73 or any sequence exhibiting at least 80% identity with SEQ ID No. 73 and a light chain of sequence SEQ ID No. 59 or any sequence exhibiting at least 80% identity with SEQ ID No. 59;
k) an antibody comprising or consisting of a heavy chain of sequence SEQ ID No. 75 or any sequence exhibiting at least 80% identity with SEQ ID No. 75 and a light chain of sequence SEQ ID No. 59 or any sequence exhibiting at least 80% identity with SEQ ID No. 59;
l) an antibody comprising or consisting of a heavy chain of sequence SEQ ID No. 77 or any sequence exhibiting at least 80% identity with SEQ ID No. 77 and a light chain of sequence SEQ ID No. 59 or any sequence exhibiting at least 80% identity with SEQ ID No. 59;
m) an antibody comprising or consisting of a heavy chain of sequence SEQ ID No. 79 or any sequence exhibiting at least 80% identity with SEQ ID No. 79 and a light chain of sequence SEQ ID No. 59 or any sequence exhibiting at least 80% identity with SEQ ID No. 59;
n) an antibody comprising or consisting of a heavy chain of sequence SEQ ID No. 79 or any sequence exhibiting at least 80% identity with SEQ ID No. 79 and a light chain of sequence SEQ ID No. 61 or any sequence exhibiting at least 80% identity with SEQ ID No. 61; and
o) an antibody comprising or consisting of a heavy chain of sequence SEQ ID No. 81 or any sequence exhibiting at least 80% identity with SEQ ID No. 81 and a light chain of sequence SEQ ID No. 59 or any sequence exhibiting at least 80% identity with SEQ ID No. 59.

In other words, the antibody can be an antibody comprising:
a) a heavy chain of sequence selected from SEQ ID Nos. 58, 63, 65, 67, 69, 71, 73, 75, 77, 79 and 81 or any sequence with at least 80% identity with SEQ ID Nos. 58, 63, 65, 67, 69, 71, 73, 75, 77, 79 and 81; and
b) a light chain of sequence selected from SEQ ID Nos. 59 and 61 or any sequence with at least 80% identity with SEQ ID Nos. 59 and 61.

In an embodiment of the invention, the antibody is selected from:
a) a heavy chain of sequence selected from SEQ ID Nos. 58, 63, 65, 67, 69, 71, 73, 75, 77, 79 and 81 or any sequence with at least 80% identity with SEQ ID Nos. 58, 63, 65, 67, 69, 71, 73, 75, 77, 79 or 81; and
b) a light chain of sequence selected from SEQ ID Nos. 59 and 61 or any sequence with at least 80% identity with SEQ ID Nos. 59 or 61.

For more clarity, the following table 6b illustrates non limitative examples of sequences of the VH and VL (variable domain and full length) for different variants of the humanized antibody hz208F2.

TABLE 6b

| | | | SEQ ID |
|---|---|---|---|
| | Heavy Chain | Light chain | NO. |
| hz208F2 | Variable domain (VH) | | 56 |
| H037/L018 | | Variable domain (VL) | 57 |
| | Full length | | 58 |
| | | Full length | 59 |
| Hz208F2 | Variable domain (VH) | | 56 |
| H037/L021 | | Variable domain (VL) | 60 |
| | Full length | | 58 |
| | | Full length | 61 |
| Hz208F2 | Variable domain (VH) | | 62 |
| H047/L018 | | Variable domain (VL) | 57 |
| | Full length | | 63 |
| | | Full length | 59 |
| Hz208F2 | Variable domain (VH) | | 64 |
| H049/L018 | | Variable domain (VL) | 57 |
| | Full length | | 65 |
| | | Full length | 59 |
| Hz208F2 | Variable domain (VH) | | 64 |
| H049/L021 | | Variable domain (VL) | 60 |
| | Full length | | 65 |
| | | Full length | 61 |
| Hz208F2 | Variable domain (VH) | | 66 |
| H051/L018 | | Variable domain (VL) | 57 |
| | Full length | | 67 |
| | | Full length | 59 |
| Hz208F2 | Variable domain (VH) | | 68 |
| H052/L018 | | Variable domain (VL) | 57 |
| | Full length | | 69 |
| | | Full length | 59 |
| Hz208F2 | Variable domain (VH) | | 68 |
| H052/L021 | | Variable domain (VL) | 60 |
| | Full length | | 69 |
| | | Full length | 61 |
| Hz208F2 | Variable domain (VH) | | 70 |
| H057/L018 | | Variable domain (VL) | 57 |
| | Full length | | 71 |
| | | Full length | 59 |
| Hz208F2 | Variable domain (VH) | | 72 |
| H068/L018 | | Variable domain (VL) | 57 |
| | Full length | | 73 |
| | | Full length | 59 |
| Hz208F2 | Variable domain (VH) | | 74 |
| H070/L018 | | Variable domain (VL) | 57 |
| | Full length | | 75 |
| | | Full length | 59 |
| Hz208F2 | Variable domain (VH) | | 76 |
| H071/L018 | | Variable domain (VL) | 57 |
| | Full length | | 77 |
| | | Full length | 59 |
| Hz208F2 | Variable domain (VH) | | 78 |
| H076/L018 | | Variable domain (VL) | 57 |
| | Full length | | 79 |
| | | Full length | 59 |
| Hz208F2 | Variable domain (VH) | | 78 |
| H076/L021 | | Variable domain (VL) | 60 |
| | Full length | | 79 |
| | | Full length | 61 |
| Hz208F2 | Variable domain (VH) | | 80 |
| H077/L018 | | Variable domain (VL) | 57 |
| | Full length | | 81 |
| | | Full length | 59 |

According to another aspect of the present invention, the antibody is an antibody selected from i) an antibody produced by the hybridoma I-4757, I-4773, I-4775, I-4736 or I-4774 deposited at the CNCM, Institut Pasteur France on the 30 May 2013, 26 Jun. 2013, 26 Jun. 2013, 24 Apr. 2013 and 26 Jun. 2013, respectively, or ii) an antibody which competes for binding to IGF-1R with the antibody of i); or iii) an antibody which binds to the same epitope of IGF-1R as does the antibody of i).

According to a particular aspect, the binding unit is an antibody, or an antigen binding fragment thereof, as above described for use as an addressing vehicle for delivering a cytotoxic agent at a host target site, said host target site consisting of an epitope localized into IGF-1R, preferably the IGF-1R extracellular domain, more preferably the human IGF-1R (SEQ ID No. 50) and still more preferably the human IGF-1R extracellular domain (SEQ ID No. 51), and still more preferably to the N-terminal of the human IGF-1R extracellular domain (SEQ ID No. 52), or any natural variant sequence thereof.

In a preferred embodiment, said host target site is a target site of a mammalian cell, more preferably of a human cell, more preferably cells which naturally or by way of genetic recombination, express IGF-1R.

In an additional embodiment, said host target site is a target site of a cell of patient, preferably human, having a cancer, preferably an IGF-1R expressing cancer, or IGF-1R related cancers.

IGF-1R expressing cancers or IGF-1R related cancers include particularly cancers wherein the tumoral cells express or over-express whole or part of the IGF-1R at their surface.

IGF-1R antibodies that can be used as binding unit in the present invention are described in particular in WO2015/162291, WO2015/162292 or WO2015/162293.

### Linker Molecule

The linker of formula (I) according to the present invention, preferably in which q=2, is useful for covalently linking a drug to a binding unit, such as an antibody (e.g. a monoclonal antibody) or an antigen binding fragment thereof.

For that, the sulfomaleimide moiety of the linker can react with thiol moieties present on the binding unit, whereas the X₃ end of the linker can react with a functional group present on the drug (QH or Q-OH).

The linker molecule can be prepared according to various synthesis methods which are exemplified in the experimental part.

When X₁ and X₂ are independently chosen among H and C₁, at least one being C₁, the linker according to the invention can be prepared from a disulphide compound of formula L-NHCO—CH₂CH₂—S—S—CH₂CH₂—CONH-L, with L representing L₁-(CO)_{c}—(W)_{w}—(Y)_{y}—X₃, optionally in a protected form, by reaction with a chlorinating agent such as SO₂Cl₂.

When X₁ and X₂ are independently chosen among H and Br, at least one being Br, the linker according to the invention can be prepared from a compound of formula

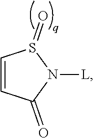

with L representing L₁-(CO)_{c}—(W)_{w}—(Y)_{y}—X₃, optionally in a protected form, by reaction with a brominating agent such as Br₂.

When at least one of X₁ and X₂ is a (C₁-C₆)alkoxy, an aryloxy optionally substituted, or —O—(CH₂CH₂O)ᵣH, the linker according to the invention can be prepared from the corresponding linker of formula (I) with at least one of X₁ and X₂ being Cl or Br, optionally in a protected form, by a nucleophilic substitution reaction with an alcohol of formula Rₐ—OH with Rₐ representing a (C₁-C₆)alkyl, an aryl optionally substituted, or —(CH₂CH₂O)ᵣH.

It can be envisaged also to form the sulfomaleimide moiety with a truncated linker moiety (e.g. with L=L₁-(CO)_{c}—X₆ with X₆ functional group such as NH₂, OH or a leaving group, optionally in a protected form) grafted on it and to complete the linker synthesis after the formation of the sulfomaleimide moiety, as illustrated notably below for the synthesis of the drug-linker conjugate.

Moreover, a step of oxidation can be performed to convert the S(O)_{q} group in the required oxidation state (i.e. preferably q=2). Such an oxidation step is well-known to the one skilled in the art. The oxidant used can be mCPBA, RuO₄ or RuCl₃/NaIO₄ for example.

Further protection/deprotection steps can be carried out in the processes described above, such steps and their reaction conditions being well known to the one skilled in the art.

The linker obtained can be separated from the reaction medium by methods well known to the person skilled in the art, such as by extraction, evaporation of the solvent or by precipitation or crystallisation (followed by filtration).

The linker can also be purified if necessary by methods well known to the person skilled in the art, such as by recrystallisation, by distillation, by chromatography on a column of silica gel or by high performance liquid chromatography (HPLC).

### Drug-Linker Conjugates

The drug-linker conjugate of formula (II) according to the present invention, preferably in which q=2, is useful for covalently linking a drug to a binding unit, such as an antibody (e.g. a monoclonal antibody) or an antigen binding fragment thereof.

For that, the sulfomaleimide moiety of the drug-linker conjugate can react with thiol moieties present on the binding unit.

The drug-linker conjugates can be prepared according to various synthesis methods. Indeed, the linker of formula (I) can react with the drug (QH or Q-OH) in order to form the conjugate. However, other possibilities can be envisaged in which the linker is formed progressively on the drug molecule, i.e. a first part of the linker is first grafted on the drug, the resulting compound being reacted with a truncated linker molecule to form the drug-linker conjugate.

The following non-limitative synthetic routes can thus be used for the preparation of the drug-linker conjugates of formula (II) according to the present invention, even if other synthetic routes could be considered.

In all these synthetic routes, further protection/deprotection/substitution steps can be carried out, such steps and their reaction conditions being well known to the one skilled in the art.

The drug-linker conjugate obtained can be separated from the reaction medium by methods well known to the person skilled in the art, such as by extraction, evaporation of the solvent or by precipitation or crystallisation (followed by filtration).

The drug-linker conjugate can also be purified if necessary by methods well known to the person skilled in the art, such as by recrystallisation, by distillation, by chromatography on a column of silica gel or by high performance liquid chromatography (HPLC).

### Synthetic Route I Represented on Scheme I

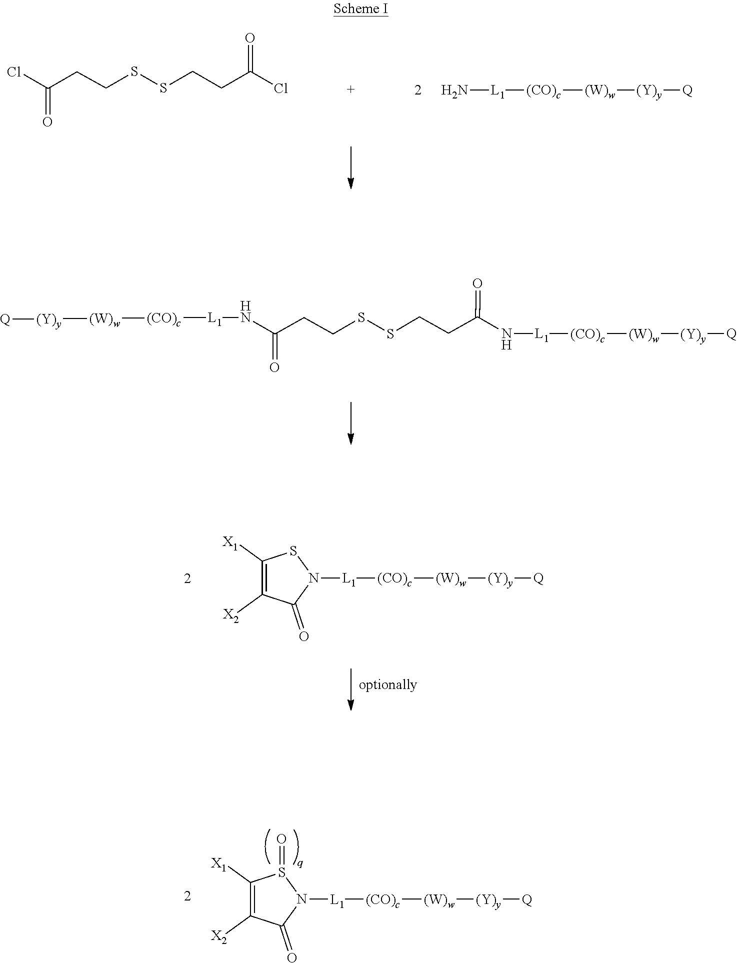

The terminal sulfomaleimide moiety can be formed from a group already present on the drug grafted with a precursor of the linker moiety, as detailed below.
Step 1: 3-(2-Chlorocarbonyl-ethyldisulfanyl)-propionyl chloride is reacted with a molecule of formula H2N-L1-(CO)°—(W)_{w}—(Y)_{y}-Q (i.e. a drug molecule on which a part of the linker has already been grafted). Such a reaction can be performed in the presence of a base such as trimethylamine. The reaction can be performed in a solvent such as DCM, notably at a temperature between 0° C. and room temperature. 3-(2-Chlorocarbonyl-ethyldisulfanyl)-propionyl chloride can be prepared from 3,3′-dithiodipropionic acid by a well-known method to form an acyl chloride such as by reaction with (COCl)₂. The reaction can be performed in a solvent such as DCM, notably at room temperature. A catalytic amount of DMF can be added.
It can be envisaged also to react the 3-(2-chlorocarbonyl-ethyldisulfanyl)-propionyl chloride with a molecule of formula H₂N-L₁-(CO)_{c}—X₃ optionally in a protected form, for example, and to complete the synthesis of the linker moiety grafted with Q in a later step, notably according to one of the other synthetic routes described below.
Step 2: The molecule obtained in step 1 can be cyclized and chlorinated in the presence of SO₂Cl₂, present notably in a large excess (for ex. 5 to 10 eq., such as about 9 eq.). The reaction can be performed in a solvent such as DCM, notably at room temperature.
The chlorine atom can be converted into another X₁ or X₂ group (other than H) by well-known methods, such as by a nucleophilic substitution.
Step 3: If necessary, the molecule obtained in step 2 will be oxidized to obtain a drug-linker of formula (IIa). Such an oxidation step can be performed in conditions well-known to the one skilled in the art, notably in the presence of mCPBA (for ex. 10 eq.). The reaction can be performed in a solvent such as DCM, notably at room temperature.

### Synthetic Route II Represented on Scheme II

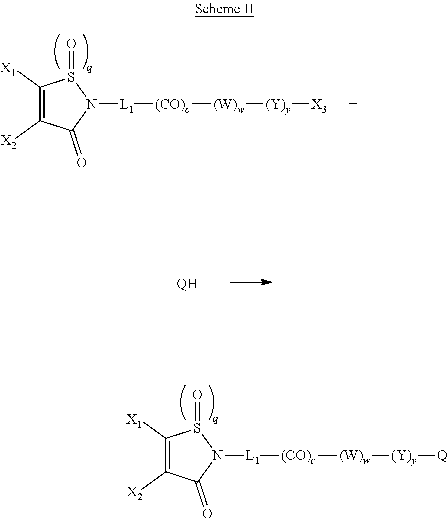

A direct coupling between a drug (QH or Q-OH) and the linker of formula (I) can be performed, the conditions of which depending on the nature of X₃ and of the functional group present on the drug.
This coupling can be a substitution, such as a nucleophilic substitution or a Mitsunobu reaction, the reaction conditions of such chemical reactions being well-known to the one skilled in the art.
When X₃═OH and at least y=1, w≠0 or c=1 (i.e. the terminal functional group of the linker of formula (I) is COOH) and QH comprises a NH function, the coupling between the linker of formula (I) and the drug (QH) can be a peptide coupling well-known to the one skilled in the art. The terminal COOH function can also be converted into an acyl chloride COCl, which could then react with a nucleophilic function present on the drug (QH) (e.g. NH or OH).
When X₃═NH₂; or X₃═H, y=z=1 and Z═—NR₄—(CH₂)ᵤ—NR₅—; or X₃═H, c=w=y=0, z′=1 and Z′ is —NR₄—(CH₂)ᵤ—NR₅— (i.e. the terminal functional group of the linker of formula (I) is NH) and Q-OH comprises a COOH function, the coupling between the linker of formula (I) and the drug (Q-OH) can be a peptide coupling well-known to the one skilled in the art. The terminal COOH function of the drug can also be converted into an acyl chloride COCl, which could then react with the NH function.
If necessary, an additional step of oxidation can be performed to convert the S(O)_{q} group in the required oxidation state (i.e. preferably q=2). Such an oxidation step is well-known to the one skilled in the art. The oxidant used can be mCPBA, RuO₄ or RuCl₃/NaIO₄ for example.

### Synthetic Route III Represented on Schemes IIIa and IIIb

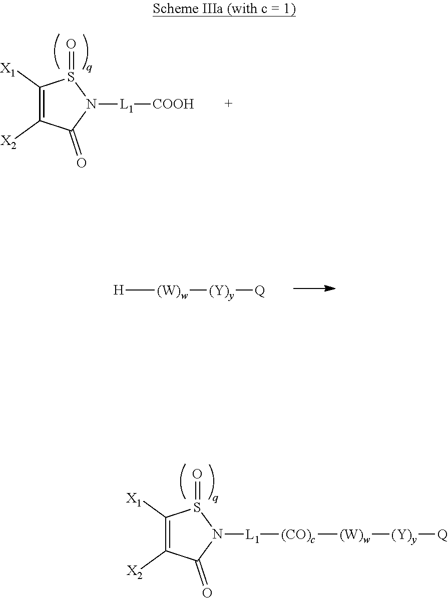

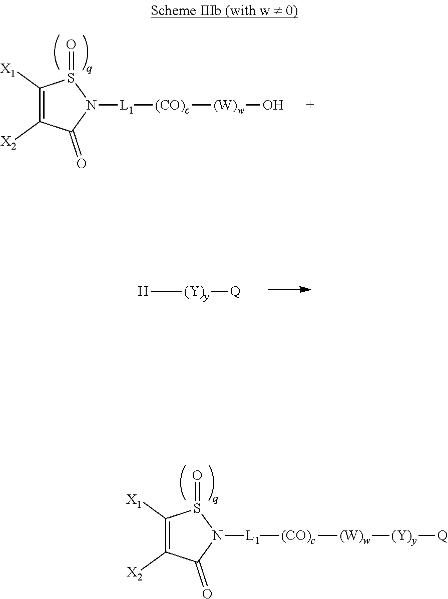

A peptide coupling can also be performed between a truncated linker bearing a COOH function and a drug moiety grafted with the other part of the linker as illustrated on Schemes IIIa and IIIb. The terminal COOH function of the truncated linker can also be converted into an acyl chloride COCl, which could then react with the NH function of the other reactant. The reaction conditions of such reactions are well known to the one skilled in the art.
If necessary, an additional step of oxidation can be performed to convert the S(O)_{q} group in the required oxidation state (i.e. preferably q=2), for example in the presence of mCPBA, RuO₄ or RuCl₃/NaIO₄.

### Synthetic Route IV Represented on Scheme IV

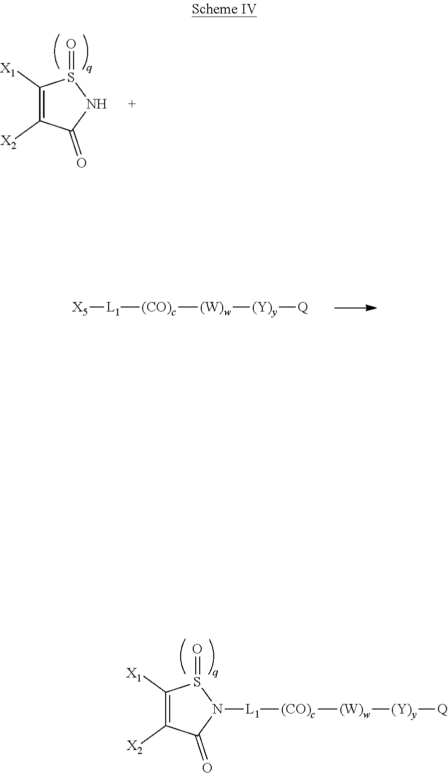

A coupling between the sulfomaleimide moiety and the drug on which the rest of the linker has already been grafted can also be performed as illustrated on Scheme IV on which X₅ represents OH or a leaving group as defined previously.
The coupling can be a substitution, such as a nucleophilic substitution.
If necessary, an additional step of oxidation can be performed to convert the S(O)_{q} group in the required oxidation state (i.e. preferably q=2), for example in the presence of mCPBA, RuO₄ or RuCl₃/NaIO₄.

### Synthetic Route V Represented on Schemes Va and Vb

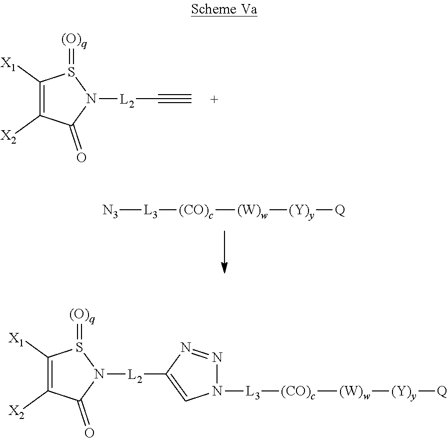

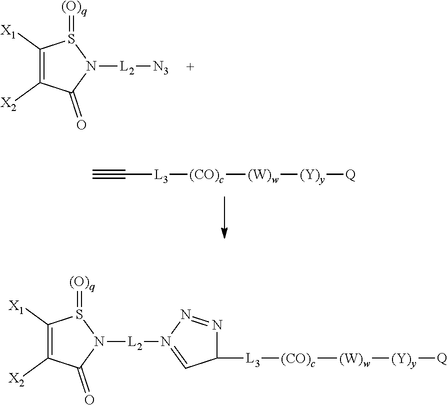

When the linker comprises a heteroarylene moiety with is a bivalent 1H-1,2,3-triazole, this heteroarylene group can be formed by click chemistry between an azide and an alkyne in conditions well known to the one skilled in the art as illustrated on Schemes Va and Vb above where L₂ represents —(CH₂)ₙ— or —(CH₂CH₂O)ₘ—CH₂—CH₂— and L₃ represents —(CH₂)ₚ— or —(CH₂CH₂O)ₘ—CH₂—CH₂—, L₂ and L₃ being not at the same time a group —(CH₂CH₂O)ₘ—CH₂—CH₂—.
If necessary, an additional step of oxidation can be performed to convert the S(O)_{q} group in the required oxidation state (i.e. preferably q=2), for example in the presence of mCPBA, RuO₄ or RuCl₃/NaIO₄.

### Binding Unit-Drug Conjugates

The binding unit-drug conjugates, such as antibody-drug conjugates, can be prepared by:
1) forming thiol functions on the binding unit, notably by reduction of disulphide bond(s); and
2) reacting said binding unit bearing thiol functions with drug-linker conjugate(s) so as covalently link drug moiety/ies onto the binding unit by reacting the sulfomaleimide function with thiol functions.

Such a method is illustrated on Scheme VI below.

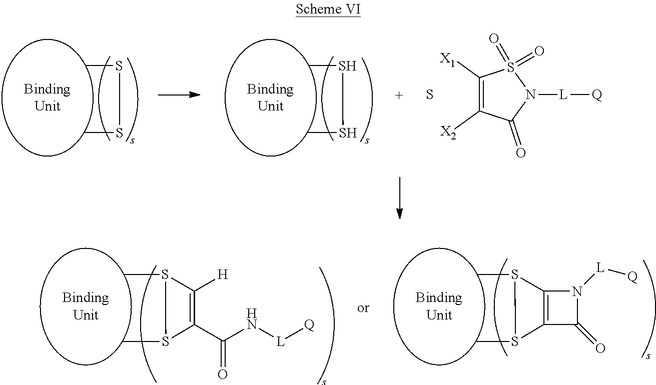

### Pharmaceutical Composition

A pharmaceutical composition according to the present invention comprises a binding unit-drug conjugate of formula (III) or (IV) and at least one pharmaceutically acceptable excipient.

The pharmaceutical compositions of the invention can be intended to enteral (e.g. oral) or parenteral (e.g. intravenous) administration, preferably oral or intravenous administration. The active ingredient can be administered in unit forms for administration, mixed with conventional pharmaceutical excipients, to animals, preferably mammals including humans.

For oral administration, the pharmaceutical composition can be in a solid or liquid (solution or suspension) form.

A solid composition can be in the form of tablets, gelatin capsules, powders, granules and the like. In tablets, the active ingredient can be mixed with pharmaceutical vehicle(s) such as gelatin, starch, lactose, magnesium stearate, talc, gum arabic and the like before being compressed. The tablets may be further coated, notably with sucrose or with other suitable materials, or they may be treated in such a way that they have a prolonged or delayed activity. In powders or granules, the active ingredient can be mixed or granulated with dispersing agents, wetting agents or suspending agents and with flavor correctors or sweeteners. In gelatin capsules, the active ingredient can be introduced into soft or hard gelatin capsules in the form of a powder or granules such as mentioned previously or in the form of a liquid composition such as mentioned below.
A liquid composition can contain the active ingredient together with a sweetener, a taste enhancer or a suitable coloring agent in a solvent such as water. The liquid composition can also be obtained by suspending or dissolving a powder or granules, as mentioned above, in a liquid such as water, juice, milk, etc. It can be for example a syrup or an elixir.

For parenteral administration, the composition can be in the form of an aqueous suspension or solution which may contain suspending agents and/or wetting agents. The composition is advantageously sterile. It can be in the form of an isotonic solution (in particular in comparison to blood).

Such parenteral compositions will contain advantageously a physiologically acceptable medium, generally based on an isotonic saline solution, i.e. 0.9% NaCl aqueous solution (normal saline). Non-aqueous water miscible co-solvent, such as ethanol, glycerin, propylene glycol or n-lactamide, can also be used.
The parenteral composition of the invention can also comprise one or more additive(s), such as suspending agents, wetting agents, preservatives, antioxidants, chelating agents, buffering agents, tonicity adjusting agents, etc. Such additives are conventional to those of skill in the art.
Suspending agents can be an alginate, sodium carboxymethyl cellulose, methyl cellulose, hydroxyl methyl cellulose, hydroxyl ethyl cellulose, hydroxylpropyl methyl cellulose, microcrystalline cellulose, a gum such as acacia, tragacanth or xanthan gum, gelatin, a carrageenan, polyvinyl pyrrolidone, etc.
Wetting agents can be glycerin, propylene glycol or also nonionic surfactants such as a lecithin, a polysorbate or a poloxamer.
Preservatives can be benzyl alcohol, phenol, cresol, chlorobutanol, a paraben such as methylparaben, propylparaben or propylparaben, benzalkonium chloride, benzethonium chloride, etc.
Antioxidants can be ascorbic acid, citric acid, acetylcysteine, sulfurous acid salts (bisulfite, metabisulfite), monothioglycerol, sodium formaldehyde sulfoxylate, thiourea, tocopherol, etc.
Chelating agents can be an ethylene diamine tetraacetic acid (EDTA) salt.
Buffering agents can be acetate, citrate, tartrate, phosphate, triethanolamine (TRIS), etc. Tonicity adjusting agents can be dextrose, glycerol, sodium chloride, glycerin, mannitol, etc.

The binding unit-drug conjugate of the invention can be used in a pharmaceutical composition at a dose ranging from 0.01 mg to 1000 mg a day, administered in only one dose once a day or in several doses along the day, for example twice a day in equal doses. The daily administered dose is advantageously comprised between 5 mg and 500 mg, and more advantageously between 10 mg and 200 mg. However, it can be necessary to use doses out of these ranges, which could be noticed by the person skilled in the art.

### Cancer Treatment

The binding unit-drug conjugate of formula (III) or (IV) or a pharmaceutical composition comprising a binding unit of formula (III) or (IV) can be used for the treatment of cancer, in particular when it comprises a drug moiety (Q) which is a residue of a drug (QH) useful in the treatment of cancer, such as a cytotoxic agent.

Binding unit-drug conjugates, such as antibody-drug conjugates (ADCs) combine the binding specificity of a binding unit, such as an antibody, with the potency of drugs such as, for example, cytotoxic agents.

The use of binding unit-drug conjugates, such as ADCs, allows the local delivery of drugs which, if administered as unconjugated drugs, may result in unacceptable levels of toxicity to normal cells. In other words, maximal efficacy with minimal toxicity is sought thereby.

The cancer can be exemplified by, but not limited to, prostate cancer, osteosarcoma, lung cancer, breast cancer, endometrial cancer, glioblastoma, colon, cancer, gastric cancer, renal cancer, pancreas cancer, head and neck cancer or any other cancer associated with expression of the antigen targeted by the antibody on the tumor cells.

The present invention is illustrated by the following non-limitative examples and figures.

### FIGURES

FIGS. 1A, 2, 3A, 4A, 5A, 6A, 7A, 21 and 22A represent mass spectra of drug-linker conjugates according to the invention.

FIGS. 1B, 3B, 4B, 5B, 6B, 7B, 8 and 9 represent ¹H-NMR spectra of drug-linker conjugates according to the invention.

FIGS. 10 and 11 represent mass spectra of drug-somatostatin conjugates according to the invention.

FIG. 12 represents the SDS-PAGE analysis of the Ab1 antibody (1) and purified ADCs according to the invention (ADC1-A (2), ADC1-B (3), ADC1-C (4), ADC1-D (5), ADC1-E (6), ADC1-F (7) and ADC1-G (8)) under reducing and non-reducing conditions. The bands observed on the gels correspond to completely bridged antibody (i.e. LHHL); partially bridged (i.e. HHL, HH, HL) and no bridging (i.e. H and L).

FIG. 13 represents the SEC analysis of the Ab1 antibody and ADCs according to the invention (ADC1-A, ADC1-B, ADC1-C, ADC1-D, ADC1-E, ADC1-F and ADC1-G).

FIGS. 14A, 14B, 14C and 14D represent ADC m/z spectra before deconvolution of ADCs according to the invention: (A) ADC1-A, (B) ADC1-B, (C) ADC1-C and (D) ADC-1D respectively.

FIGS. 15A, 15B and 15C represent DAR distribution after Maxent deconvolution for (A) ADC1-A, (B) ADC1-B and (C) ADC1-D respectively.

FIGS. 16A and 16B represent an analysis by native mass spectrometry of ADCs: (A) a reference ADC Ref-A and (B) ADC1-C according to the invention.

FIGS. 17A, 17B and 17C represent the results of the in vitro stability study by presenting the percentage of total antibody (100%) and ADC at each timepoint (D0, D3, D7 and D14) in (1) human, (2) cynomolgus, (3) mouse and (4) rat sera for (A) a reference ADC Ref-B, (B) ADC1-C and (C) ADC1-E respectively.

FIGS. 18A and 18B represent the in vitro cell cytotoxicity evaluation of different ADCs in NCI-H2122 (A) and MCF-7 (B) cells respectively.

FIGS. 19 and 20 represent the in vivo activity of ADC1-C and reference ADC Ref-A in an ovarian cancer model.

FIG. 22B represents a TOF-MS spectrum of a drug-linker conjugate according to the invention.

FIG. 23A represents the SEC analysis of the Ab1 antibody and ADCs according to the invention which are synthesized with the PNU-159682 derivatives.

FIG. 23B represents the SEC analysis of the Ab2 antibody and ADCs according to the invention which are synthesized with the PNU-159682 derivatives.

FIGS. 24: 24A, 24B, 24C and 24D represent ADC m/z spectra before deconvolution of the ADCs according to the invention: (A) hz208F2-F562524, (B) c9G4-F562524, (C) hz208F2-F562616 and (D) c9G4-F562646 respectively.

FIGS. 25: 25A, 25B, 25C and 25D represent DAR distribution, after Maxent deconvolution, for ADCs according to the invention: (A) hz208F2-F562524, (B) c9G4-F562524, (C) hz208F2-F562616 and (D) c9G4-F562646 respectively.

FIGS. 26: 26A and 26B represent the in vitro cell cytotoxicity evaluation of the ADC hz208F2-F562524, and the corresponding control ADC c9G4-F562524, in NCI-H2122 (A) and MCF-7 (B) cells respectively.

FIGS. 27: 27A and 27B represent the in vitro cell cytotoxicity evaluation of the ADC hz208F2-F562646, and the corresponding control ADC c9G4-F562646, in NCI-H2122 (A) and MCF-7 (B) cells respectively.

FIG. 28 represents the in vivo activity of the ADC hz208F2-F562524, and the corresponding control ADC c9G4-F562524, in an ovarian cancer model.

### EXAMPLES

### Abbreviations

ACN: Acetonitrile
ADC: Antibody-Drug Conjugate
aq: aqueous
BBO: Broadband Observe
BCA: Bicinchoninic acid
CDR: Complementarily Determining Region
DAR: Drug-to-Antibody Ratio
DCM: Dichloromethane
DIPEA: N,N-Diisopropylethylamine
DMF: Dimethylformamide
DMSO: Dimethylsulfoxide
EDCI: 1-Ethyl-3-(3-dimethylaminopropyl)carbodiimide
EDTA: Ethylenediaminetetraacetic acid
eq: equivalent
ES: Electrospray
ESI: Electrospray ionisation
HATU: 1-[Bis(dimethylamino)methylene]-1H-1,2,3-triazolo[4,5-b] pyridinium 3-oxid hexafluorophosphate
HOBt: 1-Hydroxybenzotriazole
HIC: Hydrophobic Interaction Chromatography
HPLC: High Performance Liquid Chromatography
HRMS: High Resolution Mass Spectrometry
LBA: Ligand Binding Assay
LC: Liquid Chromatography
LCMS: Liquid Chromatography—Mass Spectrometry
mCPBA: meta-Chloroperoxybenzoic acid
Ms: Mesyl
MS: Mass Spectrum
NMR: Nuclear Magnetic Resonance
PBS: Phosphate buffered saline
Q-TOF: Quadrupole-time-of-flight
Rf: Retardation factor
rt: Room Temperature
sat.: saturated
SDS-PAGE: Sodium Dodecyl Sulfate-PolyAcrylamide Gel Electrophoresis
SEC: Size Exclusion Chromatography
TBME: Tert-butyl methyl ether
TCEP: Tris(2-carboxyethyl)phosphine
TEA: Triethylamine
TFA: Trifluoroacetic acid
THE: Tetrahydrofuran
TLC: Thin Layer Chromatography
TOF: Time of Flight
Ts: Tosyl
UV: Ultraviolet

### Experimental Procedures

All reactions requiring anhydrous conditions were conducted in oven-dried apparatus under an atmosphere of nitrogen. Anhydrous solvents were received in sealed bottles under inert atmosphere. All reagents were used as received. Column chromatography was carried out on puriFlash® Columns with silica gel (50 μm) on an Interchim puriFlash®430 and a Grace Reveleris® X2. TLC was performed on aluminum sheets pre-coated with silica (Merck silica gel 60 F₂₅₄) which were visualized with an UV-Lamp 254 nm. Proton (¹H) and carbon (¹³C) NMR spectra were recorded in CDCl₃ and DMSO at room temperature with Bruker 500 MHz Ascend™ equipped with a BBO Prodigy probe (5 mm). Spectra were interpreted using Topspin™ 3.2 software. Chemical shifts (δ_{H} and δ_{C}) are reported in parts per million (ppm) and are referenced relative to either CDCl₃ (¹H NMR 7.26, ¹³C NMR 77.0, central signal of triplet) or DMSO (¹H NMR 2.50, ¹³C NMR 37.9, central signal of septuplet). Assignments were aided by COSY and HSQC experiments. Coupling constants (J: vicinal protons, J_{cis}: vicinal protons in cis position, Jₒ: proton in ortho position, Jₘ: proton in meta position) are given in Hertz to the nearest ±0.1 Hz. Multiplicities are given as singular (s), doublet (d), triplet (t), quartet (q), triplet of triplet (t. of t.), multiplet (m) and broad (b) where applicable. Mass spectra (m/z) were recorded on a Waters® ZQ Mass Detector spectrometer using the technique of electrospray ionization (ES+), source temperature: 120° C., desolvation temperature: 350° C., capillary voltage: 3.20 kV, cone voltage: 25 V, extractor voltage: 5 V, Rf lens voltage: 0.5 V, MS Scan range: 100-2000. HPLC analysis were performed using a Waters® X-Bridge Shield RP18 3.5 μm (3.0 mm×30 mm) column and a Waters® X-Bridge Shield RP18 3.5 μm (3.0 mm×20 mm) pre-column on a HPLC Waters® Alliance 2695 with MassLynx 4.1 software and a Waters 2996 PDA Dectector UV/vis at the appropriate wavelength for the sample under analysis. Retention times (Rₜ) are given in minutes to the nearest 0.01 min. Two methods of elution were used:

TABLE 7
Method 1 of elution for LC

| | Solvent A (Water + 0.1% | Solvent B (ACN + 0.1% |
|---|---|---|
| Time (min.) | formic acid) % | formic acid) % |
| 0.00 | 97.0 | 3.0 |
| 2.25 | 0.0 | 100.0 |
| 2.50 | 0.0 | 100.0 |
| 2.60 | 97.0 | 3.0 |
| 3.00 | 97.0 | 3.0 |

TABLE 8
Method 2 of elution for LC

| | Solvent A (Water + 0.1% | Solvent B (ACN + 0.1% |
|---|---|---|
| Time (min.) | formic acid) % | formic acid) % |
| 0.00 | 50.0 | 50.0 |
| 2.10 | 25.0 | 75.0 |
| 2.25 | 0.0 | 100.0 |
| 2.50 | 0.0 | 100.0 |
| 2.75 | 97.0 | 3.0 |
| 3.00 | 97.0 | 3.0 |

Retention times given by Method 1 are indicated by R_{t,1} and ones given by Method 2 by R_{t,2}.

### 1. Synthesis of the Linkers

### Example of a Synthetic Path for Oxoisothiazolones:

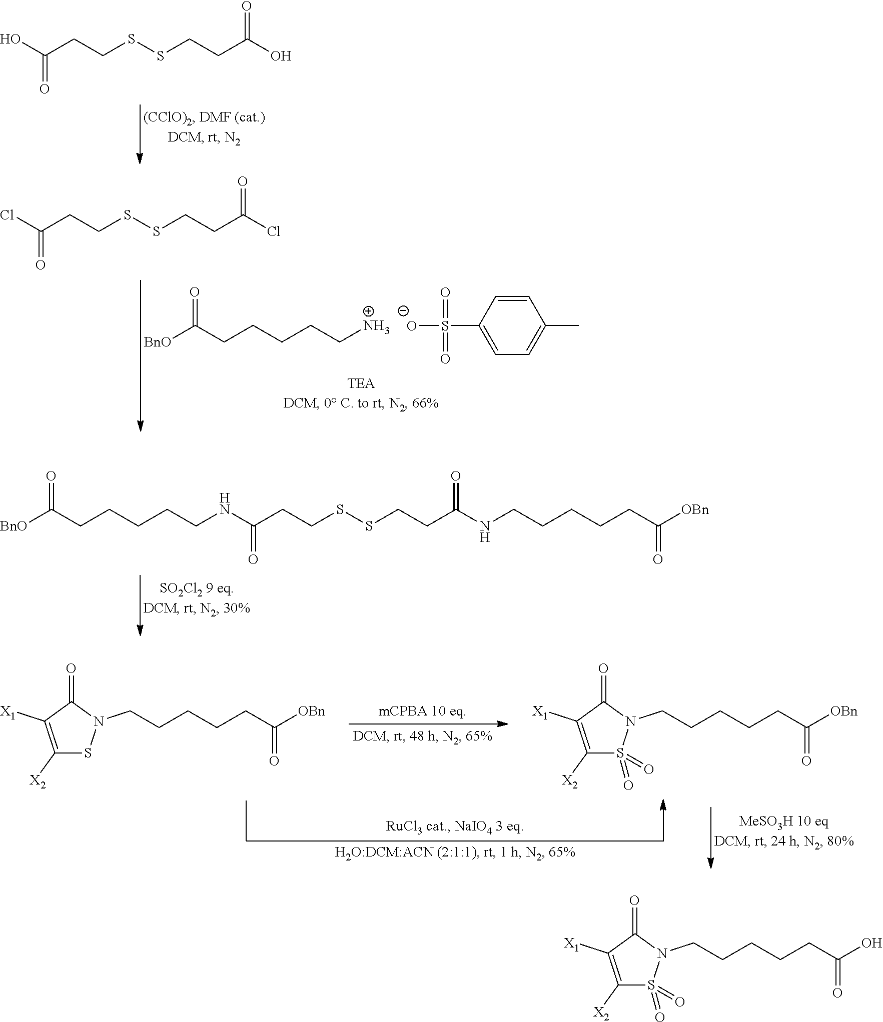

I.1. 3,3′-disulfanediyldipropanoyl chloride

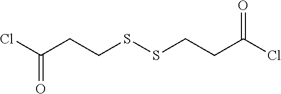 [SMILES: O=C(Cl)CCSSCCC(=O)Cl]

I.2. 3,3′-disulfanediyldipropionic acid (4 g, 0.019 mol, 1 eq.) was suspended in anhydrous DCM (100 mL) and anhydrous DMF (300 μL) was added, followed by oxalyl chloride (7.24 g, 0.057 mol, 3 eq.) at 0° C. under inert atmosphere. The solution clarified. The mixture was left for 3 h at rt until it clarified and no gas formation was longer observed. The crude was evaporated and kept under reduced pressure for another 30 min to remove remnants of oxalyl chloride. A yellow oil (4.70 g, 100%) was obtained. The crude was used without further purification; R_{t,1} (in MeOH): 2.13; MS ES+m/z: 206.86.
I.3. Dibenzyl 6,6′-((3,3′-disulfanediylbis(propanoyl))bis(azanediyl))dihexanoate

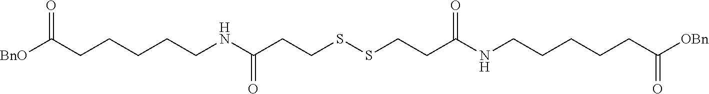 [SMILES: O=C(CCSSCCC(=O)NCCCCCC(=O)OCc1ccccc1)NCCCCCC(=O)OCc1ccccc1]

6-(benzyloxy)-6-oxohexan-1-aminium 4-methylbenzenesulfonate (12.20 g, 0.031 mol, 2.2 eq.) was suspended under vigorous stirring in anhydrous DCM (75 mL) in an ice bath at 0° C. under inert atmosphere. TEA (15.72 mL, 0.113, 8 eq.) was added to the solution. A solution of freshly prepared 3,3′-disulfanediyldipropanoyl chloride (3.88 g, 0.014 mol, 1 eq.) in DCM (25 mL) was slowly dropped into the solution maintained at 0° C. Stirring was continued for 24 hours while the solution was let to come to rt. Water was added (50 mL) and the mixture transferred to a separatory funnel. The organic layer was separated and washed with brine (1×100 mL) then washed with HCl 1M (1×100 mL), saturated solution of NaHCO₃ salt (2×100 mL) and brine (1×100 mL) again. The combined aqueous layers were extracted with DCM (2×100 mL). The organic layers were dried with MgSO₄, filtered and evaporated to dryness affording a yellow solid which was triturated in MeOH to give Dibenzyl 6,6′-((3,3′-disulfanediylbis(propanoyl)) bis(azanediyl))dihexanoate as a light yellow powder (6.0 g, 66%). ¹H NMR (500 MHz, CDCl₃), δ 7.35 (m, 10H), 6.0 (s, 2H), 5.11 (s, 4H), 3.25 (q, J=5.9 Hz, 4H), 3.00 (t, J=7.0 Hz, 4H), 2.55 (t, J=7.10 Hz, 4H), 2.36 (t, J=7.30 Hz, 4H), 1.66 (t of t., J=8.10 Hz, 4H), 1.52 (t of t., J=8.10 Hz, 4H), 1.35 (t oft, J=8.52 Hz, 4H); ¹³C NMR (500 MHz, CDCl₃), δ 173.5 (2-NH—C═O), 170.9 (—O—C═O), 136.0 (2 C_{quat} from aromatic cycles), 128.6 (2H-C_{aromatic}), 128.3 (H-C_{aromatic}), 128.2 (2H-C_{aromatic}), 66.2 (2-CH₂—O—), 39.4 (2-CH₂—N), 35.8 (2-CH₂—COO—), 34.3 (2-CH₂—S—), 34.1 (2-CH₂—CNO—), 29.1 (2 C), 26.3 (2 C), 24.4 (2 C); R_{t,1} (in MeOH): 2.50; MS ES+M/Z: 617.00.
I.4. Benzyl 6-(5-chloro-3-oxoisothiazol-2(3H)-yl)hexanoate

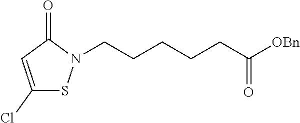 [SMILES: O=C(CCCCCn1sc(Cl)cc1=O)OCc1ccccc1]

Dibenzyl 6,6′-((3,3′-disulfanediylbis(propanoyl))bis(azanediyl))dihexanoate (2.50 g, 4.05 mmol, 1 eq.) was dissolved in anhydrous DCM (20.3 mL). SO₂Cl₂ (pur. 97%, 2.96 mL, 0.036 mol, 9 eq.) was added dropwise to the solution, and the mixture was stirred at rt for 5 h under inert atmosphere. The solution clarified and became pale yellow. Subsequently, the solution was washed with water (2×100 mL) and brine (1×100 mL). The combined aqueous layers were extracted with DCM (2×100 mL). The organic layers were dried with MgSO₄ and filtered, then concentrated under reduced pressure and purified using a chromatography column. Benzyl 6-(5-chloro-3-oxoisothiazol-2(3H)-yl)hexanoate (0.824 g, 29.9%) was obtained as a light yellow oil along with benzyl 6-(3-oxoisothiazol-2(3H)-yl)hexanoate. ¹H NMR (CDCl₃), δ 7.35 (m, 5H), 6.25 (s, 1H), 5.11 (s, 2H), 3.72 (t, J=7.34 Hz, 2H), 2.37 (t, J=7.39 Hz, 2H), 1.69 (m, 4H), 1.38 (m, 2H); ¹³C NMR (500 MHz, CDCl₃), δ 173.2 (—O—C═O), 166.9 (—N—C═O), 145.6 (Cl—HC═CH—), 136.0 (C_{quat.} from aromatic cycle), 128.6-128.3 (5H-C_{aromatic}), 114.8 (Cl—HC═CH—), 66.2 (—CH₂—O—), 43.5 (—CH₂—N—), 34.0 (—CH₂—C═O), 29.4, 25.9, 24.4; R_{t,1} (in ACN): 2.35; MS ES+m/z: 339.84.
I.5. Benzyl 6-(5-chloro-1-oxido-3-oxoisothiazol-2(3H)-yl)hexanoate

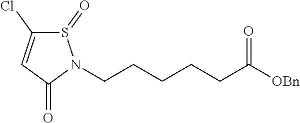 [SMILES: O=C(CCCCCN1C(=O)C=C(Cl)S1=O)OCc1ccccc1]

Benzyl 6-(5-chloro-3-oxoisothiazol-2(3H)-yl)hexanoate (802 g, 3.58 mmol) was diluted in anhydrous DCM (25 mL). 3-chlorobenzoperoxoic acid (1.2 eq.) is added. The solution was stirred for 48 h at rt under inert atmosphere. The solution was then diluted with DCM and treated by 10% aq. Na₂S₂O₃. The organic phase was then extracted successively by a saturated solution of NaHCO₃ salt (2×100 mL), followed by brine (1×100 mL). The combined aqueous layers were extracted with DCM (2×100 mL). The organic layers were dried over MgSO₄, filtered, then concentrated under reduced pressure and purified using a chromatography column. Benzyl 6-(5-chloro-1-oxido-3-oxoisothiazol-2(3H)-yl)hexanoate was then obtained (753 mg) as a colorless oil.
I.6. Benzyl 6-(5-chloro-1,1-dioxido-3-oxoisothiazol-2(3H)-yl)hexanoate

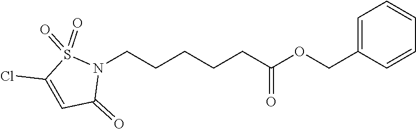 [SMILES: O=C(CCCCCN1C(=O)C=C(Cl)S1(=O)=O)OCc1ccccc1]

### Standard Procedure for the Oxidation of Mono-Chloride Compounds:

Benzyl 6-(5-chloro-3-oxoisothiazol-2(3H)-yl)hexanoate (1.11 g, 0.0036 mol, 1 eq.) was diluted in anhydrous DCM (7 mL). 3-chlorobenzoperoxoic acid (2.69 g, 0.0109 mol, 3 eq.) is added. The solution was stirred for 48 h at rt under inert atmosphere. The solution was then diluted with DCM and treated by 10% aq. Na₂S₂O₃. The organic phase was then extracted successively by a saturated solution of NaHCO₃ salt (2×100 mL), followed by brine (1×100 mL). The combined aqueous layers were extracted with DCM (2×100 mL). The organic layers were dried over MgSO₄, filtered, then concentrated under reduced pressure and purified using a chromatography column. Benzyl 6-(5-chloro-1,1-dioxido-3-oxoisothiazol-2(3H)-yl)hexanoate was obtained as a light yellow oil (0.760 g, 64.3%). ¹H NMR (CDCl₃), δ 7.36 (m, 5H), 6.68 (s, 1H), 5.11 (s, 2H), 3.67 (t, J=7.68 Hz, 2H), 2.37 (t, J=7.27 Hz, 2H), 1.78 (t oft, J=7.73 Hz, 2H), 1.70 (t oft, J=7.38 Hz, 2H), 1.40 (m, 2H); ¹³C NMR (500 MHz, CDCl₃), δ 173.2 (—O—C═O), 157.0 (—N—C═O), 144.6 (C₁—HC═CH—), 136.0 (C_{quat.} from aromatic cycle), 128.6-128.2 (H-C_{aromatics}), 123.6 (C₁—HC═CH—), 66.2 (—CH₂—O), 40.3 (—CH₂—N—), 34.0 (—CH₂—C═O), 27.9, 26.0, 24.2; R_{t,1} (in ACN): 2.49; MS ES+M/Z: 371.83.

### Example 1. 6-(5-Chloro-1,1-dioxido-3-oxoisothiazol-2(3H)-yl)hexanoic acid

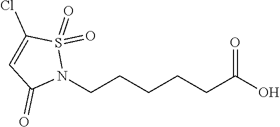 [SMILES: O=C(O)CCCCCN1C(=O)C=C(Cl)S1(=O)=O]

6-(5-Chloro-1,1-dioxido-3-oxoisothiazol-2(3H)-yl)hexanoic acid was obtained as a light white powder following the standard procedure of deprotection of benzyl esters starting from Benzyl 6-(5-chloro-1,1-dioxido-3-oxoisothiazol-2(3H)-yl)hexanoate (0.445 g, 77%). ¹H NMR (CDCl₃), δ 10.89 (br. s, 1H), 6.70 (s, 1H), 3.70 (t, J=7.43 Hz, 2H), 2.38 (t, J=7.38 Hz, 2H), 1.81 (t of t., J=7.60 Hz, 2H), 1.69 (t of t, J=7.71 Hz, 2H), 1.43 (splitted t of t, J=7.75 Hz, J=3.31 Hz, 2H); ¹³C NMR (500 MHz, CDCl₃), δ 178.7 (O═C—OH), 157.1 (—N—C═O), 144.7 (Cl—HC═CH—), 123.6 (Cl—HC═CH—), 40.2 (—CH₂—N), 33.5 (—CH₂—C═O), 27.9, 25.9, 23.9; R₁ (in ACN): 1.98; MS ES+m/z: 349.89.
I.7. Benzyl 6-(4,5-dichloro-3-oxoisothiazol-2(3H)-yl)hexanoate

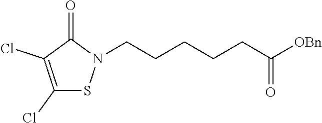 [SMILES: O=C(CCCCCn1sc(Cl)c(Cl)c1=O)OCc1ccccc1]

Dibenzyl 6,6′-((3,3′-disulfanediylbis(propanoyl))bis(azanediyl))dihexanoate (3.21 g, 0.0052 mol, 1 eq.) was dissolved in anhydrous DCM (26 mL). SO₂Cl₂ (pur. 97%, 3.80 mL, 0.047 mol, 9 eq.) was added dropwise to the solution, and the mixture was stirred at rt for 24 h under inert atmosphere. The solution clarified and became pale yellow. Subsequently, the mixture was washed with water (2×100 mL) and brine (1×100 mL). The combined aqueous layers were extracted with DCM (2×100 mL). The organic layers were dried with MgSO₄ and filtered then concentrated under reduced pressure and purified using a chromatography column. Benzyl 6-(4,5-dichloro-3-oxoisothiazol-2(3H)-yl)hexanoate was obtained as a light yellow oil (1.391 g, 35.7%). ¹H NMR (CDCl₃), δ 7.35 (m, 5H), 5.11 (s, 2H), 3.79 (t, J=7.18 Hz, 2H), 2.37 (t, J=7.33 Hz, 2H), 1.70 (m, 4H), 1.38 (m, 2H); ¹³C NMR (500 MHz, CDCl₃), δ 173.2 (—O—C═O), 161.9 (—N—C═O), 138 (—CH₂—O—), 0.3 (Cl—C—S—), 135.6 (C_{quat.} from aromatic cycle), 128.6-128.3 (5H—C_{aromatic}), 115.1 (C₁—C—C═O), 66.2, 44.9 (—CH₂—N—), 33.9 (—CH₂—C═O), 29.1, 25.9, 24.3; R_{t,1} (in ACN): 2.50; MS ES+m/z: 373.75.
I.8. Benzyl6-(4,5-dichloro-1,1-dioxido-3-oxoisothiazol-2(3H)-yl)hexanoate.

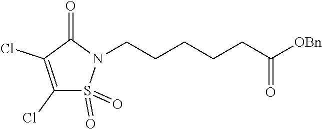 [SMILES: O=C(CCCCCN1C(=O)C(Cl)=C(Cl)S1(=O)=O)OCc1ccccc1]

### Standard Procedure for the Oxidation of Di-Chloride Compounds:

Ruthenium trichloride monohydrated (16 mg, 0.07 mmol. 0.013 eq.) was added in one portion to a stirred solution of Benzyl 6-(4,5-dichloro-3-oxoisothiazol-2(3H)-yl)hexanoate (1.94 g, 0.0052 mol, 1 eq.) in water:DCM:ACN (2:1:1, 1 ml). Sodium periodate (3.33 g, 0.0156 mol, 3 eq.) was then added over 5 min and the resulting mixture stirred at rt for 90 minutes under inert atmosphere. The solids were filtered and the filtrate was diluted with water (50 mL), extracted with EtOAc (2×100 mL), dried with MgSO₄, filtered, and concentrated under reduced pressure. The grey solid obtained was then purified using a chromatography column and Benzyl6-(4,5-dichloro-1,1-dioxido-3-oxoisothiazol-2(3H)-yl)hexanoate was obtained as a pale yellow oil (0.930 g, 44.2%). ¹H NMR (CDCl₃), δ 7.36 (m, 5H), 5.12 (s, 2H), 3.72 (t, J=7.53 Hz, 2H), 2.73 (t, J=7.50 Hz, 2H), 1.80 (t of t, J=7.53 Hz, 2H), 1.70 (t of t, J=7.70 Hz), 1.41 (m, 2H); ¹³C NMR (500 MHz, CDCl₃), δ 173.1 (—O—C═O—), 154.1 (N—C═O), 138.0 (Cl—C—SO₂—), 136.0 (C_{quat.} from aromatic cycle), 130.7 1 (Cl—C—C═O), 128.6-128.3 (5H-C_{aromatic}), 66.2 (—CH₂—O—), 41.1 (—CH₂—N—), 33.9 (—CH₂—C═O), 27.9, 26.0, 24.2; R_{t,1} (in ACN): 2.59; MS ES+m/z: 405.76.

### Example 2. 6-(4,5-Dichloro-1,1-dioxido-3-oxoisothiazol-2(3H)-yl)hexanoic acid

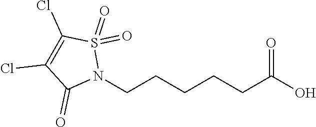 [SMILES: O=C(O)CCCCCN1C(=O)C(Cl)=C(Cl)S1(=O)=O]

### Standard Procedure of Deprotection of Benzyl Esters:

Benzyl6-(4,5-dichloro-1,1-dioxido-3-oxoisothiazol-2(3H)-yl)hexanoate (0.930 g, 2.23 mmol, 1 eq.) was diluted in anhydrous DCM (11.5 mL). Methanesulfonic acid (1.5 mL, 0.023 mol, 10 eq.) was added. The solution was stirred for 24 h at rt under inert atmosphere. The solution was then diluted with DCM, and treated with water (50 mL). The organic layer was extracted with water (2×100 mL) then brine (1×100 mL). The combined aqueous layers were extracted with DCM (2×100 mL). The organic layers were dried over MgSO₄ and concentrated under reduced pressure, then purified using a chromatography column. 6-(4,5-Dichloro-1,1-dioxido-3-oxoisothiazol-2(3H)-yl)hexanoic acid was obtained as a light white powder (0.563 g, 78%). ¹H NMR (500 MHz, CDCl₃), δ=3.76 (t, J=7.34 Hz, 2H), 2.38 (t, J=7.32 Hz, 2H), 1.83 (t oft, J=7.65 Hz, 2H), 1.70 (t of t, J=7.77 Hz, 2H), 1.45 (t oft, J=7.54 Hz, J=3.31 Hz, 2H); ¹³C NMR (500 MHz, CDCl₃), δ 178.2 (O═C—OH), 154.2 (N—C═O), 138.1 (Cl—C—SO₂—), 130.9 (C₁—C—C═O), 41.1 (—CH₂—N—), 33.4 (—CH₂—C═O), 27.9, 25.9, 23.9; R_{t,1} (in ACN): 2.09; MS ES+m/z: 315.76.
I.9. Benzyl 6-(4-bromo-1,1-dioxido-3-oxoisothiazol-2(3H)-yl)hexanoate

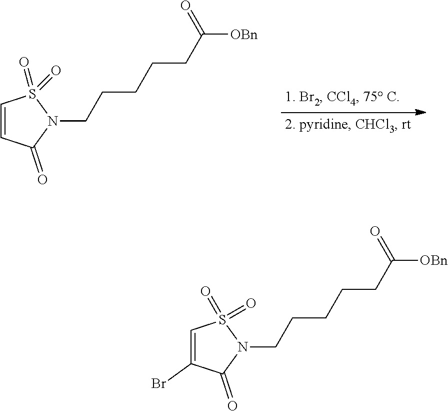 [SMILES: O=C(CCCCCN1C(=O)C=CS1(=O)=O)OCc1ccccc1 ; O=C(CCCCCN1C(=O)C(Br)=CS1(=O)=O)OCc1ccccc1]

To a solution of benzyl 6-(1,1-dioxido-3-oxoisothiazol-2(3H)-yl)hexanoate (obtained following the standard procedure for the oxidation of mono-chloride compounds starting from benzyl 6-(3-oxoisothiazol-2(3H)-yl)hexanoate) (3 g, 8.89 mmol, 1.00 equiv) in CCl₄ (40 mL) was added Bra (1.2 mL, 19.58 mmol, 2.2 equiv) dropwise with stirring at ambient temperature over 30 min and stirred overnight at 75° C. The reaction mixture was concentrated under vacuum and diluted with CHCl₃ (40 mL), which was followed by the addition of pyridine (0.9 g). The resulting solution was stirred for 30 min at ambient temperature and then quenched by the addition of 50 ml saturated NaHCO₃ solution. The resulting mixture was washed with saturated sodium carbonate (2×50 mL) and 50 mL of brine. The resulting mixture was concentrated under vacuum and the residue was purified by a silica gel column with ethyl acetate/petroleum ether (1:5) to afford 0.4 g (10.8%) of benzyl 6-(4-bromo-1,1-dioxido-3-oxoisothiazol-2(3H)-yl)hexanoate as a light yellow oil. LC-MS (ES, m/z): 416 [M+H]⁺, 433 [M+NH₄]; ¹H-NMR (400 MHz, Chloroform-d) δ 7.42-7.28 (m, 2H), 5.12 (s, 1H), 3.69 (t, J=7.4 Hz, 1H), 2.38 (t, J=7.4 Hz, 1H), 1.86-1.64 (m, 2H), 1.47-1.34 (m, 1H).

### Example 3. 6-(4-bromo-1,1-dioxido-3-oxoisothiazol-2(3H)-yl)hexanoic acid

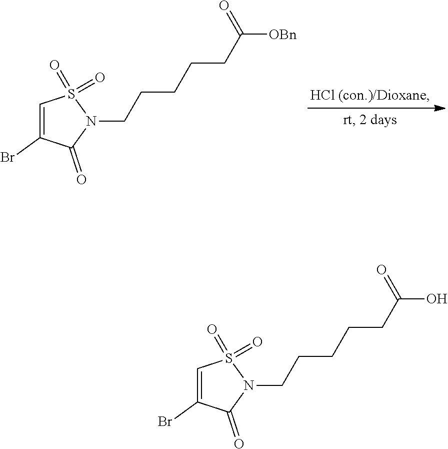 [SMILES: O=C(CCCCCN1C(=O)C(Br)=CS1(=O)=O)OCc1ccccc1 ; O=C(O)CCCCCN1C(=O)C(Br)=CS1(=O)=O]

To a solution of benzyl 6-(4-bromo-1,1-dioxido-3-oxoisothiazol-2(3H)-yl)hexanoate (1 g, 2.40 mmol, 1.00 equiv) in dioxane (10 mL) was added 4N HCl (10 mL) dropwise with stirring at 0° C. The resulting solution was stirred for 2 days at room temperature. The resulting mixture was concentrated under vacuum and extracted with dichloromethane (3×50 mL). The combined organic layer was washed with brine (2×100 mL), dried over anhydrous sodium sulfate and concentrated under vacuum. The residue was purified via a silica gel column with DCM/MeOH (10:1) to afford 100 mg (13%) of 6-(4-bromo-1,1-dioxido-3-oxoisothiazol-2(3H)-yl)hexanoic acid as a white solid. LC-MS (ES, m/z): 308[M+NH₄]⁺, 326/328[M+H]⁺; ¹H-NMR (300 MHz, Chloroform-d) δ 7.58 (s, 1H), 3.75 (t, J=7.4 Hz, 2H), 2.41 (t, J=7.4 Hz, 2H), 1.78 (dq, J=34.6, 7.4 Hz, 4H), 1.47 (t, J=7.7 Hz, 2H).
Example of a Synthetic Path for the Synthesis of a Linker with X₁═OR:

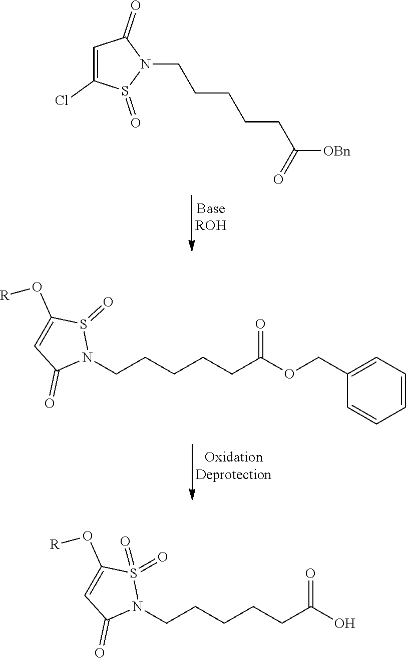

I.10. Benzyl 6-(5-(4-cyanophenoxy)-1-oxido-3-oxoisothiazol-2(3H)-yl)hexanoate

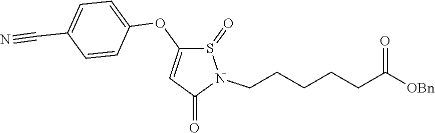 [SMILES: N#Cc1ccc(OC2=CC(=O)N(CCCCCC(=O)OCc3ccccc3)S2=O)cc1]

A solution of 4-hydroxybenzenecarbonitrile (76 mg, 0639 mmol) in THF (2 mL) was added at 0° C. to a mixture of NaH (0.639 mmol) in THF (2 mL). After 30 minutes under stirring, benzyl 6-(5-chloro-1-oxido-3-oxoisothiazol-2(3H)-yl)hexanoate (250 mg, 0,703 mmol) in THF (2 mL) was added. The reaction mixture was then stirred at room temperature for 18 h. The reaction mixture was diluted with AcOEt and NH₄Cl (10% aqueous) was added. The organic phase was then washed with brine and dried over MgSO4, filtered and concentrated. The crude product was purified over silica gel column using DCM/MeOH mixture (80/20) to afford Benzyl 6-(5-(4-cyanophenoxy)-1-oxido-3-oxoisothiazol-2(3H)-yl)hexanoate (210 mg, 75% yield) as a colorless oil. LC-MS (ES, m/z): 439.0 [M+H]+; ¹H-NMR (300 MHz, Chloroform-d) δ 7.79 (m, 2H), 7.35 (m, 7H), 5.59 (s, 1H), 5.12 (s, 2H), 3.69 (m, 2H), 2.37 (m, 2H), 1.71 (m, 4H), 1.39 (m, 2H).
I.11. Benzyl 6-(5-(4-cyanophenoxy)-1,1-dioxido-3-oxoisothiazol-2(3H)-yl)hexanoate

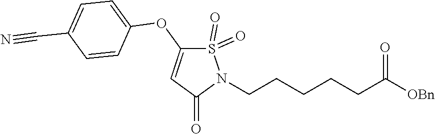 [SMILES: N#Cc1ccc(OC2=CC(=O)N(CCCCCC(=O)OCc3ccccc3)S2(=O)=O)cc1]

Obtained as a colorless oil following the standard procedure for the oxidation of mono-chloride compounds (136 mg, 48% yield) starting from Benzyl 6-(5-(4-cyanophenoxy)-1-oxido-3-oxoisothiazol-2(3H)-yl)hexanoate. LC-MS (ES, m/z): 455.0 [M+H]+; ¹H-NMR (300 MHz, Chloroform-d) δ 7.82 (m, 2H), 7.40 (m, 2H), 7.35 (m, 5H), 5.63 (s, 1H), 5.12 (s, 2H), 3.65 (m, 2H), 2.38 (m, 2H), 1.79 (m, 2H), 1.70 (m, 2H), 1.41 (m, 2H).

### Example 4. 6-(5-(4-cyanophenoxy)-1,1-dioxido-3-oxoisothiazol-2(3H)-yl)hexanoic acid

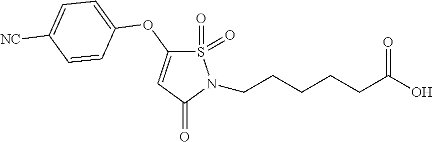 [SMILES: N#Cc1ccc(OC2=CC(=O)N(CCCCCC(=O)O)S2(=O)=O)cc1]

Obtained as a white solid following the standard procedure for deprotection of benzyl esters (38 mg, 35% yield) starting from Benzyl 6-(5-(4-cyanophenoxy)-1,1-dioxido-3-oxoisothiazol-2(3H)-yl)hexanoate. LC-MS (ES, m/z): 365.0 [M+H]+; ¹H-MR (300 MHz, Chloroform-d) 7.82 (m, 2H), 7.41 (m, 2H), 5.65 (s, 1H), 3.67 (m, 2H), 2.38 (m, 2H), 1.80 (m, 2H), 1.69 (m, 2H), 1.44 (m, 2H).
I.12. Benzyl 6-(5-methoxy-1-oxido-3-oxoisothiazol-2(3H)-yl)hexanoate

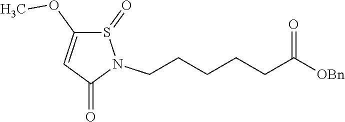 [SMILES: COC1=CC(=O)N(CCCCCC(=O)OCc2ccccc2)S1=O]

A mixture of benzyl 6-(5-chloro-1-oxido-3-oxoisothiazol-2(3H)-yl)hexanoate (270 mg, 0,759 mmol) in methanol (5 mL) and triethylamine (113 μl, 0,835 mmol) was stirred at room temperature for 18 h. Volatiles were then removed under vacuum and the residue purified over a silica column cyclohexane/AcOEt (1/1) to afford Benzyl 6-(5-methoxy-1-oxido-3-oxoisothiazol-2(3H)-yl)hexanoate (107 mg, 40%) as a yellow oil. LC-MS (ES, m/z): 352.0 [M+H]+; ¹H-NMR (300 MHz, Chloroform-d) δ 7.36 (m, 5H), 5.57 (s, 1H), 5.11 (s, 2H), 4.04 (s, 3H), 3.60 (m, 2H), 2.37 (m, 2H), 1.75 (m, 2H), 1.69 (m, 2H), 1.39 (m, 2H).
I.13. Benzyl 6-(5-methoxy-1,1-dioxido-3-oxoisothiazol-2(3H)-yl)hexanoate

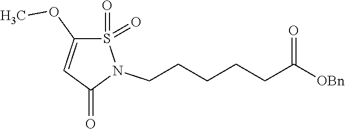 [SMILES: COC1=CC(=O)N(CCCCCC(=O)OCc2ccccc2)S1(=O)=O]

Obtained as a white solid following the standard procedure for the oxidation of mono-chloride compounds (62 mg, 36% yield) starting from benzyl 6-(5-methoxy-1-oxido-3-oxoisothiazol-2(3H)-yl)hexanoate. LC-MS (ES, m/z): 368.0 [M+H]+; ¹H-NMR (300 MHz, Chloroform-d) δ 7.36 (m, 5H), 5.57 (s, 1H), 5.11 (s, 2H), 4.04 (s, 3H), 3.60 (m, 2H), 2.37 (m, 2H), 1.75 (m, 2H), 1.69 (m, 2H), 1.39 (m, 2H).

### Example 5. 6-(5-Methoxy-1,1-dioxido-3-oxoisothiazol-2(3H)-yl)hexanoic acid

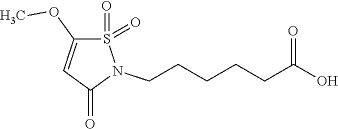

Obtained as a white solid following the standard procedure for deprotection of benzyl esters (37 mg, 67% yield) starting from benzyl 6-(5-methoxy-1,1-dioxido-3-oxoisothiazol-2(3H)-yl)hexanoate. HRMS (ES, m/z): [M+H] found 278.0691 for 278.0698 calculated; ¹H-NMR (300 MHz, Chloroform-d) δ 5.60 (s, 1H), 4.05 (s, 3H), 3.62 (m, 2H), 2.36 (m, 2H), 1.77 (m, 2H), 1.68 (m, 2H), 1.42 (m, 2H).
I.14. Benzyl 4-(aminomethyl)cyclohexane-1-carboxylate 4-methylbenzenesulfonate

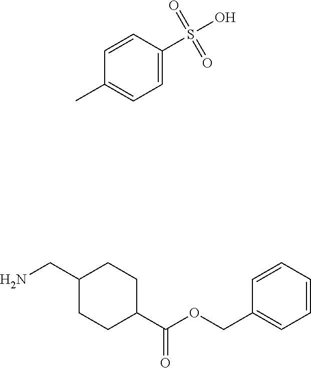 [SMILES: Cc1ccc(S(=O)(=O)O)cc1 ; NCC1CCC(C(=O)OCc2ccccc2)CC1]

A mixture of 4-(aminomethyl)cyclohexanecarboxylic acid (10 g, 63.61 mmol, 1 eq), phenylmethanol (55.03 g, 508.87 mmol, 52.91 mL, 8 eq) and TsOH·H₂O (12.70 g, 66.79 mmol, 1.05 eq) in toluene (50 mL) was stirred at 140° C. for 16 hours using a Dean and Stark apparatus to collect the water of condensation and from the toluene sulphonic acid monohydrate. The reaction turned to clear after refluxing for several hours. The clear reaction mixture was poured into TBME (500 mL) and the resultant white solid filtered off, washed with TBME (200 mL) and dried in vacuum. Benzyl 4-(aminomethyl)cyclohexanecarboxylate; 4-methylbenzenesulfonic acid (26.6 g, 63.40 mmol, 99.68% yield) was obtained as a white solid; ¹H NMR (400 MHz, METHANOL-d4) δ ppm 7.71 (d, J=8.16 Hz, 2H) 7.28-7.40 (m, 5H) 7.23 (d, J=7.94 Hz, 2H) 5.11 (s, 2H) 2.77 (d, J=7.06 Hz, 2H) 2.29-2.39 (m, 4H) 1.99-2.08 (m, 2H) 1.85 (brd, J=11.25 Hz, 2H) 1.53-1.65 (m, 1H) 1.43 (qd, J=12.97, 3.20 Hz, 2H) 1.06 (qd, J=12.75, 3.20 Hz, 2H).
I.15. Dibenzyl 4,4′-(((3,3′-disulfanediylbis(propanoyl))bis(azanediyl))bis(methylene))bis (cyclohexane-1-carboxylate)

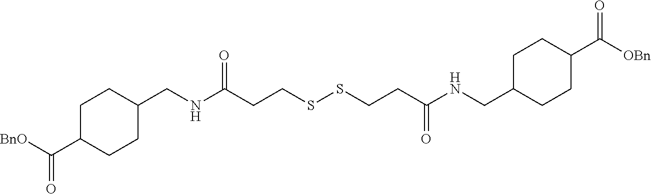 [SMILES: O=C(CCSSCCC(=O)NCC1CCC(C(=O)OCc2ccccc2)CC1)NCC1CCC(C(=O)OCc2ccccc2)CC1]

To a solution of 3-(2-carboxyethyldisulfanyl)propanoic acid (6.64 g, 31.58 mmol, 1 eq), HOBt (9.39 g, 69.48 mmol, 2.2 eq) and TEA (12.78 g, 126.33 mmol, 17.58 mL, 4 eq) in DCM (300 mL) was added EDCI (13.32 g, 69.48 mmol, 2.2 eq) at 0° C. Then benzyl 4-(aminomethyl)cyclohexanecarboxylate; 4-methylbenzenesulfonic acid (26.5 g, 63.17 mmol, 2 eq) was added at this temperature. The mixture was stirred at 0-20° C. for 4 hrs. TLC (Petroleum ether:Ethyl acetate=2:1, R_{f}=0.5) indicated the reaction was completed. The mixture was poured into sat. NaHCO₃ (100 mL) and H₂O (100 mL) and the organic layer was separated. The aqueous layer was extracted with DCM (200 mL). The combined organic layers were washed with H₂O (100 mL), brine (100 mL), dried over Na₂SO₄, filtered and concentrated in vacuum. The residue was dissolved into DCM (50 mL), added petroleum ether very slowly until the white precipitate was formed. Filtered and washed with petroleum ether, dried over vacuum. Benzyl 4-[[3-[[3-[(4-benzyloxycarbonylcyclohexyl)methylamino]-3-oxo-propyl]disulfanyl]propanoylamino]methyl]cyclohexanecarboxylate (19 g, 28.40 mmol, 89.94% yield) was obtained as a white solid; ¹H NMR (400 MHz, CHLOROFORM-d) δ ppm 7.27-7.41 (m, 10H) 6.05 (br s, 2H) 5.11 (d, J=1.54 Hz, 4 H) 3.10-3.17 (m, 4H) 2.96-3.02 (m, 4H) 2.54-2.63 (m, 4H) 2.30 (td, J=12.24, 1.76 Hz, 2H) 2.04 (br d, J=12.57 Hz, 4H) 1.85 (br d, J=12.79 Hz, 4H) 1.38-1.54 (m, 6H) 0.99 (q, J=12.72 Hz, 4H).
I.16. Benzyl 4-((5-chloro-3-oxoisothiazol-2(3H)-yl)methyl)cyclohexane-1-carboxylate

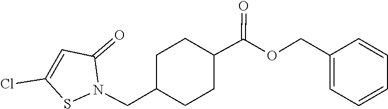 [SMILES: O=C(OCc1ccccc1)C1CCC(Cn2sc(Cl)cc2=O)CC1]

I.17. Benzyl 4-((4,5-dichloro-3-oxoisothiazol-2(3H)-yl)methyl)cyclohexane-1-carboxylate

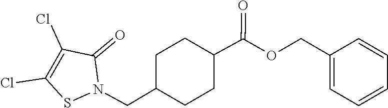 [SMILES: O=C(OCc1ccccc1)C1CCC(Cn2sc(Cl)c(Cl)c2=O)CC1]

To a solution of benzyl 4-[[3-[[3-[(4-benzyloxycarbonylcyclohexyl)methylamino]-3-oxo-propyl] disulfanyl]propanoylamino]methyl]cyclohexanecarboxylate (10 g, 14.95 mmol, 1 eq) in DCM (100 mL) was added dropwise sulfuryl chloride (10.09 g, 74.75 mmol, 7.47 mL, 5 eq) at 0° C. The mixture was stirred at 0-20° C. for 12 hrs. The clear solution was obtained after the addition of the sulfuryl chloride. TLC (Petroleum ether:Ethyl acetate=2:1, Rf. (major)=0.5) indicated the reaction was completed. The mixture was poured into H₂O (30 mL), extracted with DCM (50 mL*2). The combined organic layers were washed with H₂O (50 mL), brine (50 mL), dried over Na₂SO₄, filtered and concentrated in vacuum. Purification over silica gel column afforded Benzyl 4-[(3-oxoisothiazol-2-yl)methyl]cyclohexanecarboxylate (2.1 g, 3.42 mmol, 11.44% yield, 54% purity) obtained as a brown solid, Benzyl 4-[(5-chloro-3-oxo-isothiazol-2-yl)methyl]cyclohexanecarboxylate (7.1 g, 18.82 mmol, 62.96% yield, 97% purity) obtained as an off-white solid and Benzyl 4-[(4,5-dichloro-3-oxo-isothiazol-2-yl)methyl]cyclohexanecarboxylate (0.4 g, 869.31 umol, 2.91% yield, 87% purity) obtained as a brown oil.

I.18. Benzyl 4-((5-chloro-1,1-dioxido-3-oxoisothiazol-2(3H)-yl)methyl)cyclohexane-1-carboxylate

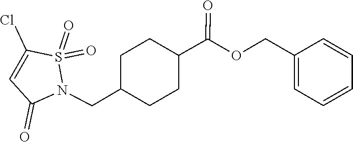 [SMILES: O=C(OCc1ccccc1)C1CCC(CN2C(=O)C=C(Cl)S2(=O)=O)CC1]

To a mixture of benzyl 4-[(5-chloro-3-oxo-isothiazol-2-yl)methyl]cyclohexanecarboxylate (2 g, 5.01 mmol, 1 eq) in H₂O (20 mL), ACN (10 mL) and DCM (10 mL) was added RuCl₃·H₂O (22.58 mg, 100.14 umol, 0.02 eq) and NaIO₄ (6.43 g, 30.04 mmol, 1.66 mL, 6 eq) in one portion at 0° C. under N₂. The mixture then heated to 20° C. and stirred for 16 hours. TLC showed the reaction was completed (Petroleum ether:Ethyl acetate=2:1, Rf-p1=0.6). The mixture was filtered, the filtrate was concentrated by nitrogen flow, and the solid that appeared again was filtered, the filtrate was concentrated by nitrogen. The residue was purified by preparative TLC (column height: 250 mm, diameter: 100 mm, 100-200 mesh silica gel, Petroleum ether:Ethyl acetate=2:1) to afford benzyl 4-[(5-chloro-1,1,3-trioxo-isothiazol-2-yl)methyl]cyclohexanecarboxylate (1.4 g, 3.17 mmol, 63.25% yield, 90% purity) as white solid.

### Example 6. 4-((5-chloro-1,1-dioxido-3-oxoisothiazol-2(3H)-yl)methyl)cyclohexane-1-carboxylic acid

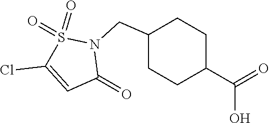 [SMILES: O=C(O)C1CCC(CN2C(=O)C=C(Cl)S2(=O)=O)CC1]

To a mixture of benzyl 4-[(5-chloro-1,1,3-trioxo-isothiazol-2-yl)methyl]cyclohexanecarboxylate (0.1 g, 226.20 umol, 1 eq) in DCM (5 mL) was added MsOH (217.39 mg, 2.26 mmol, 161.03 uL, 10 eq) in one portion at 30° C. under N₂. The mixture was stirred at 30° C. for 16 hours. TLC showed the reaction was completed. LCMS (ET17992-54-P1A) showed desired MS detected. The mixture was poured into ice-water (5 mL) and stirred for 5 min. The aqueous phase was extracted with DCM (3 mL*2). The combined organic phase was washed with brine (3 mL), dried with anhydrous Na₂SO₄, filtered and concentrated in vacuum. The residue was purified by preparative TLC (column height: 250 mm, diameter: 100 mm, 100-200 mesh silica gel, Petroleum ether:/Ethyl acetate=2:1) to afford 4-[(5-chloro-1,1,3-trioxo-isothiazol-2-yl)methyl]cyclohexanecarboxylic acid (0.02 g, 64.99 umol, 28.73% yield) as colorless oil; LC-MS (ES, m/z): 306.0 [M−H]⁻; ¹H-NMR (300 MHz, DMSO-D6) δ 12.01 (bs, 1H), 7.62 (s, 1H), 3.45 (m, 2H), 2.11 (m, 1H), 1.90 (m, 2H), 1.76 (m, 2H), 1.73 (m, 1H), 1.23 (m, 2H), 0.96 (m, 2H).

I.19 Benzyl 4-((4,5-dichloro-1,1-dioxido-3-oxoisothiazol-2(3H)-yl)methyl)cyclohexane-1-carboxylate

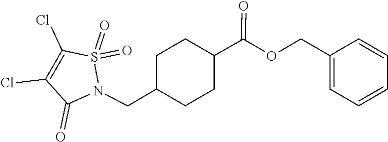 [SMILES: O=C(OCc1ccccc1)C1CCC(CN2C(=O)C(Cl)=C(Cl)S2(=O)=O)CC1]

To a mixture of benzyl 4-[(4,5-dichloro-3-oxo-isothiazol-2-yl)methyl]cyclohexanecarboxylate (1.2 g, 2.70 mmol, 1 eq) in H₂O (20 mL), DCM (10 mL) and ACN (10 mL) was added RuCl₃·H₂O (12.16 mg, 53.96 umol, 0.02 eq) and NaIO₄ (3.46 g, 16.19 mmol, 896.96 uL, 6 eq) in one portion at 0° C. under N₂. The mixture was stirred at 20° C. for 2 hours. TLC showed the reaction was completed (Petroleum ether:Ethyl acetate=2:1, Rf-pl=0.7). The mixture was poured into ice-water (30 mL) and stirred for 5 min. The aqueous phase was extracted with ethyl acetate (20 mL*2). The combined organic phases were washed with brine (20 mL), dried with anhydrous Na₂SO₄, filtered and concentrated in vacuum. The residue was purified by silica gel chromatography (column height: 250 mm, diameter: 100 mm, 100-200 mesh silica gel, Petroleum ether:Ethyl acetate=5:1 to 3:1) to afford benzyl 4-[(4,5-dichloro-1,1,3-trioxo-isothiazol-2-yl)methyl]cyclohexanecarboxylate (0.8 g, 1.67 mmol, 61.73% yield, 90% purity) as white solid.

### Example 7. 4-((4,5-dichloro-1,1-dioxido-3-oxoisothiazol-2(3H)-yl)methyl)cyclohexane-1-carboxylic acid

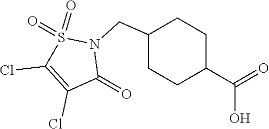 [SMILES: O=C(O)C1CCC(CN2C(=O)C(Cl)=C(Cl)S2(=O)=O)CC1]

To a mixture of benzyl 4-[(4,5-dichloro-1,1,3-trioxo-isothiazol-2-yl)methyl]cyclohexanecarboxylate (0.8 g, 1.67 mmol, 1 eq) in DCM (20 mL) was added MsOH (1.60 g, 16.65 mmol, 1.19 mL, 10 eq) in one portion at 30° C. under N₂. The mixture was stirred at 30° C. for 16 hours. LCMS showed the reaction was completed. The mixture was poured into ice-water (5 mL) and concentrated under reduced pressure then a solid appeared. The solution was filtered and trituration by EtOAc (2 mL*3) and the filter cake was dried in vacuum to afford 4-[(4,5-dichloro-1,1,3-trioxo-isothiazol-2-yl)methyl]cyclohexanecarboxylic acid (0.170 g, 486.86 umol, 29.23% yield, 98% purity) as white solid; LC-MS (ES, m/z): 364.0 [M+Na]⁺; ¹H-NMR (300 MHz, DMSO-D6) δ 11.99 (bs, 1H), 3.49 (m, 2H), 2.11 (m, 1H), 1.88 (m, 2H), 1.79 (m, 2H), 1.66 (m, 1H), 1.23 (m, 2H), 0.96 (m, 2H).
I.20 Dibenzyl 3,3′-(((3,3′-disulfanediylbis(propanoyl))bis(azanediyl))bis(4,1-phenylene)) dipropionate

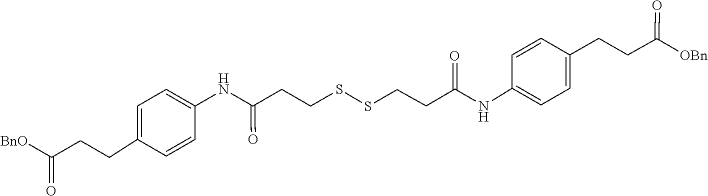 [SMILES: O=C(CCSSCCC(=O)Nc1ccc(CCC(=O)OCc2ccccc2)cc1)Nc1ccc(CCC(=O)OCc2ccccc2)cc1]

To a solution of 3-(2-carboxyethyldisulfanyl)propanoic acid (651.56 mg, 3.10 mmol, 1 eq), HOBt (921.15 mg, 6.82 mmol, 2.2 eq) and TEA (1.25 g, 12.39 mmol, 1.73 mL, 4 eq) in DCM (50 mL) was added EDCI (1.31 g, 6.82 mmol, 2.2 eq) at 0° C. Then benzyl 4-(aminomethyl)cyclohexanecarboxylate; 4-methylbenzenesulfonic acid (2.6 g, 6.20 mmol, 2 eq) was added at this temperature. The mixture was stirred at 0-20° C. for 12 hrs. TLC (Petroleum ether:Ethyl acetate=2:1, R_{f}=0.5) indicated the reaction was completed. The mixture was poured into sat. NaHCO₃ (20 mL) and H₂O (20 mL) and the organic layer was separated. The aqueous layer was extracted with DCM (50 mL). The combined organic layers were washed with H₂O (50 mL), brine (50 mL), dried over Na₂SO₄, filtered and concentrated in vacuum. Benzyl 4-[[3-[[3-[(4-benzyloxycarbonylcyclohexyl)methylamino]-3-oxo-propyl]disulfanyl]propanoyl amino]methyl]cyclohexanecarboxylate (1.1 g, 1.64 mmol, 53.07% yield) was obtained as a white solid; ¹H NMR (400 MHz, CHLOROFORM-d) δ ppm 7.28-7.41 (m, 10H) 5.96 (br s, 2H) 5.10 (s, 4H) 3.13 (t, J=6.39 Hz, 4H) 2.98 (t, J=6.84 Hz, 4H) 2.57 (t, J=6.95 Hz, 4H) 2.23-2.34 (m, 2H) 2.03 (br d, J=12.79 Hz, 4H) 1.84 (br d, J=12.13 Hz, 4H) 1.37-1.63 (m, 6H) 0.91-1.05 (m, 4H).
I.21. Benzyl 3-(4-(5-chloro-3-oxoisothiazol-2(3H)-yl)phenyl)propanoate

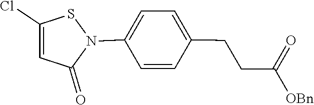 [SMILES: O=C(CCc1ccc(-n2sc(Cl)cc2=O)cc1)OCc1ccccc1]

To a solution of benzyl 3-[4-[3-[[3-[4-(3-benzyloxy-3-oxo-propyl)anilino]-3-oxo-propyl]disulfanyl]propanoylamino]phenyl]propanoate (10 g, 14.60 mmol, 1 eq) in DCM (200 mL) was added sulfuryl chloride (5.91 g, 43.80 mmol, 4.38 mL, 3 eq) dropwise at 25° C. under N₂. The solution was stirred at 25° C. for 8 hours. The color of solution changed from colorless to black when sulfuryl chloride was added and then changed to yellow after 1 hour. TLC (Petroleum ether:Ethyl acetate=2:1, Rf=0.60) showed starting material was consumed and two new main spots were generated. The residue was poured into ice-water (200 ml) and then concentrated in vacuum to remove DCM. After concentration, the aqueous phase was extracted with ethyl acetate (200 mL*3) and then the combined organic phase was washed with water (200 mL*1), dried with anhydrous Na₂SO₄, filtered and concentrated in vacuum. The residue was purified by column chromatography (SiO₂, Petroleum ether:Ethyl acetate=5:1 to 1:1) to get benzyl 3-[4-(3-oxoisothiazol-2-yl)phenyl]propanoate (2.5 g, 7.37 mmol, 50.47% yield) ¹H NMR (400 MHz, METHANOL-d4) 6=8.56 (d, J=6.2 Hz, 1H), 7.44-7.39 (m, 2H), 7.37-7.26 (m, 7H), 6.30 (d, J=6.4 Hz, 1H), 5.09 (s, 2H), 2.98 (t, J=7.4 Hz, 2H), 2.78-2.66 (m, 2H); and benzyl 3-[4-(5-chloro-3-oxo-isothiazol-2-yl)phenyl]propanoate (5 g, 13.37 mmol, 91.60% yield) was a yellow solid; ¹H NMR (ET17992-22-P2A) confirmed ET17992-22-P2. ¹H NMR (400 MHz, METHANOL-d4) 6=7.42-7.36 (m, 2H), 7.35-7.25 (m, 7H), 6.49-6.45 (m, 1H), 5.08 (s, 2H), 2.99-2.91 (m, 2H), 2.69 (t, J=7.5 Hz, 2H).
I.22. Benzyl 3-(4-(5-chloro-1,1-dioxido-3-oxoisothiazol-2(3H)-yl)phenyl)propanoate

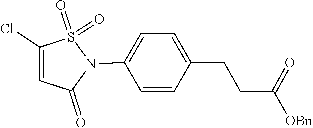 [SMILES: O=C(CCc1ccc(N2C(=O)C=C(Cl)S2(=O)=O)cc1)OCc1ccccc1]

To a mixture of benzyl 3-[4-(5-chloro-3-oxo-isothiazol-2-yl)phenyl]propanoate (1.4 g, 3.74 mmol, 1 eq) in H₂O (12 mL), DCM (6 mL) and ACN (6 mL) was added NaIO₄ (4.81 g, 22.47 mmol, 1.25 mL, 6 eq) in one portion at 25° C. and then the mixture was purged with N₂ three times. Then, RuCl₃·H₂O (42.21 mg, 187.24 umol, 0.05 eq) was added under N₂. The mixture was stirred at 25° C. for 12 hrs. The mixture turned turbidity and the color become to gray. The residue was poured into Ethyl acetate (100 ml) and then filtered. The filtrate was concentrated in vacuum. The residue was purified by column chromatography (SiO₂, Petroleum ether:Ethyl acetate=10:1 to 4:1) to give benzyl 3-[4-(5-chloro-1,1,3-trioxo-isothiazol-2-yl)phenyl]propanoate (460 mg, 1.08 mmol, 28.75% yield, 95% purity) as a yellow solid; ¹H NMR (ET17992-63-P1A) confirmed ET17992-63-P1. ¹H NMR (400 MHz, CHLOROFORM-d) 6=7.41-7.30 (m, 7H), 6.84 (s, 1H), 5.13 (s, 2H), 3.04 (t, J=7.6 Hz, 2H), 2.72 (t, J=7.7 Hz, 2H).

### Example 8. 3-(4-(5-chloro-1,1-dioxido-3-oxoisothiazol-2(3H)-yl)phenyl)propanoic acid

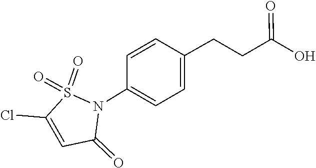 [SMILES: O=C(O)CCc1ccc(N2C(=O)C=C(Cl)S2(=O)=O)cc1]

A solution of benzyl 3-[4-(5-chloro-1,1,3-trioxo-isothiazol-2-yl)phenyl]propanoate (460 mg, 1.13 mmol, 1 eq) in DCM (15 mL) was added methanesulfonic acid (1.09 g, 11.33 mmol, 806.87 uL, 10 eq) dropwise at 10° C. Then, the solution was heated to 35° C. and stirred for 12 hrs. The residue was washed with water (15 ml*3), dried with anhydrous Na₂SO₄, filtered and concentrated in vacuum. The residue was poured into water (20 ml) and then filtered. The filter cake was dissolved in DCM (5 ml) and then petroleum ether (30 ml) was poured into the residue, the solution was stirred at 10° C. for 2 min, and then filtered, the filter cake was dried in vacuum to give 3-[4-(5-chloro-1,1,3-trioxo-isothiazol-2-yl)phenyl]propanoic acid (162 mg, 501.81 umol, 44.27% yield, 97.8% purity) as a white solid; LC-MS (ES, m/z): 313.9 [M−H]⁻; ¹H-NMR (300 MHz, DMSO-D6) δ 12.18 (bs, 1H), 7.81 (s, 1H), 7.45 (m, 2H), 7.37 (m, 2H), 2.89 (m, 2H), 2.58 (m, 2H).
I.23. Benzyl 3-(4-(4,5-dichloro-3-oxoisothiazol-2(3H)-yl)phenyl)propanoate

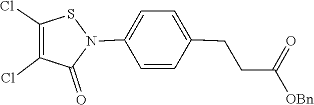 [SMILES: O=C(CCc1ccc(-n2sc(Cl)c(Cl)c2=O)cc1)OCc1ccccc1]

To a mixture of benzyl 4-[(3-oxoisothiazol-2-yl)methyl]cyclohexanecarboxylate (1.2 g, 3.32 mmol, 1 eq) in H₂O (20 mL), ACN (10 mL) and DCM (10 mL) was added RuCl₃·H₂O (14.95 mg, 66.33 umol, 0.02 eq) and NaIO₄ (4.26 g, 19.90 mmol, 1.10 mL, 6 eq) in one portion at 0° C. under N₂. The mixture then heated to 20° C. and stirred for 16 hours. The mixture was filtered, and the filtrate was concentrated by nitrogen flow, and a solid appeared again and filtered, the filtrate was concentrated by nitrogen. The residue was purified by prep-TLC (column height: 250 mm, diameter: 100 mm, 100-200 mesh silica gel, Petroleum ether:Ethyl acetate=2:1) to afford benzyl 4-[(1,1,3-trioxoisothiazol-2-yl)methyl]cyclohexanecarboxylate (0.45 g, 1.11 mmol, 33.60% yield, 90% purity) as a colorless oil; ¹H NMR (400 MHz, CHLOROFORM-d) δ=7.53 (dd, J=3.2, 8.7 Hz, 2H), 7.17 (ddd, J=3.2, 7.6, 9.0 Hz, 2H), 6.97-6.89 (m, 2H), 5.56 (br s, 1H), 4.12-4.06 (m, 4H), 3.92 (s, 5H), 3.55 (q, J=5.1 Hz, 5H).
I.24. Benzyl 3-(4-(4,5-dichloro-1,1-dioxido-3-oxoisothiazol-2(3H)-yl)phenyl)propanoate

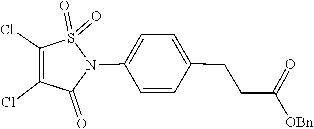 [SMILES: O=C(CCc1ccc(N2C(=O)C(Cl)=C(Cl)S2(=O)=O)cc1)OCc1ccccc1]

To a mixture of benzyl 3-[4-(4,5-dichloro-3-oxo-isothiazol-2-yl)phenyl]propanoate (650 mg, 1.59 mmol, 1 eq) and NaIO₄ (1.36 g, 6.37 mmol, 352.86 uL, 4 eq) in H₂O (10 mL), DCM (5 mL), ACN (5 mL) was added RuCl₃·H₂O (7.18 mg, 31.84 umol, 0.02 eq) in one portion at 0° C. under N₂. The mixture was stirred at 20° C. for 2 hrs. The residue was extracted with ethyl acetate (30 mL*2). The combined organic phase was dried with anhydrous Na₂SO₄, filtered and concentrated in vacuum. The residue was purified by column chromatography (SiO₂, Petroleum ether:Ethyl acetate=30:1 to 5:1) to give benzyl 3-[4-(4,5-dichloro-1,1,3-trioxo-isothiazol-2-yl)phenyl]propanoate (180 mg, 25.68% yield) as a yellow solid; ¹H NMR (400 MHz, CHLOROFORM-d) δ=7.43-7.29 (m, 9H), 5.13 (s, 2H), 3.05 (t, J=7.6 Hz, 2H), 2.73 (t, J=7.6 Hz, 2H).

### Example 9. 3-(4-(4,5-dichloro-1,1-dioxido-3-oxoisothiazol-2(3H)-yl)phenyl)propanoic acid

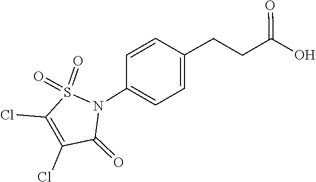 [SMILES: O=C(O)CCc1ccc(N2C(=O)C(Cl)=C(Cl)S2(=O)=O)cc1]

A solution of benzyl 3-[4-(4,5-dichloro-1,1,3-trioxo-isothiazol-2-yl)phenyl]propanoate (180 mg, 408.81 umol, 1 eq) in DCM (5 mL) was added methanesulfonic acid (392.89 mg, 4.09 mmol, 291.03 uL, 10 eq) dropwise at 10° C. The solution was stirred at 35° C. for 12 hrs. The residue was poured into water (5 ml) and then filtered. The filter cake was washed with water (10 ml*3) and then dried in vacuum. The residue was dissolved into Dichloromethane:Methanol (5.5 ml, v/v=10:1) and then purified by prep-TLC (Ethyl acetate, Rf=0.26) to give 3-[4-(4,5-dichloro-1,1,3-trioxo-isothiazol-2-yl)phenyl]propanoic acid (57.64 mg, 156.83 umol, 38.36% yield, 95.278% purity) as a white solid; LC-MS (ES, m/z): 347.9 [M−H]—; ¹H NMR (400 MHz, DMSO-d6) δ=12.22 (br s, 1H), 7.51-7.40 (m, 4H), 2.90 (br t, J=7.6 Hz, 2H), 2.60 (br t, J=7.6 Hz, 2H).
I.25. Dibenzyl 4,4′-((3,3′-disulfanediylbis(propanoyl))bis(azanediyl))dibenzoate

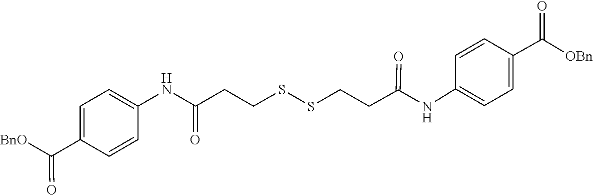 [SMILES: O=C(CCSSCCC(=O)Nc1ccc(C(=O)OCc2ccccc2)cc1)Nc1ccc(C(=O)OCc2ccccc2)cc1]

To a solution of 3-(2-carboxyethyldisulfanyl)propanoic acid (6.01 g, 28.60 mmol, 1 eq) and pyridine (14.93 g, 188.77 mmol, 15.24 mL, 6.6 eq) in DMF (120 mL) was added EDCI (12.06 g, 62.92 mmol, 2.2 eq) and benzyl 4-aminobenzoate (13 g, 57.20 mmol, 2 eq) at 10° C. Then, the mixture was stirred at 50° C. for 12 hrs. The residue was poured into ice-water (200 mL) and stirred for 20 min. The aqueous phase was extracted with ethyl acetate (200 mL*3). The combined organic phase was washed with sat. NaCl (200 mL*3), dried with anhydrous Na₂SO₄, filtered and concentrated in vacuum. Then, the residue was recrystallized from Petroleum ether:DCM=50:1 to get the solid. The solid was washed petroleum three times (150 ml*3), and then dried in vacuum to give benzyl 4-[3-[[3-(4-benzyloxycarbonylanilino)-3-oxo-propyl]disulfanyl]propanoylamino]benzoate (14 g, crude) as a white solid; ¹H NMR (400 MHz, DMSO-d6) δ=10.37 (s, 2H), 8.02-7.83 (m, 4H), 7.72 (d, J=8.8 Hz, 4H), 7.48-7.32 (m, 10H), 5.31 (s, 4H), 3.05-2.98 (m, 4H), 2.81-2.75 (m, 4H).
I.26. Benzyl 4-(5-chloro-3-oxoisothiazol-2(3H)-yl)benzoate

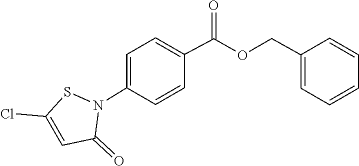 [SMILES: O=C(OCc1ccccc1)c1ccc(-n2sc(Cl)cc2=O)cc1]

I.27. benzyl 4-(4,5-dichloro-3-oxoisothiazol-2(3H)-yl)benzoate

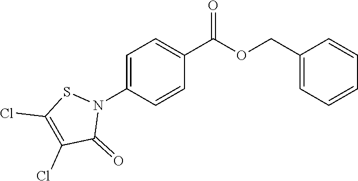 [SMILES: O=C(OCc1ccccc1)c1ccc(-n2sc(Cl)c(Cl)c2=O)cc1]

To a solution of benzyl 4-[3-[[3-(4-benzyloxycarbonylanilino)-3-oxo-propyl]disulfanyl]propanoylamino]benzoate (9.4 g, 14.95 mmol, 1 eq) in DCM (120 mL) was added dropwise sulfuryl chloride (10.09 g, 74.75 mmol, 7.47 mL, 5 eq) at 0° C. The mixture was stirred at 0-20° C. for 12 hrs. The mixture turned to clear after stirring for several minutes. TLC (Petroleum ether:Ethyl acetate=2:1) indicated the reaction was completed. The mixture was poured into H₂O (200 mL), extracted with DCM (200 mL*2). The combined organic layers were washed with H₂O (200 mL*2), dried over Na₂SO₄, filtered and concentrated in vacuum. The residue was purified by column chromatography on silica gel (Petroleum ether:Ethyl acetate=5:1 to 1:1) to give benzyl 4-(3-oxoisothiazol-2-yl)benzoate (1.2 g, 3.43 mmol, 11.47% yield, 89% purity) was obtained as an off-white solid; ¹H NMR (400 MHz, CHLOROFORM-d) δ ppm 8.09-8.27 (m, 3H) 7.63-7.82 (m, 2H) 7.32-7.52 (m, 5H) 6.34 (br d, J=6.36 Hz, 1H) 5.39 (s, 2H); Benzyl 4-(5-chloro-3-oxo-isothiazol-2-yl)benzoate (3.5 g, 10.01 mmol, 33.49% yield, 98.92% purity) was obtained as an off-white solid; ¹H NMR (400 MHz, CHLOROFORM-d) δ ppm 8.15 (d, J=8.60 Hz, 2H) 7.69 (d, J=8.82 Hz, 2H) 7.33-7.49 (m, 4H) 6.38 (s, 1H) 5.38 (s, 2H) and Benzyl 4-(4,5-dichloro-3-oxo-isothiazol-2-yl)benzoate (2.8 g, 7.19 mmol, 24.06% yield, 97.68% purity) was obtained as an off-white solid; ¹H NMR (400 MHz, CHLOROFORM-d) δ ppm 8.18 (d, J=8.60 Hz, 2H) 7.71 (d, J=8.60 Hz, 2H) 7.33-7.50 (m, 4H) 5.39 (s, 2H).

I.28. Benzyl 4-(5-chloro-1,1-dioxido-3-oxoisothiazol-2(3H)-yl)benzoate

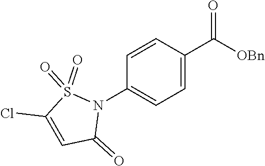 [SMILES: O=C(OCc1ccccc1)c1ccc(N2C(=O)C=C(Cl)S2(=O)=O)cc1]

To a mixture of benzyl 4-(5-chloro-3-oxo-isothiazol-2-yl)benzoate (1 g, 2.89 mmol, 1 eq) in H₂O (10 mL), ACN (5 mL) and DCM (5 mL) was added RuCl₃·H₂O (13.04 mg, 57.84 umol, 0.02 eq) and NaIO₄ (2.47 g, 11.57 mmol, 640.97 uL, 4 eq) in one portion at 0° C. under N₂. The mixture then heated to 20° C. and stirred for 2 hours. The residue was filtered and the filtrate was poured into water (40 ml). The aqueous phase was extracted with ethyl acetate (50 mL*1). The organic phase was washed with sat. NaCl (30 mL*3), dried with anhydrous Na₂SO₄, filtered and concentrated in vacuum. The residue was purified by column chromatography (SiO₂, Petroleum ether:Ethyl acetate=15:1 to 5:1) to give benzyl 4-(5-chloro-1,1,3-trioxo-isothiazol-2-yl)benzoate (500 mg, 1.32 mmol, 45.77% yield) as a yellow oil; ¹H NMR (400 MHz, CHLOROFORM-d) δ=8.27-8.21 (m, 2H), 7.61-7.55 (m, 2H), 7.49-7.34 (m, 5H), 6.87 (s, 1H), 5.40 (s, 2H).

### Example 10. 4-(5-chloro-1,1-dioxido-3-oxoisothiazol-2(3H)-yl)benzoic acid

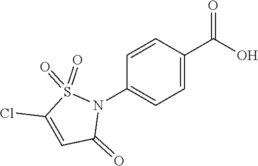 [SMILES: O=C(O)c1ccc(N2C(=O)C=C(Cl)S2(=O)=O)cc1]

A solution of benzyl 4-(5-chloro-1,1,3-trioxo-isothiazol-2-yl)benzoate (450 mg, 1.19 mmol, 1 eq) in DCM (20 mL) was added methanesulfonic acid (1.14 g, 11.91 mmol, 847.94 uL, 10 eq) dropwise at 10° C. The solution was stirred at 35° C. for 10 hrs. The residue was concentrated in vacuum to remove DCM. Then, the residue was dissolved into Ethyl acetate (10 ml) and then the organic phase was washed with water (20 mL*5), dried with anhydrous Na₂SO₄, filtered and concentrated in vacuum. The residue was dissolved into methanol (3 ml) and then petroleum ether (30 ml) was poured into the residue, the solution was stirred at 10° C. for 2 min, and then filtered, the filter cake was dried in vacuum to give 4-(5-chloro-1,1,3-trioxo-isothiazol-2-yl)benzoic acid (112.56 mg, 379.48 umol, 31.86% yield, 96.986% purity) as a white solid; LC-MS (ES, m/z): 285.9 [M−H]—; ¹H NMR (400 MHz, DMSO-d6) δ=13.33 (br s, 1H), 8.17-8.11 (m, 2H), 7.87 (d, J=1.5 Hz, 1H), 7.67-7.61 (m, 2H).
I.29. Benzyl 4-(4,5-dichloro-1,1-dioxido-3-oxoisothiazol-2(3H)-yl)benzoate

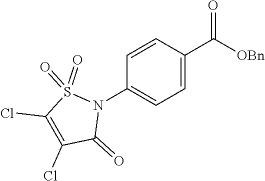 [SMILES: O=C(OCc1ccccc1)c1ccc(N2C(=O)C(Cl)=C(Cl)S2(=O)=O)cc1]

To a mixture of benzyl 4-(4,5-dichloro-3-oxo-isothiazol-2-yl)benzoate (1 g, 2.63 mmol, 1 eq) in H₂O (10 mL), ACN (5 mL) and DCM (5 mL) was added RuCl₃·H₂O (11.86 mg, 52.60 umol, 0.02 eq) and NaIO₄ (2.25 g, 10.52 mmol, 582.91 uL, 4 eq) in one portion at 0° C. under N₂. The mixture then heated to 20° C. and stirred for 2 hours. The residue was filtered and the filtrate was concentrated in vacuum. The residue was purified by column chromatography (SiO₂, Petroleum ether:Ethyl acetate=20:1 to 5:1) to give benzyl 4-(4,5-dichloro-1,1,3-trioxo-isothiazol-2-yl)benzoate (130 mg, 315.35 umol, 11.99% yield) as a white solid; ¹H NMR (400 MHz, CHLOROFORM-d) δ=8.25 (d, J=8.6 Hz, 2H), 7.58 (d, J=8.8 Hz, 2H), 7.49-7.34 (m, 5H), 5.41 (s, 2H).

### Example 11. 4-(4,5-dichloro-1,1-dioxido-3-oxoisothiazol-2(3H)-yl)benzoic acid

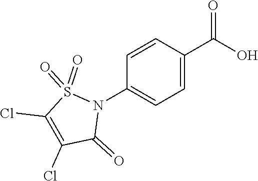 [SMILES: O=C(O)c1ccc(N2C(=O)C(Cl)=C(Cl)S2(=O)=O)cc1]

A solution of benzyl 4-(4,5-dichloro-1,1,3-trioxo-isothiazol-2-yl)benzoate (130 mg, 315.35 umol, 1 eq) in DCM (5 mL) was added methanesulfonic acid (303.07 mg, 3.15 mmol, 224.49 uL, 10 eq) dropwise at 10° C. The solution was stirred at 10° C. for 5 min, then the solution was heated to 35° C. and stirred for 10 hours. The color of solution changed from colorless to yellow. The reaction solution was poured into water (20 ml) and then filtered. The filter cake was washed with water (10 ml*3) and DCM (10 ml*3) three times respectively. Then the filter cake was dried in vacuum. The residue was dispersed with DCM (10 ml) and then petroleum ether (30 ml) was poured into the residue, the mixture was stirred at 10° C. for 2 min, and then filtered, the filter cake was dried in vacuum to give 4-(4,5-dichloro-1,1,3-trioxo-isothiazol-2-yl)benzoic acid (93.4 mg, 282.97 umol, 89.73% yield, 97.590% purity) as a white solid; LC-MS (ES, m/z): 319.9 [M−H]—; ¹H NMR (400 MHz, DMSO-d6) 5=8.16 (d, J=8.4 Hz, 2H), 7.68 (d, J=8.4 Hz, 2H).
I.30. Benzyl 3-(2-(2-(2-aminoethoxy)ethoxy)ethoxy)propanoate 4-methylbenzenesulfonate

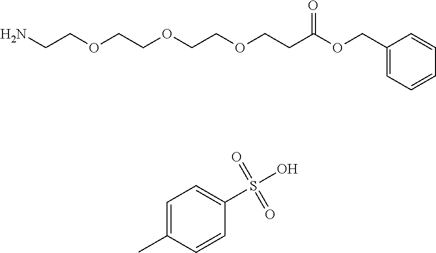 [SMILES: NCCOCCOCCOCCC(=O)OCc1ccccc1 ; Cc1ccc(S(=O)(=O)O)cc1]

A mixture of 3-[2-[2-(2-aminoethoxy)ethoxy]ethoxy]propanoic acid (4 g, 18.08 mmol, 1 eq), phenylmethanol (15.64 g, 144.63 mmol, 15.04 mL, 8 eq) and TsOH·H₂O (3.61 g, 18.98 mmol, 1.05 eq) in toluene (30 mL) was stirred at 140° C. for 8 hours using a Dean and Stark apparatus to collect the water of condensation. The reaction turned to clear after refluxing for several hours. TLC (Dichloromethane:Methanol=10:1, Rf=0.3) indicated the reaction was complete. The clear reaction mixture was poured into TBME:Petroleum ether (1:1, 50 mL) and removed the clear solution. The residue was washed with TBME:Petroleum ether (1:1, 50 mL) for 2 times and dried in vacuum. The crude product Benzyl 3-[2-[2-(2-aminoethoxy)ethoxy]ethoxy]propanoate; 4-methylbenzenesulfonic acid (8.9 g, crude) was obtained as a yellow oil; ¹H NMR (400 MHz, CHLOROFORM-d) 6 ppm 7.76 (br d, J=8.07 Hz, 2H) 7.33-7.38 (m, 5H) 7.15 (d, J=7.95 Hz, 2H) 5.11 (s, 2H) 3.72 (q, J=6.11 Hz, 4H) 3.53-3.64 (m, 8H) 3.11-3.24 (m, 2H) 2.53-2.69 (m, 2H) 2.30-2.41 (m, 1H) 2.34 (s, 3H).
I.31. Benzyl 3-[2-[2-[2-[3-[[3-[2-[2-[2-(3-benzyloxy-3-oxo-propoxy)ethoxy]ethoxy]ethyl amino]-3-oxopropyl]disulfanyl]propanoylamino]ethoxy]ethoxy]ethoxy]propanoate

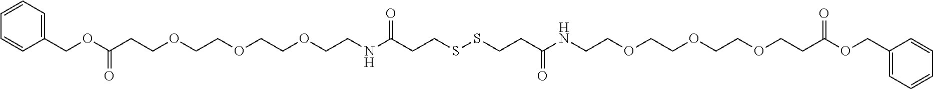 [SMILES: O=C(CCSSCCC(=O)NCCOCCOCCOCCC(=O)OCc1ccccc1)NCCOCCOCCOCCC(=O)OCc1ccccc1]

To a mixture of 3-2-carboxyethyldisulfanyl)propanoic acid (1.909.04 mmol 1 eq), HOBt (2.69 g, 19.88 mmol, 2.2 eq) and TEA (4.57 g, 45.18 mmol, 6.29 mL, 5 eq) in DCM (100 mL) was added EDCI (3.81 g, 19.88 mmol, 2.2 eq) at 20° C. Then benzyl 3-[2-[2-(2-aminoethoxy)ethoxy]ethoxy]propanoate; 4-methylbenzenesulfonic acid (8.74 g, 18.07 mmol, 2 eq) was added to the above solution. The mixture was stirred at 20° C. for 12 hours. TLC (Petroleum ether:Ethyl acetate=2:1, Rf=0.25) indicated that the reaction was complete. MeOH was added and the solution was concentrated under reduced pressure and dried over vacuum. The residue was poured into H₂O (20 mL), extracted with EtOAc (50 mL). The organic layer was dried over Na₂SO₄, filtered and concentrated in vacuum. The crude product was purified by prep-TLC (Petroleum ether:Ethyl acetate=1:1, Rf=0.5) to give Benzyl 3-[2-[2-[2-[3-[[3-[2-[2-[2-(3-benzyloxy-3-oxo-propoxy)ethoxy]ethoxy]ethylamino]-3-oxo-propyl]disulfanyl]propanoyl amino]ethoxy]ethoxy]ethoxy]propanoate (6.52 g, 7.94 mmol, 87.81% yield, 97% purity) as a yellow oil; ¹H NMR (400 MHz, CHLOROFORM-d) 6 ppm 7.29-7.42 (m, 10H) 6.42 (br s, 2H) 5.15 (s, 4H) 3.79 (t, J=6.42 Hz, 4H) 3.53-3.69 (m, 18H) 2.92-3.00 (m, 4H) 2.62-2.71 (m, 4H) 2.53-2.62 (m, 4H).
I.32. Benzyl 3-(2-(2-(2-(5-chloro-3-oxoisothiazol-2(3H)-yl)ethoxy)ethoxy)ethoxy)propanoate

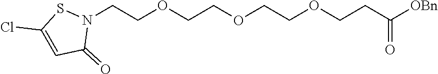 [SMILES: O=C(CCOCCOCCOCCn1sc(Cl)cc1=O)OCc1ccccc1]

To a solution of benzyl 3-[2-[2-[2-[3-[[3-[2-[2-[2-(3-benzyloxy-3-oxo-propoxy)ethoxy]ethoxy]ethylamino]-3-oxopropyl]disulfanyl]propanoylamino]ethoxy] ethoxy]ethoxy]propanoate (6.4 g, 8.03 mmol, 1 eq) in DCM (50 mL) was added dropwise the solution of sulfuryl chloride (4.34 g, 32.12 mmol, 3.21 mL, 4 eq) in DCM (10 mL) at 0° C. The mixture was stirred at 0-10° C. for 12 hrs. TLC (Petroleum ether:Ethyl acetate=0:1, R_{f}=0.15, 0.35) indicated the reaction was complete. The mixture was poured into ice/water (100 mL), extracted with DCM (200 mL*2). The combined organic layers were washed with H₂O (100 mL*2), brine (100 mL), dried over Na₂SO₄. Filtration and concentrated in vacuum. The residue was purified by column chromatography on silica gel (Petroleum ether:Ethyl acetate=1:1 to 0:1) to give Benzyl 3-[2-[2-[2-(3-oxoisothiazol-2-yl)ethoxy]ethoxy]ethoxy]propanoate (820 mg, 1.66 mmol, 10.33% yield, 80% purity) as a brown oil; ¹H NMR (400 MHz, CHLOROFORM-d) δ ppm 8.06 (d, J=6.17 Hz, 1H) 7.30-7.41 (m, 5H) 6.24 (d, J=6.39 Hz, 1H) 5.15 (s, 2H) 3.95-4.03 (m, 2H) 3.79 (t, J=6.39 Hz, 2H) 3.68-3.75 (m, 2H) 3.59-3.68 (m, 8H) 2.63-2.70 (m, 2H) and Benzyl 3-[2-[2-[2-(5-chloro-3-oxo-isothiazol-2-yl)ethoxy]ethoxy]ethoxy]propanoate (2.5 g, 4.30 mmol, 26.79% yield, 74% purity) as a colorless oil; ¹H NMR (400 MHz, CHLOROFORM-d) δ ppm 7.28-7.43 (m, 5H) 6.25 (s, 1H) 5.15 (s, 2H) 3.92-3.98 (m, 2H) 3.77-3.81 (m, 2H) 3.59-3.71 (m, 10H) 2.66 (t, J=6.39 Hz, 2H).
I.33. Benzyl 3-(2-(2-(2-(5-chloro-1,1-dioxido-3-oxoisothiazol-2(3H)-yl)ethoxy)ethoxy) ethoxy)propanoate

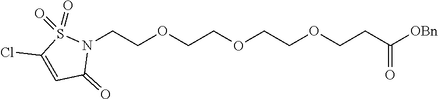 [SMILES: O=C(CCOCCOCCOCCN1C(=O)C=C(Cl)S1(=O)=O)OCc1ccccc1]

To a mixture of benzyl 3-[2-[2-[2-(5-chloro-3-oxo-isothiazol-2-yl)ethoxy]ethoxy] ethoxy]propanoate (1 g, 2.33 mmol, 1 eq) and NaIO₄ (1.99 g, 9.30 mmol, 515.57 uL, 4 eq) in H₂O (20 mL), DCM (10 mL), ACN (10 mL) was added RuCl₃·H₂O (26.22 mg, 116.30 umol, 0.05 eq) in one portion at 20° C. under N₂. Then, the mixture was stirred at 20° C. for 1 hr. The residue was extracted with ethyl acetate (20 mL*2). The combined organic phase was dried with anhydrous Na₂SO₄, filtered and concentrated in vacuum. The residue was purified by column chromatography (SiO₂, Petroleum ether:Ethyl acetate=5:1 to 1:1) to give benzyl 3-[2-[2-[2-(5-chloro-1,1,3-trioxo-isothiazol-2-yl)ethoxy]ethoxy]ethoxy]propanoate (560 mg, 1.21 mmol, 52.12% yield, 100% purity) as a purple oil; ¹H NMR (400 MHz, CHLOROFORM-d) δ=7.42-7.29 (m, 5H), 6.70 (s, 1H), 5.15 (s, 2H), 3.92-3.86 (m, 2H), 3.77 (td, J=6.0, 11.9 Hz, 4H), 3.68-3.58 (m, 8H), 2.67 (t, J=6.5 Hz, 2H).

### Example 12. 3-(2-(2-(2-(5-chloro-1,1-dioxido-3-oxoisothiazol-2(3H)-yl)ethoxy)ethoxy)ethoxy) propanoic acid

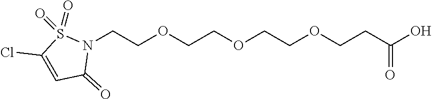 [SMILES: O=C(O)CCOCCOCCOCCN1C(=O)C=C(Cl)S1(=O)=O]

A solution of benzyl 3-[2-[2-[2-(5-chloro-1,1,3-trioxo-isothiazol-2-yl)ethoxy] ethoxy]ethoxy]propanoate (560 mg, 1.21 mmol, 1 eq) in DCM (30 mL) was added methanesulfonic acid (1.17 g, 12.12 mmol, 863.06 uL, 10 eq) dropwise at 10° C. The mixture was heated to 35° C. and stirred for 10 hrs. The color of solution turned to yellow. The residue was washed with water (30 ml*3) and then the organic phase was dried with anhydrous Na₂SO₄, filtered and concentrated in vacuum. The residue was purified by prep-TLC (Ethyl acetate:Ethyl acetate:Methanol:Acetic acid=40:8:1, Rf=0.77) to give 3-[2-[2-[2-(5-chloro-1,1,3-trioxo-isothiazol-2-yl)ethoxy]ethoxy]ethoxy] propanoic acid (95.61 mg, 240.16 umol, 19.81% yield, 93.389% purity) as a colorless oil; LC-MS (ES, m/z): 372.1 [M+H]⁺; ¹H NMR (400 MHz, DMSO-d6) δ=12.13 (br s, 1H), 7.64 (s, 1H), 3.86-3.74 (m, 2H), 3.61 (td, J=6.0, 16.9 Hz, 4H), 3.54-3.46 (m, 8H), 2.43 (t, J=6.4 Hz, 2H).
I.34. Benzyl 3-(2-(2-(2-(4,5-dichloro-3-oxoisothiazol-2(3H)-yl)ethoxy)ethoxy)ethoxy) propanoate

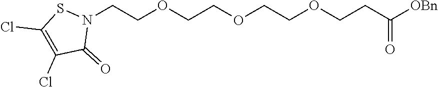 [SMILES: O=C(CCOCCOCCOCCn1sc(Cl)c(Cl)c1=O)OCc1ccccc1]

To a solution of benzyl 3-[2-[2-[2-(5-chloro-3-oxo-isothiazol-2-yl)ethoxy]ethoxy]ethoxy] propanoate (1.6 g, 3.72 mmol, 1 eq) in DCM (30 mL) was added dropwise sulfuryl chloride (1.00 g, 7.44 mmol, 744.17 uL, 2 eq) at 0° C. The mixture was stirred at 0-10° C. for 12 hrs. A clear pale yellow solution was obtained after the addition of sulfuryl chloride. TLC (Ethyl acetate:Petroleum ether=2:1, Rf=0.5) indicated the reaction was complete. The mixture was concentrated in vacuum to give a crude product. The residue was poured into H₂O (50 mL), extracted with DCM (50 mL*2). The combined organic layers were washed with H₂O (50 mL), dried over Na₂SO₄, filtered and concentrated in vacuum. The residue was purified by column chromatography on silica gel (Ethyl acetate:Petroleum ether=1:2 to 2:1) to give Benzyl 3-[2-[2-[2-(4,5-dichloro-3-oxo-isothiazol-2-yl)ethoxy]ethoxy]ethoxy] propanoate (1.06 g, 1.76 mmol, 47.17% yield, 76.9% purity) as a colorless oil; ¹H NMR (400 MHz, CHLOROFORM-d) δ ppm 7.28-7.40 (m, 4H) 5.13 (s, 2H) 3.98-4.04 (m, 2H) 3.77 (t, J=6.39 Hz, 2H) 3.67-3.72 (m, 2H) 3.57-3.67 (m, 8H) 2.65 (t, J=6.39 Hz, 2H).

I.35. Benzyl 3-(2-(2-(2-(4,5-dichloro-1,1-dioxido-3-oxoisothiazol-2(3H)-yl)ethoxy)ethoxy) ethoxy)propanoate

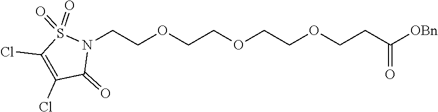 [SMILES: O=C(CCOCCOCCOCCN1C(=O)C(Cl)=C(Cl)S1(=O)=O)OCc1ccccc1]

To a mixture of benzyl 3-[2-[2-[2-(4,5-dichloro-3-oxo-isothiazol-2-yl)ethoxy]ethoxy]ethoxy] propanoate (1 g, 2.15 mmol, 1 eq) and NaIO₄ (1.84 g, 8.61 mmol, 477.32 uL, 4 eq) in H₂O (20 mL), CH₃CN (10 mL) and DCM (10 mL) was added RuCl₃·H₂O (7.28 mg, 32.30 umol, 0.015 eq) under N₂ at 0° C. The mixture was stirred at 0-10 for 2 hrs. TLC indicated the reaction was complete. The mixture was diluted with EtOAc (50 mL), filtered to remove the unsoluble solid. The organic layer was separated and concentrated in vacuum. The residue was purified by prep-TLC (Petroleum ether:Ethyl acetate=1:1, Rf=0.6) to give Benzyl 3-[2-[2-[2-(4,5-dichloro-1,1,3-trioxo-isothiazol-2-yl)ethoxy]ethoxy]ethoxy]propanoate (830 mg, 1.61 mmol, 74.96% yield, 96.533% purity) as a colorless oil; ¹H NMR (400 MHz, CHLOROFORM-d) δ ppm 7.28-7.41 (m, 5H) 5.15 (s, 2H) 3.91-3.97 (m, 2H) 3.75-3.83 (m, 4H) 3.59-3.68 (m, 8H) 2.66 (t, J=6.50 Hz, 2H).

### Example 13. 3-(2-(2-(2-(4,5-dichloro-1,1-dioxido-3-oxoisothiazol-2(3H)-yl)ethoxy)ethoxy)ethoxy) propanoic acid

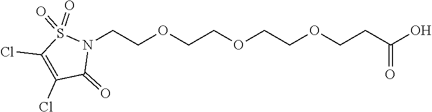 [SMILES: O=C(O)CCOCCOCCOCCN1C(=O)C(Cl)=C(Cl)S1(=O)=O]

A solution of benzyl 3-[2-[2-[2-(4,5-dichloro-1,1,3-trioxo-isothiazol-2-yl)ethoxy]ethoxy]ethoxy]propanoate (820.00 mg, 1.65 mmol, 1 eq) in DCM (10 mL) was added methanesulfonic acid (1.59 g, 16.52 mmol, 1.18 mL, 10 eq) dropwise at 10° C. Then, the solution was heated at 35° C. and stirred for 10 hrs. The residue was diluted by DCM (20 ml) and then the solution was washed with water (15 ml*3), the organic phase was dried with anhydrous Na₂SO₄, filtered and concentrated in vacuum. The residue was purified by prep-TLC (Ethyl acetate:Acetic acid=250:1, Rf=0.55) to 3-[2-[2-[2-(4,5-dichloro-1,1,3-trioxo-isothiazol-2-yl)ethoxy]ethoxy]ethoxy]propanoic acid (249.4 mg, 602.76 umol, 36.49% yield, 98.180% purity) as a yellow oil; LC-MS (ES, m/z): 406.0 [M+H]⁺; ¹H NMR (400 MHz, DMSO-d6) δ=12.16 (br s, 1H), 3.85 (t, J=5.5 Hz, 2H), 3.65 (br t, J=5.4 Hz, 2H), 3.61-3.45 (m, 10H), 2.43 (t, J=6.3 Hz, 2H).
I.36. Benzyl 1-amino-3,6,9,12,15,18-hexaoxahenicosan-21-oate 4-methylbenzenesulfonate

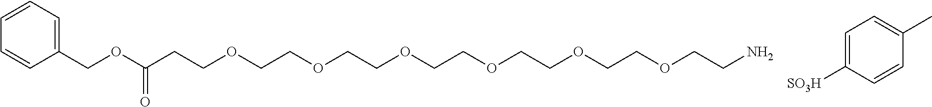 [SMILES: NCCOCCOCCOCCOCCOCCOCCC(=O)OCc1ccccc1 ; Cc1ccc(S(=O)(=O)O)cc1]

A mixture of phenylmethanol (2.45 g, 22.64 mmol, 2.35 mL, 8 eq), 3-[2-[2-[2-[2-[2-(2-aminoethoxy)ethoxy]ethoxy]ethoxy]ethoxy]ethoxy]propanoic acid (1 g, 2.83 mmol, 1 eq) and TsOH·H₂O (565.16 mg, 2.97 mmol, 1.05 eq) in toluene (30 mL) was stirred at 140° C. with a Dean-Stark trap for 14 hrs. The mixture was changed from turbidity to clearly several hours later. The residue was concentrated in vacuum to remove toluene, and then TBME (50 ml) was poured into the residue and stirred for 1 min. Then, supernatant was remove and dried to give benzyl 3-[2-[2-[2-[2-[2-(2-aminoethoxy)ethoxy]ethoxy]ethoxy]ethoxy]ethoxy]propanoate; 4-methylbenzene sulfonic acid (1.45 g, crude) as a yellow oil; ¹H NMR (400 MHz, CHLOROFORM-d) δ=7.80 (d, J=8.1 Hz, 2H), 7.67-7.46 (m, 2H), 7.39-7.31 (m, 5H), 7.15 (d, J=7.8 Hz, 2H), 5.13 (s, 2H), 3.96-3.83 (m, 2H), 3.75-3.50 (m, 22H), 3.24-3.14 (m, 2H), 2.63 (t, J=6.2 Hz, 2H), 2.34 (s, 3H).
I.37. Dibenzyl 23,30-dioxo-4,7,10,13,16,19,34,37,40,43,46,49-dodecaoxa-26,27-dithia-22,31-diazadopentacontanedioate

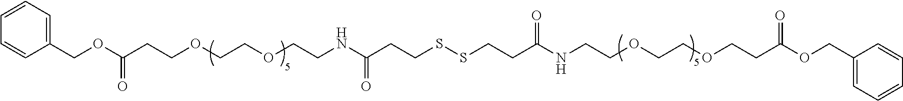

To a mixture of 3-(2-carboxyethyldisulfanyl)propanoic acid (47.41 mg, 225.47 umol, 1 eq) and TEA (91.26 mg, 901.86 umol, 125.53 uL, 4 eq), HOBt (91.40 mg, 676.40 umol, 3 eq), EDCI (129.67 mg, 676.40 umol, 3 eq) in DCM (5 mL) was added benzyl 3-[2-[2-[2-[2-[2-(2-aminoethoxy)ethoxy]ethoxy]ethoxy]ethoxy]ethoxy]propanoate; 4-methyl benzenesulfonic acid (277.65 mg, 450.93 umol, 2 eq) dropwise at 25° C. After addition, the mixture was stirred at 25° C. for 8 hours. TLC (Ethyl acetate:Methanol=3:1, R_{f}=0.33) showed starting material was consumed and a new main spot was generated. The residue was poured into sat. NaCl (10 ml) and stirred for 2 min. Then, the aqueous phase was extracted with DCM (5 mL*3). The combined organic phase was washed with sat. NaCl (10 mL*2), dried with anhydrous Na₂SO₄, filtered and concentrated in vacuum. The residue was purified by prep-HPLC (column: Waters Xbridge 150*25 5 u; mobile phase: [water (10 mM NH4HCO3)-ACN]; B %: 32%-62%, 12 min) to give benzyl 3-[2-[2-[2-[2-[2-[2-[3-[[3-[2-[2-[2-[2-[2-[2-(3-benzyloxy-3-oxo-propoxy)ethoxy]ethoxy]ethoxy]ethoxy]ethoxy]ethylamino]-3-oxo-propyl]disulfanyl]propanoylamino] ethoxy]ethoxy]ethoxy]ethoxy]ethoxy]ethoxy] propanoate (70 mg, 65.96 umol, 29.25% yield) as a colorless oil; ¹H NMR (400 MHz, CHLOROFORM-d) δ=7.41-7.29 (m, 10H), 6.50 (br s, 2H), 5.14 (s, 4H), 3.78 (t, J=6.5 Hz, 4H), 3.67-3.61 (m, 40H), 3.59-3.55 (m, 4H), 3.45 (q, J=5.1 Hz, 4H), 2.97 (t, J=7.2 Hz, 4H), 2.66 (t, J=6.5 Hz, 4H), 2.60 (t, J=7.1 Hz, 4H).
I.38. Benzyl 1-(5-chloro-3-oxoisothiazol-2(3H)-yl)-3,6,9,12,15,18-hexaoxahenicosan-21-oate

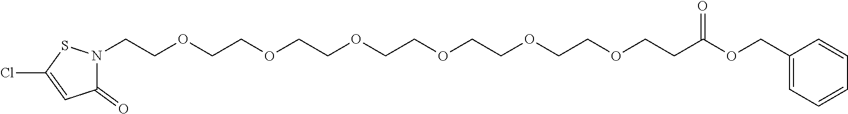 [SMILES: O=C(CCOCCOCCOCCOCCOCCOCCn1sc(Cl)cc1=O)OCc1ccccc1]

To a solution of benzyl 3-[2-[2-[2-[2-[2-[2-[3-[[3-[2-[2-[2-[2-[2-[2-(3-benzyloxy-3-oxo- propoxy)ethoxy]ethoxy]ethoxy]ethoxy]ethoxy]ethylamino]-3-oxo-propyl]disulfanyl]propanoylamino]ethoxy]ethoxy]ethoxy]ethoxy]ethoxy]ethoxy]-propanoate (5.5 g, 5.18 mmol, 1 eq) in DCM (60 mL) was added dropwise sulfuryl chloride (3.50 g, 25.91 mmol, 2.59 mL, 5 eq) at 0° C. The mixture was stirred at 0-20° C. for 12 hrs. TLC (Ethyl acetate:Methanol=10:1, Rf=0.3, 0.5) indicated the reaction was completed. The mixture was poured into ice/water (10 mL), extracted with DCM (20 mL*2). The combined organic layers were washed with H₂O (20 mL*2), brine (20 mL), dried over Na₂SO₄. Filtration and concentrated in vacuum. The residue was purified by column chromatography on silica gel (Petroleum ether:Ethyl acetate=1:1 to 0:1) to give Benzyl 3-[2-[2-[2-[2-[2-[2-(3-oxoisothiazol-2-yl)ethoxy]ethoxy]ethoxy]ethoxy]ethoxy]ethoxy]propanoate (1.4 g, 2.29 mmol, 22.05% yield, 86.148% purity) as a brown oil; ¹H NMR (400 MHz, CHLOROFORM-d) δ ppm 8.02 (d, J=6.17 Hz, 1H) 7.22-7.32 (m, 5H) 6.17 (br d, J=6.17 Hz, 1H) 5.07 (s, 2H) 3.92 (br t, J=4.30 Hz, 2H) 3.51-3.73 (m, 24H) 2.58 (t, J=6.39 Hz, 2H) and Benzyl 3-[2-[2-[2-[2-[2-[2-(5-chloro-3-oxo-isothiazol-2-yl)ethoxy]ethoxy]ethoxy]ethoxy] ethoxy]ethoxy]propanoate (3.1 g, 4.44 mmol, 42.85% yield, 80.532% purity) as a colorless oil; ¹H NMR (400 MHz, CHLOROFORM-d) δ ppm 7.30-7.40 (m, 5H) 6.26 (s, 1H) 5.15 (s, 2H) 3.93-3.99 (m, 2H) 3.78 (t, J=6.50 Hz, 2H) 3.60-3.72 (m, 23H) 2.66 (t, J=6.50 Hz, 2H).
I.39 Benzyl 1-(5-chloro-1,1-dioxido-3-oxoisothiazol-2(3H)-yl)-3,6,9,12,15,18-hexaoxahenicosan-21-oate

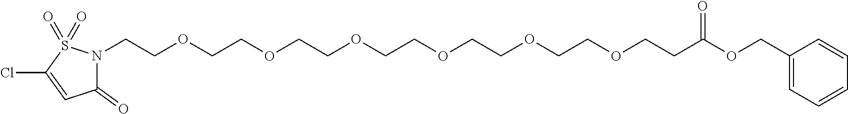 [SMILES: O=C(CCOCCOCCOCCOCCOCCOCCN1C(=O)C=C(Cl)S1(=O)=O)OCc1ccccc1]

To a mixture of benzyl 3-[2-[2-[2-[2-[2-[2-(5-chloro-3-oxo-isothiazol-2-yl)ethoxy]ethoxy]ethoxy]ethoxy]ethoxy]ethoxy]propanoate (1 g, 1.78 mmol, 1 eq) and NaIO₄ (1.52 g, 7.12 mmol, 394.34 uL, 4 eq) in H₂O (20 mL), DCM (10 mL), ACN (10 mL) was added RuCl₃·H₂O (20.05 mg, 88.96 umol, 0.05 eq) in one portion at 20° C. under N₂. Then, the mixture was stirred at 20° C. for 1 hr. The residue was extracted with ethyl acetate (20 mL*2). The combined organic phase was dried with anhydrous Na₂SO₄, filtered and concentrated in vacuum. The residue was purified by column chromatography (SiO₂, Petroleum ether:Ethyl acetate=1:1 to Ethyl acetate:Methanol=10:1) to give benzyl 3-[2-[2-[2-[2-[2-[2-(5-chloro-1,1,3-trioxo-isothiazol-2-yl)ethoxy]ethoxy] ethoxy]ethoxy]ethoxy]ethoxy]propanoate (520 mg, 849.06 umol, 47.72% yield, 97% purity) as a yellow oil; ¹H NMR (400 MHz, CHLOROFORM-d) δ=7.44-7.29 (m, 5H), 6.72 (s, 1H), 5.14 (s, 2H), 3.92-3.86 (m, 2H), 3.80-3.73 (m, 4H), 3.69-3.59 (m, 20H), 2.66 (t, J=6.4 Hz, 2H).

### Example 14. 1-(5-chloro-1,1-dioxido-3-oxoisothiazol-2(3H)-yl)-3,6,9,12,15,18-hexaoxahenicosan-21-oic acid

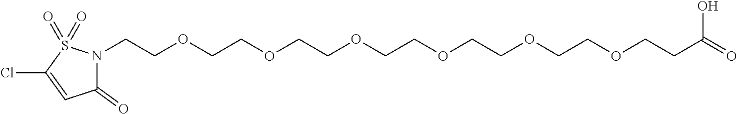 [SMILES: O=C(O)CCOCCOCCOCCOCCOCCOCCN1C(=O)C=C(Cl)S1(=O)=O]

A solution of benzyl 3-[2-[2-[2-[2-[2-[2-(5-chloro-1,1,3-trioxo-isothiazol-2-yl)ethoxy]ethoxy]ethoxy]ethoxy]ethoxy]ethoxy]propanoate (520 mg, 875.32 umol, 1 eq) in DCM (30 mL) was added methanesulfonic acid (841.23 mg, 8.75 mmol, 623.13 uL, 10 eq) dropwise at 10° C. The solution was heated to 35° C. and stirred for 10 hrs. The residue was washed with water (30 ml*3) and then the organic phase was dried with anhydrous Na₂SO₄, filtered and concentrated in vacuum. The residue was purified by prep-TLC (Ethyl acetate:Methanol:Acetic acid=40:8:1, Rf=0.58). The residue was purified again by prep-HPLC (column: Nano-micro Kromasil C18 100*30 mm 5 um; mobile phase: [water (0.05% HCl)-ACN]; B %: 1%-30%, 10 min) to give 3-[2-[2-[2-[2-[2-[2-(5-chloro-1,1,3-trioxo-isothiazol-2-yl)ethoxy]ethoxy]ethoxy]ethoxy]ethoxy]ethoxy]propanoic acid (55.93 mg, 106.24 umol, 12.14% yield, 95.726% purity) as a colorless oil; LC-MS (ES, m/z): 504.2 [M+H]⁺; ¹H NMR (400 MHz, DMSO-d6) δ=12.30-11.96 (m, 1H), 7.64 (s, 1H), 3.83-3.76 (m, 2H), 3.65-3.58 (m, 4H), 3.54-3.52 (m, 2H), 3.52-3.48 (m, 18H), 2.44-2.42 (m, 2H).
I.40 Benzyl 3-[2-[2-[2-[2-[2-[2-(4,5-dichloro-3-oxo-isothiazol-2-yl)ethoxy]ethoxy]ethoxy] ethoxy]ethoxy]ethoxy]propanoate

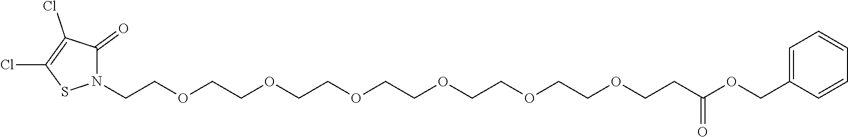 [SMILES: O=C(CCOCCOCCOCCOCCOCCOCCn1sc(Cl)c(Cl)c1=O)OCc1ccccc1]

A solution of benzyl 3-[2-[2-[2-[2-[2-[2-(5-chloro-3-oxo-isothiazol-2-yl)ethoxy]ethoxy]ethoxy]ethoxy]ethoxy]ethoxy]propanoate (1.3 g, 2.31 mmol, 1 eq) in DCM (30 mL) was added sulfuryl chloride (624.34 mg, 4.63 mmol, 462.47 uL, 2 eq) dropwise at 20° C. The solution was stirred at 20° C. for 2 hrs. The solution turned to yellow. The residue was poured into ice-water (30 ml) and stirred for 30 min. The DCM phase was washed with water (50 mL*6), dried with anhydrous Na₂SO₄, filtered and concentrated in vacuum. The residue was purified by prep-TLC (Ethyl acetate:Methanol=10:1, Rf=0.50) to give benzyl 3-[2-[2-[2-[2-[2-[2-(4,5-dichloro-3-oxo-isothiazol-2-yl)ethoxy]ethoxy]ethoxy]ethoxy]ethoxy]ethoxy]propanoate (800 mg, 1.21 mmol, 52.19% yield, 90% purity) as a yellow oil; ¹H NMR (400 MHz, CHLOROFORM-d) δ=7.40-7.29 (m, 5H), 5.15 (s, 2H), 4.04 (t, J=4.7 Hz, 2H), 3.78 (t, J=6.4 Hz, 2H), 3.72 (t, J=4.7 Hz, 2H), 3.69-3.63 (m, 16H), 3.62 (s, 4H), 2.66 (t, J=6.5 Hz, 2H).
I.41 Benzyl 1-(4,5-dichloro-1,1-dioxido-3-oxoisothiazol-2(3H)-yl)-3,6,9,12,15,18-hexaoxahenicosan-21-oate

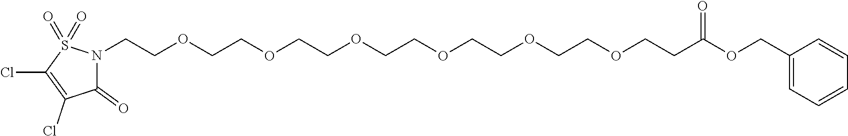 [SMILES: O=C(CCOCCOCCOCCOCCOCCOCCN1C(=O)C(Cl)=C(Cl)S1(=O)=O)OCc1ccccc1]

To a mixture of benzyl 3-[2-[2-[2-[2-[2-[2-(4,5-dichloro-3-oxo-isothiazol-2-yl)ethoxy]ethoxy]ethoxy]ethoxy]ethoxy]ethoxy]propanoate (600 mg, 1.01 mmol, 1 eq) and NaIO₄ (860.56 mg, 4.02 mmol, 222.94 uL, 4 eq) in H₂O (20 mL), DCM (10 mL), ACN (10 mL) was added RuCl₃·H₂O (11.34 mg, 50.29 umol, 0.05 eq) in one portion at 0° C. under N₂. The mixture was stirred at 0° C. for 2 min, then heated to 25° C. and stirred for 1 hour. The residue was poured into Ethyl acetate (30 ml), and then filtered. The filtrate was extracted with ethyl acetate (30 mL*3). The combined organic phase was concentrated in vacuum. The residue was purified by prep-TLC (Ethyl acetate, Rf=0.50) to give benzyl 3-[2-[2-[2-[2-[2-[2-(4,5-dichloro-1,1,3-trioxo-isothiazol-2-yl)ethoxy]ethoxy]ethoxy]ethoxy]ethoxy]ethoxy]propanoate (220 mg, 350.03 umol, 34.80% yield, 100% purity) as a colorless oil; ¹H NMR (400 MHz, CHLOROFORM-d) δ=7.40-7.27 (m, 4H), 5.15 (s, 2H), 3.99-3.90 (m, 2H), 3.78 (t, J=6.2 Hz, 4H), 3.70-3.58 (m, 20H), 2.66 (t, J=6.4 Hz, 2H).

### Example 15. 1-(4,5-dichloro-1,1-dioxido-3-oxoisothiazol-2(3H)-yl)-3,6,9,12,15,18-hexaoxahenicosan-21-oic acid

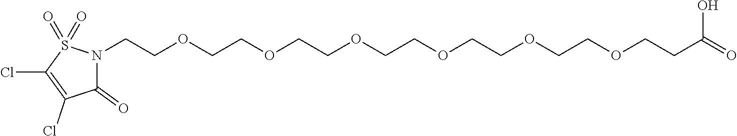 [SMILES: O=C(O)CCOCCOCCOCCOCCOCCOCCN1C(=O)C(Cl)=C(Cl)S1(=O)=O]

A solution of benzyl 3-[2-[2-[2-[2-[2-[2-(4,5-dichloro-1,1,3-trioxo-isothiazol-2-yl)ethoxy]ethoxy]ethoxy]ethoxy]ethoxy]ethoxy]propanoate (220 mg, 350.03 umol, 1 eq) in DCM (5 mL) was added methanesulfonic acid (504.60 mg, 5.25 mmol, 373.78 uL, 15 eq) dropwise at 10° C. Then, the solution was heated to 40° C. and stirred for 20 hrs. The residue was diluted by DCM (20 ml) and then the solution was washed with water (15 ml*3), the organic phase was dried with anhydrous Na₂SO₄, filtered and concentrated in vacuum. The residue was purified by prep-HPLC (column: Nano-micro Kromasil C18 100*30 mm 5 um; mobile phase: [water (0.05% HCl)-ACN]; B %: 25%-55%, 10 min) to give 3-[2-[2-[2-[2-[2-[2-(4,5-dichloro-1,1,3-trioxo-isothiazol-2-yl)ethoxy]ethoxy]ethoxy]ethoxy]ethoxy]ethoxy]propanoic acid (53.79 mg, 98.63 umol, 28.18% yield, 98.724% purity) as a yellow oil; LC-MS (ES, m/z): 538.2 [M+H]⁺; 1H NMR (400 MHz, DMSO-d6) δ=12.13 (br s, 1H), 3.89-3.81 (m, 2H), 3.65 (t, J=5.5 Hz, 2H), 3.59 (t, J=6.4 Hz, 2H), 3.56-3.48 (m, 20H), 2.43 (t, J=6.4 Hz, 2H).

### Example 16

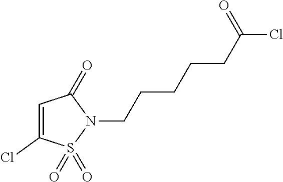 [SMILES: O=C(Cl)CCCCCN1C(=O)C=C(Cl)S1(=O)=O]

In a flask under argon was added product 6-(5-chloro-1,1-dioxido-3-oxoisothiazol-2(3H)-yl) hexanoic acid (12.58 mg, 0.045 mmol), DCM (2 mL) and DMF (10 μl). The mixture was cooled to 0° C., then oxalyl dichloride (11.65 μl, 0.136 mmol) was added dropwise. The mixture was warmed up to rt and was stirred until complete conversion was observed by LCMS (follow-up by LCMS by adding to the aliquot dry MeOH to form the methyl ester). The crude was evaporated under vacuo. The residue was taken in DCM and dried again under vacuo to give a yellow solid. The crude material was used without further purification for the next step.

### 2. Synthesis of the Drug-Linker Conjugates

### Example A. ((2R,3R)-3-((S)-1-((3R,4S,5S)-4-((S)-2-((S)-2-((4-(6-(5-chloro-1,1-dioxido-3-oxoisothiazol-2(3H)-yl)-N-methylhexanamido)phenethyl)(methyl)amino)-3-methylbutanamido)-N,3-dimethylbutanamido)-3-methoxy-5-methylheptanoyl)pyrrolidin-2-yl)-3-methoxy-2-methylpropanoyl)-L-phenylalanine

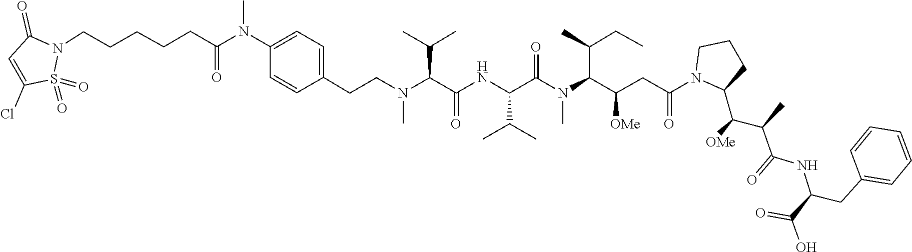 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)CCc1ccc(N(C)C(=O)CCCCCN2C(=O)C=C(Cl)S2(=O)=O)cc1)C(C)C]

### Standard Procedure for the Synthesis of Drug-Linkers:

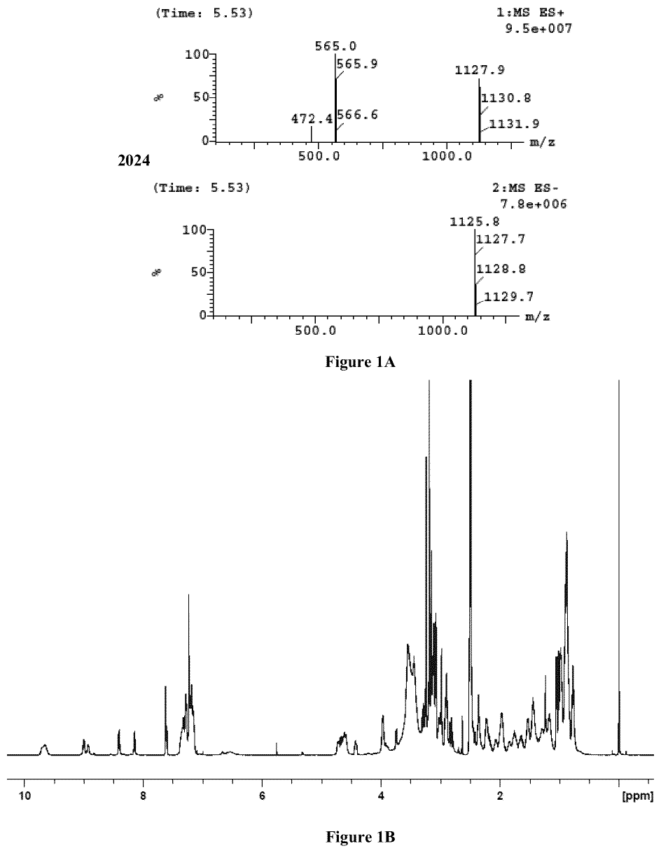

In a flask under nitrogen, were added at rt 6-(5-chloro-1,1-dioxido-3-oxoisothiazol-2(3H)-yl)hexanoic acid (205 mg, 0.73 mmol) (Example 1), dichloromethane (10 mL) and DMF (100 μl). The mixture was cooled to 0° C. using an ice bath, then oxalyl chloride was added (190.4 μl, 2.18 mmol). The mixture was warmed up to rt and was stirred for 2 h. The reaction mixture was evaporated under vacuum. The residue was taken in CH₂Cl₂ and dried again under vacuum to give 6-(5-chloro-1,1-dioxido-3-oxoisothiazol-2(3H)-yl)hexanoyl chloride as a yellow solid. In a vial under N₂ at rt were introduced (S)-2-((2R,3R)-3-((S)-1-((3R,4S,5S)-4-((S)-N,3-dimethyl-2-((S)-3-methyl-2-(methyl(4-(methylamino)phenethyl)amino)butanamido)butanamido)-3-methoxy-5-methylheptanoyl)pyrrolidin-2-yl)-3-methoxy-2-methylpropanamido)-3-phenylpropanoic acid, (S)-2-((2R,3R)-3-((S)-1-((3R,4S,5S)-4-((S)-N,3-dimethyl-2-((S)-3-methyl-2-(methyl(4-(methylamino)phenethyl)amino)butanamido)butanamido)-3-methoxy-5-methylheptanoyl)pyrrolidin-2-yl)-3-methoxy-2-methylpropanamido)-3-phenylpropanoic acid compound with 2,2,2-trifluoroacetic acid (1:1) (111 mg, 0,102 mmol) and dichloromethane (3.7 mL). The mixture was cooled to 0° C. and DIPEA (70.9 μl, 0,406 mmol) was added. The reaction mixture was stirred for 10 min at 0° C., then 6-(5-chloro-1,1-dioxido-3-oxoisothiazol-2(3H)-yl)hexanoyl chloride (36.6 mg, 0,122 mmol) was added dropwise as a solution in DCM (248 mg of acid chloride in 2 mL of DCM). The mixture was stirred at 0° C. for 1 h15. The reaction was stopped by adding trifluoroacetic acid (32.9 μl, 0,426 mmol), acetonitrile (2.1 mL) and water (0.3 mL) into the mixture at 0° C. The crude material was concentrated in vacuum and the residue purified by preparative HPLC (Column X-Bridge C18 (100*30) using a gradient of ACN and water with 0.1% TFA as a mobile phase) to give ((2R,3R)-3-((S)-1-((3R,4S,5S)-4-((S)-2-((S)-2-((4-(6-(5-chloro-1,1-dioxido-3-oxoisothiazol-2(3H)-yl)-N-methylhexanamido)phenethyl)(methyl)amino)-3-methylbutanamido)-N,3-dimethylbutanamido)-3-methoxy-5-methylheptanoyl)pyrrolidin-2-yl)-3-methoxy-2-methylpropanoyl)-L-phenylalanine. The mass spectrum and the ¹H-NMR spectrum of this drug-linker conjugate are represented respectively on FIGS. 1A and 1B.

### Example B. ((2R,3R)-3-((S)-1-((3R,4S,5S)-4-((S)-2-((S)-2-((4-(6-(4,5-dichloro-1,1-dioxido-3-oxoisothiazol-2(3H)-yl)-N-methylhexanamido)phenethyl)(methyl)amino)-3-methylbutanamido)-N,3-dimethylbutanamido)-3-methoxy-5-methylheptanoyl)pyrrolidin-2-yl)-3-methoxy-2-methylpropanoyl)-L-phenylalanine

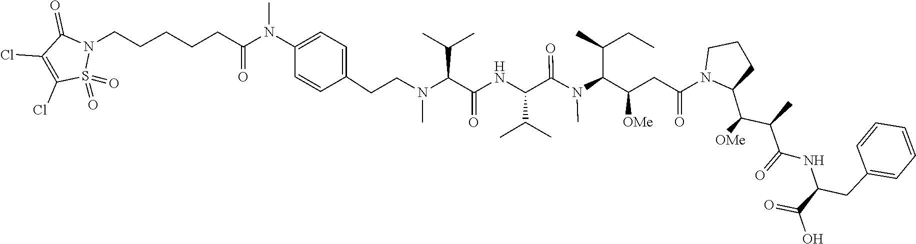 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)CCc1ccc(N(C)C(=O)CCCCCN2C(=O)C(Cl)=C(Cl)S2(=O)=O)cc1)C(C)C]

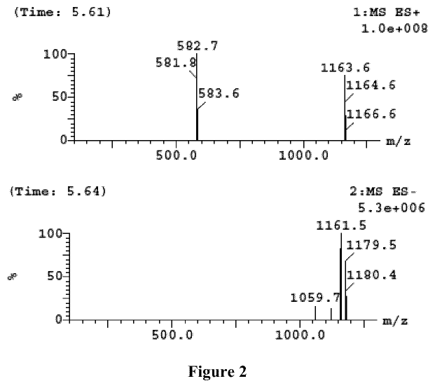

It was synthesized following the standard procedure for the synthesis of drug-linkers using 6-(4,5-dichloro-1,1-dioxido-3-oxoisothiazol-2(3H)-yl)hexanoic acid (Example 2) and ((2R,3R)-3-((S)-1-((3R,4S,5S)-4-((S)-N,3-dimethyl-2-((S)-3-methyl-2-(methyl(4-(methylamino)phenethyl)amino)butanamido)butanamido)-3-methoxy-5-methylheptanoyl)pyrrolidin-2-yl)-3-methoxy-2-methylpropanoyl)-L-phenylalanine compound with 2,2,2-trifluoroacetic acid (1:1) as starting materials.
The mass spectrum of this drug-linker conjugate is represented on FIG. 2.

### Example C. ((2R,3R)-3-((S)-1-((3R,4S,5S)-4-((S)-2-((S)-2-((4-((((4-((S)-2-((S)-2-(6-(5-chloro-1,1-dioxido-3-oxoisothiazol-2(3H)-yl)hexanamido)-3-methylbutanamido)-5-ureidopentanamido)benzyl)oxy)carbonyl)(methyl)amino)phenethyl)(methyl)amino)-3-methylbutanamido)-N,3-dimethylbutanamido)-3-methoxy-5-methylheptanoyl)pyrrolidin-2-yl)-3-methoxy-2-methylpropanoyl)-L-phenylalanine

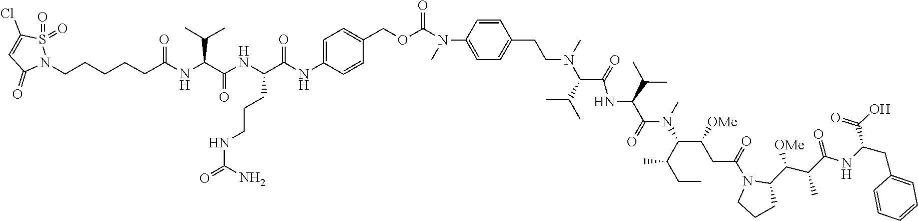

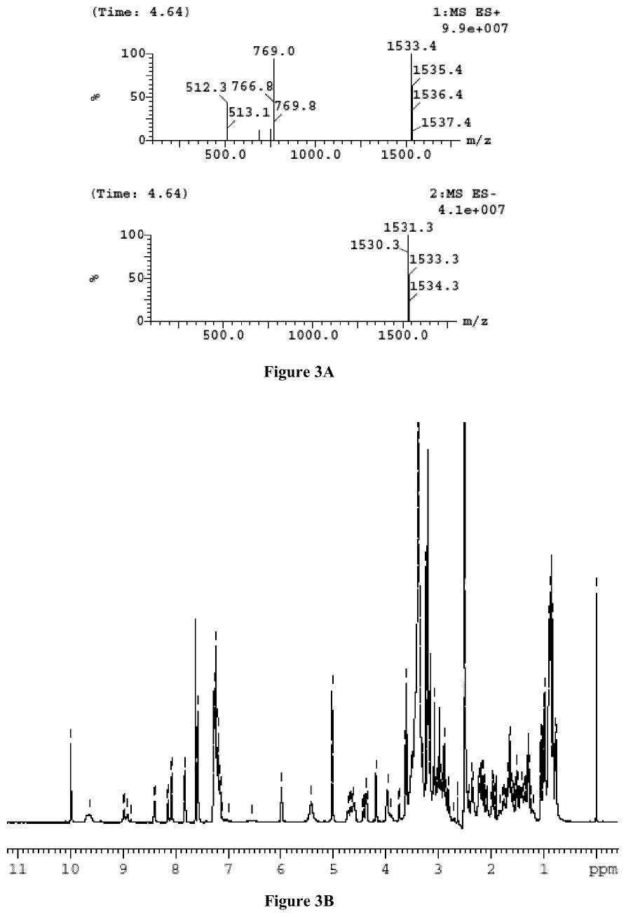

It was obtained following the standard procedure for drug-linker synthesis using 6-(5-chloro-1,1-dioxido-3-oxoisothiazol-2(3H)-yl)hexanoic acid (Example 1) and ((2R,3R)-3-((S)-1-((3R,4S,5S)-4-((S)-2-((S)-2-((4-((((4-((S)-2-((S)-2-amino-3-methylbutanamido)-5-ureidopentanamido)benzyl)oxy)carbonyl) (methyl)amino)phenethyl)(methyl)amino)-3-methylbutanamido)-N,3-dimethylbutanamido)-3-methoxy-5-methylheptanoyl)pyrrolidin-2-yl)-3-methoxy-2-methylpropanoyl)-L-phenylalanine as starting materials.
The mass spectrum and the ¹H-NMR spectrum of this drug-linker conjugate are represented respectively on FIGS. 3A and 3B.

### Example D. ((2R,3R)-3-((S)-1-((3R,4S,5S)-4-((S)-2-((S)-2-((4-((((4-((S)-2-((S)-2-(6-(4,5-dichloro-1,1-dioxido-3-oxoisothiazol-2(3H)-yl)hexanamido)-3-methylbutanamido)-5-ureidopentanamido)benzyl)oxy)carbonyl)(methyl)amino)phenethyl)(methyl)amino)-3-methylbutanamido)-N,3-dimethylbutanamido)-3-methoxy-5-methylheptanoyl)pyrrolidin-2-yl)-3-methoxy-2-methylpropanoyl)-L-phenylalanine

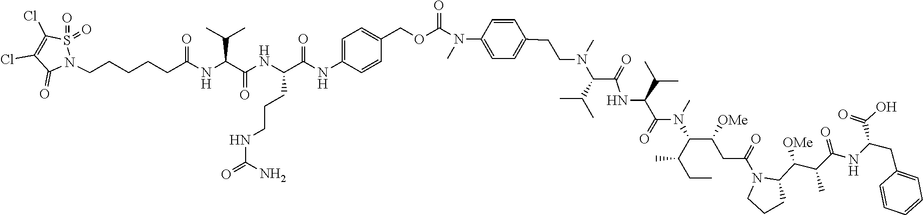 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)CCc1ccc(N(C)C(=O)OCc2ccc(NC(=O)[C@H](CCCNC(N)=O)NC(=O)[C@@H](NC(=O)CCCCCN3C(=O)C(Cl)=C(Cl)S3(=O)=O)C(C)C)cc2)cc1)C(C)C]

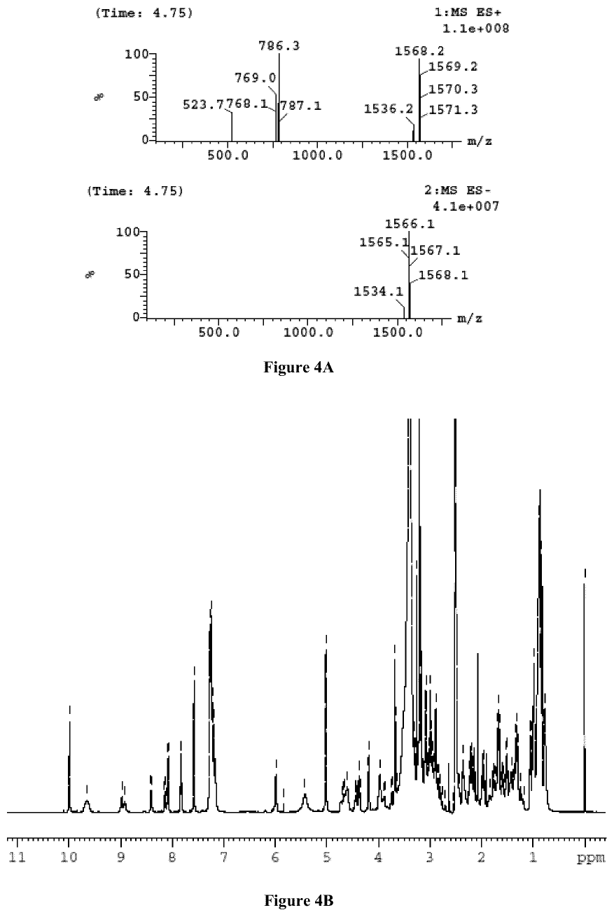

It was obtained following the standard procedure for drug-linker synthesis using 6-(4,5-dichloro-1,1-dioxido-3-oxoisothiazol-2(3H)-yl)hexanoic acid (Example 2) and ((2R,3R)-3-((S)-1-((3R,4S,5S)-4-((S)-2-((S)-2-((4-((((4-((S)-2-((S)-2-amino-3-methylbutanamido)-5-ureidopentanamido)benzyl)oxy)carbonyl)(methyl)amino)phenethyl)(methyl)amino)-3-methylbutanamido)-N,3-dimethylbutanamido)-3-methoxy-5-methylheptanoyl)pyrrolidin-2-yl)-3-methoxy-2-methylpropanoyl)-L-phenylalanine as starting materials.
The mass spectrum and the ¹H-NMR spectrum of this drug-linker conjugate are represented respectively on FIGS. 4A and 4B.

### Example E. ((2R,3R)-3-((S)-1-((3R,4S,5S)-4-((S)-2-((S)-2-((4-((((4-((S)-2-((S)-2-(3-(2-(2-(2-(5-chloro-1,1-dioxido-3-oxoisothiazol-2(3H)-yl)ethoxy)ethoxy)ethoxy)propanamido)-3-methylbutanamido)-5-ureidopentanamido)benzyl)oxy)carbonyl)(methyl)amino)phenethyl)(methyl)amino)-3-methylbutanamido)-N,3-dimethylbutanamido)-3-methoxy-5-methylheptanoyl)pyrrolidin-2-yl)-3-methoxy-2-methylpropanoyl)-L-phenylalanine 2,2,2-trifluoroacetic acid salt

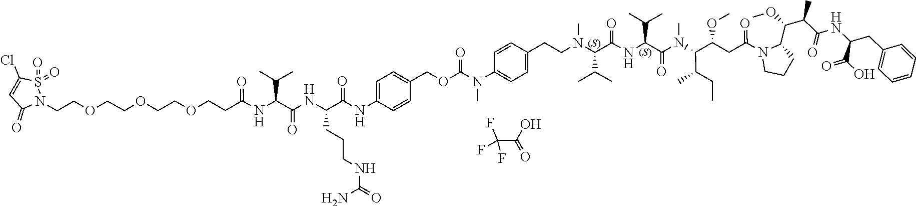

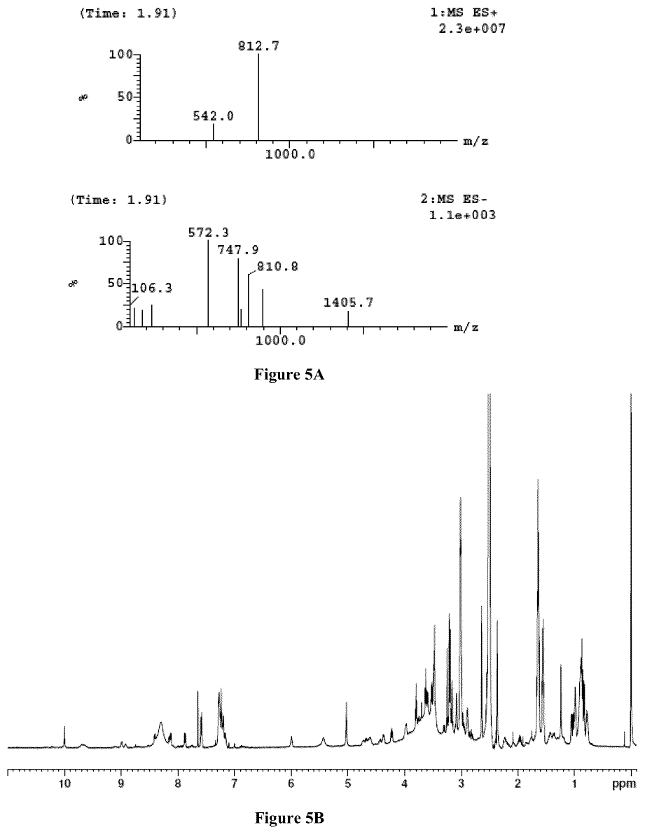

Synthesized following the standard procedure for the synthesis of drug-linkers using 3-(2-(2-(2-(5-chloro-1,1-dioxido-3-oxoisothiazol-2(3H)-yl)ethoxy)ethoxy)ethoxy) propanoic acid (Example 12) and (S)-2-((2R,3R)-3-((S)-1-((3R,4S,5S)-4-((S)-2-((S)-2-((4-((((4-((S)-2-((S)-2-amino-3-methylbutanamido)-5 ureidopentanamido)benzyl)oxy)carbonyl)(methyl)amino)phenethyl)(methyl)amino)-3-methylbutanamido)-N,3-dimethylbutanamido)-3-methoxy-5-methylheptanoyl)pyrrolidin-2-yl)-3-methoxy-2-methylpropanamido)-3-phenylpropanoic acid as starting materials.
The mass spectrum and the ¹H-NMR spectrum of this drug-linker conjugate are represented respectively on FIGS. 5A and 5B.

### Example F. ((2R,3R)-3-(1-((3R,4R,5S)-4-((S)-2-((S)-2-((4-((S)-2-((S)-2-(3-(2-(2-(2-(5-chloro-1,1-dioxido-3-oxoisothiazol-2(3H)-yl)ethoxy)ethoxy)ethoxy)propanamido)-3-methylbutanamido)-N-methylpropanamido)phenethyl)(methyl)amino)-3-methylbutanamido)-N,3-dimethylbutanamido)-3-methoxy-5-methylheptanoyl)pyrrolidin-2-yl)-3-methoxy-2-methylpropanoyl)-D-phenylalanine

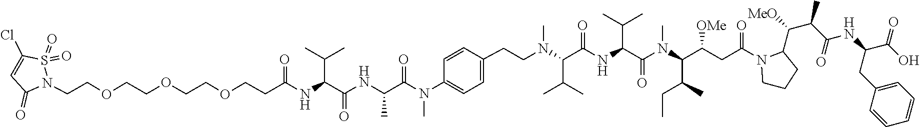

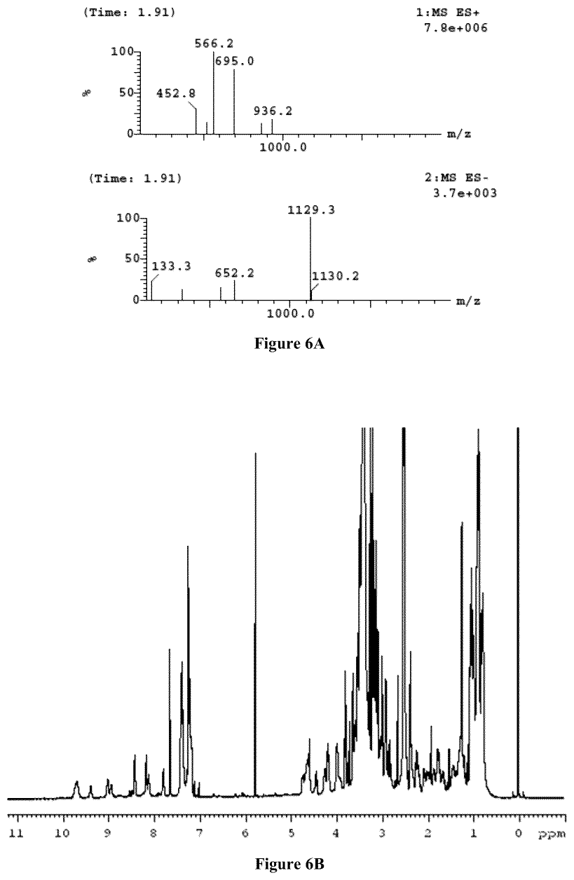

Synthesized following the standard procedure for the synthesis of drug-linkers using 3-(2-(2-(2-(5-chloro-1,1-dioxido-3-oxoisothiazol-2(3H)-yl)ethoxy)ethoxy)ethoxy) propanoic acid (Example 12) and ((2R,3R)-3-(1-((3R,4R,5S)-4-((S)-2-((S)-2-((4-((S)-2-((S)-2-amino-3-methylbutanamido)-N-methylpropanamido)phenethyl)(methyl)amino)-3-methylbutanamido)-N,3-dimethylbutanamido)-3-methoxy-5-methylheptanoyl)pyrrolidin-2-yl)-3-methoxy-2-methylpropanoyl)-D-phenylalanine as starting materials.
The mass spectrum and the ¹H-NMR spectrum of this drug-linker conjugate are represented respectively on FIGS. 6A and 6B.

### Example G. ((2R,3R)-3-(1-((3R,4R,5S)-4-((S)-2-((S)-2-((4-((S)-2-((S)-2-(6-(5-chloro-1,1-dioxido-3-oxoisothiazol-2(3H)-yl)hexanamido)-3-methylbutanamido)-N-methylpropanamido)phenethyl)(methyl)amino)-3-methylbutanamido)-N,3-dimethylbutanamido)-3-methoxy-5-methylheptanoyl)pyrrolidin-2-yl)-3-methoxy-2-methylpropanoyl-D-phenylalanine

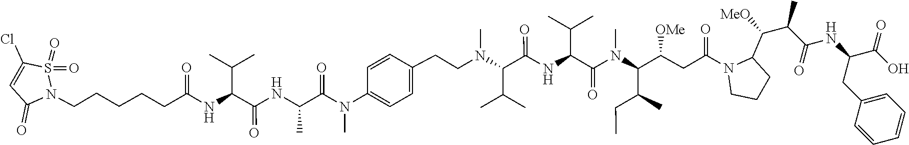 [SMILES: CC[C@H](C)[C@H]([C@@H](CC(=O)N1CCCC1[C@H](OC)[C@@H](C)C(=O)N[C@H](Cc1ccccc1)C(=O)O)OC)N(C)C(=O)[C@@H](NC(=O)[C@H](C(C)C)N(C)CCc1ccc(N(C)C(=O)[C@H](C)NC(=O)[C@@H](NC(=O)CCCCCN2C(=O)C=C(Cl)S2(=O)=O)C(C)C)cc1)C(C)C]

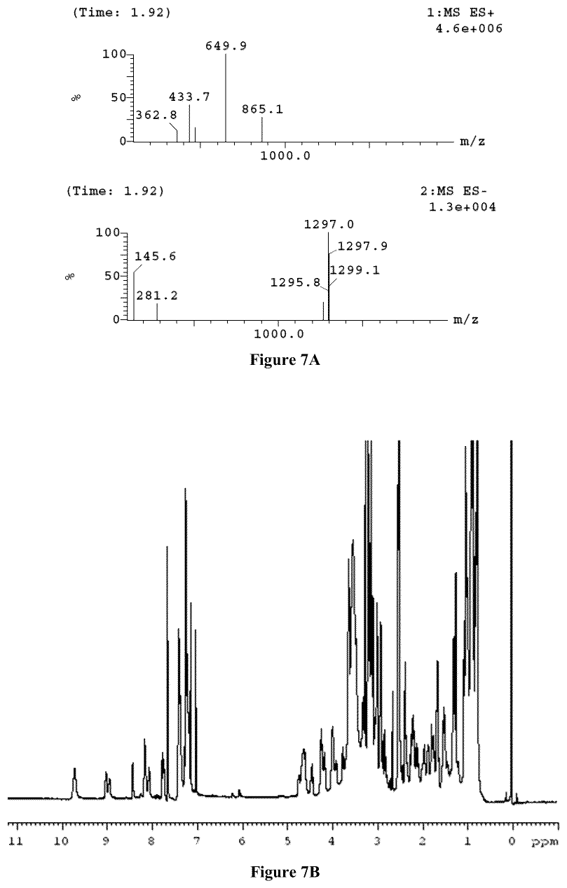

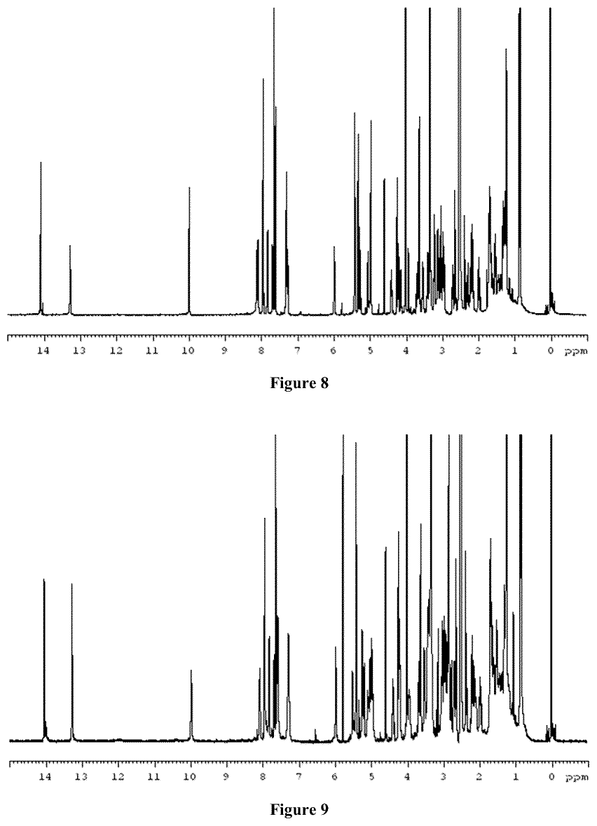

Synthesized following the standard procedure for the synthesis of drug-linkers using 6-(5-chloro-1,1-dioxido-3-oxoisothiazol-2(3H)-yl)hexanoic acid (Example 1) and ((2R,3R)-3-(1-((3R,4R,5S)-4-((S)-2-((S)-2-((4-((S)-2-((S)-2-amino-3-methylbutanamido)-N-methylpropanamido)phenethyl)(methyl)amino)-3-methylbutanamido)-N,3-dimethylbutanamido)-3-methoxy-5-methylheptanoyl)pyrrolidin-2-yl)-3-methoxy-2-methylpropanoyl)-D-phenylalanine as starting materials.
The mass spectrum and the ¹H-NMR spectrum of this drug-linker conjugate are represented respectively on FIGS. 7A and 7B.

### Example H. 4-((S)-2-((S)-2-(6-(5-chloro-1,1-dioxido-3-oxoisothiazol-2(3H)-yl)hexanamido)-3-methylbutanamido)-5-ureidopentanamido)benzyl (2-((2S,4S)-2,5,12-trihydroxy-7-methoxy-4-(((1S,3R,4aS,9S,9aR,10aS)-9-methoxy-1-methyloctahydro-1H-pyrano[4′,3′:4,5]oxazolo[2,3-c][1,4]oxazin-3-yl)oxy)-6,11-dioxo-1,2,3,4,6,11-hexahydrotetracene-2-carboxamido)ethyl)carbamate

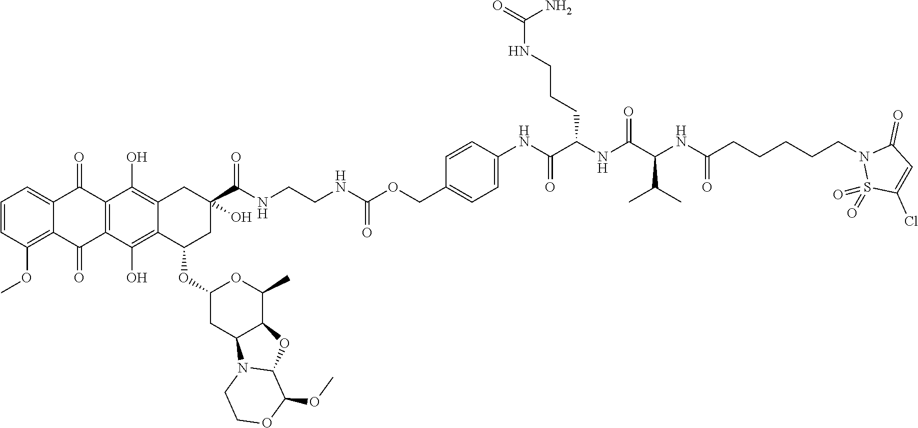

Example H has been synthesized according to the following synthetic path:

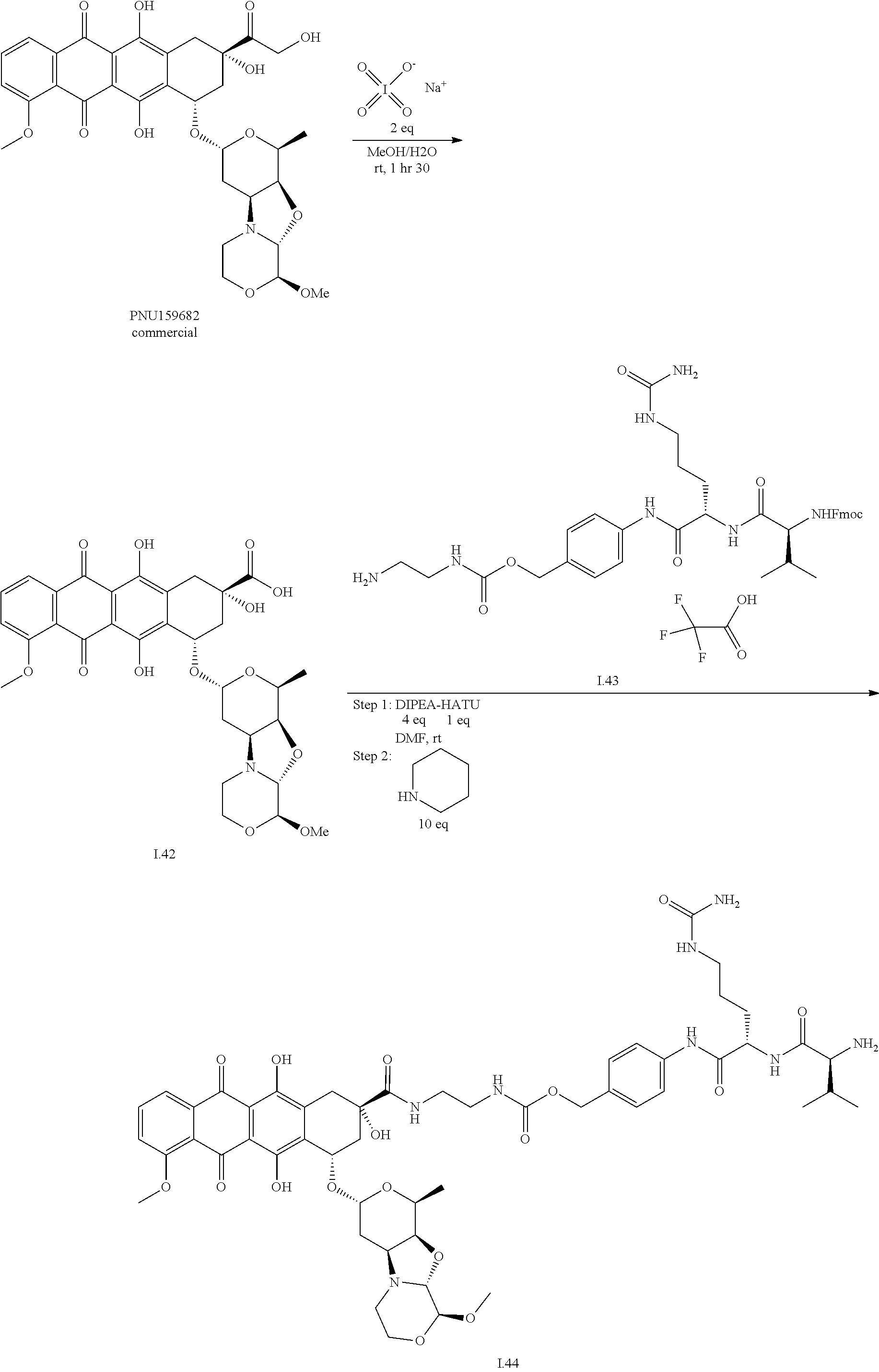

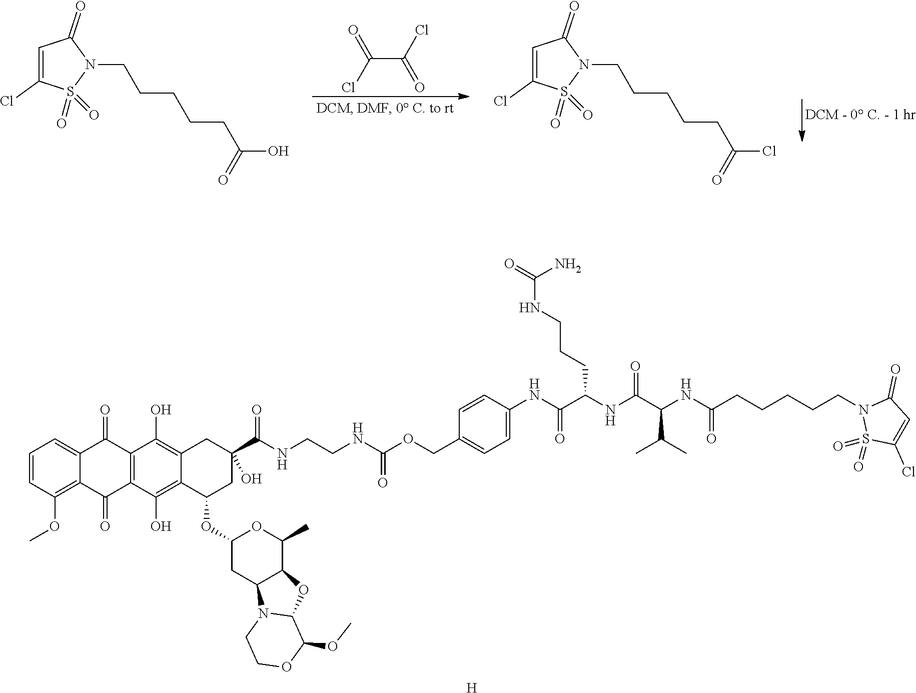

I.42. (2S,4S)-2,5,12-trihydroxy-7-methoxy-4-(((1S,3R,4aS,9S,9aR,10aS)-9-methoxy-1-methyloctahydro-1H-pyrano[4′,3′:4,5]oxazolo[2,3-c][1,4]oxazin-3-yl)oxy)-6,11-dioxo-1,2,3,4,6, 11-hexahydrotetracene-2-carboxylic acid
In a flask was added PNU-159682 (52 mg, 0.081 mmol) in a mixture of methanol (15 mL) and water (10 mL). A solution of NaI0₄ (34.7 mg, 0.162 mmol) in water (5 mL) was added. The reaction mixture was stirred at rt until complete conversion was observed by LCMS. The solvents were removed under vacuo to give I.42 as a red solid which was used directly in the next step.
I.43 (9H-fluoren-9-yl)methyl ((S)-1-(((S)-1-((4-((((2-aminoethyl)carbamoyl)oxy) methyl)phenyl) amino)-1-oxo-5-ureidopentan-2-yl)amino)-3-methyl-1-oxobutan-2-yl)carbamate
In a flask under argon were added (9H-fluoren-9-yl)methyl ((S)-1-(((S)-1-((4-(hydroxymethyl)phenyl)amino)-1-oxo-5-ureidopentan-2-yl)amino)-3-methyl-1-oxobutan-2-yl)carbamate (1 g, 1.662 mmol) and bis(4-nitrophenyl) carbonate (1.011 g, 3.32 mmol) in DMF (0.443 mol/L). Then, the mixture was cooled at 0° C. and DIPEA (639 μL, 3.66 mmol) was added dropwise. The reaction mixture was warmed up to rt and stirred for 18 h. The crude mixture was concentrated under vacuo. The crude product was taken in 1:1 mixture of Et₂O/EtOAc and filtered. The precipitate was washed with Et₂O, citric acid 5%, H₂O then Et₂O again to obtain a yellow solid. This solid was purified by automatic column chromatography silica gel (100 DCM:0 MeOH to 80 DCM:20 MeOH) to give 345 mg of (9H-fluoren-9-yl)methyl ((S)-3-methyl-1-(((S)-1-((4-((((4-nitrophenoxy)carbonyl)oxy)methyl)phenyl)amino)-1-oxo-5-ureidopentan-2-yl)amino)-1-oxobutan-2-yl)carbamate (white solid), 27.1% yield.
To a solution of the previous product (150 mg, 0.196 mmol) in DMF (6 mL) were added HOBt (34.4 mg, 0.254 mmol) and pyridine (63.3 μl, 0.782 mmol) at 0° C. After 5 min, tert-butyl (2-aminoethyl)carbamate 1-2 (40.7 mg, 0.254 mmol) in DMF (1.5 mL) was added to the mixture, followed by DIPEA (102 μl, 0.587 mmol). The mixture was warmed to rt and stirred for 2 h. The crude was concentrated under vacuo to give a white solid which was purified by automatic column chromatography silica gel (100 DCM:0 MeOH to 80 DCM:20 MeOH) to give 129 mg of 4-((S)-2-((S)-2-((((9H-fluoren-9-yl)methoxy)carbonyl)amino)-3-methylbutanamido)-5-ureidopentanamido)benzyl tert-butyl ethane-1,2-diyldicarbamate (white solid), 84% yield.
In a flask was placed the previous product (154 mg, 0.195 mmol) in DCM (6 mL). The mixture was cooled at 0° C. and TFA (753 μL, 9.77 mmol) was added and the mixture was stirred at 0° C. until complete conversion was observed by LCMS. The crude mixture was concentrated in vacuo to give I.43 as a white solid (quantitative yield).
I.44 4-((S)-2-((S)-2-amino-3-methylbutanamido)-5-ureidopentanamido)benzyl (2-((2S,4S)-2,5,12-trihydroxy-7-methoxy-4-(((1S,3R,4aS,9S,9aR,10aS)-9-methoxy-1-methyloctahydro-1H-pyrano[4′,3′:4,5]oxazolo[2,3-c][1,4]oxazin-3-yl)oxy)-6,11-dioxo-1,2,3,4,6,11-hexahydrotetra cene-2-carboxamido)ethyl)carbamate
In a flask were added I.42 (50.9 mg, 0.081 mmol), L₄₃ (78.0 mg, 0.097 mmol) and DMF (8 mL) followed by HATU (30.8 mg, 0.081 mmol) and DIPEA (56.7 μl, 0.324 mmol). The reaction mixture was stirred at rt for 18 h. To this mixture was then added piperidine (80 μl, 0.811 mmol). The reaction mixture was stirred for 1 h (until complete conversion was observed by LCMS). The mixture was concentrated under vacuo. The crude product obtained was immediately purified by automatic column chromatography silica gel (100 DCM:0 MeOH/NH₃ aq to 85 DCM:25 MeOH/NH₃aq) to give 20 mg of I.44 (red oil), 23% yield.

### Example H

In a flask under N₂ was added 6-(5-Chloro-1,1-dioxido-3-oxoisothiazol-2(3H)-yl)hexanoic acid (example 1) (7.86 mg, 0.028 mmol) in DCM (1 mL) and DMF (10 μl). The mixture was cooled at 0° C., then oxalyl chloride (7.28 μl, 0.085 mmol) was added dropwise. The mixture was warmed up to rt and was stirred until complete conversion was observed by LCMS (follow-up by LCMS by adding to the aliquot dry MeOH to form the methyl ester). The crude mixture was evaporated under vacuo. The residue was taken in DCM and dried again under vacuo to give 6-(5-chloro-1,1-dioxido-3-oxoisothiazol-2(3H)-yl)hexanoyl chloride as a yellow solid (yield quantitative). The crude material was used without further purification for the next step.
In a flask under N₂ were introduced at rt I.44 (20 mg, 0.019 mmol) in DCM (2 mL). The mixture was cooled to 0° C. and DIPEA (12.96 μl, 0.074 mmol) was added. The mixture was stirred at 0° C. for 10 min then the product of previous step (8.40 mg, 0.028 mmol) diluted in DCM (1 mL) was added. The mixture was then stirred at 0° C. for 2 h (until complete conversion was observed by LCMS). The crude mixture was concentrated under vacuo and purified by automatic column chromatography, silica gel (100 DCM:0 MeOH to 85 DCM:15 MeOH) to give 6.85 mg of example H (also named compound F562524) as a red solid, 27% yield.
The ¹H-NMR spectrum of this drug-linker conjugate is represented on FIG. 8.

### Example I. 4-((S)-2-((S)-2-(6-(5-chloro-1,1-dioxido-3-oxoisothiazol-2(3H)-yl)hexanamido)-3-methylbutanamido)-5-ureidopentanamido)benzyl (2-oxo-2-((2S,4S)-2,5,12-trihydroxy-7-methoxy-4-(((1S,3R,4aS,9S,9aR,10aS)-9-methoxy-1-methyloctahydro-1H-pyrano[4′,3′:4,5]oxazolo[2,3-c][1,4]oxazin-3-yl)oxy)-6,11-dioxo-1,2,3,4,6,11-hexahydrotetracen-2-yl)ethyl) ethane-1,2-diylbis(methylcarbamate)

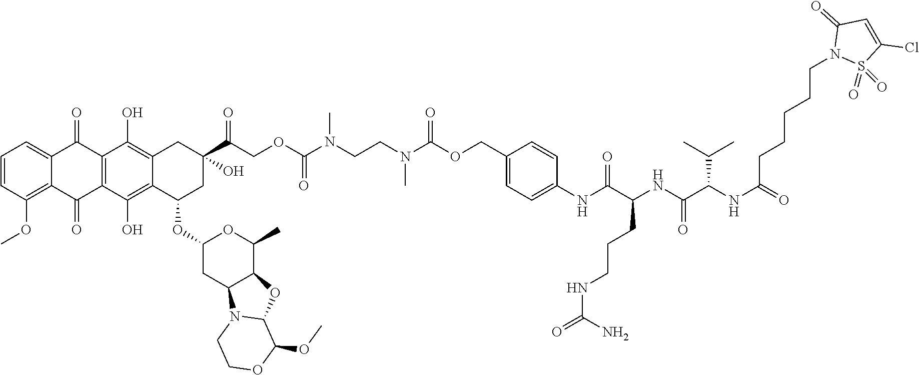 [SMILES: COc1cccc2c1C(=O)c1c(O)c3c(c(O)c1C2=O)C[C@@](O)(C(=O)COC(=O)N(C)CCN(C)C(=O)OCc1ccc(NC(=O)[C@H](CCCNC(N)=O)NC(=O)[C@@H](NC(=O)CCCCCN2C(=O)C=C(Cl)S2(=O)=O)C(C)C)cc1)C[C@@H]3O[C@H]1C[C@H]2[C@H](O[C@@H]3[C@@H](OC)OCCN32)[C@H](C)O1]

Example I has been synthesized according to the following synthetic path:

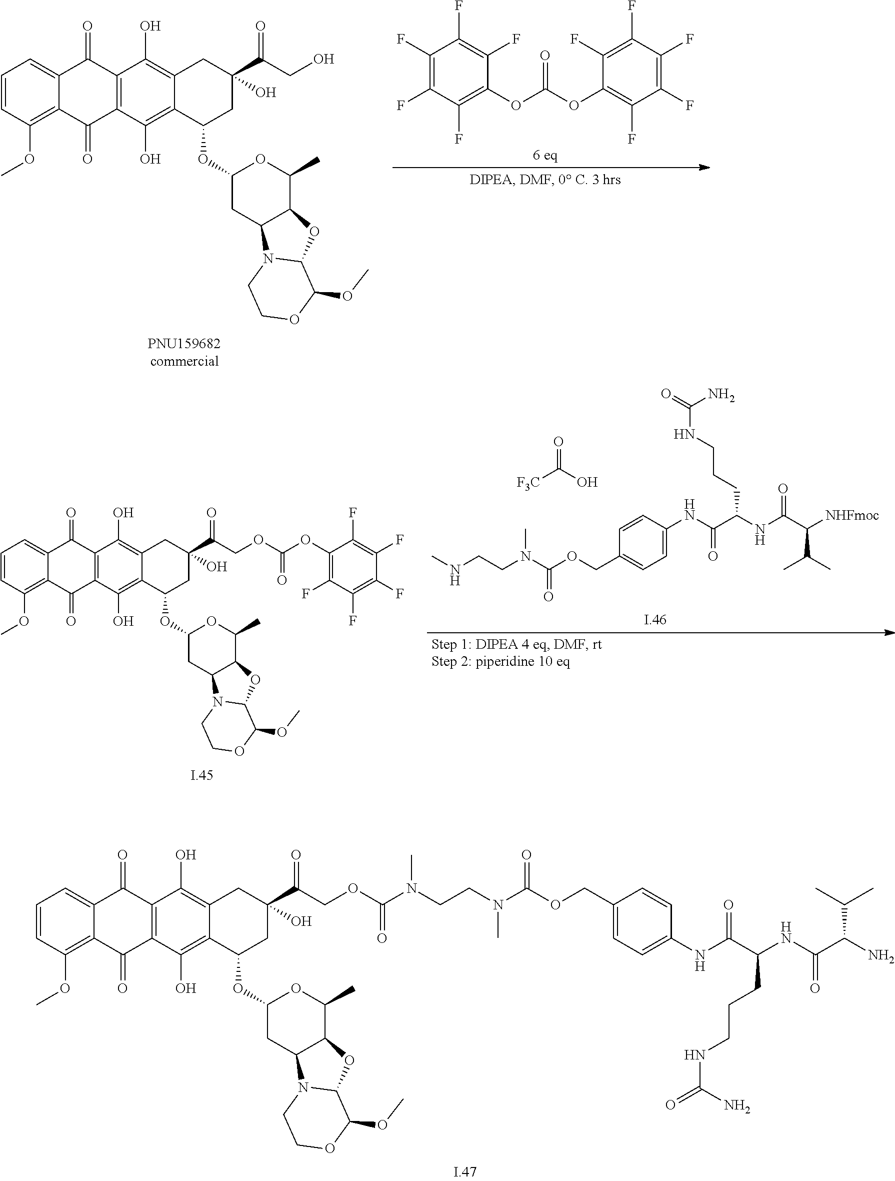

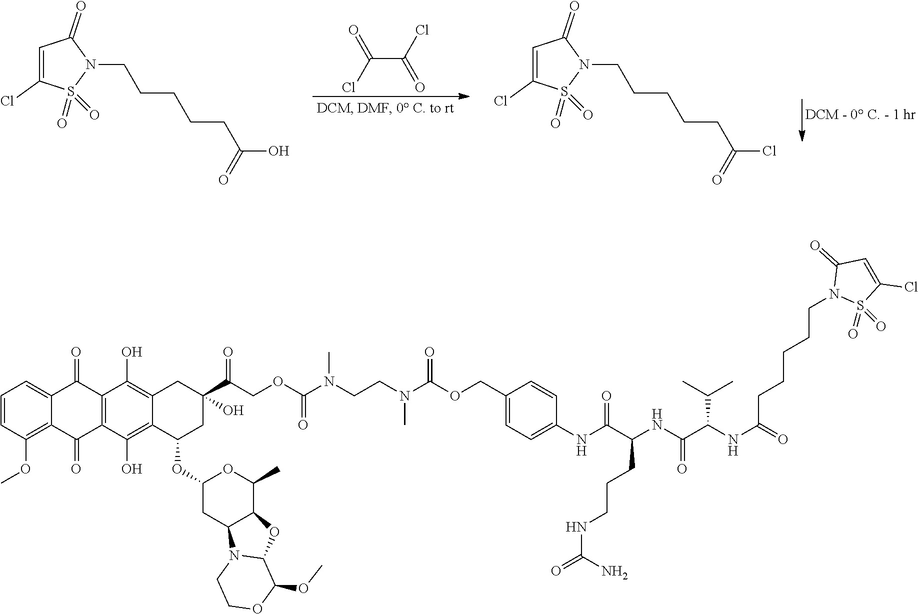 [SMILES: O=C(O)CCCCCN1C(=O)C=C(Cl)S1(=O)=O ; O=C(Cl)C(=O)Cl ; O=C(Cl)CCCCCN1C(=O)C=C(Cl)S1(=O)=O ; COc1cccc2c1C(=O)c1c(O)c3c(c(O)c1C2=O)C[C@@](O)(C(=O)COC(=O)N(C)CCN(C)C(=O)OCc1ccc(NC(=O)[C@H](CCCNC(N)=O)NC(=O)[C@@H](NC(=O)CCCCCN2C(=O)C=C(Cl)S2(=O)=O)C(C)C)cc1)C[C@@H]3O[C@H]1C[C@H]2[C@H](O[C@@H]3[C@@H](OC)OCCN32)[C@H](C)O1]

I.45 2-oxo-2-((2S,4S)-2,5,12-trihydroxy-7-methoxy-4-(((1S,3R,4aS,9S,9aR,10aS)-9-methoxy-1-methyloctahydro-1H-pyrano[4′,3′:4,5]oxazolo[2,3-c][1,4]oxazin-3-yl)oxy)-6,11-dioxo-1,2,3,4,6,11-hexahydrotetracen-2-yl)ethyl (perfluorophenyl) carbonate
In a flask under argon were added PNU-159682 (12 mg, 0.0180 mmol) and DMF (1.5 mL). The mixture was cooled at 0° C. and bis(perfluorophenyl) carbonate (36.9 mg, 0.094 mmol) was added. Then a solution of DIPEA (9.80 μl, 0.056 mmol) in DMF (0.5 mL) was slowly added over a period of 5 min. The mixture was finally stirred for 3 h at 0° C. (conversion observed by LCMS). The crude mixture was concentrated in vacuo and purified by automatic column chromatography, silica gel (100 DCM:0 [80 DCM:20 MeOH] to 50 DCM:50 [80 DCM:2 0MeOH]) to give 5.52 mg of I.45 as a red oil, 36% yield.
I.46 (9H-fluoren-9-yl)methyl ((S)-3-methyl-1-(((S)-1-((4-((((4-nitrophenoxy)carbonyl) oxy)methyl)phenyl)amino)-1-oxo-5-ureidopentan-2-yl)amino)-1-oxobutan-2-yl)carbamate 2,2,2-trifluoroacetate
In a flask under argon were added (9H-fluoren-9-yl)methyl ((S)-1-(((S)-1-((4-(hydroxymethyl)phenyl)amino)-1-oxo-5-ureidopentan-2-yl)amino)-3-methyl-1-oxobutan-2-yl)carbamate (1 g, 1.662 mmol) and bis(4-nitrophenyl) carbonate (1.011 g, 3.32 mmol) in DMF (0.443 mol/L). Then, the mixture was cooled at 0° C. and DIPEA (639 μL, 3.66 mmol) was added dropwise. The reaction mixture was warmed up to rt and stirred for 18 h. The crude mixture was concentrated under vacuo, taken in Et₂O/EtOAc (1/1) and filtered. The precipitate was washed with Et₂O, citric acid 5%, H₂O then Et₂O again to obtain a yellow solid. This solid was purified by automatic column chromatography, silica gel (100 DCM:0 MeOH to 80 DCM:20 MeOH) to give 345 mg of a white solid, 27.1% yield. To a solution of this compound (118 mg, 0,154 mmol) in DMF (6 mL) were added HOBt (27.0 mg, 0.200 mmol) and pyridine (49.8 μl, 0.616 mmol) at 0° C. After 5 min, tert-butyl methyl(2-(methylamino)ethyl)carbamate (37.7 mg, 0.200 mmol) in DMF (1.5 mL) was added to the mixture, followed by DIPEA (81.0 μl, 0.462 mmol). The mixture was then warmed to rt and stirred for 2 h (until complete conversion was observed by LCMS). The crude mixture was concentrated under vacuo to give a yellow oil which was purified by automatic column chromatography, silica gel (100 DCM:0 MeOH to 80 DCM:20 MeOH) to give 103 mg of a white solid, 82% yield.
In a flask was placed this product (198 mg, 0.243 mmol) in DCM (12 mL). The mixture was cooled to 0° C. and TFA (935 μl, 12.13 mmol) was added and the mixture was stirred for 4 h at 0° C. (until complete conversion was observed by LCMS). The crude mixture was concentrated under vacuo to give 220 mg of I.46 as a clear yellow solid (quantitative yield).
I.47 4-((S)-2-((S)-2-amino-3-methylbutanamido)-5-ureidopentanamido)benzyl (2-oxo-2-((2S,4S)-2,5,12-trihydroxy-7-methoxy-4-(((1S,3R,4aS,9S,9aR,10aS)-9-methoxy-1-methyloctahydro-1H-pyrano[4′,3′:4,5]oxazolo[2,3-c][1,4]oxazin-3-yl)oxy)-6,11-dioxo-1,2,3,4,6,11-hexahydrotetracen-2-yl)ethyl) ethane-1,2-diylbis(methylcarbamate) To a solution of product I.45 (19 mg, 0.022 mmol) in DMF (1 mL) was added at rt a solution of product I.46 (22.2 mg, 0.027 mmol) and DIPEA (15.59 μl, 0.089 mmol) in DMF (1 mL). The reaction mixture was stirred at rt for 3 h (until complete conversion was observed by LCMS). Then, to the mixture was added piperidine (22.09 μl, 0.223 mmol). The reaction mixture was stirred for 1 h (complete conversion observed by LCMS). The crude mixture was concentrated under vacuo and purified by automatic column chromatography, silica gel (100 DCM:0 MeOH/NH₃ (9/1) to 75 DCM:25 MeOH/NH₃ (9/1)) to give 10 mg of I,47 as a red oil, 39% yield.

### Example I

In a flask under N₂ was added 6-(5-Chloro-1,1-dioxido-3-oxoisothiazol-2(3H)-yl)hexanoic acid (example 1) (7.86 mg, 0.028 mmol) in DCM (1 mL) and DMF (10 μl).
The mixture was cooled at 0° C., then oxalyl chloride (7.28 μl, 0.085 mmol) was added dropwise. The mixture was warmed up to rt and was stirred until complete conversion was observed by LCMS (follow-up by LCMS by adding to the aliquot dry MeOH to form the methyl ester). The crude mixture was evaporated under vacuo. The residue was taken in DCM and dried again under vacuo to give 6-(5-chloro-1,1-dioxido-3-oxoisothiazol-2(3H)-yl)hexanoyl chloride as a yellow solid (yield quantitative). The crude material was used without further purification for the next step.
In a flask under N₂ at rt was introduced product I.47 (10 mg, 0.0086 mmol) in DCM (2 mL). The mixture was cooled at 0° C. and DIPEA (6.0 μl, 0.034 mmol) was added. The mixture was stirred at 0° C. for 10 min then addition of previous product (3.90 mg, 0.013 mmol) diluted in DCM (1 mL). The mixture was then stirred at 0° C. for 2 h (until complete conversion was observed by LCMS). The crude was concentrated in vacuo and purified by automatic column chromatography, silica gel (100 DCM:0 MeOH to 85 DCM:15 MeOH) to give 2.45 mg of example I as a red solid, 19% yield.
The ¹H-NMR spectrum of this drug-linker conjugate is represented on FIG. 9.

### Example J

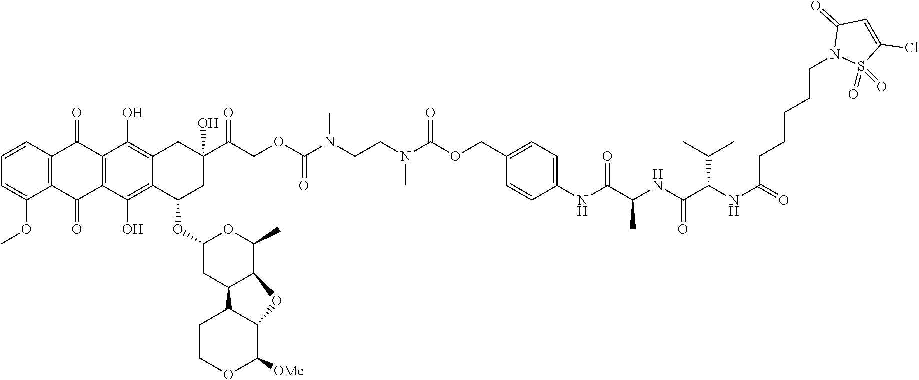

Example J has been synthesized according to the following synthetic path:

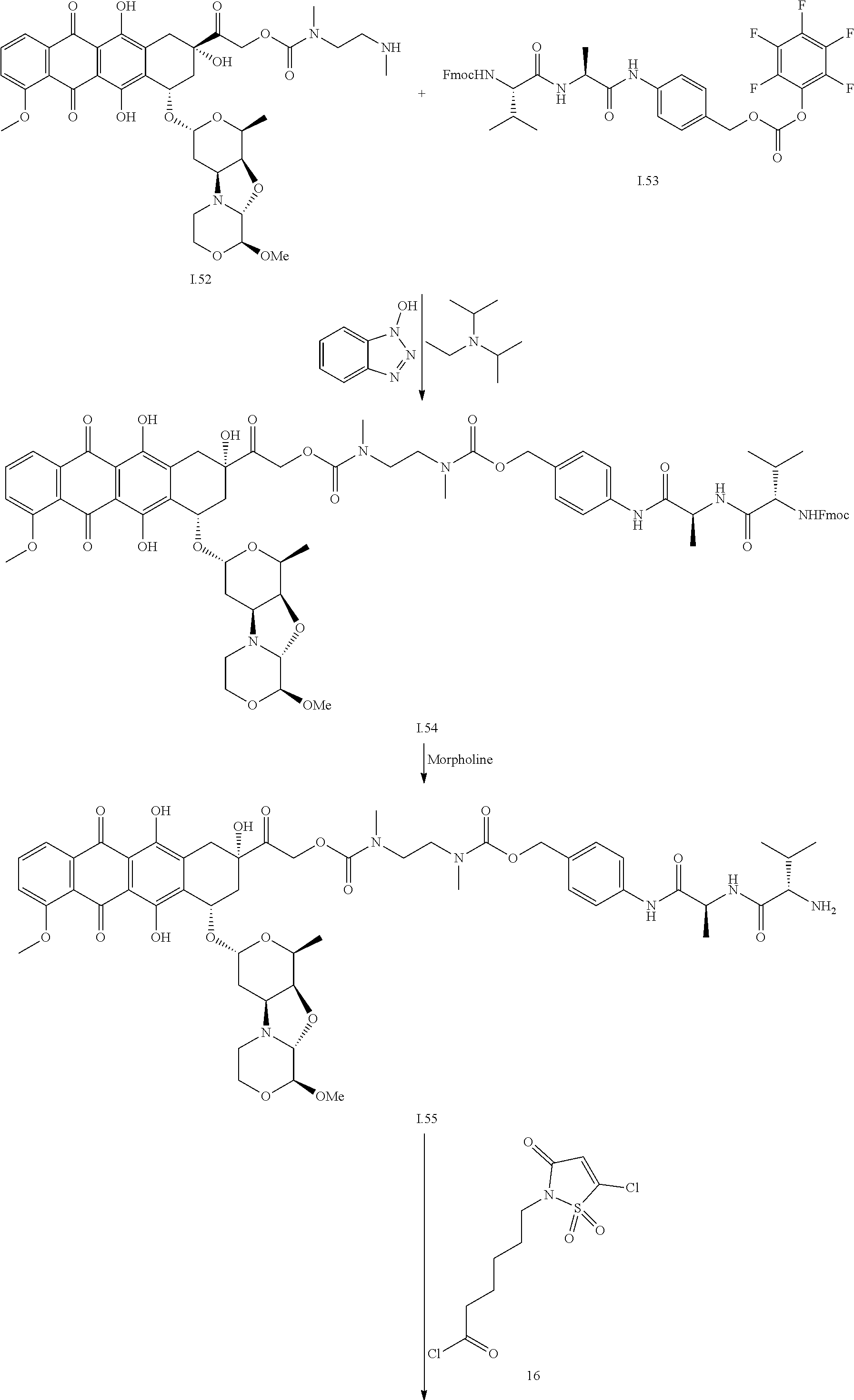

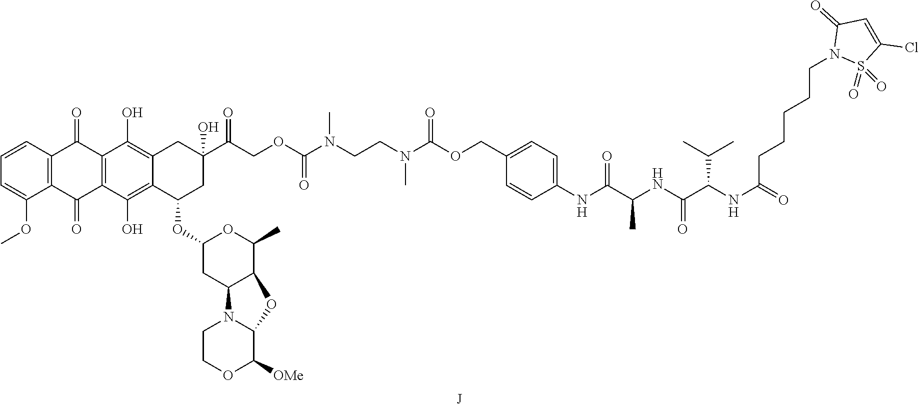 [SMILES: COc1cccc2c1C(=O)c1c(O)c3c(c(O)c1C2=O)C[C@@](O)(C(=O)COC(=O)N(C)CCN(C)C(=O)OCc1ccc(NC(=O)[C@H](C)NC(=O)[C@@H](NC(=O)CCCCCN2C(=O)C=C(Cl)S2(=O)=O)C(C)C)cc1)C[C@@H]3O[C@H]1C[C@H]2[C@H](O[C@@H]3[C@@H](OC)OCCN32)[C@H](C)O1]

Compound I.50 has been prepared according to the following synthetic path:

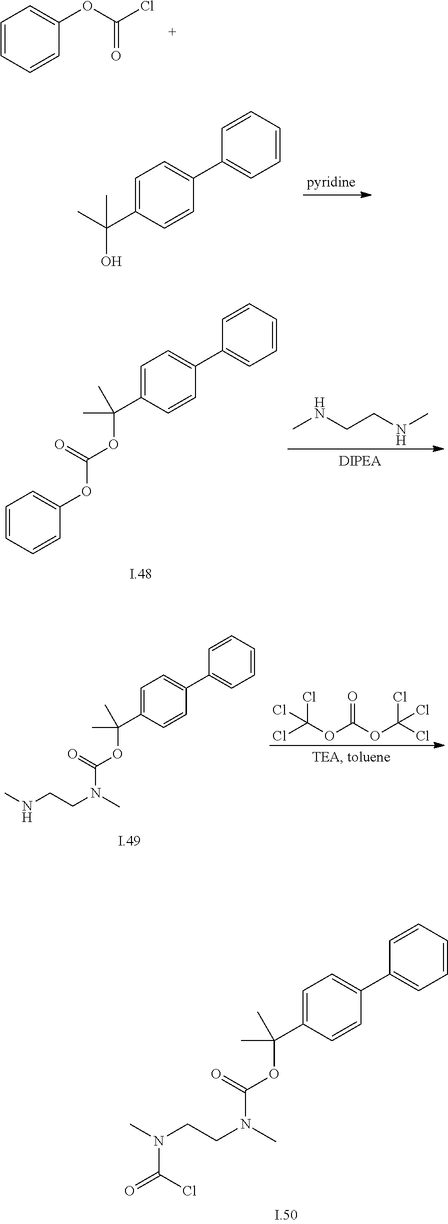

### Compound I.48:

In a flask under argon were added 2-([1,1′-biphenyl]-4-yl) propan-2-ol (1 g, 4.71 mmol) and pyridine (0.465 ml, 5.75 mmol) in DCM (5 mL). Then, the mixture was cooled to 0° C. and phenyl chloroformate (0.662 ml, 5.28 mmol) in DCM dry (2.4 mL) were added dropwise. The reaction mixture was warmed up to rt and stirred for 18 h (check by LCMS). The crude was concentrated in vacuo. The solid mixture was dissolved in DCM and washed with brine 3 times. The organic layer was dried over Na₂SO₄, filtered and concentrated in vacuo to give the desired compound I.48, Yield 880 mg, 56% as white solid. LCMS (ESI): 333.40 (MH+).

### Compound I.49:

In a flask under argon containing N,N′-dimethyl-1,2-ethanediamine (2791 μl, 26.2 mmol), N-ethyl-N-isopropylpropan-2-amine (305 μl, 1.748 mmol) and DMF (4 mL), a solution of 2-([1,1′-biphenyl]-4-yl)propan-2-yl phenyl carbonate (581 mg, 1.748 mmol) in DMF (1.5 mL) was added at 0° C. The reaction mixture was warmed up to rt and stirred for 24 h (check by LCMS). The crude was concentrated in vacuo and the residue was purified by automatic column chromatography (Interchim, solid deposit): DCM/MeOH: 9/1. The desired fractions were concentrated in vacuo to give the desired compound I.49, Yield 433 mg, 76% as yellow oil. LCMS (ESI): 327.43 (MH+).

### Compound I.50:

In a flask under argon containing bis(trichloromethyl)carbonate (157 mg, 0.531 mmol) and toluene (4.3 mL), a solution of 2-([1,1′-biphenyl]-4-yl)propan-2-yl methyl(2-(methylamino)ethyl)carbamate (433 mg, 1,326 mmol) and triethyl amine (368 μl, 2.65 mmol) in toluene (2.9 mL) was added at 0° C. The reaction mixture was warmed up to rt and stirred for 1 h (check by LCMS). The solution was filtered, and the solvent was concentrated in vacuo and the residue was purified by automatic column chromatography (Interchim, solid deposit): cyclohexane/ethyl acetate: 7/3. The desired fractions were concentrated in vacuo to give the desired compound I.50, Yield 166 mg, 33% as white solid. LCMS (ESI): 405.60 (MH+).
Compound I.52 has been prepared according to the following synthetic path:

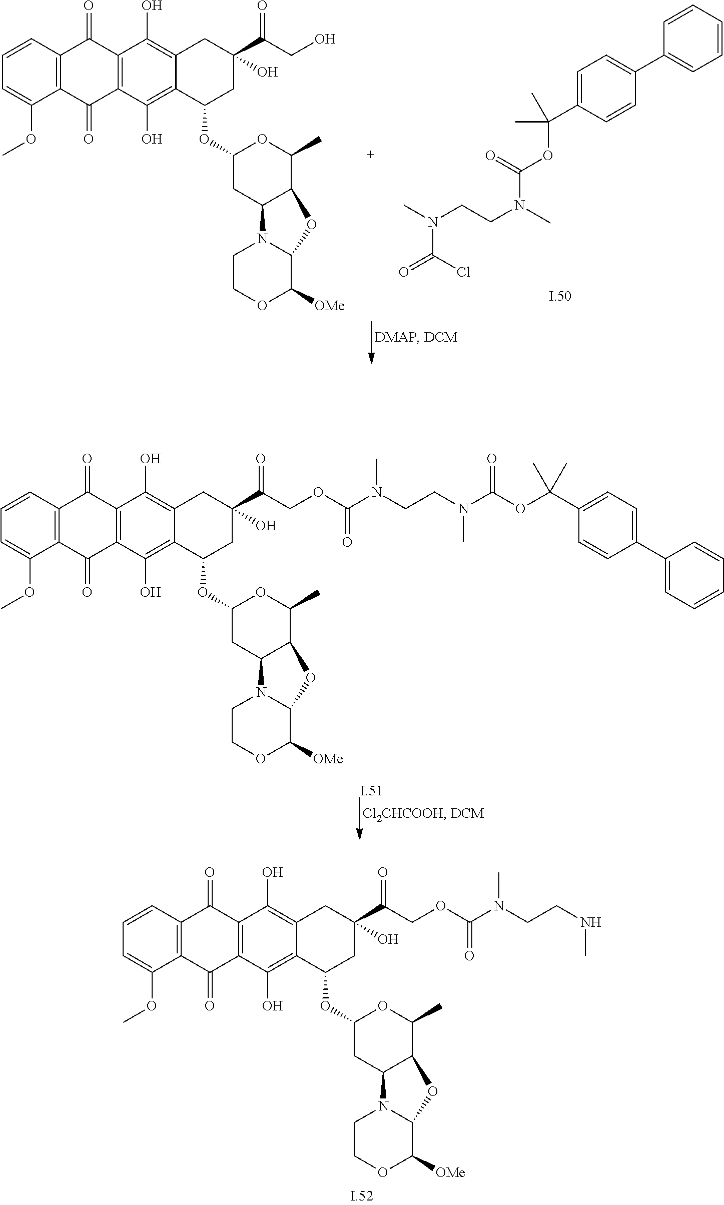 [SMILES: COc1cccc2c1C(=O)c1c(O)c3c(c(O)c1C2=O)C[C@@](O)(C(=O)CO)C[C@@H]3O[C@H]1C[C@H]2[C@H](O[C@@H]3[C@@H](OC)OCCN32)[C@H](C)O1 ; CN(CCN(C)C(=O)OC(C)(C)c1ccc(-c2ccccc2)cc1)C(=O)Cl ; COc1cccc2c1C(=O)c1c(O)c3c(c(O)c1C2=O)C[C@@](O)(C(=O)COC(=O)N(C)CCN(C)C(=O)OC(C)(C)c1ccc(-c2ccccc2)cc1)C[C@@H]3O[C@H]1C[C@H]2[C@H](O[C@@H]3[C@@H](OC)OCCN32)[C@H](C)O1 ; CNCCN(C)C(=O)OCC(=O)[C@]1(O)Cc2c(O)c3c(c(O)c2[C@@H](O[C@H]2C[C@H]4[C@H](O[C@@H]5[C@@H](OC)OCCN54)[C@H](C)O2)C1)C(=O)c1c(OC)cccc1C3=O]

### Compound I.51:

In a flask under argon containing (8S,10S)-6,8,11-trihydroxy-8-(2-hydroxyacetyl)-1-methoxy-10-(((1S,3R,4aS,9S,9aR,10aS)-9-methoxy-1-methyloctahydro-1H-pyrano[4′,3′:4,5]oxazolo[2,3-c][1,4]oxazin-3-yl)oxy)-7,8,9,10-tetrahydrotetracene-5,12-dione (50 mg, 0.078 mmol), 4-dimethylaminopyridine (47.6 mg, 0,390 mmol), molecular sieves 0.4 nm (33 mg) and DCM (1 mL), a solution of 2-([1,1′-biphenyl]-4-yl)propan-2-yl (2-((chlorocarbonyl)(methyl)amino)ethyl)(methyl)carbamate (91 mg, 0.234 mmol)) in DCM (0.5 mL) were added. This mixture was stirred in the dark at 25° C. for 5 days. The solution was filtered, and the solvent was concentrated in vacuo and the residue was used without further purification in the next step.

### Compound I.52:

To a solution of product I.51 in DCM (1 ml) in ice bath, a solution of dichloroacetic acid (96 μl, 1.169 mmol) in 0.5 mL of DCM was added. The solution was stirred at rt for 2 h. The solvent was concentrated in vacuo and the residue was purified by automatic column chromatography (Interchim, solid deposit): DCM/MeOH: 9/1. The desired fractions were concentrated in vacuo to give the desired compound I.52, Yield 13 mg, 22% as red solid. LCMS (ESI): 756.76 (MH+).

### Compound I.53:

In a flask under argon were added (9H-fluoren-9-yl)methyl ((S)-1-(((S)-1-((4-(hydroxymethyl)phenyl)amino)-1-oxopropan-2-yl)amino)-3-methyl-1-oxobutan-2-yl)carbamate (250 mg, 0.485 mmol), bis(perfluorophenyl) carbonate (382 mg, 0.970 mmol) and DMF (4 mL). Then, the mixture was cooled to 0° C. and N-ethyl-N-isopropylpropan-2-amine (127 μl, 0.727 mmol) was added dropwise. The reaction mixture was warmed up to rt and stirred for 2 h (check by LCMS). The crude was concentrated in vacuo. The crude was purified by automatic column chromatography (Interchim, solid deposit): DCM/MeOH: 9/1. The desired fractions were concentrated in vacuo to give the desired compound I.53, Yield 281 mg, 80% as yellow oil. LCMS (ESI): 726.65 (MH+).

### Compound I.54:

In a flask under argon were added 2-oxo-2-((2S,4S)-2,5,12-trihydroxy-7-methoxy-4-(((1S,3R,4aS,9S,9aR,10aS)-9-methoxy-1-methyloctahydro-1H-pyrano[4′,3′:4,5]oxazolo[2,3-c][1,4]oxazin-3-yl)oxy)-6,11-dioxo-1,2,3,4,6,11-hexahydrotetracen-2-yl)ethyl methyl(2-(methylamino)ethyl)carbamate (78 mg, 0.103 mmol), 1-hydroxybenzotriazole (27.9 mg, 0.206 mmol), N,N′-diisopropylethylamine (35.1 μl, 0.206 mmol) and DMF (2 mL). Then, the mixture was cooled to 0° C. and (9H-fluoren-9-yl)methyl ((S)-3-methyl-1-oxo-1-(((S)-1-oxo-1-((4-((((perfluorophenoxy)carbonyl)oxy)methyl)phenyl)amino)propan-2-yl)amino)butan-2-yl)carbamate (112 mg, 0.155 mmol) was added dropwise. The reaction mixture was warmed up to rt and stirred for 2 h (check by LCMS). The crude was concentrated in vacuo. The crude was purified by automatic column chromatography (Interchim, solid deposit): DCM/MeOH: 9/1. The desired fractions were concentrated in vacuo to give the desired compound I.54, Yield 77 mg, 58% as red oil. LCMS (ESI): 1298.0 (MH+).

### Compound I.55:

In a flask under argon were added 4-((S)-2-((S)-2-((((9H-fluoren-9-yl)methoxy)carbonyl)amino)-3-methylbutanamido)propanamido)benzyl (2-oxo-2-((2S,4S)-2,5,12-trihydroxy-7-methoxy-4-(((1S,3R,4aS,9S,9aR,10aS)-9-methoxy-1-methyloctahydro-1H-pyrano[4′,3′:4,5]oxazolo[2,3-c][1,4]oxazin-3-yl)oxy)-6,11-dioxo-1,2,3,4,6,11-hexahydrotetracen-2-yl)ethyl) ethane-1,2-diylbis(methylcarbamate) (77.7 mg, 0.060 mmol) and DMF (2 mL). Then, the mixture was cooled to 0° C. and morpholine (259 μl, 2.99 mmol) was added dropwise. The reaction mixture was warmed up to rt and stirred for 2 h (check by LCMS). The crude was concentrated in vacuo. The crude was purified by automatic column chromatography (Interchim, solid deposit): DCM/MeOH: 9/1. The desired fractions were concentrated in vacuo to give the desired compound I.55, Yield 32 mg, 50% as red oil. LCMS (ESI): 1075.80 (MH+).

### Example J

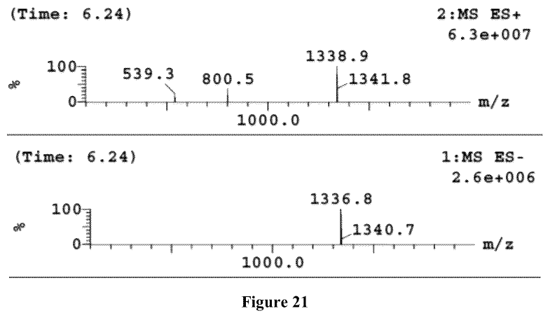

In a flask under argon at 25° C. were introduced 4-((S)-2-((S)-2-amino-3-methylbutanamido)propanamido) benzyl(2-oxo-2-((2S,4S)-2,5,12-trihydroxy-7-methoxy-4-(((1S,3R,4aS,9S,9aR,10aS)-9-methoxy-1-methyloctahydro-1H-pyrano[4′,3′:4,5]oxazolo[2,3-c][1,4]oxazin-3-yl)oxy)-6,11-dioxo-1,2,3,4,6,11-hexahydrotetracen-2-yl)ethyl) ethane-1,2-diylbis(methylcarbamate) (32 mg, 0.030 mmol, 1 eq) in dichloromethane (2 mL). The mixture was cooled to 0° C. and N-ethyl-N-isopropylpropan-2-amine (20.74 μl, 0.119 mmol) was added. The mixture was stirred at 0° C. for 10 min then addition of Example 16 diluted in dichloromethane (2 mL). The mixture was then stirred at 0° C. for 2 h (until complete conversion was observed by LCMS). The crude was concentrated in vacuo and purified by automatic column chromatography (Interchim, 12 g, solid deposit): DCM/MeOH: 9/1. The desired fractions were concentrated in vacuo to give the desired Example J (also named compound F562646), Yield 17.4 mg, 40% as red oil. LCMS (ESI): 1338.41 (MH+).
The mass spectrum of this drug-linker conjugate is represented on FIG. 21.

### Example K

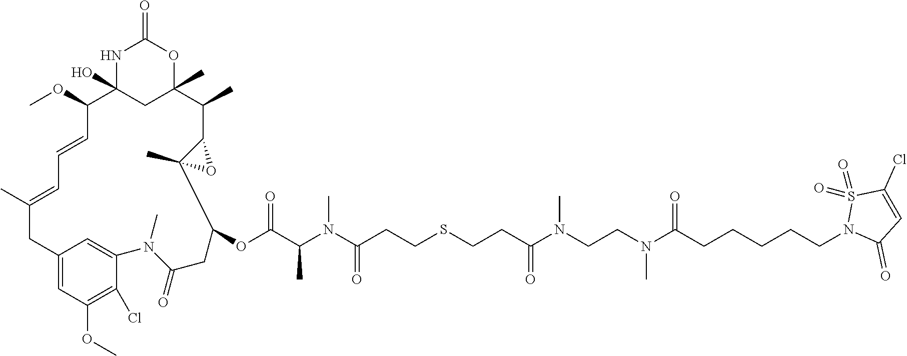

Example K has been synthesized according to the following synthetic path:

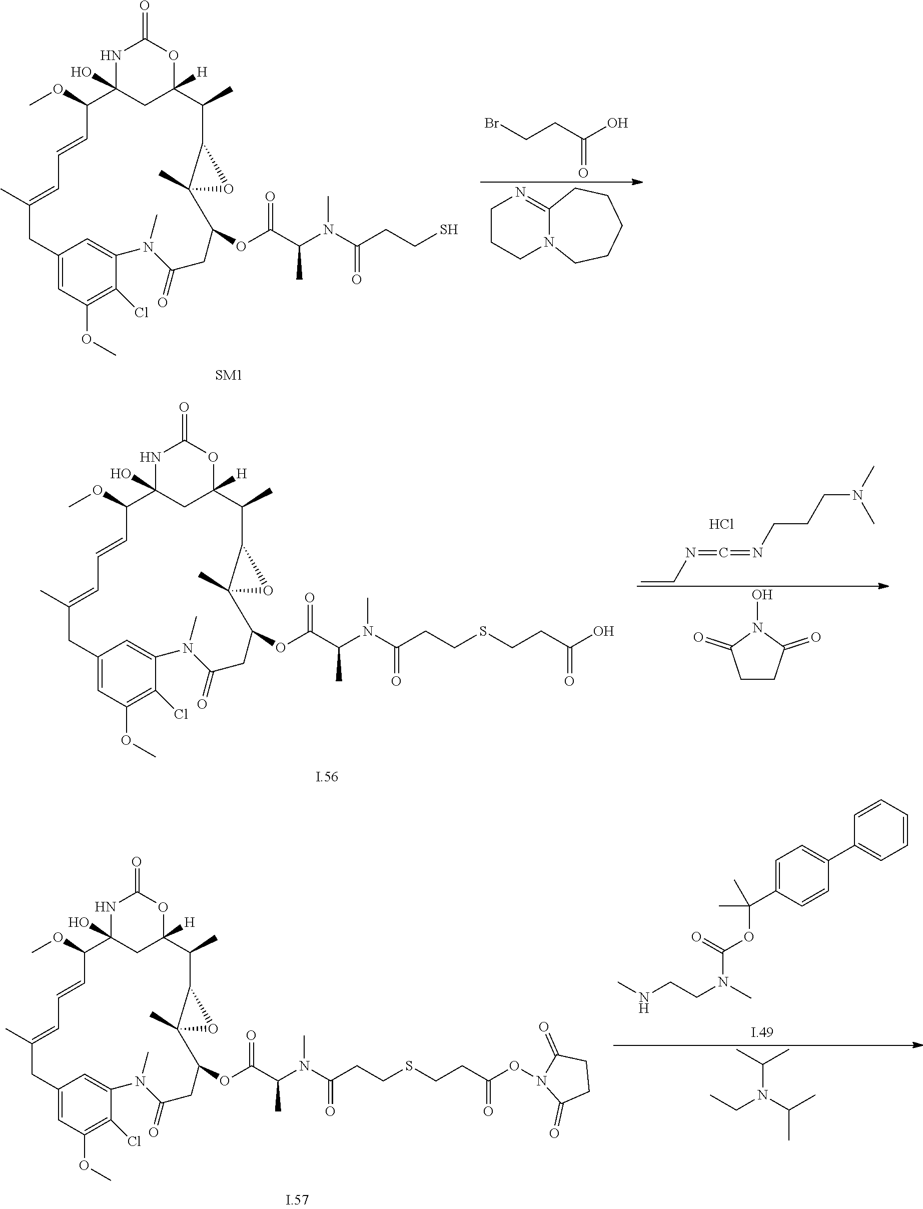

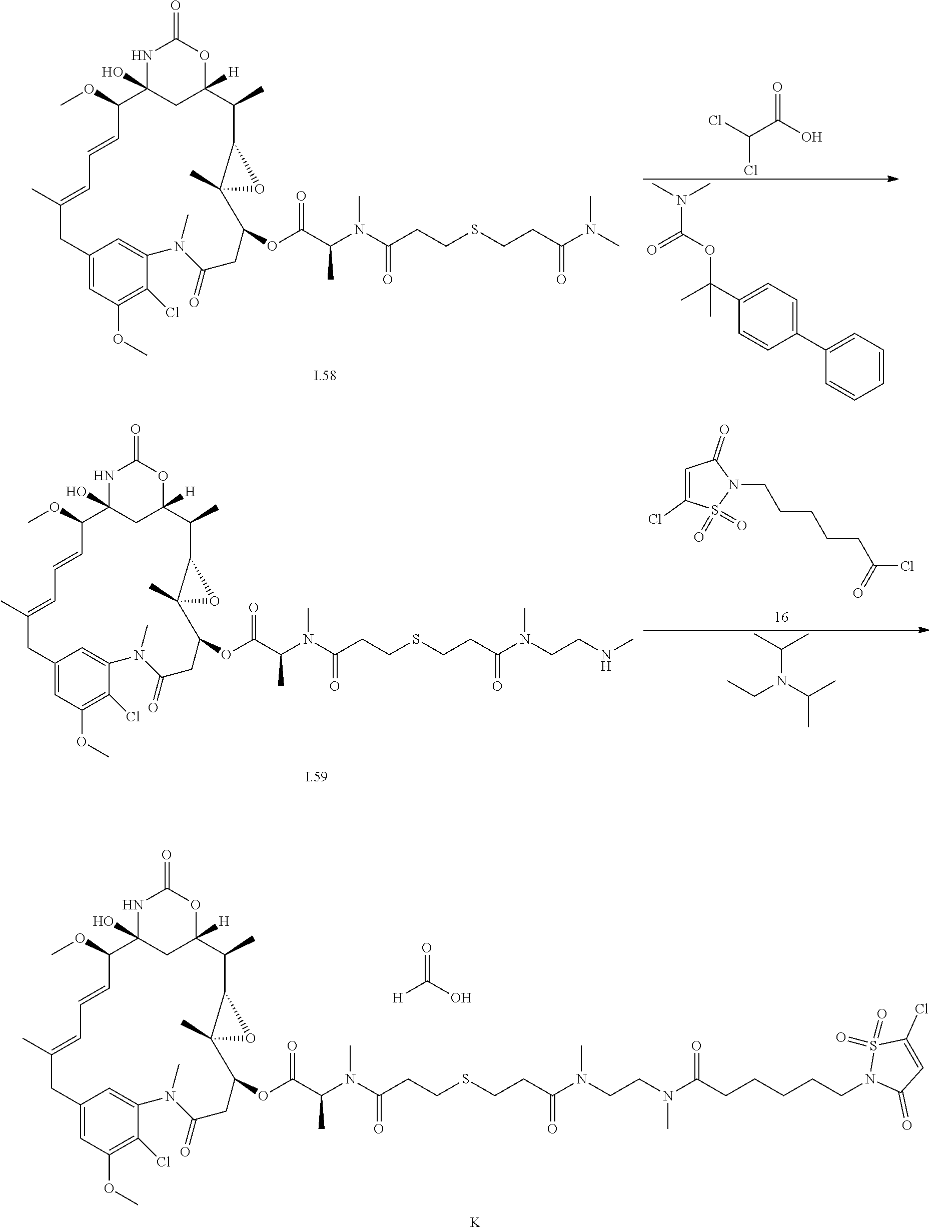

### Compound I.56:

In a flask under argon was added starting material SM1 (100 mg, 0.135 mmol) and DMF (1 ml). The mixture was cooled to 0° C., then a solution of 3-bromopropanoic acid (22.79 mg, 0.149 mmol) and 2,3,4,6,7,8,9,10-octahydropyrimido[1,2-a]azepine (40.5 μl, 0.271 mmol) in DMF (0.5 mL) was added dropwise. Then The mixture was warmed up to rt and was stirred until complete conversion was observed by LCMS. The crude was concentrated in vacuo and purified by automatic column chromatography (Interchim, 12 g, solid deposit): DCM/MeOH: 80/20. The desired fractions were concentrated in vacuo to give the desired compound I.56, Yield 108 mg, 98% as white solid. LCMS (ESI): 811.35 (MH+).

### Compound I.57:

In a flask under argon were added compound I.56 (87.0 mg, 0.107 mmol) and DCM (2 mL). Then, the mixture was cooled to 0° C., N-hydroxysuccinimide (13.59 mg, 0.118 mmol) and 1-(3-dimethylaminopropyl)-3-ethylcarbodiimide hydrochloride (30.9 mg, 0.161 mmol) were added. The reaction mixture was warmed up to rt and stirred for 2 h (CHECK LCMS). The crude was concentrated in vacuo. The crude material was used without further purification for the next step.

### Compound I.58:

In a flask under argon was added compound I.57 (97 mg, 0.107 mmol) and DCM (1 ml), then a solution of compound I.49 in 1 mL of DCM and N, N′-diisopropylethylamine (37.3 μl, 0.214 mmol) was added dropwise. The mixture was stirred until complete conversion was observed by LCMS. The crude was concentrated in vacuo and used without further purification for the next step.

### Compound I.59:

In a flask under argon was added compound I.58 (58 mg, 0.052 mmol) and DCM (1 ml). The mixture was cooled to 0° C., then dichloroacetic acid (86 μl, 1.037 mmol) was added dropwise. Then the mixture was warmed up to rt and was stirred until complete conversion was observed by LCMS. The crude was concentrated in vacuo and purified by automatic column chromatography (Interchim, 12 g, solid deposit): DCM/MeOH: 80/20. The desired fractions were concentrated in vacuo to give the desired compound I.59, Yield 39 mg, 86% as white solid. LCMS (ESI): 882.6 (MH+).

### Example K

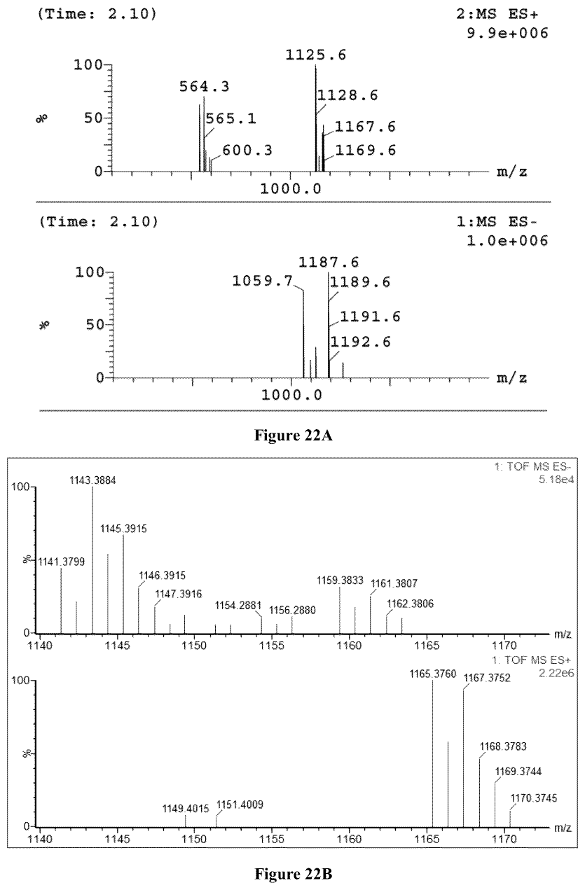

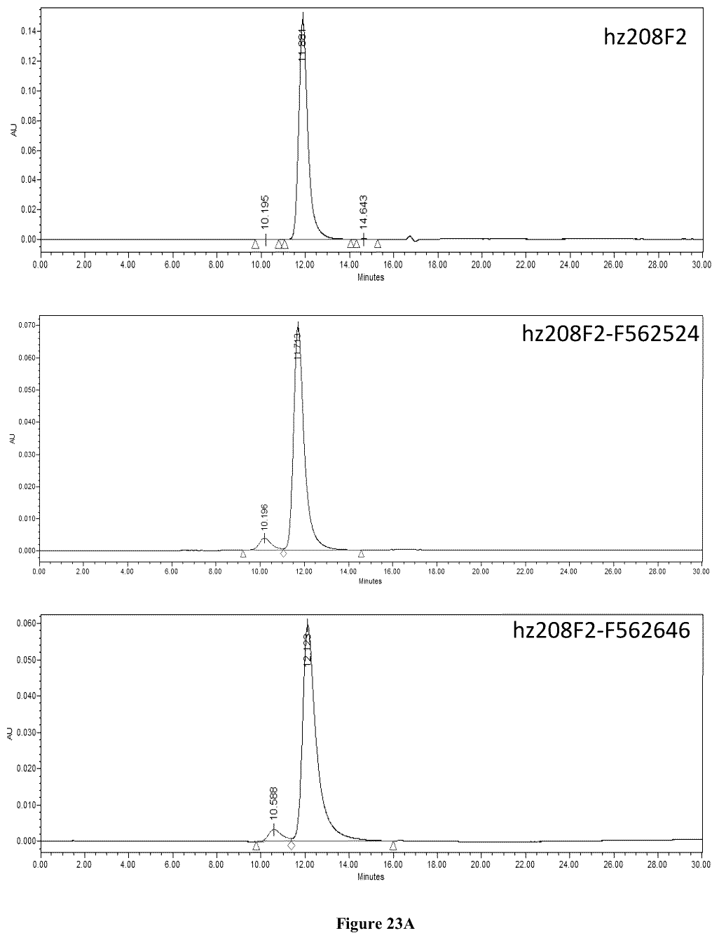

In a flask under argon at 25° C. were introduced compound I.59 in dichloromethane (1 mL). The mixture was cooled to 0° C. and N-ethyl-N-isopropylpropan-2-amine (15.83 μl, 0.091 mmol) was added. The mixture was stirred at 0° C. for 10 min then addition of Example 16 diluted in dichloromethane (2 mL). The mixture was then stirred at 0° C. for 2 h (until complete conversion was observed by LCMS). The crude was concentrated in vacuo and purified by preparative HPLC (HCOOH conditions) to give the desired Example K, Yield 6 mg, 22% as white solid. LCMS (ESI): 1165.37 (M+Na)⁺.
The mass spectrum and the TOF-MS spectrum of this drug-linker conjugate are represented respectively on FIGS. 22A and 22B.
3. Conjugation with Somatostatin
VI1. Reaction of Somatostatin with Benzyl 6-(5-chloro-1,1-dioxido-3-oxoisothiazol-2(3H)-yl)hexanoate

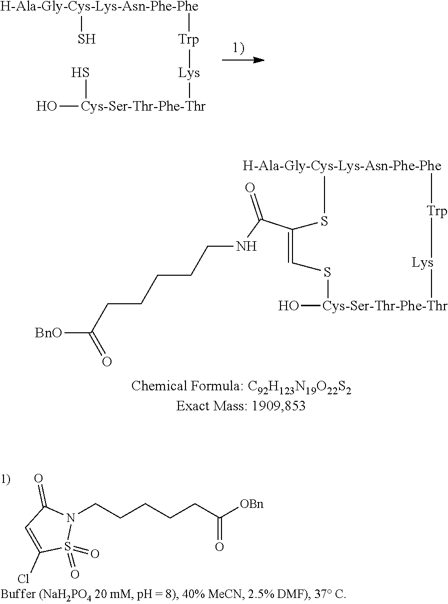

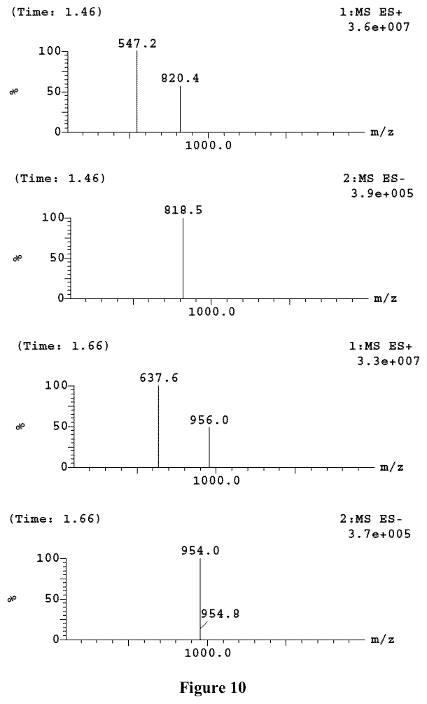

1 mg of lyophilized somatostatin (m_{exact}=1636.72) was solubilized in 4 mL of buffer (57.5% NaH₂PO₄ 20 mM, pH 6.5, 40% ACN, 2.5% DMF) to yield a concentration of 153 μM (0.25 mg/mL). 33.5 mg of TCEP were dissolved in 4 mL of buffer (57.5% NaH₂PO₄ 20 mM, pH 6.5, 40% ACN, 2.5% DMF). To 300 μL of somatostatin solution (1 eq.) were added 3 μL of TCEP solution (1.1 eq.). The solution is stirred at 37° C.; for 1 h. Commercial somatostatin: R_{t,1} (in ACN): 1.57; MS ES+: M⁺³/3=546.4, M⁺²/2=819.2. Reduced disulfide bond somatostatin: R_{t,1} (in ACN): 1.50; MS ES+: M⁺³/3=547.2, M⁺²/2=820.3. 5 mg of Benzyl 6-(5-chloro-1,1-dioxido-3-oxoisothiazol-2(3H)-yl)hexanoate (I.6) were solubilized in 800 μL of ACN. 3 μL of Benzyl 6-(5-chloro-1,1-dioxido-3-oxoisothiazol-2(3H)-yl)hexanoate (1.1 eq.) in solution were added to the somatostatin solution. The solution was stirred at 37° C. R_{t,1} (in ACN): 1.66; MS ES+: M⁺³/3=637.6, M⁺²/2=956.0.
The same reaction was performed in buffer pH 8 (57.5% NaH₂PO₄ 20 mM, pH 8, 40% ACN, 2.5% DMF).
The mass spectrum of the obtained conjugate is represented on FIG. 10.
VI2. Reaction of Somatostatin with Benzyl 6-(4,5-dichloro-1,1-dioxido-3-oxoisothiazol-2(3H)-yl)hexanoate:

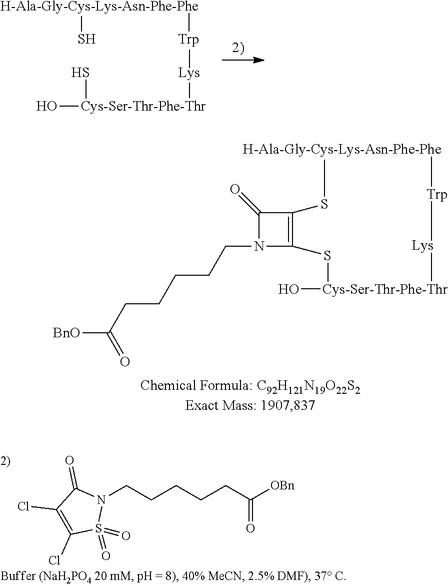

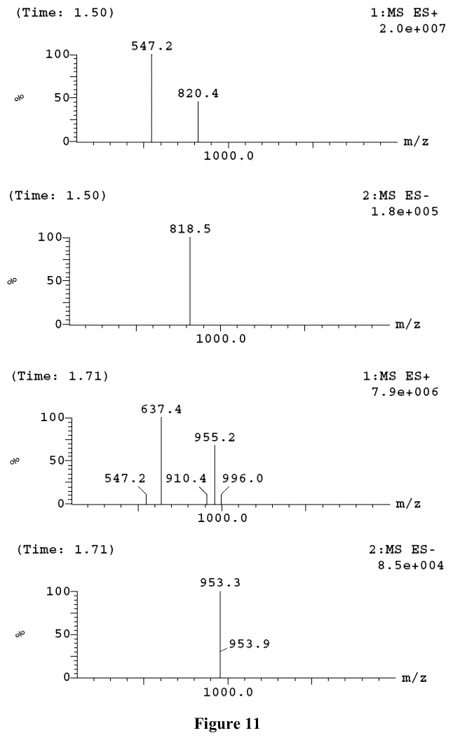

1 mg of lyophilized somatostatin (m_{exact}=1636.72) was solubilized in 4 mL of buffer (57.5% NaH₂PO₄ 20 mM, pH 6.5, 40% ACN, 2.5% DMF) to yield a concentration of 153 μM (0.25 mg/mL). 33.5 mg of TCEP were dissolved in 4 mL of buffer (57.5% NaH₂PO₄ 20 mM, pH 6.5, 40% ACN, 2.5% DMF). To 300 μL of somatostatin solution (1 eq.) were added 3 μL of TCEP solution (1.1 eq.). The solution is stirred at 37° C. for 1 h. Commercial somatostatin: R_{t,1} (in ACN): 1.57; MS ES+: M⁺³/3=546.4, M⁺²/2=819.2. Reduced disulfide bond somatostatin: R_{t,1} (in ACN): 1.50; MS ES+: M⁺³/3=547.2, M⁺²/2=820.3. 5.4 mg of Benzyl 6-(4,5-dichloro-1,1-dioxido-3-oxoisothiazol-2(3H)-yl)hexanoate (I.7) were solubilized in 800 μL of ACN. 3 μL of Benzyl 6-(4,5-dichloro-1,1-dioxido-3-oxoisothiazol-2(3H)-yl)hexanoate (1.1 eq.) in solution were added to the somatostatin solution. The solution was stirred at 37° C. R_{t,1} (in ACN): 1.65; MS ES+: M⁺³/3=637.3, M⁺²/2=955.5.
The same reaction was performed in buffer pH 8 (57.5% NaH₂PO₄ 20 mM, pH 8, 40% ACN, 2.5% DMF).
The mass spectrum of the obtained conjugate is represented on FIG. 11.
4. Conjugation with Monoclonal Antibodies

### 4.1. ADC Synthesis, Purification and Characterization

The procedure described below applies to chimeric, humanized and human IgG1 forms. It must be understood that for any other forms, such as IgG2, IgG4, etc., the person skilled in the art would be capable of adapting this procedure using the general knowledge.
Ab1 antibody is an anti-IGF1R IgG1 monoclonal antibody. This antibody corresponds to antibody 208F2 of WO2015162291 (see table 3, page 36) for which the three light chain CDRs have sequences SEQ ID Nos. 9, 5 and 11; the three heavy chain CDRs have sequences SEQ ID Nos. 7, 2 and 3; the light chain variable domain has sequence SEQ ID No. 18; and the heavy chain variable domain has sequence SEQ ID No. 13.
Ab2 antibody is an irrelevant chimeric (IgG1) antibody directed at a bacterial protein, which is the outer membrane protein A from E. coli, and called c9G4 (Haeuw J. F. and Beck A. Proteomics for development of immunotherapies, In Proteomics: Biomedical and Pharmaceutical Applications, Kluwer Academic Publishers, Ed. Hondermarck H., 2014, pages 243-278; WO2015162291).
Antibodies (1-5 mg/ml) were partially reduced with TCEP hydrochloride in 10 mM borate buffer pH 8.4 containing 150 mM NaCl and 2 mM EDTA for 2-4 hours at 37° C. Typically, 6-20 molar equivalents of TCEP were used to target a DAR of around 4. The partial antibody reduction was confirmed by SDS-PAGE analysis under non-reducing conditions. The antibody concentration was then adjusted to 1 mg/ml with 10 mM borate buffer pH 8.4 containing 150 mM NaCl, 2 mM EDTA, 6% sucrose and a 5-20 molar excess of drug-linker conjugate to antibody was added from a 10 mM solution in DMSO.
Seven examples of drug-linker conjugate according to the invention were coupled to Ab1:
Example A and Example B giving respectively ADC1-A and ADC1-B (non-cleavable linkers);
Example C, Example D, Example E, Example F and Example G giving respectively ADC1-C, ADC1-D, ADC1-E, ADC1-F and ADC1-G (cleavable linkers).
   The final DMSO concentration was adjusted to 10% to maintain the solubility of the drug in the aqueous medium during coupling. The reaction was carried out for 1-4 h at room temperature or 37° C. The drug excess was quenched by addition of 2.5 moles of N-acetylcysteine per mole of drug and incubation for 1 h at room temperature.
   After dialysis against 25 mM His buffer pH 6.5 containing 150 mM NaCl and 6% sucrose overnight at 4° C., the re-bridged antibody-drug conjugates were purified by using methods known to persons skilled in the art with commercial chromatography columns and ultrafiltration units. The purified ADCs were stored at 4° C. after sterile filtration on 0.2 μm filter.

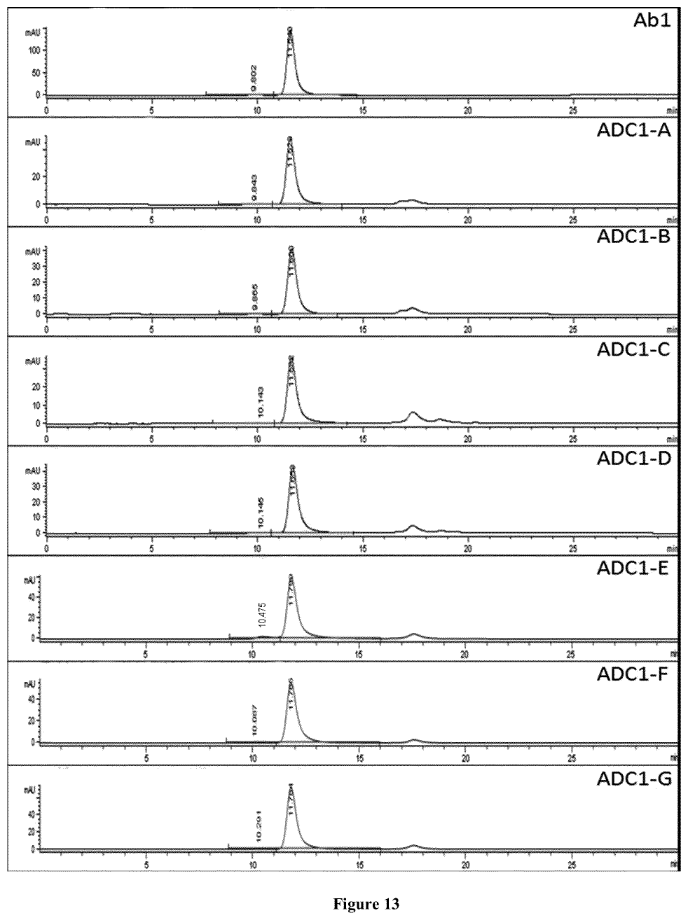

They were further analyzed by SDS-PAGE under reducing and non-reducing conditions to confirm drug conjugation and by SEC on analytical TSK G3000 SWXL column to determine the content of monomers and aggregated forms. The content of aggregated forms deduced from the SEC chromatograms (FIG. 13) was lower than 5% as shown in Table 9.

TABLE 9
Content of aggregated forms

| Ab/ADC | % monomer |
|---|---|
| Ab1 | 99.6 |
| ADC1-A | 99.0 |
| ADC1-B | 99.3 |
| ADC1-C | 98.1 |
| ADC1-D | 99.5 |
| ADC1-E | 99.4 |
| ADC1-F | 99.6 |
| ADC1-G | 95.2 |

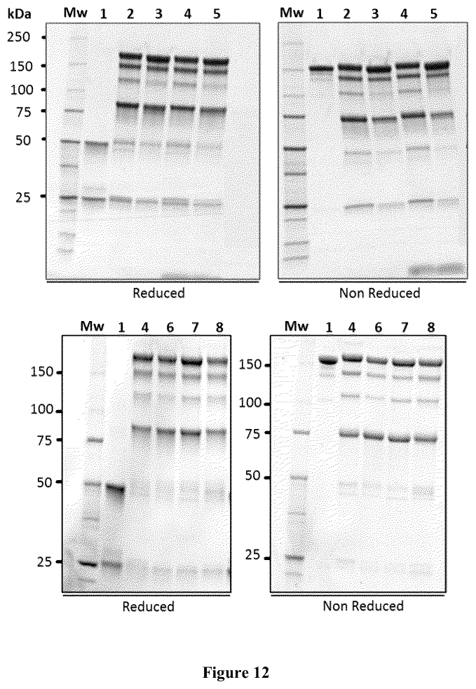

SDS-PAGE analyses confirm formation of fully bridged antibody H2L2 (FIG. 12). However other species (H2L, H2 and HL), corresponding to partially bridged antibody, were also detected. It's important to note that these species were visible when samples were heat-treated in reducing conditions before the run to ensure full dissociation of heavy and light chains (H and L), not connected by an intact interchain bridge.
The protein concentrations were determined by using the BCA assay with IgG as standard. The DAR was estimated for each purified ADC by HIC using a TSK-Butyl-NPR column. It was comprised between 3.5 and 4.3 (Table 10). HIC profiles revealed that no DAR0 and a major peak of DAR 4 were observed for most of the ADCs synthesised. Indeed, only ADC1-C and E show trace of DAR0. Moreover, for ADC1-A, B, C and G only DAR3, DAR4 and DAR5 were observed. Excepted for ADC1-D, the major peak is a DAR4. Compare to a second-generation ADC, these ADCs are more homogeneous as shown in Table 10.

TABLE 10
DAR distribution estimated by HIC using a TSK-Butyl-NPR column

| | DAR % | | | | | | | | |
|---|---|---|---|---|---|---|---|---|---|
| | DAR0 | DAR1 | DAR2 | DAR3 | DAR4 | DAR5 | DAR6 | DAR7 | DAR8 |
| Adcetris | 6.0 | 1.34 | 25.2 | 3.7 | 32.9 | 0 | 22.5 | 1.5 | 6.9 |
| ADC1-A | 0 | 0 | 0 | 0 | 64.9 | 35.1 | 0 | 0 | 0 |
| ADC1-B | 0 | 0 | 0 | 39.8 | 47.6 | 12.5 | 0 | 0 | 0 |
| ADC1-C | 0.6 | 1.0 | 0.8 | 8.2 | 63.1 | 26.3 | 0 | 0 | 0 |
| ADC1-D | 0 | 0 | 0 | 44.6 | 39.5 | 10.6 | 3.8 | 1.5 | 0 |
| ADC1-E | 2.1 | 4.5 | 4.0 | 11.9 | 56.3 | 21.1 | 0 | 0 | 0 |
| ADC1-F | 0 | 0.5 | 3.2 | 22.6 | 73.7 | 0 | 0 | 0 | 0 |
| ADC1-G | 0.1 | 0.4 | 2.6 | 18.6 | 68.1 | 10.2 | 0 | 0 | 0 |

Adcetris® (brentuximab vedotin) has been used as a reference since it uses a second generation maleimide linker conjugated to the cysteines of the antibody. It is the best representative example of second generation ADC; the technology described in this demand being can be considered as the third generation.

### 4.2. ADC Analysis by Native Mass Spectrometry

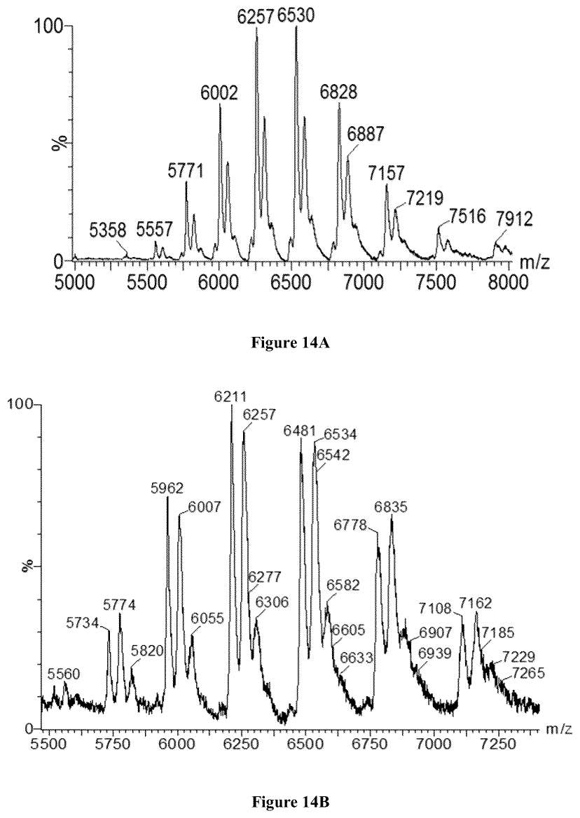

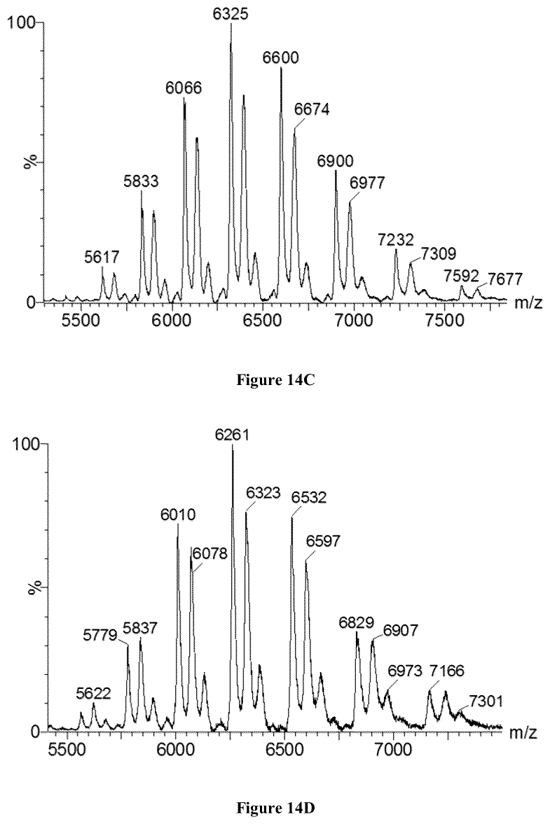

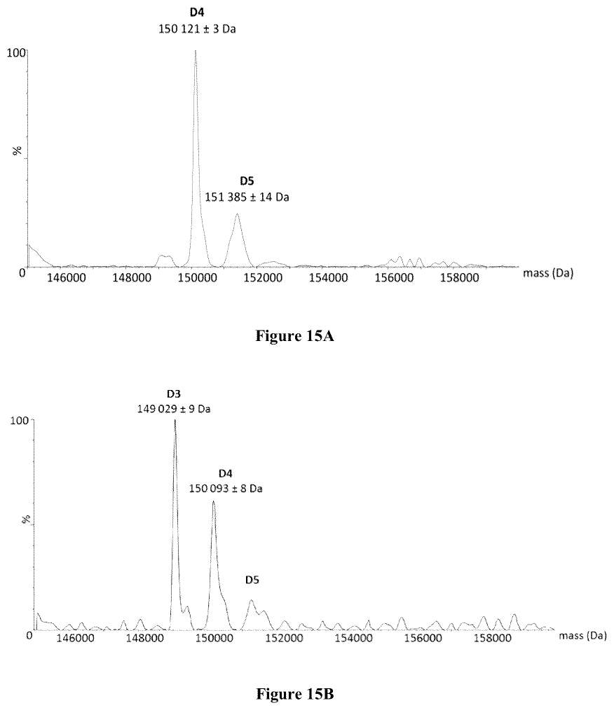

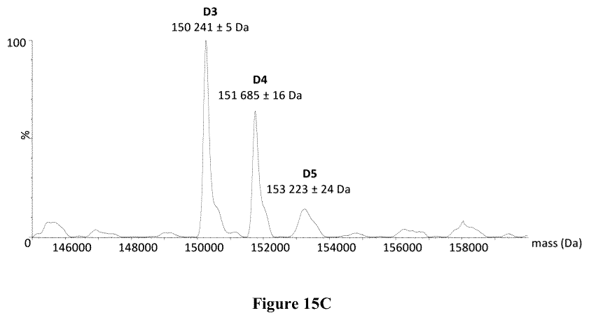

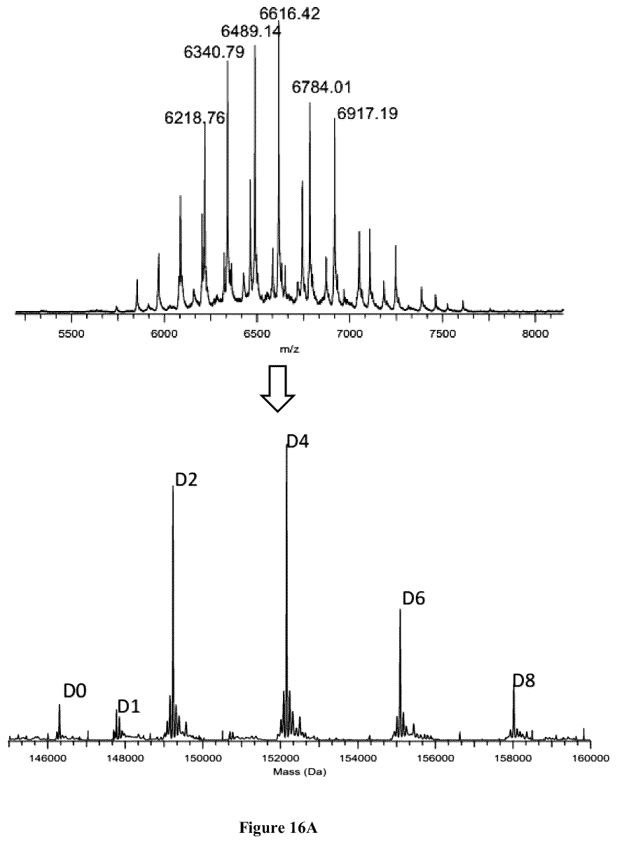

All chemicals were purchased from Sigma-Aldrich: ammonium acetate (A₁₅₄₂), caesium iodide (21004), 2-propanol (19516). IgGZERO (A0-IZ1-010) enzyme was obtained from Genovis. Aqueous solutions were prepared using an ultra-pure water system (Sartorius, Göttingen, Germany).
ADC1-A to ADC1-G were deglycosylated prior to native MS experiments. This was performed by incubating one unit of IgGZERO per microgram of ADC for 30 min at 37° C. Then, ADCs were buffer exchanged against a 150 mM ammonium acetate solution (pH 6.9) using six cycles of concentration/dilution using a microconcentrator (Vivaspin, 10-kD cutoff, Sartorius, Gottingen, Germany). Protein concentration was determined by UV absorbance using a NanoDrop spectrophotometer (Thermo Fisher Scientific, France). Non-denaturing (native) mass spectrometry of ADCs was performed on a Q-TOF (Synapt G2 HDMS, Waters, Manchester, UK) mass spectrometer operating in the positive ion mode both coupled to an automated chip-based nanoelectrospray device (Triversa Nanomate, Advion, Ithaca, USA). Analyses were performed in the m/z 1000-10 000 range. Samples were diluted in 150 mM NH₄OAc at pH 6.9 and infused at 10 μM. External calibration was performed using singly charged ions produced by a 2 g/L solution of caesium iodide in 2-propanol/water (50/50 v/v).
The voltage of the nanoelectrospray was set at 1.75 kV and nitrogen nanoflow at 0.75 psi. The cone voltage was set to 180 volts and the backing pressure to 6 mbar.
FIG. 14 presents examples of non-deconvoluted MS spectrum.
The DAR distribution (FIG. 15) was determined after deconvolution using MaxEnt™ algorithm from Mass Lynx 4.1 (Waters, Manchester, UK). The parameters of the software were optimized for each spectrum.
Average DAR values (FIG. 15) were calculated by using the following equation (where j is the maximum number of drug load).

$$D A R = \frac{( {\sum }_{i = 0}^{j} i * intensity Di )}{{\sum }_{i = 0}^{j} intensity Di}$$

The results were derived from the relative peak intensities of each charge states in the raw spectra and are presented in Table 11 below.

TABLE 11
DAR distribution calculated using MaxEnt ™ algorithm from Mass Lynx 4.1

| | DAR distribution (%) | | | | | | | | | Average |
|---|---|---|---|---|---|---|---|---|---|---|
| ADC | DAR0 | DAR1 | DAR2 | DAR3 | DAR4 | DAR5 | DAR6 | DAR7 | DAR8 | DAR |
| ADC1-A | 0 | 0 | 0 | 0 | 64 | 36 | 0 | 0 | 0 | 4.4 ± 0.1 |
| ADC1-B | 0 | 0 | 0 | 42 | 39 | 19 | 0 | 0 | 0 | 3.8 ± 0.1 |
| ADC1-D | 0 | 0 | 0 | 47 | 40 | 13 | 0 | 0 | 0 | 3.7 ± 0.1 |

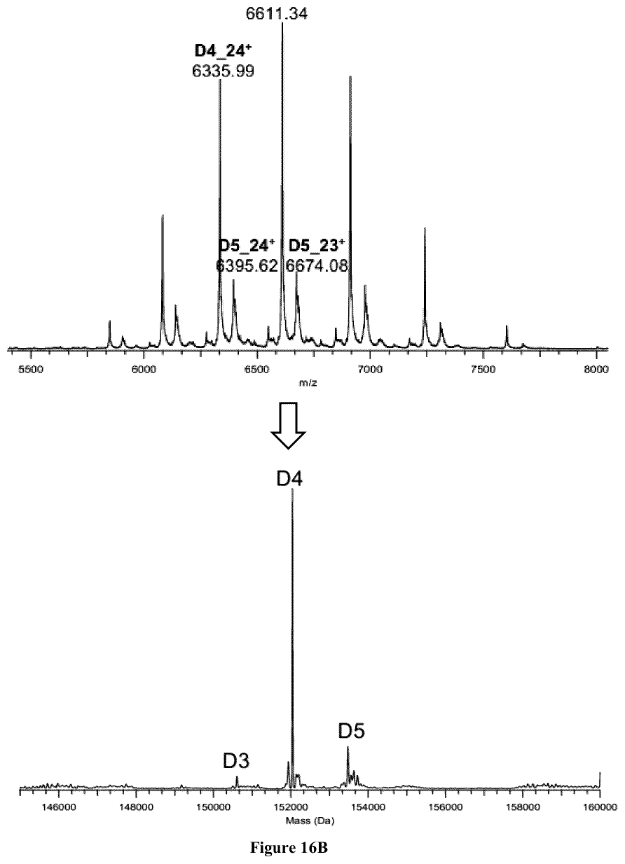

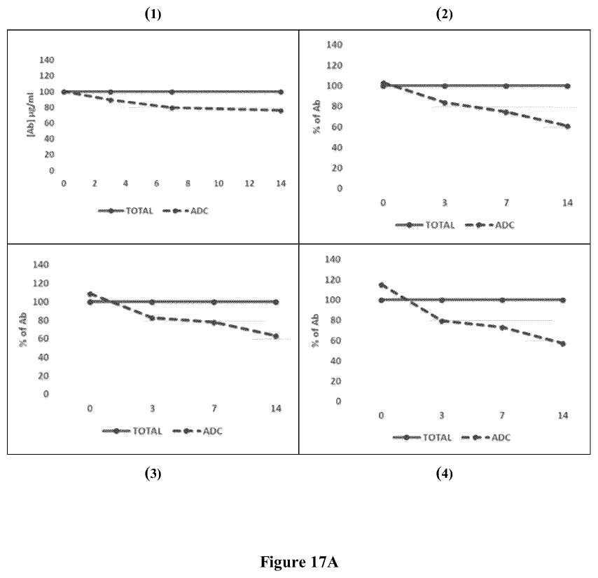

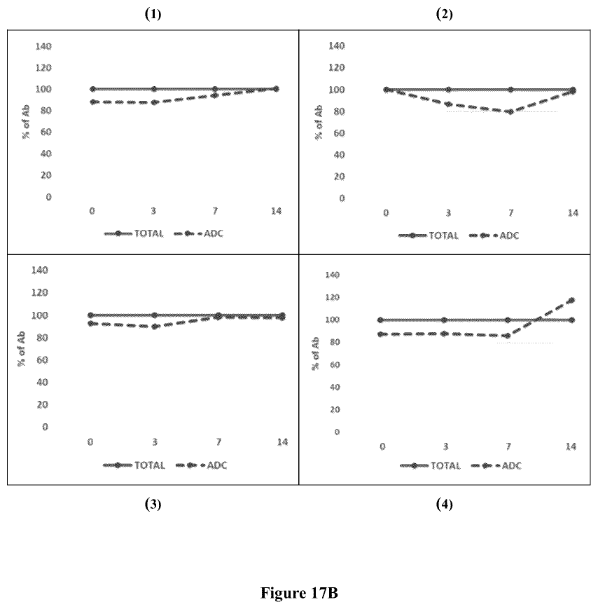

FIG. 16 compares the DAR distribution, determined from raw spectra after mass deconvolution, for 2 different ADCs, i.e.:
ADC1-C according to the invention prepared from Ab1 antibody and the drug-linker conjugate C (FIG. 16B) and
a reference ADC Ref-A which is a comparative ADC synthesized from the same antibody (Ab1) and from a drug-linker conjugate corresponding to the drug-linker conjugate C in which the sulfomaleimide moiety

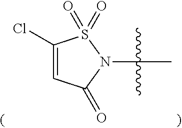 [SMILES: *N1C(=O)C=C(Cl)S1(=O)=O]

has been replaced by a maleimide moiety

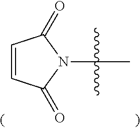 [SMILES: *N1C(=O)C=CC1=O]

(FIG. 16A).
   A heterogeneous distribution from DAR 0 to DAR 8 is observed for the ADC synthesized by using the classical maleimide chemistry to link the drug to the antibody (FIG. 16A), whereas the ADC generated by using the sulfomaleimide chemistry according to the invention is highly homogeneous with 75% of DAR 4 and no DAR 0/2 and 6/8 species (FIG. 16B). These results are summarized in Table 12 below.

TABLE 12
DAR distribution calculated using MaxEnt ™ algorithm from Mass Lynx 4.1

| | DAR distribution (%) | | | | | | | | | Average |
|---|---|---|---|---|---|---|---|---|---|---|
| ADC | DAR0 | DAR1 | DAR2 | DAR3 | DAR4 | DAR5 | DAR6 | DAR7 | DAR8 | DAR |
| ADC Ref-A | 6 | 4 | 29 | 0 | 33 | 0 | 18 | 0 | 10 | 3.8 |
| ADC1-C | 0 | 0 | 0 | 5 | 76 | 19 | 0 | 0 | 0 | 4.1 |

### 4.3. In Vitro Stability Study of ADCs in 4 Mammalian Sera Using a Ligand Binding Assay Method

To establish the gain of stability, an in vitro stability study was conducted. It consists in the incubation of the ADCs at 37° C. for a period of 14 days. Samples were collected at day 0, 3, 7 and 14. The various samples (D0, D3, D7 and D14) were then analyzed by LBA to determine the concentration of total antibody versus the concentration of ADC. In practice, a solution of each ADC is prepared at 100 μg/ml in 4 sera (human, cynomolgus, mouse and rat) and incubated at 37° C. for a maximum of 14 days. Then aliquots are collected at D0, D3, D7 and D14, and stored at −80° C. until dosage. For total Ab and ADC quantification, the plates are thawed at room temperature with shaking and both LBA assays are run in parallel. Briefly, standard microtiter plates (MSD, Gaithersburg, USA) are coated using 50 μl of an anti-His antibody solution at 2 μg/ml prepared in PBS 1×. After an overnight incubation at 4° C., assay plates are treated with blocking buffer (3% MSD Blocker A (MSD, Gaithersburg, USA)) for 1 hour at 37° C. Then the recombinant His-tagged antigen is added for 1 hour at 37° C. at the concentration of 2.5 μg/ml in assay buffer. After a washing step, samples are analyzed as duplicates at the 1/5000° dilution and incubated for 1 hour at 37° C. while standard ADCs are loaded in duplicate onto the assay plate. The detection step is done using either a goat anti-human Ig Kappa sulfo-tag solution at 1 μg/ml for the detection of total Ab or a mouse monoclonal anti-Drug antibody labelled with sulfotag for ADC detection. After a 1-hour incubation period at 37° C., the detection is realized using 150 μL of a 2×MSD-read T buffer containing surfactant (MSD, Gaithersburg, USA) just before reading using MSD Sector Imager.

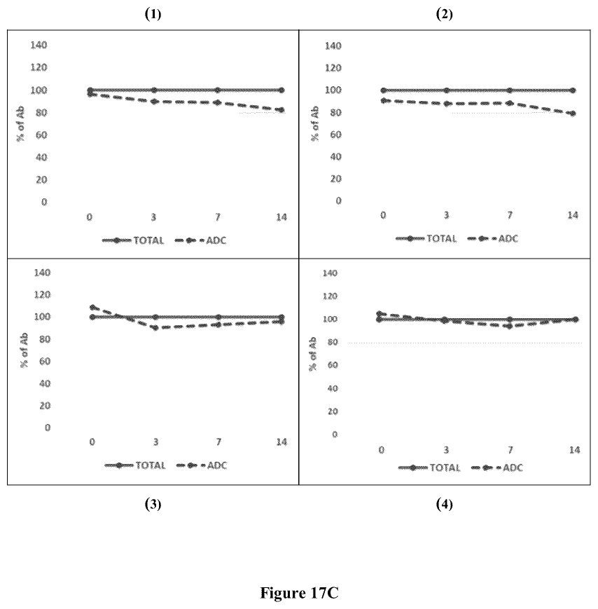

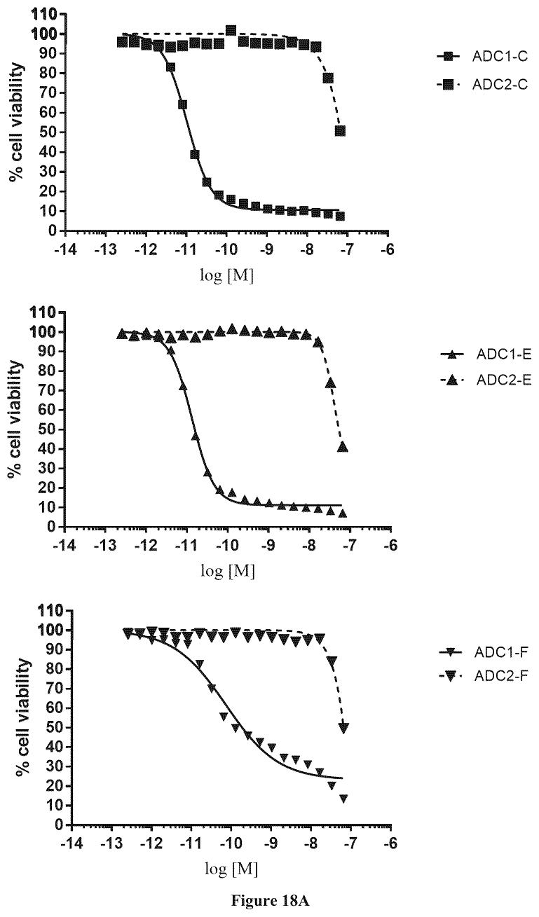

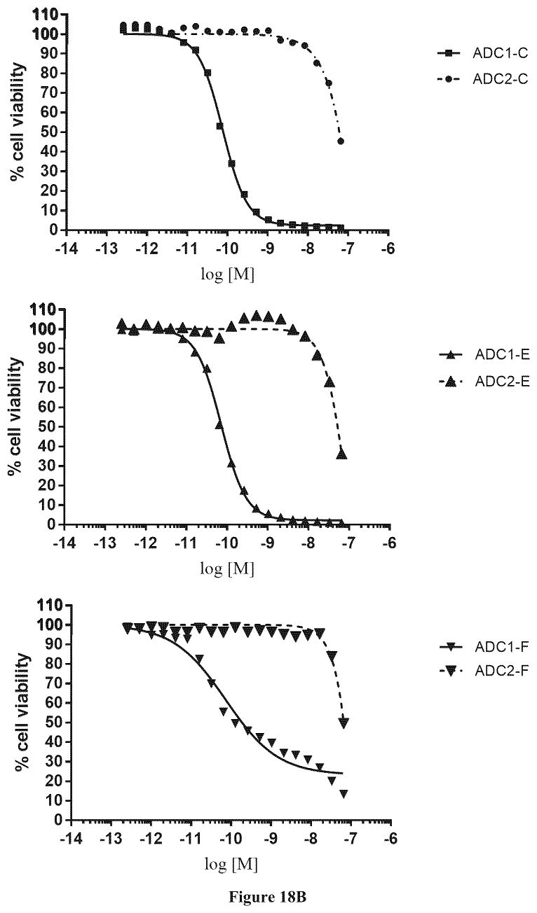

The total antibody and ADC concentrations are determined at each timepoint and transformed in percentage, taking 100% as the quantity of total ADCs or antibody at each timepoint.
Data are illustrated in FIGS. 17A, 17B and 17C for 3 ADCs: ADC1-C (FIG. 17B) and ADC1-E (FIG. 17C) in which the drug has been linked to the Ab1 antibody using the sulfomaleimide chemistry according to the invention (by means of the drug-linker conjugate C or E respectively), in comparison to a reference ADC Ref-B in which the drug has been linked to the antibody using a classical maleimide chemistry (FIG. 17A).

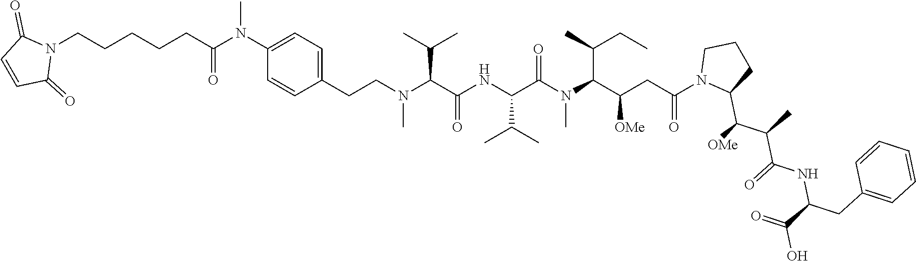 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)CCc1ccc(N(C)C(=O)CCCCCN2C(=O)C=CC2=O)cc1)C(C)C]

### Drug-Linker Moiety Used to Prepare Reference ADC Ref-B

As a comparator, a drug-linker using the same payload and a non-cleavable linker was chosen (drug-linker of ADC Ref-B). It was conjugated to the same antibody using a maleimide chemistry. The choice of this comparator limits “the instability” of the reference ADC in the sera by deconjugation from the antibody through a retro-Michael reaction. Compared to our constructs based on a cleavable linker, this comparator is thus favoured which makes the stability improvement of our drug-linkers even more spectacular.

As expected a decrease in ADC concentration is observed for the ADC synthesized using classical maleimide chemistry (ADC Ref-B), whereas the ADCs generated using sulfomaleimide chemistry according to the invention (ADC1-C & ADC1-E) surprisingly are much more stable over the 14-day period.

### 4.4. In Vitro Cytotoxicity of ADCs

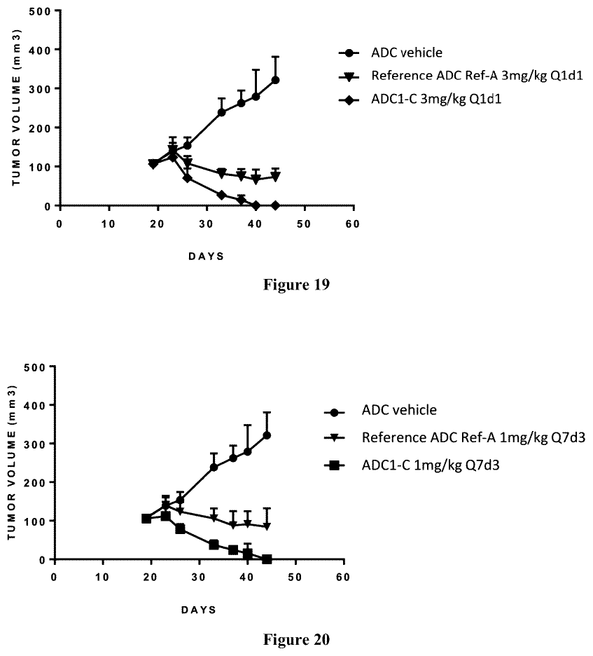

The in vitro cytotoxicity of ADC according to the invention was evaluated. In order to evaluate the non-specific cytotoxicity, the compounds were also coupled to an irrelevant chimeric antibody (Ab2), called c9G4, at the same DAR and using the same drug-linker conjugates to give ADC2-C with Example C, ADC2-E with Example E and ADC2-F with Example F.
MCF-7 and NCI-H2122 cells were plated on 96 well plates (2500 cells per well) in complete growth media. The day after, serial dilutions of the tested ADCs were added to the corresponding wells and incubated at 37° C. for 6 days. Six days after the addition of the ADCs, a Cell Titer Glo assay (PROMEGA) was performed on the plates to check the viability of the cells.
The results obtained, expressed in percentage of viability, are shown in FIGS. 18A and 18B. As expected, the ADCs synthesized with the irrelevant antibody showed no or modest cytotoxic activity on both MCF-7 and NCI-H2122 cells. On the opposite, the ADCs of the invention: ADC1-C, ADC1-E and ADC1-F decreased dramatically cell viability. EC₅₀ values of 7,61.10⁻¹¹, 7,16.10⁻¹¹ and 3,64.10⁻¹¹ M were obtained for ADC1-C, ADC1-E and ADC1-F respectively on NCI-H2122 and EC₅₀ values of 1,04.10⁻¹¹ 1,33.10⁻¹¹ and 7,39.10⁻¹¹ M were obtained for ADC1-C, ADC1-E and ADC1-F respectively on MCF7, indicating potent cytotoxic activity.
4.5. In vivo
All experimental protocols were approved by Pierre Fabre's Institutional Animal Care and use Committee.
For ovarian cancer model, 7 weeks old female SCID mice (Charles RIVER Laboratories) were engrafted subcutaneously with 10.10⁶ CaoV3 cells (6 animals per groups). Treatment by intravenous administration of ADC1-C according to the invention, reference ADC Ref-A or ADC vehicle was initiated when tumors reached approximately 150 mm³. The animals were either treated by one injection (Q1d1) or by 3 injections (once weekly) (Q7d3). Tumor volume (Length×width×height×0.52) was measured by electronic caliper at least twice weekly during approximatively 25 days after the first injection. The results are presented on FIG. 19 (animals treated at Q1d1) and FIG. 20 (animals treated at Q7d3).
As can be seen on FIGS. 19 and 20, the ADCs according to the invention have a great efficacy with a complete tumor regression even after a single injection.

### 5. Conjugation of PNU-159682 Derivatives to Monoclonal Antibodies

### 5.1. ADC Synthesis, Purification and Characterization

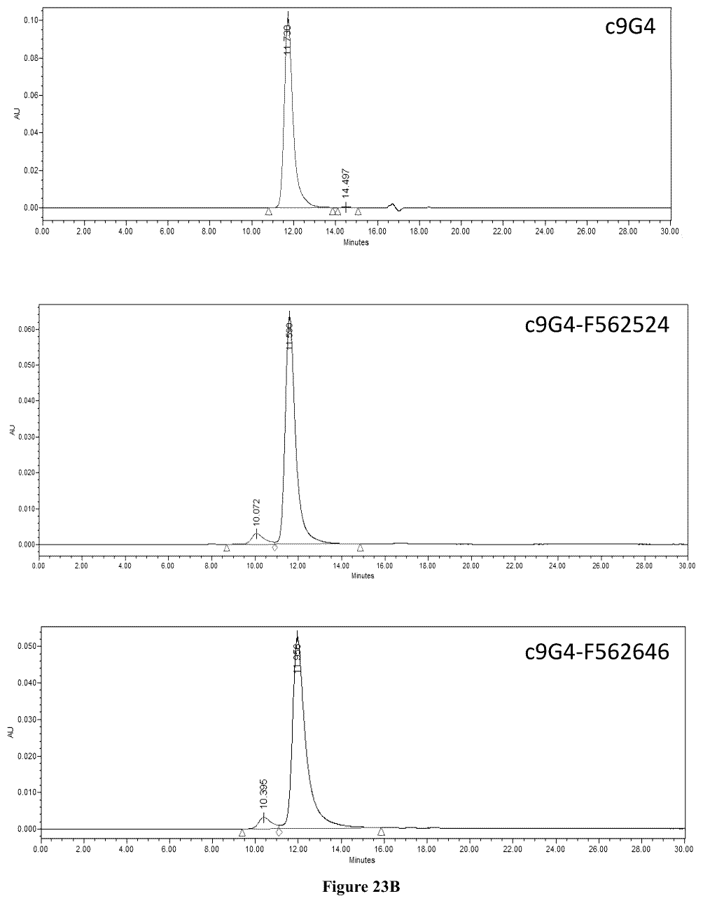

Two PNU-159682 derivatives, namely F562524 (example J) and F562646 (example H), were coupled to the antibodies 208F2 (Ab1) and c9G4 (Ab2), under the conditions previously described in example 4. 208F2 (Ab1) and c9G4 (Ab2) are as disclosed in example 4. Briefly, antibodies (1-5 mg/ml) were partially reduced with 6-20 equivalents of TCEP hydrochloride in 10 mM borate buffer pH 8.4 containing 150 mM NaCl and 2 mM EDTA for 2-4 hours at 37° C. The antibody concentration was then adjusted to 1 mg/ml with 10 mM borate buffer pH 8.4 containing 150 mM NaCl, 2 mM EDTA, 6% sucrose and a 5-20 molar excess of drug-linker conjugate to antibody was added from a 10 mM solution in DMSO. The reaction was carried out for 1-4 h at room temperature or 37° C. in the presence of 10% DMSO. The drug excess was quenched by addition of 2.5 moles of N-acetylcysteine per mole of drug and incubation for 1 h at room temperature. After dialysis against 25 mM His buffer pH 6.5 containing 150 mM NaCl and 6% sucrose overnight at 4° C., the ADCs were purified by chromatography or ultrafiltration. The ADC concentrations were determined by using the BCA assay with IgG as standard and the purified ADCs were stored at 4° C. after sterile filtration on 0.2 till filter.
ADCs were further analyzed by SDS-PAGE and SEC (TSK G3000 SWXL column), as previously described in example 4, to confirm drug conjugation and rebridging, and to determine the content of monomers and aggregates. The content of monomers was around 95% (FIG. 23 and table 13).

TABLE 13
Content of monomers

| ADC | % of monomers |
|---|---|
| Ab1 (208F2) | 99.8 |
| Ab1- F562524 (208F2-F562524) | 94.1 |
| Ab1- F562646 (208F2-F562646) | 94.8 |
| Ab2 (c9G4) | 99.7 |
| Ab2- F562524 (c9G4-F562524) | 94.4 |
| Ab2- F562646 (c9G4-F562646) | 94.7 |

### 5.2. ADC Analysis by Native LC-MS

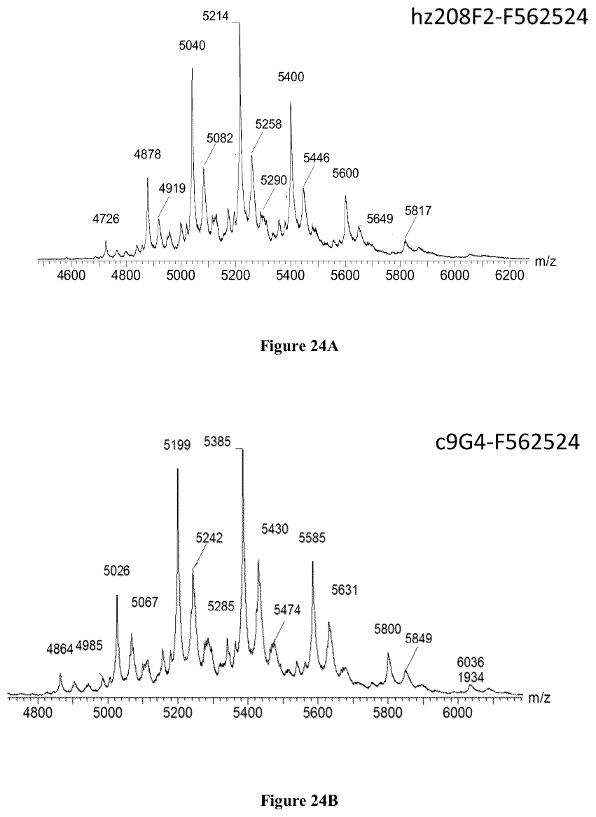

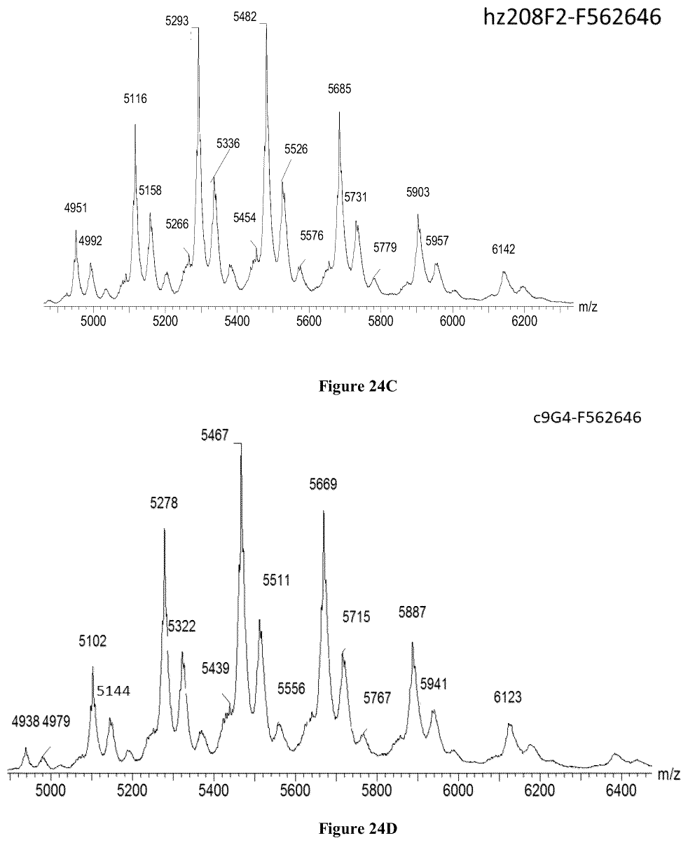

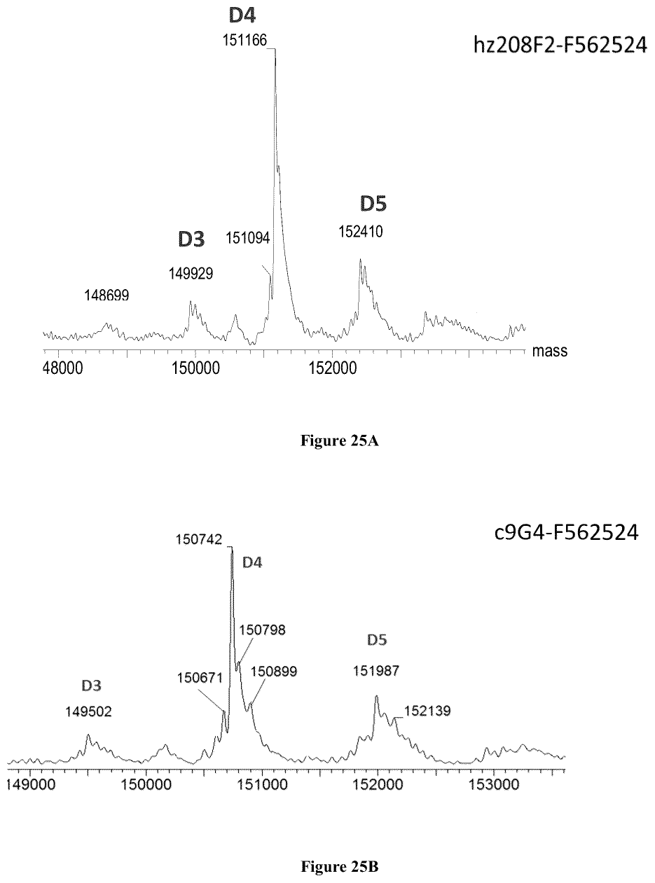

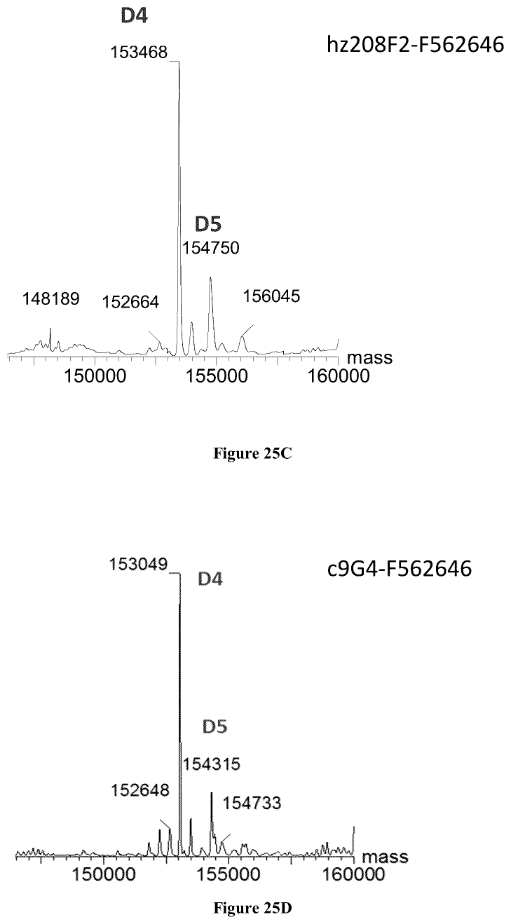

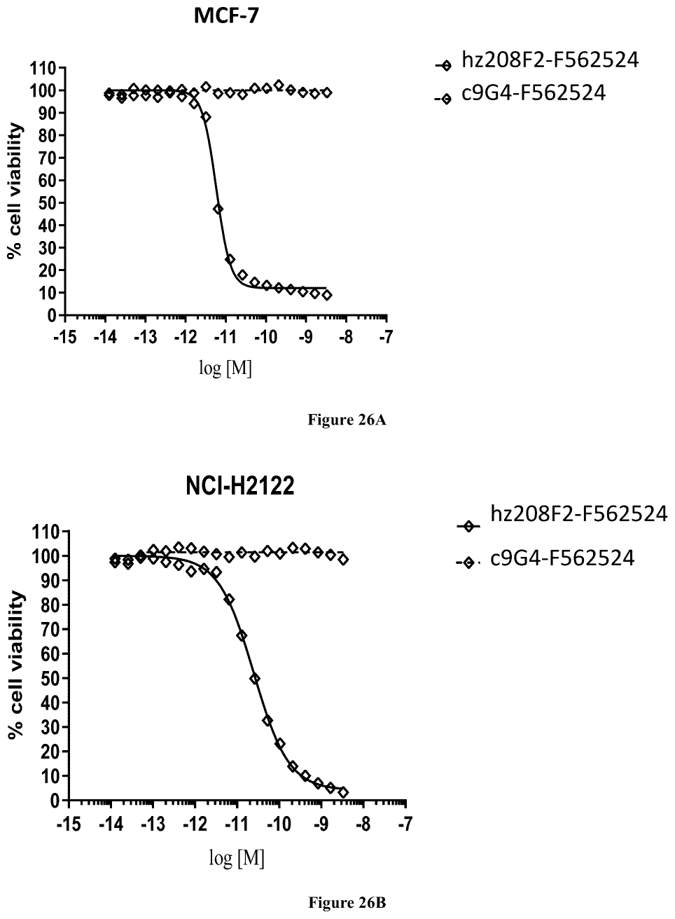

ADCs were analyzed by native liquid chromatography-mass spectrometry on a UPLC Acquity H Class Bio system coupled to a Synapt G2Si mass spectrometer (Waters). LC separation was performed on 2 Polyhydroxyethyl A columns (Poly-LC, 150×1 mm, 300 A, 5 μm) connected in series. Samples were diluted to 0.2 mg/ml with the eluant buffer (150 mM ammonium acetate). Four μg of sample were injected and eluted at a flow rate of 40 μL/min. The mass spectrometer was operated in positive mode with a capillary voltage of 2.9 kV. The sample cone was set at 150 V. Analyses were performed in the range of m/z 1000-8000 with a 1 sec scan time. FIG. 24 shows the m/z spectra before deconvolution. The DAR distribution was determined after deconvolution of MS spectra using MaxEnt™ algorithm from Mass Lynx software (Waters) (FIG. 25). Average DAR values were calculated by using the following equation (where j is the maximum number of drug load):

$$DAR = \frac{( {\sum }_{i = 0}^{j} i * intensity Di )}{{\sum }_{i = 0}^{j} intensity Di}$$

The results are presented in the Table 14 below.

TABLE 14
ADC analysis by native LC-MS analysis: DAR distribution and average DAR

| | DAR distribution (%) | | | | | | | | | Average |
|---|---|---|---|---|---|---|---|---|---|---|
| ADC | DAR0 | DAR1 | DAR2 | DAR3 | DAR4 | DAR5 | DAR6 | DAR7 | DAR8 | DAR |
| hz208F2- | 0 | 0 | 0 | 11 | 68 | 21 | 0 | 0 | 0 | 4.1 |
| F562524 | | | | | | | | | | |
| hz208F2- | 0 | 0 | 0 | 0 | 79 | 21 | 0 | 0 | 0 | 4.2 |
| F562646 | | | | | | | | | | |
| c9G4- | 0 | 0 | 0 | 11 | 67 | 23 | 0 | 0 | 0 | 4.1 |
| F562524 | | | | | | | | | | |
| c9G4- | 0 | 0 | 0 | 0 | 82 | 18 | 0 | 0 | 0 | 4.2 |
| F562646 | | | | | | | | | | |

### 5.3. In Vitro Stability

The ADC hz208F2-F562524 was incubated at 200 μg/ml in cynomolgus serum at 37° C. for a period of 14 days. Samples were collected at day 0, 3, 7 and 14, and stored at −80° C. until LC-MS analysis to determine the average DAR.
Before LC-MS analysis, the samples were immunopurified by using Streptavidin magnetic beads (M-280, Invitrogen) coated with a Capture Select anti-human IgG-Biotin conjugate (Life Technologies, 8 μg antibody/200 μL beads). Samples were incubated with the anti-IgG-coated beads for 2 h at room temperature (100 μL sample/200 μL beads) before acidic elution with 40 μL of 0.4% trifluoroacetic acid. The pH was increased by adding 4 μL of a 3 M Tris/HCL pH 8.8 solution. The immunopurified samples were incubated with 2 μL of IgGZero for 30 minutes at 37° C. before LC-MS analysis in native conditions as described above. The DAR distribution was determined after deconvolution of MS spectra using MaxEnt™ algorithm from Mass Lynx software (Waters), and average DAR values were calculated by using the following equation (where j is the maximum number of drug load):

$$DAR = \frac{( {\sum }_{i = 0}^{j} i * intensity Di )}{{\sum }_{i = 0}^{j} intensity Di}$$

The results are presented in the Table 15 below. The ADC hz208F2-F562524 was shown to be highly stable up to 14 days after in vitro incubation in cynomolgus serum.

TABLE 15
In vitro stability study of hz208F2-F562524: DAR distribution and average DAR

| | DAR distribution (%) | | | | | | | | | Average |
|---|---|---|---|---|---|---|---|---|---|---|
| Day | DAR0 | DAR1 | DAR2 | DAR3 | DAR4 | DAR5 | DAR6 | DAR7 | DAR8 | DAR |
| 0 | 0 | 0 | 0 | 18 | 57 | 25 | 0 | 0 | 0 | 4.1 |
| 3 | 0 | 0 | 0 | 19 | 54 | 27 | 0 | 0 | 0 | 4.1 |
| 7 | 0 | 0 | 0 | 18 | 50 | 32 | 0 | 0 | 0 | 4.1 |
| 10 | 0 | 0 | 0 | 19 | 54 | 28 | 0 | 0 | 0 | 4.1 |

### 5.4. In Vitro Cytotoxicity

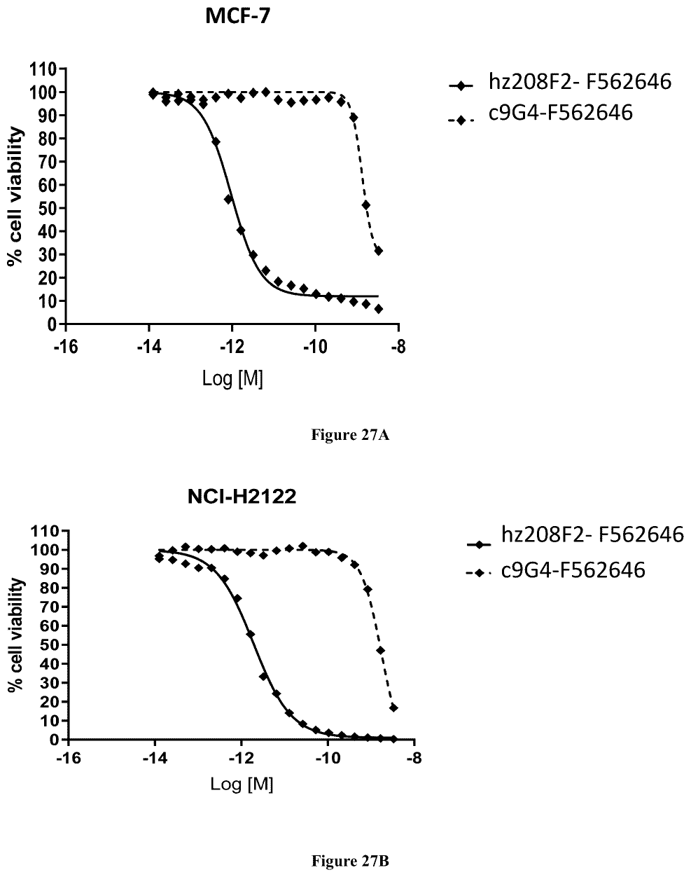

The cytotoxicity of the ADCs was evaluated in MCF-7 and NCI-H2122 cells. Cells were plated on 96 well plates (2500 cells per well) in complete growth media. The day after, serial dilutions of the tested ADCs were added to the corresponding wells and incubated at 37° C. for 6 days. Cell viability was determined by measuring ATP using the cell Titer Glo kit (Promega). Luminescence was read using the plate reader Mithras from Berthold Company. The results obtained, expressed in percentage of viability, are shown in FIGS. 26 and 27. The viability in the non-treated wells was considered as 100%.
As expected, the ADCs hz208F2-F562524 and hz208F2-F562646 decreased dramatically cell viability. EC₅₀ values of 2.48.10⁻¹¹ and 1.92.10⁻¹² M were determined on NCI-H2122 cells for hz208F2-F562524 and hz208F2-F562646, respectively, and EC₅₀ values of 5.86.10⁻¹² and 9.45.10⁻¹³ M were obtained on MCF-7 cells for hz208F2-F562524 and hz208F2-F562646, respectively, indicating potent cytotoxic activity. On the opposite, the corresponding ADCs synthesized with the irrelevant antibody showed a modest cytotoxic activity on both MCF-7 and NCI-H2122 cells.

### 5.5. In Vivo Anti-Tumoral Activity

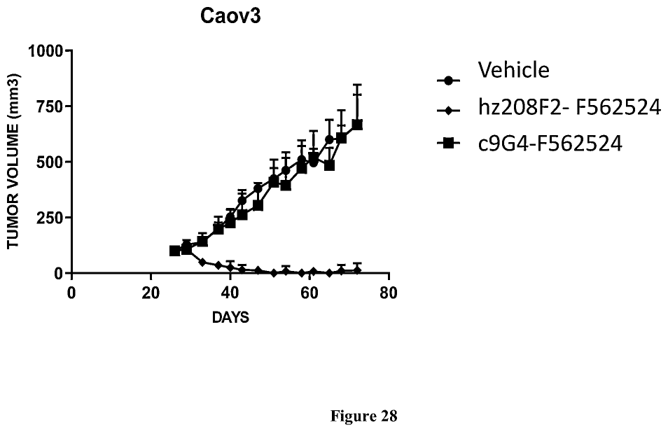

Seven weeks old female SCID mice (Charles River Laboratories) were engrafted subcutaneously with 10.106 Caov3 cells (6 animals per groups). Treatment by intravenous administration (Q7d2) of the ADC hz208F2-F562524 (0.3 mg/kg), the corresponding control ADC c9G4-F562524 (0.3 mg/kg), or the vehicle was initiated when tumors reached approximately 150 mm3. Tumor volume (Length×Width×Height×0.52) was measured by electronic caliper twice weekly during approximatively 50 days after the first injection. The results are presented on FIG. 28. A complete tumor regression can be observed after 2 injections of the ADC hz208F2-F562524 whereas no anti-tumoral effect was observed with the control ADC or the vehicle.

### 5.6. Conclusion

The ADCs synthesized with PNU-159682 derivatives by using the sulfomaleimide-linker technology are highly homogeneous and stable in serum. Their efficacy was demonstrated in different in vitro and in vivo models.

### 6. Overall Conclusions

Overall, the sulfomaleimide-based linker technology described in this invention give a better stability in the plasma of different species and a better efficacy in in vitro models compared to usual maleimide-based linkers used for compounds in the market such as Adcetris. These properties have translated in a clear improvement of in vivo efficacy in different cell lines and more notably for cell lines with a lower expression of the antigen (CAOV3).
A better tolerability is also expected since the ADCs according to the invention are more stable in the circulation associated with an improved efficacy and safety margin in human treatment.

1. A linker of the following formula (I): 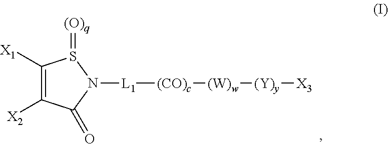
or a salt thereof,
wherein:
X₁ and X₂ represent, independently of each other, H, a halogen atom, a (C₁-C₆) alkoxy, an optionally substituted aryloxy, or —O—(CH₂CH₂O)ᵣH, provided that X₁ and X₂ do not represent H at the same time;
L₁ represents a group of formula L₁′-(CO—Z′)_{z}; with L₁′ being-(CH₂)ₙ—, —(CH₂CH₂O)ₘ—CH₂—CH₂—, arylene, heteroarylene, cycloalkanediyl, —(CH₂)ₙ-arylene-, —(CH₂)ₙ-heteroarylene-, —(CH₂)ₙ-cycloalkanediyl-, -arylene-(CH₂)ₚ—, -heteroarylene-(CH₂)ₚ—, -cycloalkanediyl-(CH₂)ₚ—, —(CH₂)ₙ-arylene-(CH₂)ₚ—, —(CH₂)ₙ-heteroarylene-(CH₂)ₚ—, —(CH₂)ₙ-cycloalkanediyl-(CH₂)ₚ—, —(CH₂CH₂O)ₘ—CH₂—CH₂-arylene-(CH₂)ₚ—, —(CH₂CH₂O)ₘ—CH₂—CH₂-heteroarylene-(CH₂)ₚ—, —(CH₂CH₂O)ₘ—CH₂—CH₂-cycloalkanediyl-(CH₂)ₚ—, —(CH₂)ₙ-arylene-CH₂—CH₂—(OCH₂CH₂)ₘ—, —(CH₂)ₙ-heteroarylene-CH₂—CH₂—(OCH₂CH₂)ₘ—, or —(CH₂)ₙ-cycloalkanediyl-CH₂—CH₂—(OCH₂CH₂)ₘ—;
each W independently represents an amino acid unit; 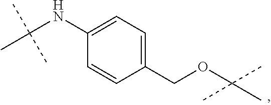
Y is PAB—CO—(Z)_{z}—, with PAB being the oxygen of the PAB unit being linked to CO—(Z)_{z};
Z is —NR₄—(CH₂)ᵤ—NR₅—, —NR₄—(CH₂)ᵤ—NR₅—CO—, —NR₄—(CH₂)ᵤ—NR₅—CO—(CH₂)ᵥ—, or —NR₄—(CH₂)ᵤ—NR₅—CO—(CH₂)ᵥ—CO—, the NR₄ group being linked to the CO group of PAB—CO;
Z′ is-NR₄—(CH₂)ᵤ—NR₅- or —NR₄—(CH₂)ᵤ—NR₅—CO—(CH₂)ᵥ—, the NR₄ group being linked to the CO group of CO—Z′;
R₄ and R₅ are independently H or a (C₁-C₆)alkyl group;
c is 0 or 1;
m is an integer from 1 to 15;
n is an integer from 1 to 6;
p is an integer from 1 to 6;
q is 0, 1 or 2;
r is an integer from 1 to 24;
u is an integer from 1 to 6;
v is an integer from 1 to 6;
w is an integer from 0 to 5;
y is 0 or 1;
z is 0 or 1;
z′ is 0 or 1; and
X₃ represents H when y=z=1 and Z is —NR₄—(CH₂)ᵤ—NR₅- or when c=w=y=0, z′=1 and Z′ is-NR₄—(CH₂)ᵤ—NR₅- and in the other cases, X₃ represents OH, NH₂ or a leaving group, wherein the leaving group is a halogen atom, a sulfonate of formula —OSO₂—R_{LG}, N-succinimidyloxy, 4-nitro-phenyloxy, pentafluorophenoxy or N-benzotriazoloxy, R_{LG} representing a (C₁-C₆)alkyl, aryl, aryl-(C₁-C₆)alkyl or (C₁-C₆)alkyl-aryl group, the said group being optionally substituted with one or several halogen atoms such as fluorine atoms,
with the proviso that it is not a compound of formula (I) for which:
X₁ is Cl, X₂ is H, q is 0, L₁ is 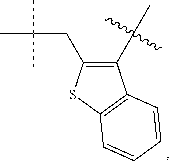
c is 0, w is 0, y is 0 and X₃ is Cl;
X₁ is Cl, X₂ is Cl, q is 0, L₁ is 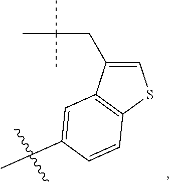
c is 0, w is 0, y is 0 and X₃ is Cl;
X₁ is Cl, X₂ is H, q is 0, L₁ is 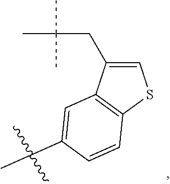
c is 0, w is 0, y is 0 and X₃ is Cl;
X₁ is Cl, X₂ is H, q is 0, L₁ is 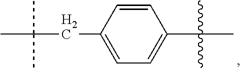
c is 0, w is 0, y is 0 and X₃ is Cl;
X₁ is Cl, X₂ is H, q is 0, L₁ is 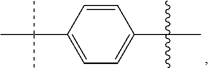
c is 0, w is 0, y is 0 and X₃ is Cl;
X₁ is Cl, X₂ is H, q is 0, L₁ is 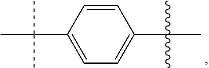
c is 0, w is 0, y is 0 and X₃ is Br;
X₁ is Cl, X₂ is H, q is 0, L₁ is 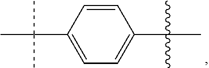
c is 0, w is 0, y is 0 and X₃ is I;
X₁ is H, X₂ is Cl, q is 0, L₁ is 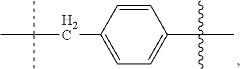
c is 0, w is 0, y is 0 and X₃ is Cl;
X₁ is H, X₂ is Br, q is 0, L₁ is 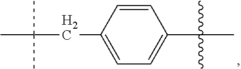
c is 0, w is 0, y is 0 and X₃ is Cl;
X₁ is H, X₂ is Br, q is 0, L₁ is 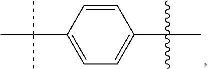
c is 0, w is 0, y is 0 and X₃ is Cl;
X₁ is Cl, X₂ is Cl, q is 0, L₁ is 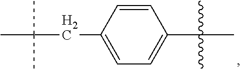
c is 0, w is 0, y is 0 and X₃ is Cl;
X₁ is Cl, X₂ is Cl, q is 0, L₁ is 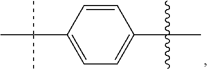
c is 0, w is 0, y is 0 and X₃ is Cl;
X₁ is Cl, X₂ is Cl, q is 0, L₁ is 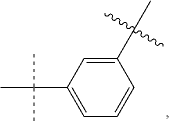
c is 0, w is 0, y is 0 and X₃ is Cl;
X₁ is Cl, X₂ is Br, q is 0, L₁ is 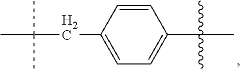
c is 0, w is 0, y is 0 and X₃ is Cl;
X₁ is Cl, X₂ is Br, q is 0, L₁ is 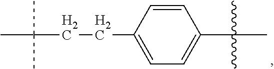
c is 0, w is 0, y is 0 and X₃ is Cl;
X₁ is Cl, X₂ is Br, q is 0, L₁ is 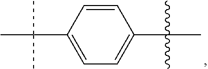
c is 0, w is 0, y is 0 and X₃ is Cl; or
X₁ is Br, X₂ is Br, q is 0, L₁ is 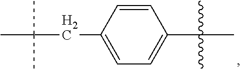
c is 0, w is 0, y is 0 and X₃ is Cl;
wherein the dashed line indicates the point of attachment of L₁ to the nitrogen atom of 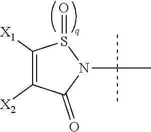
and the wavy line indicates the point of attachment of L₁ to X₃.

2. The linker according to claim 1, wherein it has the following formula (Ia): 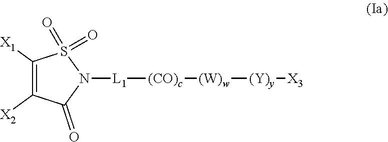
or a salt thereof,
wherein:
y is 0 when w is 0 and y is 0 or 1 when w is an integer from 1 to 5.

3. The linker according to claim 2, wherein at least X₁ or X₂ represents a halogen atom.

4. The linker according to claim 3, wherein at least X₁ or X₂ represents Br or Cl.

5. The linker according to claim 4, wherein one of X₁ and X₂ represents Br or Cl and the other group represents H, Cl or Br.

6. The linker according to claim 1, wherein L₁′ represents-(CH₂)ₙ—, —(CH₂CH₂O)ₘ—CH₂—CH₂—, arylene, -cycloalkanediyl-, —(CH₂)ₙ-arylene-, -arylene-(CH₂)ₙ—, —(CH₂)ₙ-cycloalkanediyl-, -cycloalkanediyl-(CH₂)ₙ—,

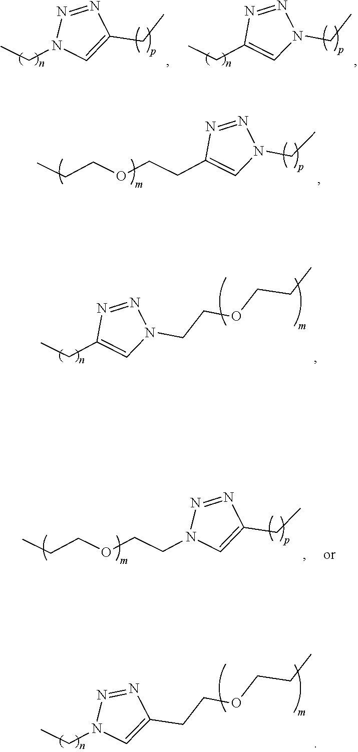

7. The linker according to claim 6, wherein L₁′ is —(CH₂)ₙ— or —(CH₂CH₂O)ₘ—CH₂—CH₂—.

8. The linker according to claim 1, wherein each W is selected from alanine, valine, leucine, isoleucine, methionine, phenylalanine, tryptophan, proline, lysine, lysine protected with acetyl or formyl, arginine, arginine protected with tosyl or nitro group(s), histidine, ornithine, ornithine protected with acetyl or formyl, and citrulline.

9. The linker according to claim 1, wherein:
w=0 and (W) w is a bond, or
w=2 and (W) w is Val-Cit or Val-Ala.

10. The linker according to claim 1, wherein:
X₁ and X₂ are identical and are selected from Cl, Br, (C₁-C₆)alkoxy and an aryloxy optionally substituted with one or several groups selected from halogen, CN, NO₂ and an aryloxy optionally substituted with one or several halogen atoms, or
one of X₁ and X₂ is H and the other is selected from Cl, Br, (C₁-C₆)alkoxy and an aryloxy optionally substituted with one or several groups selected from halogen, CN, NO₂ and an aryloxy optionally substituted with one or several halogen atoms.

11. The linker according to claim 1, wherein X₃ is H when y=z=1 and Z is-NR₄—(CH₂)ᵤ—NR₅- or when c=w=y=0, z′=1 and Z′ is-NR₄—(CH₂)ᵤ—NR₅- and in the other cases, X₃ is OH, Cl or N-succinimidyloxy.

12. A binding unit-drug conjugate of the following formula (III) or (IV): 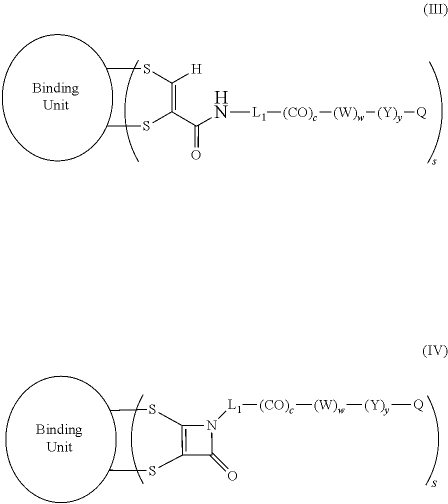
or a salt thereof,
wherein:
the binding unit is a peptide, a protein, an antibody, or an antigen binding fragment thereof;
L₁ represents a group of formula L₁′-(CO—Z′)_{z}; with L₁′ being-(CH₂)ₙ—, —(CH₂CH₂O)ₘ—CH₂—CH₂—, arylene, heteroarylene, cycloalkanediyl, —(CH₂)ₙ-arylene-, —(CH₂)ₙ-heteroarylene-, —(CH₂)ₙ-cycloalkanediyl-, -arylene-(CH₂)ₚ—, -heteroarylene-(CH₂)ₚ—, -cycloalkanediyl-(CH₂)ₚ—, —(CH₂)ₙ-arylene-(CH₂)ₚ—, —(CH₂)ₙ-heteroarylene-(CH₂)ₚ—, —(CH₂)ₙ-cycloalkanediyl-(CH₂)ₚ—, —(CH₂CH₂O)ₘ—CH₂—CH₂-arylene-(CH₂)ₚ—, —(CH₂CH₂O)ₘ—CH₂—CH₂-heteroarylene-(CH₂)ₚ—, —(CH₂CH₂O)ₘ—CH₂—CH₂—, cycloalkanediyl-(CH₂)ₚ—, —(CH₂)ₙ-arylene-CH₂—CH₂—(CH₂CH₂O)ₘ—, —(CH₂)ₙ-heteroarylene-CH₂—CH₂—(CH₂CH₂O)ₘ—, or —(CH₂)ₙ-cycloalkanediyl-CH₂—CH₂—(CH₂CH₂O)ₘ—;
each W independently represents an amino acid unit;
Y is PAB—CO—(Z)_{z}—, with PAB being 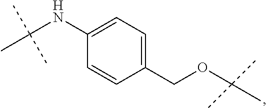
the oxygen of the PAB unit being linked to CO—(Z)_{z};
Z is-NR₄—(CH₂)ᵤ—NR₅—, —NR₄—(CH₂)ᵤ—NR₅—CO—, —NR₄—(CH₂)ᵤ—NR₅—CO—(CH₂)ᵥ—, or —NR₄—(CH₂)ᵤ—NR₅—CO—(CH₂)ᵥ—CO—, the NR₄ group being linked to the CO group of PAB—CO;
Z′ is-NR₄—(CH₂)ᵤ—NR₅- or —NR₄—(CH₂)ᵤ—NR₅—CO—(CH₂)ᵥ—, the NR₄ group being linked to the CO group of CO—Z′;
R₄ and R₅ are independently H or a (C₁-C₆)alkyl group;
Q represents a drug moiety;
c is 0 or 1;
m is an integer from 1 to 15;
n is an integer from 1 to 6;
p is an integer from 1 to 6;
s is an integer from 1 to 8;
u is an integer from 1 to 6;
v is an integer from 1 to 6;
w is an integer from 0 to 5;
y is 0 or 1;
z is 0 or 1; and
z′ is 0 or 1.

13. The binding unit-drug conjugate according to claim 12, wherein the binding unit is an antibody, or an antigen binding fragment thereof.

14. The binding unit-drug conjugate according to claim 13, wherein the antibody is an IGF-1R antibody or a HER2 antibody.

15. A pharmaceutical composition comprising a binding unit-drug conjugate according to claim 12 and at least one pharmaceutically acceptable excipient.

16. A method for covalently linking a drug to a binding unit by a linker according to claim 1,
wherein the binding unit is selected from a peptide, a protein, an antibody and an antigen binding fragment thereof.

17. The method according to claim 16, wherein q is 2.

18. A method for treating cancer comprising the administration to a person in need thereof of an effective amount of a binding unit-drug conjugate according to claim 12.

19. The method according to claim 12, wherein the cancer is a prostate cancer, an osteosarcoma, a lung cancer, a breast cancer, an endometrial cancer, a glioblastoma, a colon cancer, a gastric cancer, a renal cancer, a pancreas cancer, or a head and neck cancer.

20. A method for treating cancer comprising the administration to a person in need thereof of an effective amount of a pharmaceutical composition according to claim 15.
